(19) United States
(12) Patent Application Publication
(10) Pub. No.: US 2024/0408190 A1
(43) Pub. Date: Dec. 12, 2024

(54) INFLUENZA VACCINES

(71) Applicants: DIOSynVax Ltd, Hertford Hertfordshire (GB); The Chancellor, Masters and Scholars of the University of Cambridge, Cambridge Cambridgeshire (GB); Universitaet Regensburg, Regensburg (DE)

(72) Inventors: Jonathan Luke HEENEY, Cambridge Cambridgeshire (GB); Sneha VISHWANATH, Cambridge Cambridgeshire (GB); George CARNELL, Cambridge Cambridgeshire (GB); David WELLS, Hertford Hertfordshire (GB); Simon FROST, Cambridge Cambridgeshire (GB); Matteo FERRARI, Hertford Hertfordshire (GB); Benedikt ASBACH, Regensburg (DE); Ralf WAGNER, Regensburg (DE)

(21) Appl. No.: 18/699,032

(22) PCT Filed: Oct. 6, 2022
(86) PCT No.: PCT/GB2022/052534

§ 371 Date: Apr. 5, 2024

(30) Foreign Application Priority Data
Oct. 6, 2021 (GB) 2114328.4
May 31, 2022 (GB) 2208070.9
Sep. 23, 2022 (GB) 2213958.8

(51) Int. Cl. A61K 39/145; A61K 39/00; A61P 31/16; C07K 14/005

(52) U.S. Cl. CPC A61K 39/145; A61P 31/16; C07K 14/005; A61K 2039/53; A61K 2039/575; C12N 2760/16122; C12N 2760/16134

(57) ABSTRACT

Polypeptides, nucleic acid molecules, vectors, cells, fusion proteins, pharmaceutical compositions, combined preparations, and their use as vaccines against influenza are described. The polypeptides comprise a haemagluttinin subtype 5 (H5) globular head domain, and optionally a haemagluttinin stem domain, wherein the polypeptide comprises an amino acid sequence in which an amino acid residue at a position corresponding to residue position 144 or 145 of a wild-type H5 globular head domain has been deleted, or wherein the polypeptide comprises an amino acid sequence with an E amino acid residue at a position corresponding to residue position 238 of a wild-type H5 globular head domain.

This invention relates to nucleic acid molecules, polypeptides, vectors, cells, fusion proteins, pharmaceutical compositions, combined preparations, and their use as vaccines against influenza.

Influenza is a highly contagious respiratory illness caused by the influenza virus infecting the epithelial cells within the upper respiratory tract. The infection is characterised by a sudden onset of high fever, headache, muscle ache and fatigue, sore throat, cough and rhinitis. For the majority of cases, influenza rarely lasts for over a week and is usually restricted to the upper respiratory tract. However, in medically vulnerable people, such as people over 65 years old and people with certain chronic medical conditions, influenza can cause complications and even result in death. There are around 9 million-45 million human infections. WHO estimates that seasonal influenza may result in 290 000-650 000 deaths each year due to respiratory diseases alone. Thus, the development of an effective flu vaccine is critical to the health of millions of people around the world.

The fundamental principal of a vaccine is to prepare the immune system for an encounter with a pathogen. A vaccine triggers the immune system to produce antibodies and T-cell responses, which helps to combat infection. Historically, once a pathogen was isolated and grown, it was either mass produced and killed or attenuated, and used as a vaccine. Later recombinant genes from isolated pathogens were used to generate recombinant proteins that were mixed with adjuvants to stimulate immune responses. More recently the pathogen genes were cloned into vector systems (attenuated bacteria or viral delivery systems) to express and deliver the antigen in vivo. All of these strategies are dependent on pathogens isolated from past outbreaks to prevent future ones. For pathogens which do not change significantly, or slowly, this conventional technology is effective. However, some pathogens, are prone to accelerated mutation rate and previously generated antibodies do not always recognise evolved strains of the same pathogen. New emerging and re-emerging pathogens often hide or disguise their vulnerable antigens from the immune system to escape the immune response.

Influenza is one of the best characterised re-emerging pathogens, and re-emerges each season infecting up to 100 million people worldwide. Influenza is a member of the Orthomyxoviridae family and has a single-stranded negative sense RNA genome. RNA viruses generally have very high mutation rates compared to DNA viruses, because viral RNA polymerases lack the proofreading ability of DNA polymerases. This contributes towards antigenic drift, a continuous process of the accumulation of mutations in the genome of an infectious agent resulting in minor changes in antigens presented to the immune system of the host organism. Changes to antigenic regions of the proteins on the influenza virion result in its evasion of the host immune system and potentially increased pathogenicity and infectiousness. This is one reason why it is difficult to make effective vaccines to prevent influenza. Influenza can undergo antigenic shift, a process wherein there is a dramatic change in the antigens presented on the influenza virus. Gene segments from different subtypes of influenza can reassort and package into a new virion particle containing the genetic information from both of the subtypes. This can result in a virus that has antigenic characteristics not before seen in a human setting, to which we are naïve immunologically. The new quasispecies of the virus can cause a pandemic if no neutralising, or inhibitory antibodies to the new influenza virus are present in the human population.

There are multiple types of influenza viruses, the most common in humans being influenza A, influenza B, and influenza C. Influenza A viruses infect a wide variety of birds and mammals, including humans, horses, marine mammals, pigs, ferrets, and chickens. In their natural reservoirs in aquatic birds and bats, influenza A viruses show minimal evolution and cause unapparent disease; but once they transfer to a different species, influenza A viruses can evolve rapidly as they adapt to the new host, possibly causing pandemics or epidemics of acute respiratory disease in domestic poultry, lower animals and humans. In animals, most influenza A viruses cause mild localized infections of the respiratory and intestinal tract. However, highly pathogenic influenza A strains, such as some within the H5N1 subtype, can cause systemic infections in poultry with spill-over human cases, which can have high mortality rates. Influenza B and C are restricted to infecting humans, with no known animal reservoirs. Influenza B causes epidemic seasonal infections, with similar pathogenicity as influenza A. Influenza C viruses are usually associated with very mild or asymptomatic infections in humans.

At just over 100 years since the devastating 1918 influenza pandemic, there is still no optimal preventative or treatment against influenza A and B. Although they share some degree of similarity with antigen presentation on their surface, the highly heterologous nature of these antigens presents significant challenges in developing vaccines and treatments. During the 2019-2020 seasonal flu epidemic, quadrivalent vaccines were widely distributed. These gave protection against two influenza A viruses and two influenza B viruses. However, to prevent a potential outbreak of influenza in which the virus has rapidly evolved and hence unrecognisable by the host immune system, it is crucial that an influenza vaccine protects against many if not all potential influenza strains.

Influenza A has an outer envelope that is studded with three integral membrane proteins: hemagglutinin (HA); neuraminidase (NA); and matrix ion channel (M2), which overlay a matrix protein (M1). The organisation of influenza B is similar, with HA and NA scattered across the lipid envelope, but with NB and BM2 transmembrane ion channels instead of M2.

Influenza A viruses are subtyped based on their combination of surface glycoproteins (GPs) namely HA and NA. Influenza B viruses, having much less antigenic variation than influenza A, are not. HA and NA are membrane bound envelope GPs, responsible for virus attachment, penetration of the viral particles into the cell, and release of the viral particle from the cell. They are the sources of the major immunodominant epitopes for virus neutralisation and protective immunity. Hence, both HA and NA proteins are considered the most important components for prophylactic influenza vaccines. During HA-mediated entry, binding of the GP to sialic acid-containing receptors on the host cell membrane initiates endocytosis of the virion into the cell. The low pH within the endosome induces a conformational change in HA to expose a hydrophobic region, termed the fusion peptide. The newly exposed fusion peptide then inserts into the endosomal membrane, thereby bringing the viral and endosomal membranes in close contact to allow membrane fusion and entry of the virus into the cytoplasm. This release into the cytoplasm allows viral proteins and RNA molecules to enter the nucleus for viral transcription and subsequent replication. Transcribed, positive sense mRNAs are exported from the nucleus to be translated into viral proteins, and replicated negative sense RNA is exported from the nucleus to re-assemble with the newly synthesised viral proteins to form a progeny virus particle. The virus buds from the apical cell membrane, taking with it host membrane to form a virion capable of infecting another cell.

HA exists as a homo-trimer on the virus surface, forming a cylinder-shaped molecule which projects externally from the virion and forms a type I transmembrane glycoprotein. Each monomer of the HA molecule consists of a single HA0 polypeptide chain with HA1 and HA2 regions linked by two disulphide bridges. Each HA0 polypeptide forms a globular head domain and a stem domain. The globular head domain comprises the most dominant epitopes, while the stem domain has less dominant, but important epitopes for broader antibody recognition. The amino acid sequence of these epitopes determines the binding affinity and specificity towards antibodies. The globular head domain consists of a part of HA1, including a receptor binding domain and an esterase domain, whereas the stem domain consists of parts of HA1 and HA2. Amino acid residues of HA1 that form the globular head domain fold into a motif of eight stranded antiparallel β-sheets which sits in a shallow pocket at the distal tip acting as the receptor binding site which is surrounded by antigenic sites. The remaining parts of the HA1 domain run down to the stem domain mainly comprising β-sheets. HA2 forms the majority of the stem domain and is folded into a helical coiled-coil structure forming the stem backbone. HA2 also contains the hydrophobic region required for membrane fusion, and a long helical chain anchored to the surface membrane and a short cytosolic tail.

There are 18 different HA subtypes and 11 different NA subtypes within influenza A. Theoretically, there are potentially 198 different influenza A subtype combinations, some of which may be virulent in humans and other animals. As a result, there is significant concern that viruses from these subtypes could reassort with human transmissible viruses and initiate the next pandemic. In recent years, avian viruses of the H5, H7, H9, and H10 subtypes have caused zoonotic infections with H5 and H7 viruses often causing severe disease. The highly pathogenic Asian influenza (HPAI) outbreak of H5N1 of 1997 resulted in the killing of the entire domestic poultry population within Hong Kong. This panzootic also resulted in 860 confirmed infections and 454 fatalities in humans, demonstrating the ability of the avian-derived virus to transmit to humans and result in a high mortality rate. This HPAI of the H5N1 subtype frequently re-emerges and is of particular concern because of its 60% mortality rate, and because it continues to evolve and diversify. The last influenza pandemic, in 2009, was caused by a novel H1N1 influenza A virus, generated by circulating human influenza reassorting with human, porcine, and avian influenza. The virus was very different from H1N1 viruses that were circulating at the time of the pandemic. As a result, very few young people had any existing immunity to the virus, and around a third of people over the age of 60 had antibodies against the virus from past exposure of similar H1N1 viruses. The CDC (Centre for Disease Control and Prevention) estimate that the total number of deaths worldwide caused by the 2009 outbreak is ranged between 150,000 to 575,400. Influenza A is constantly evolving in multiple species and to prepare for this, virus characterization and translation into effective vaccines must be done in a timely manner.

Although they have less antigenic variation than influenza A viruses, influenza B viruses have recently emerged into two antigenically distinct lineages (B/Victoria/2/1987-like and B/Yamagata/16/1988-like), illustrating the fluidity with which influenza B can evolve, and how it is also now imperative to include viruses of both type A and B in seasonal flu vaccinations.

There is a need to provide improved influenza vaccines that protect against far more influenza strains than current vaccines. In particular, there is a need to provide vaccines against influenza A and B viruses that protect against several influenza A and B variants. In particular, there is a need to provide improved vaccines that elicit more broadly neutralising immune responses to influenza A H5 viruses. There is also a need to provide neutralising antibody protection against the H1N1 subtype of influenza A.

In particular, new vaccine strategies are needed to 1) successfully combat vaccine escape, and, 2) prevent the emergence and spread of new influenza pathogens in the human population. Envisioned herein is the use of large databases of different influenza virus sequences from not only humans, but also animals which are the source of new influenza virus re-assortments which give rise to new human pathogens.

### Influenza A H5 Viruses

While most viral genes have been replaced through reassortment yielding many different genotypes, the specific H5 gene has remained present in all influenza A isolates identified since its discovery in 1996. Thus, H5 provides a constant to which the evolving strains of influenza A may be effectively compared. A clade nomenclature system for H5 HA was developed to compare the evolutionary pattern of this gene. Circulating H5N1 viruses are grouped into numerous virus clades based on the characterisation and sequence homology of the HA gene. Clades will have a single common ancestor from which particular genetic changes have arisen. As the viruses within these clades continue to evolve, sub-lineages periodically emerge. Vaccines against influenza A H5 exist, however either these vaccines are unable to induce a neutralising immune response against the important H5 clades, or the affinity of the antigen to its neutralising antibody is sub-optimal. The computationally optimised broadly reactive antigen (COBRA) Tier 2 vaccine design (Nunez et al, Vaccines, 2020, 38(4):830-839) is developed by consensus sequence alignment techniques using full-length sequences from H5N1 clade 2 infections isolated from both humans and birds. However, this design did not produce haemagluttinin inhibition (HAI) antibodies or protection against newer reassorted viruses across all H5N1 clades and sub-clades that were tested against the vaccine. The risk of human infection with avian influenza A(H5Nx), particularly from clade 2.3.4.4, is on the rise due to increasing human and avian contact and poor biosafety practices.

There is also a need, therefore, to provide improved vaccines that elicit more broadly neutralising immune responses to influenza A H5 viruses. In particular, there is a need to provide vaccines that elicit antibody responses that effectively neutralise influenza A clade 2.3.4.4.

The Applicant has identified amino acid sequences and their encoding nucleic acid molecules that induce a broadly neutralising immune response against important H5 clades of influenza A, including clade 2.3.4.4. The Applicant has further identified amino acid sequences and their encoding nucleic acid molecules responsible for stabilising the stem region of the H5 molecule both in the pre-fusion and post-fusion state.

H5 embodiments of the invention are described below.

According to the invention there is provided an isolated polypeptide comprising a haemagluttinin subtype 5 (H5) globular head domain, and optionally a haemagluttinin stem domain, with the following amino acid residues at positions 156, 157, 171, 172, and 205 of the head domain:
156: R;
157: P or S, preferably P;
171: D or N;
172: T or A, preferably T; and
205: K or R, preferably K

The applicant has found that such polypeptides elicit broadly neutralising antibody responses to a diverse panel of H5 influenza viruses, including viruses of several different clades.

Optionally a polypeptide of the invention comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:7, 8, 10, 11, 1, or 3.

Optionally a polypeptide of the invention comprises the following amino acid residues at positions 156, 157, 171, 172, and 205 of the head domain:
156: R;
157: P;
171: D;
172: T; and
205: K

Optionally a polypeptide of the invention comprises an amino acid sequence of SEQ ID NO:7 or 8, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:7 or 8 and which has the following amino acid residues at positions corresponding to positions 156, 157, 171, 172, and 205 of SEQ ID NO:7 or 8:
156: R;
157: P;
171: D;
172: T; and
205: K

Optionally a polypeptide of the invention comprises an amino acid sequence of SEQ ID NO:7 (FLU_T3_HA_1) (see Example 4 below).

Such polypeptides are particularly advantageous as they elicit broadly neutralising antibody responses to a diverse panel of H5 influenza viruses, including H5 influenza viruses of clades 2.3.4 and 7.1 arising from the Goose Guangdong (A/Goose/Guangdong/1/1996, GS/GD) lineage, which are currently in circulation in birds and humans.

Optionally a polypeptide of the invention comprises the following amino acid residues at positions 156, 157, 171, 172, and 205 of the head domain:
156: R;
157: P;
171: N;
172: T; and
205: K

Optionally a polypeptide of the invention comprises an amino acid sequence of SEQ ID NO:10 or 11, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:10 or 11 and which has the following amino acid residues at positions corresponding to positions 156, 157, 171, 172, and 205 of SEQ ID NO:10 or 11:
156: R;
157: P;
171: N;
172: T; and
205: K

Optionally a polypeptide of the invention comprises an amino acid sequence of SEQ ID NO:10 (FLU_T3_HA_2) (see Example 5 below).

Such polypeptides are particularly advantageous as they elicit broadly neutralising antibody responses to a diverse panel of H5 influenza viruses, including H5 influenza viruses of GS/GD clades 2.3.4 and 7.1, which are currently in circulation in birds.

Optionally a polypeptide of the invention comprises the following amino acid residues at positions 156, 157, 171, 172, and 205 of the head domain:
156: R;
157: S;
171: N;
172: A; and
205: R

Optionally a polypeptide of the invention comprises an amino acid sequence of SEQ ID NO:1 or 3, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:1 or 3 and which has the following amino acid residues at positions corresponding to positions 156, 157, 171, 172, and 205 of SEQ ID NO:1 or 3:
156: R;
157: S;
171: N;
172: A; and
205: R

Optionally a polypeptide of the invention comprises an amino acid sequence of SEQ ID NO:1 (FLU_T2_HA_1) (see Example 1 below).

Such polypeptides are particularly advantageous as they elicit broadly neutralising antibody responses to a diverse panel of H5 influenza viruses, including viruses of several different GS/GD clades.

Table 1 below summarises differences in amino acid sequence at positions A-E of the influenza haemagluttinin H5 for different embodiments of the invention, and differences at those positions compared with prior art COBRA sequences.

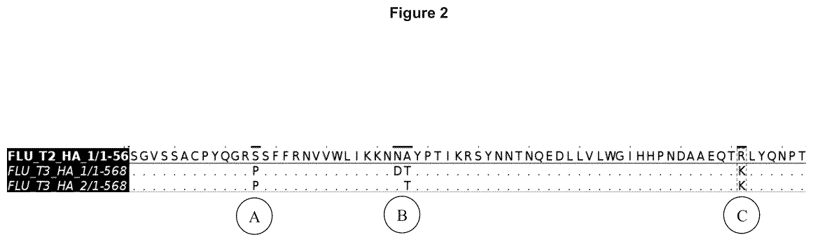

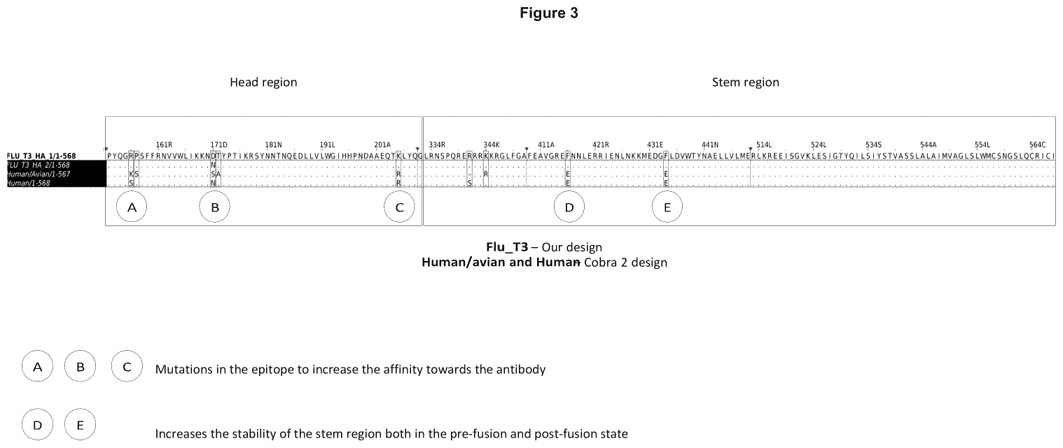

TABLE 1

| | Region | | | | | | | |
|---|---|---|---|---|---|---|---|---|
| | A | | B | | C | D | E | |
| H5 Residue | 156 | 157 | 171 | 172 | 205 | 416 | 434 | Example/FIG. |
| FLU_T2_HA_1 | R | S | N | A | R | F | F | Ex1/FIG. 2 |
| FLU_T3_HA_1 | R | P | D | T | K | F | F | Ex4/FIG. 2 |
| FLU_T3_HA_2 | R | P | N | T | K | F | F | Ex5/FIG. 2 |
| COBRA (Human/Avian) | K | S | S | A | R | E | E | FIG. 3 |
| COBRA (Human) | S | P | N | T | R | E | E | FIG. 3 |

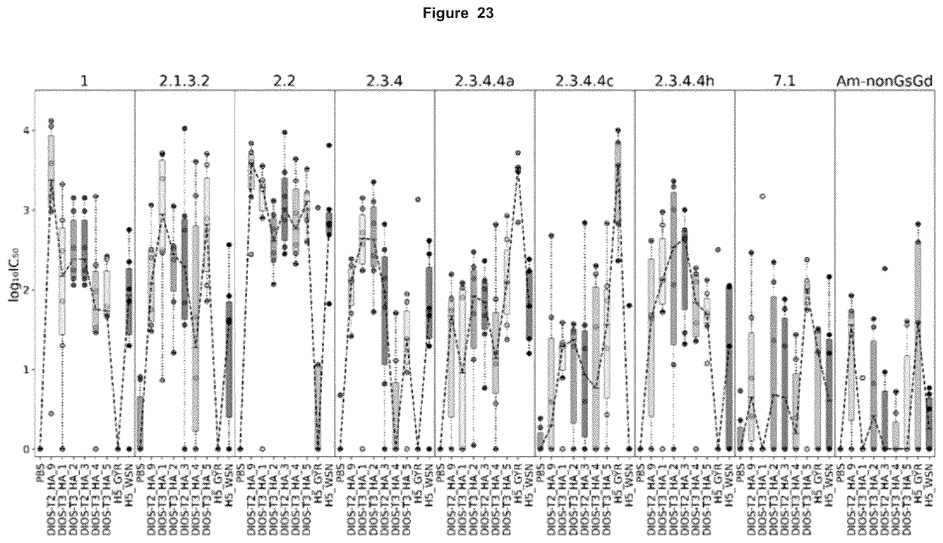

The Applicant has also designed additional amino acid sequences and their encoding nucleic acid molecules that induce a broadly neutralising immune response against important H5 clades of influenza A. These polypeptides are referred to herein as FLU_T3_HA_3, FLU_T3_HA_4, and FLU_T3_HA_5. Such polypeptides are particularly advantageous as they elicit broadly neutralising antibody responses to a diverse panel of H5 influenza viruses, as demonstrated by the results described Example 24, and FIG. 23.

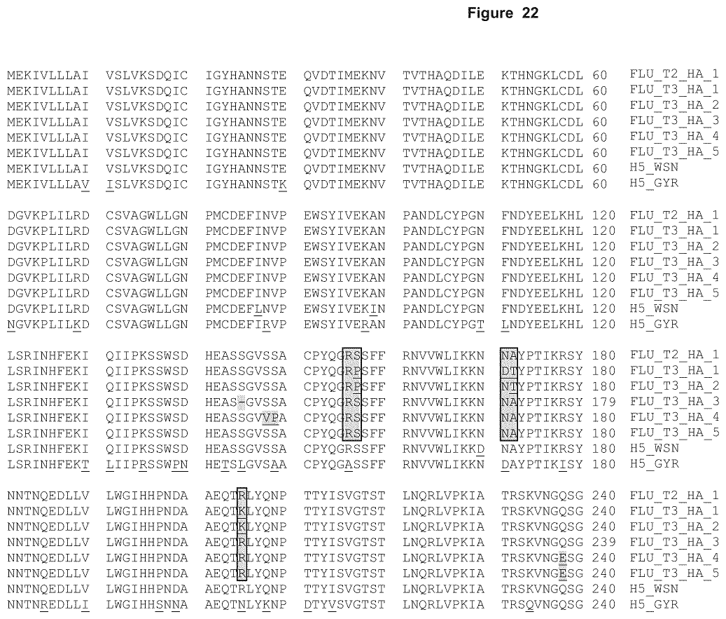

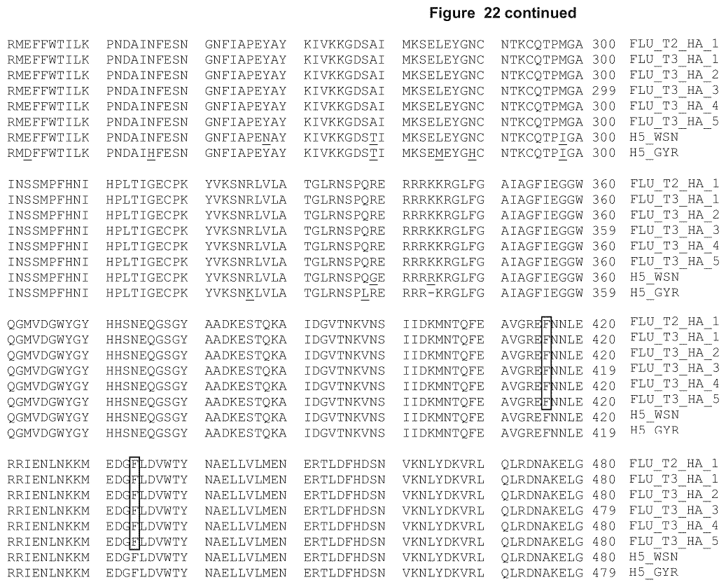

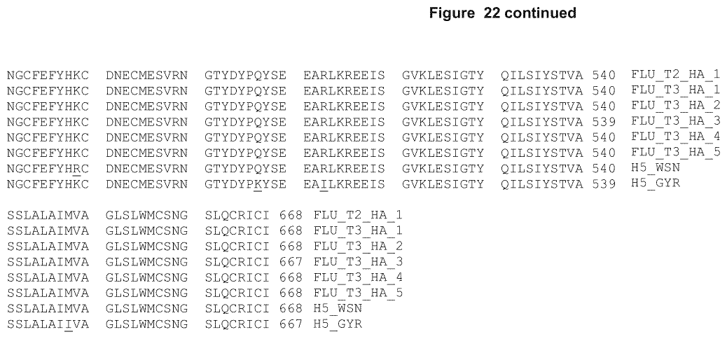

FIG. 22 shows the amino acid sequences of FLU_T3_HA_3 (SEQ ID NO:27), FLU_T3_HA_4 (SEQ ID NO:35), and FLU_T3_HA_5 (SEQ ID NO:43) in alignment with the amino acid sequences of FLU_T2_HA_1 (also referred to as FLU_T2_HA_9), FLU_T3_HA_1, and FLU_T3_HA_2, and with the HA amino acid sequence of influenza A H5N1 strains A/whooper swan/Mongolia/244/2005 (H5_WSN) (SEQ ID NO:64), and A/gyrfalcon/Washington/41088-6/2014 (H5GYR) (SEQ ID NO:65).

FIG. 21 summarises differences in amino acid sequence at positions A-E of the influenza haemagluttinin H5 for FLU_T2_HA_1 (also known as FLU_T2_HA_9), FLU_T3_HA_1, FLU_T3_HA_2, FLU_T3_HA_3, FLU_T3_HA_4, FLU_T3_HA_5).

According to the invention there is also provided an isolated polypeptide comprising a haemagluttinin subtype 5 (H5) globular head domain, and optionally a haemagluttinin stem domain, wherein the polypeptide comprises an amino acid sequence in which an amino acid residue at a position corresponding to residue position 144 or 145 of a wild-type H5 globular head domain has been deleted.

Optionally the polypeptide comprises an amino acid sequence in which an amino acid residue at a position corresponding to residue position 144 of the wild-type H5 globular head domain has been deleted.

Optionally the polypeptide comprises an amino acid sequence in which an amino acid residue at a position corresponding to residue position 145 of the wild-type H5 globular head domain has been deleted.

Optionally a polypeptide of the invention in which an amino acid residue at a position corresponding to residue position 144 or 145 of a wild-type H5 globular head domain has been deleted comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:3.

Optionally an isolated polypeptide of the invention which comprises an H5 globular head domain retains at least some HA activity of a wild-type H5 globular head domain (for example, of an H5 WSN isolate—SEQ ID NO:64).

HA activity can be determined, for example, by blood agglutination assays, or by binding assays with sialic acid (SA). Suitable blood agglutination assays are referred to in Ustinov et al. (Biochemistry (Moscow), 2017, Vol. 82, No. 11, pp. 1234-1248: The Power and Limitations of Influenza Virus Hemagglutinin Assays). A suitable binding assay is described by Takemoto et al (VIROLOGY 217, 452-458 (1996): A Surface Plasmon Resonance Assay for the Binding of Influenza Virus).

Influenza virions can agglutinate erythrocytes with the formation of a viscous gel. The agglutination occurs through the binding of virion-embedded HA to sialylated surface proteins of several erythrocytes at once. The number of agglutinated erythrocytes is proportional to the HA content and can be used for estimating the functional activity of the protein itself. The classical procedure uses 0.5-1.0% suspension of erythrocytes mixed and incubated with the virus suspension, with negative control containing erythrocytes only, and positive control containing erythrocytes and virions (Salk, J. E. (1944) A simplified procedure for titrating hemagglutinating capacity of influenza virus and the corresponding antibody, J. Immunol., 49, 87-98).

A hemagglutination test can be performed not only for influenza virions, but for isolated HA molecules as well if these molecules are in the form of trimers to provide the formation of a multiple-contact network. Thus, the HA ectodomain that exists in solely monomeric form does not agglutinate erythrocytes, while oligomerization-prone HA1 (a.a. 1-330) does (Khurana, et al., (2010) Properly folded bacterially expressed H1N1 hemagglutinin globular head and ectodomain vaccines protect ferrets against H1N1 pandemic influenza virus, PLoS One, 5, e11548.). Removal of the HA1 N-terminal fragment (a.a. 1-8) that contains the oligomerization signal Ile-Cys-Ile results in complete loss of the HA1 activity, while removal of the C-terminal portion (a.a. 321-330), on the contrary, stabilizes the trimer and facilitates hemagglutination. The larger HA1 fragment (a.a. 1-104) is also capable of oligomerization but does not agglutinate erythrocytes because of the absence of the SA binding site.

Optionally an isolated polypeptide of the invention which comprises an H5 globular head domain retains at least 25%, at least 50%, or at least 75% of HA activity of a wild-type H5 globular head domain (for example, of an H5 WSN isolate—SEQ ID NO:64).

Optionally an isolated polypeptide of the invention in which an amino acid residue at a position corresponding to residue position 144 or 145 of a wild-type H5 globular head domain has been deleted comprises an amino acid sequence with the following amino acid residues at positions corresponding to residues 156, 157, 171, 172, and 205 of the wild-type H5 globular head domain:
156: R;
157: S;
171: N;
172: A; and
205: R

Optionally an isolated polypeptide of the invention in which an amino acid residue at a position corresponding to residue position 144 or 145 of a wild-type H5 globular head domain has been deleted comprises an amino acid sequence of SEQ ID NO:27 or 29, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:27 or 29.

Optionally an isolated polypeptide of the invention in which an amino acid residue at a position corresponding to residue position 144 or 145 of a wild-type H5 globular head domain has been deleted comprises an amino acid sequence of SEQ ID NO:29.

Optionally an isolated polypeptide of the invention in which an amino acid residue at a position corresponding to residue position 144 or 145 of a wild-type H5 globular head domain has been deleted comprises an amino acid sequence of SEQ ID NO:27.

Optionally an isolated polypeptide of the invention in which an amino acid residue at a position corresponding to residue position 144 or 145 of a wild-type H5 globular head domain has been deleted comprises a haemagluttinin stem domain, and wherein the polypeptide comprises an amino acid sequence with the following amino acid residues at positions corresponding to residue positions 416 and 434 of a wild-type H5 sequence:
416: F; and
434: F

Optionally the polypeptide comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:27.

According to the invention there is also provided an isolated polypeptide comprising a haemagluttinin subtype 5 (H5) globular head domain, and optionally a haemagluttinin stem domain, wherein the polypeptide comprises an amino acid sequence with the following amino acid residues at positions corresponding to residue positions 148 and 149 of a wild-type H5 globular head domain:
148: V;
149: P.

Optionally a polypeptide of the invention which comprises an amino acid sequence with V at a position corresponding to residue position 148, and P at a position 149 corresponding to residue position 149, comprises an amino acid sequence with the following amino acid residue at a position corresponding to residue position 238 of a wild-type H5 globular head domain:
238: E.

Optionally a polypeptide of the invention which comprises an amino acid sequence with V at a position corresponding to residue position 148, and P at a position 149 corresponding to residue position 149, comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:3.

Optionally an isolated polypeptide of the invention which comprises an H5 globular head domain retains at least some HA activity of a wild-type H5 globular head domain (for example, of an H5 WSN isolate—SEQ ID NO:64).

Optionally an isolated polypeptide of the invention which comprises an H5 globular head domain retains at least 25%, at least 50%, or at least 75% of HA activity of a wild-type H5 globular head domain (for example, of an H5 WSN isolate—SEQ ID NO:64).

Optionally a polypeptide of the invention which comprises an amino acid sequence with V at a position corresponding to residue position 148, and P at a position 149 corresponding to residue position 149, has reduced affinity for its receptor compared with the wild-type H5 globular head domain.

Optionally a polypeptide of the invention which comprises an amino acid sequence with V at a position corresponding to residue position 148, and P at a position 149 corresponding to residue position 149, comprises an amino acid sequence with the following amino acid residues at positions corresponding to residues 156, 157, 171, 172, and 205 of the wild-type H5 globular head domain:
156: R;
157: S;
171: N;
172: A; and
205: R.

Optionally a polypeptide of the invention which comprises an amino acid sequence with V at a position corresponding to residue position 148, and P at a position 149 corresponding to residue position 149, comprises an amino acid sequence of SEQ ID NO:35 or 37, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:35 or 37.

Optionally an isolated polypeptide of the invention comprises an amino acid sequence of SEQ ID NO:37.

Optionally an isolated polypeptide of the invention comprises an amino acid sequence of SEQ ID NO:35.

Optionally a polypeptide of the invention which comprises an amino acid sequence with V at a position corresponding to residue position 148, and P at a position 149 corresponding to residue position 149, comprises a haemagluttinin stem domain, and wherein the polypeptide comprises an amino acid sequence with the following amino acid residues at positions corresponding to residue positions 416 and 434 of a wild-type H5 sequence:
416: F; and
434: F

Optionally the polypeptide comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:35.

There is also provided according to the invention an isolated polypeptide comprising a haemagluttinin subtype 5 (H5) globular head domain, and optionally a haemagluttinin stem domain, wherein the polypeptide comprises an amino acid sequence with the following amino acid residue at a position corresponding to residue position 238 of a wild-type H5 globular head domain:
238: E

Optionally an isolated polypeptide of the invention which comprises an amino acid sequence with an E residue at a position corresponding to residue position 238 of a wild-type H5 globular head domain comprises an amino acid sequence with the following amino acid residues at positions corresponding to residue positions 148 and 149 of a wild-type H5 globular head domain:
148: S;
149: S.

Optionally an isolated polypeptide of the invention which comprises an amino acid sequence with an E residue at a position corresponding to residue position 238 of a wild-type H5 globular head domain comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:3.

Optionally an isolated polypeptide of the invention which comprises an H5 globular head domain retains at least some HA activity of a wild-type H5 globular head domain (for example, of an H5 WSN isolate—SEQ ID NO:64).

Optionally an isolated polypeptide of the invention which comprises an H5 globular head domain retains at least 25%, at least 50%, or at least 75% of HA activity of a wild-type H5 globular head domain (for example, of an H5 WSN isolate—SEQ ID NO:64).

Optionally an isolated polypeptide of the invention which comprises an amino acid sequence with an E residue at a position corresponding to residue position 238 of a wild-type H5 globular head domain has reduced affinity for its receptor compared with the wild-type H5 globular head domain.

Optionally an isolated polypeptide of the invention which comprises an amino acid sequence with an E residue at a position corresponding to residue position 238 of a wild-type H5 globular head domain comprises an amino acid sequence with the following amino acid residues at positions corresponding to residues 156, 157, 171, 172, and 205 of the wild-type H5 globular head domain:
156: R;
157: S;
171: N;
172: A; and
205: R.

Optionally an isolated polypeptide of the invention which comprises an amino acid sequence with an E residue at a position corresponding to residue position 238 of a wild-type H5 globular head domain comprises an amino acid sequence of SEQ ID NO:43 or 45, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:43 or 45.

Optionally an isolated polypeptide of the invention comprises an amino acid sequence of SEQ ID NO:45.

Optionally an isolated polypeptide of the invention comprises an amino acid sequence of SEQ ID NO:43.

Optionally an isolated polypeptide of the invention comprises an amino acid sequence with the following amino acid residues at positions corresponding to residue positions 279 and 298 of the wild-type H5 globular head domain:
279 A; and
298 M

Optionally the polypeptide comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of any of SEQ ID NO: 27, 35, or 43.

A polypeptide of the invention may comprise any suitable haemagluttinin stem domain, including a stem domain of any suitable influenza haemagluttinin subtype, including a non-H5 subtype. Optionally the stem domain is an H5 stem domain.

Optionally a polypeptide of the invention comprises the following amino acid residues at positions 416 and 434 of the stem domain:
416: F; and
434: F

Optionally a polypeptide of the invention is up to 10,000, 9,000, 8,000, 7,000, 6,000, 5,000, 4,000, 3000, 2000, 1500, 1000, 900, 800, 700, 600, 590, 580, 570, 560, 550, 540, 530, 520, 510, 500, 490, 480, 470, 460, 450, 440, 430, 420, 410, 400, 390, 380, 370,360, 350, 340, 330, 320, 310, 300, 290, 280, or 270 amino acid residues in length.

The Applicant has also appreciated that a polypeptide that includes a fragment of the H5 globular head domain with amino acid residues from positions A-C can also elicit an antibody response against H5 influenza viruses. For example, such a polypeptide may be used on its own, or grafted onto other HA subtype heads, or other proteins (for example with a similar folding motif) to generate a suitable antibody response.

Accordingly, there is also provided according to the invention an isolated polypeptide which comprises the following amino acid sequence:

R(P/S)SFFRNVWLIWKKN(D/N)(T/A)YPTIKRSYNNTNQEDLLVLWGIHHPNDAAEQT(K/R) (SEQ ID NO:13), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:13 and which has the following amino acid residues at positions corresponding to positions 1, 2, 16, 17, and 50 of SEQ ID NO:13:
1: R;
2: P or S, preferably P;
16: D or N;
17: T or A, preferably T; and
50: K or R, preferably K.

Optionally a polypeptide of the invention which comprises an amino acid sequence of SEQ ID NO:13, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:13, comprises the following amino acid residues at positions 1, 2, 16, 17, and 50 of the amino acid sequence, or at positions corresponding to positions 1, 2, 16, 17, and 50 of SEQ ID NO:13:
1: R;
2: P;
16: D;
17: T; and
50: K

Optionally a polypeptide of the invention which comprises an amino acid sequence of SEQ ID NO:13, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:13, comprises the following amino acid residues at positions 1, 2, 16, 17, and 50 of the amino acid sequence, or at positions corresponding to positions 1, 2, 16, 17, and 50 of SEQ ID NO:13:
1: R;
2: P;
16: N;
17: T; and
50: K

Optionally a polypeptide of the invention which comprises an amino acid sequence of SEQ ID NO:13, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:13, comprises the following amino acid residues at positions 1, 2, 16, 17, and 50 of the amino acid sequence, or at positions corresponding to positions 1, 2, 16, 17, and 50 of SEQ ID NO:13:
1: R;
2: S;
16: N;
17: A; and
50: R

Optionally a polypeptide of the invention which comprises an amino acid sequence of SEQ ID NO:13, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:13, is up to 570, 560, 550, 500, 450, 400, 350, 300, 250, 200, 150, 100, 90, 80, 70, 60, or 50 amino acid residues in length.

According to the invention there is also provided an isolated polypeptide which comprises an amino acid sequence of any of SEQ ID NOs:5, 9, or 12, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of any of SEQ ID NOs:5, 9, or 12 and which has the following amino acid residues at positions corresponding to positions 148 and 166 of SEQ ID NO:5, 9, or 12:
148: F; and
166: F

The applicant has found that such polypeptides, when forming a stem region of a haemagluttinin molecule, stabilise the stem region in both the pre- and post-fusion state. Such polypeptides may, for example, be provided with an H5 haemagluttinin head domain or a non-H5 head domain.

Optionally a polypeptide of the invention which comprises an amino acid sequence of any of SEQ ID NOs:5, 9, or 12, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of any of SEQ ID NO:5, 9, or 12, is up to 10,000, 9,000, 8,000, 7,000, 6,000, 5,000, 4,000, 3000, 2000, 1500, 1000, 900, 800, 700, 600, 590, 580, 570, 560, 550, 540, 530, 520, 510, 500, 490, 480, 470, 460, 450, 440, 430, 420, 410, 400, 390, 380, 370, 360, 350, 340, 330, 320, 310, or 300 amino acid residues in length.

A polypeptide of the invention may include one or more conservative amino acid substitutions. Conservative amino acid substitutions are those substitutions that, when made, least interfere with the properties of the original protein, that is, the structure and especially the function of the protein is conserved and not significantly changed by such substitutions. Examples of conservative substitutions are shown below:

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Conservative substitutions generally maintain (a) the structure of the polypeptide backbone in the area of the substitution, for example, as a sheet or helical conformation, (b) the charge or hydrophobicity of the molecule at the target site, or (c) the bulk of the side chain.

The substitutions which in general are expected to produce the greatest changes in protein properties will be non-conservative, for instance changes in which (a) a hydrophilic residue, for example, serine or threonine, is substituted for (or by) a hydrophobic residue, for example, leucine, isoleucine, phenylalanine, valine or alanine; (b) a cysteine or proline is substituted for (or by) any other residue; (c) a residue having an electropositive side chain, for example, lysine, arginine, or histidine, is substituted for (or by) an electronegative residue, for example, glutamate or aspartate; or (d) a residue having a bulky side chain, for example, phenylalanine, is substituted for (or by) one not having a side chain, for example, glycine.

There is also provided according to the invention an isolated nucleic acid molecule encoding a polypeptide of the invention, or the complement thereof.

There is also provided according to the invention an isolated nucleic acid molecule comprising a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length to a nucleic acid molecule of the invention encoding polypeptide of the invention, or the complement thereof.

Optionally nucleic acid molecule of the invention comprises a nucleotide sequence of SEQ ID NO:2, 4, or 6, or the complement thereof.

There is also provided according to the invention an isolated nucleic acid molecule comprising a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length with SEQ ID NO:2, 4, or 6, or the complement thereof.

There is also provided according to the invention an isolated nucleic acid molecule, which comprises a nucleotide sequence of SEQ ID NO:28, 30, 32, or 34, or which comprises nucleotide sequence of SEQ ID NOs:32 and 34, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO: 28, 30, 32, 34, or with SEQ ID NO:32 and 34, over its entire length, or the complement thereof.

Optionally an isolated nucleic acid molecule of the invention comprises a nucleotide sequence of SEQ ID NO:28, 30, 32, or 34, or nucleotide sequence of SEQ ID NOs:32 and 34, or the complement thereof.

Optionally an isolated nucleic acid molecule of the invention comprises a nucleotide sequence of SEQ ID NO:28, or the complement thereof.

Optionally an isolated nucleic acid molecule of the invention comprises a nucleotide sequence of SEQ ID NO:36, 38, 40, or 42, or which comprises nucleotide sequence of SEQ ID NOs:40 and 42, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO: 36, 38, 40, or 42, or with SEQ ID NO:40 and 42, over its entire length, or the complement thereof.

Optionally an isolated nucleic acid molecule of the invention comprises a nucleotide sequence of SEQ ID NO:36, 38, 40, or 42, or nucleotide sequence of SEQ ID NOs:40 and 42, or the complement thereof.

Optionally an isolated nucleic acid molecule of the invention comprises a nucleotide sequence of SEQ ID NO:36, or the complement thereof.

Optionally an isolated nucleic acid molecule of the invention comprises a nucleotide sequence of SEQ ID NO:44, 46, 48, or 50, or nucleotide sequence of SEQ ID NOs 48 and 50, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO: 44, 46, 48, or 50, or with SEQ ID NO: 48 and 50, over its entire length, or the complement thereof.

Optionally an isolated nucleic acid molecule of the invention comprises a nucleotide sequence of SEQ ID NO:44, 46, 48, or 50, or nucleotide sequence of SEQ ID NOs 48 and 50, or the complement thereof.

Optionally an isolated nucleic acid molecule of the invention comprises a nucleotide sequence of SEQ ID NO:44, or the complement thereof.

Optionally an isolated nucleic acid molecule of the invention comprises a nucleotide sequence of SEQ ID NO:52, 54, 55, 56, or which comprises nucleotide sequence of SEQ ID NOs:52 and 54, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO: 52, 54, 55, 56, or with SEQ ID NO:52 and 54, over its entire length, or the complement thereof.

Optionally an isolated nucleic acid molecule of the invention comprises a nucleotide sequence of SEQ ID NO:52, 54, 55, 56, or nucleotide sequence of SEQ ID NOs:52 and 54, or the complement thereof.

Optionally an isolated nucleic acid molecule of the invention comprises a nucleotide sequence of SEQ ID NO:55, or the complement thereof.

Optionally an isolated nucleic acid molecule of the invention comprises a nucleotide sequence of SEQ ID NO:58, 60, 61, 62, or nucleotide sequence of SEQ ID NOs:58 and 60, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO: 58, 60, 61, 62, or with SEQ ID NO:58 and 60, over its entire length, or the complement thereof.

Optionally an isolated nucleic acid molecule of the invention comprises a nucleotide sequence of SEQ ID NO:58, 60, 61, 62, or which comprises nucleotide sequence of SEQ ID NOs:58 and 60, or the complement thereof.

Optionally an isolated nucleic acid molecule of the invention comprises a nucleotide sequence of SEQ ID NO:61, or the complement thereof.

Optionally an isolated nucleic acid molecule of the invention comprises a messenger RNA (mRNA) molecule.

The term “broadly neutralising immune response” is used herein in respect of influenza A to include an immune response elicited in a subject that is sufficient to inhibit (i.e. reduce), neutralise or prevent infection, and/or progress of infection, of at least 3 antigenically distinct clades of influenza A. Optionally a broadly neutralising immune response is sufficient to inhibit, neutralise or prevent infection, and/or progress of infection, of different H5 clades of influenza A. Optionally, advantageously the different clades include clades 2.3.4 and/or 7.1. Optionally, the different clades include clade 2.3.4.4.

### Additional H5 Embodiments of the Invention:

The Applicant has also designed additional amino acid sequences and their encoding nucleic acid molecules that induce a broadly neutralising immune response against important H5 clades of influenza A. The polypeptides comprising such amino acid sequence are referred to herein as FLU_T4_HA_1, FLU_T4_HA_2, and FLU_T4_HA_3 polypeptides. Such polypeptides are particularly advantageous as they elicit broadly neutralising antibody responses to a diverse panel of H5 clade 2.3.4.4 influenza viruses influenza viruses, as discussed below.

### Clade 2.3.4.4

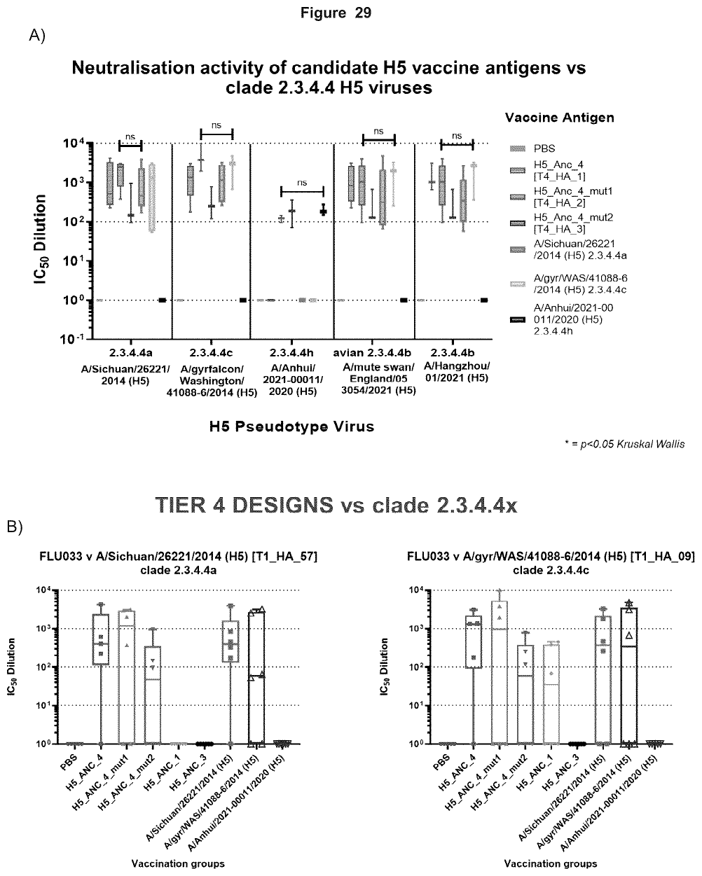

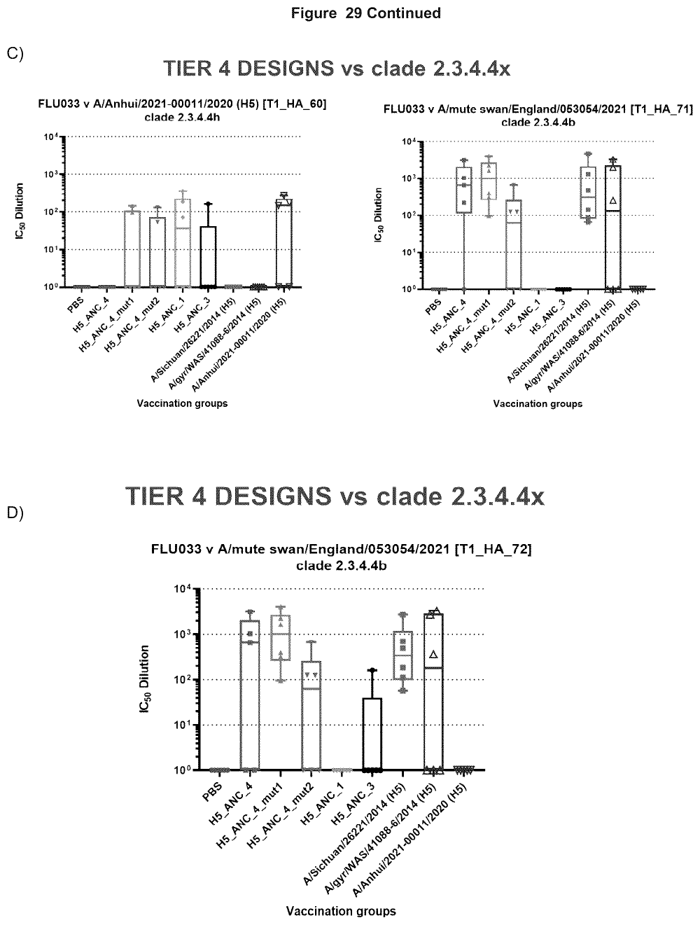

The Applicant has designed additional amino acid sequences and their encoding nucleic acid sequences that induce a broadly neutralising immune response against strains of clade 2.3.4.4 of influenza A. These polypeptides are referred to herein as FLU_T4_HA_1, FLU_T4_HA_2, and FLU_T4_HA_3. Such polypeptides are particularly advantageous as they elicit broadly neutralising antibody responses to a diverse panel of clade 2.3.4.4 influenza viruses, as demonstrated by the results described FIGS. 29 to 34 and Example 34.

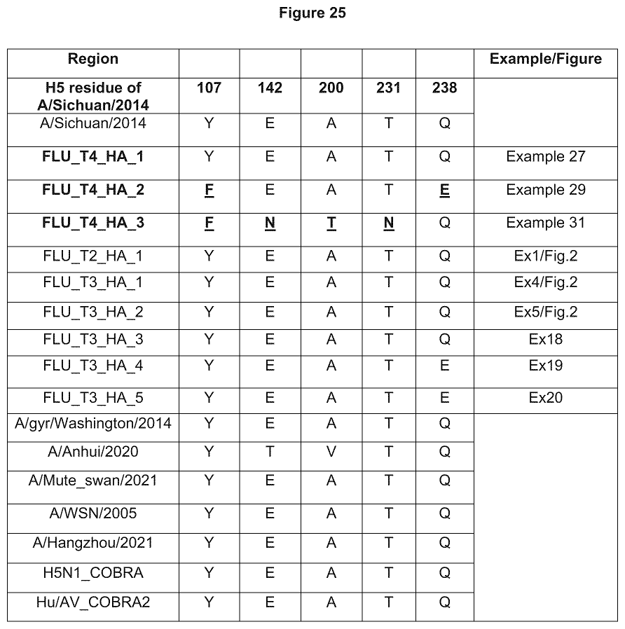

FIG. 25 summarises novel differences in amino acid sequence for new H5 designs FLU_T4_HA_1, FLU_T4_HA_2, and FLU_T4_HA_3.

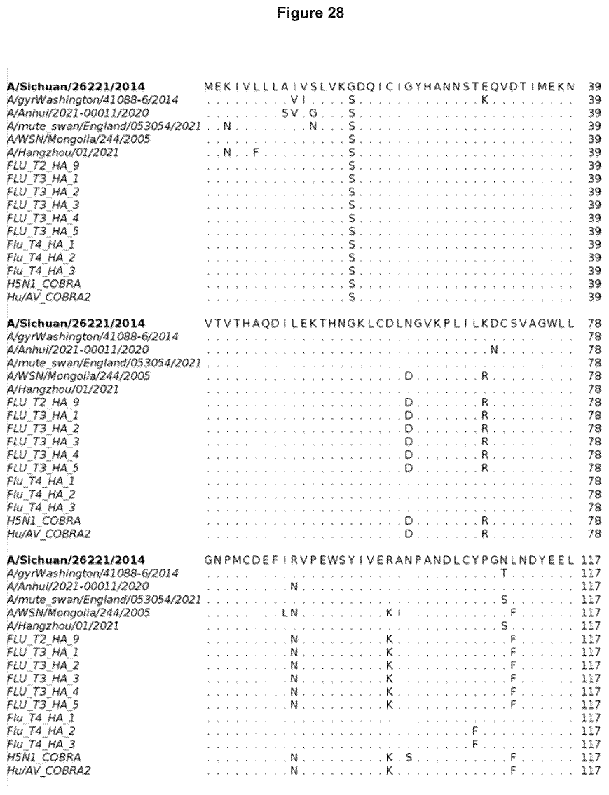

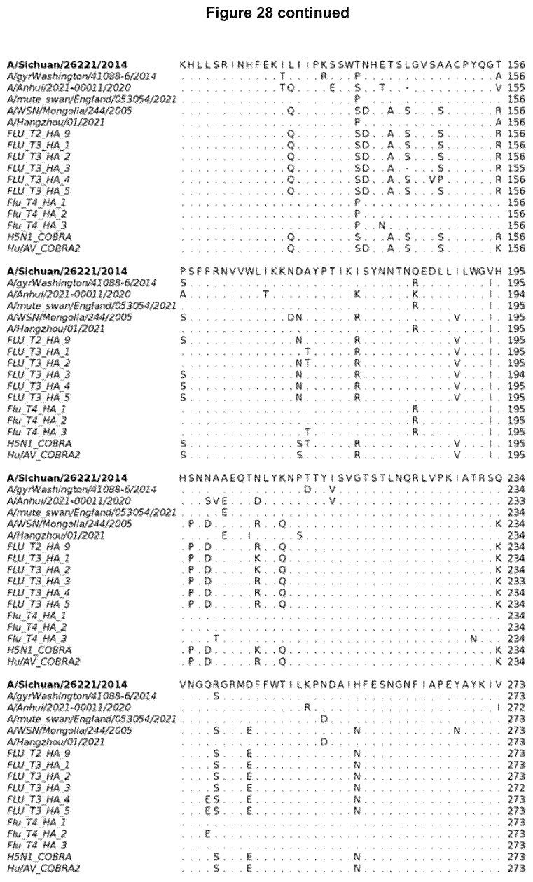

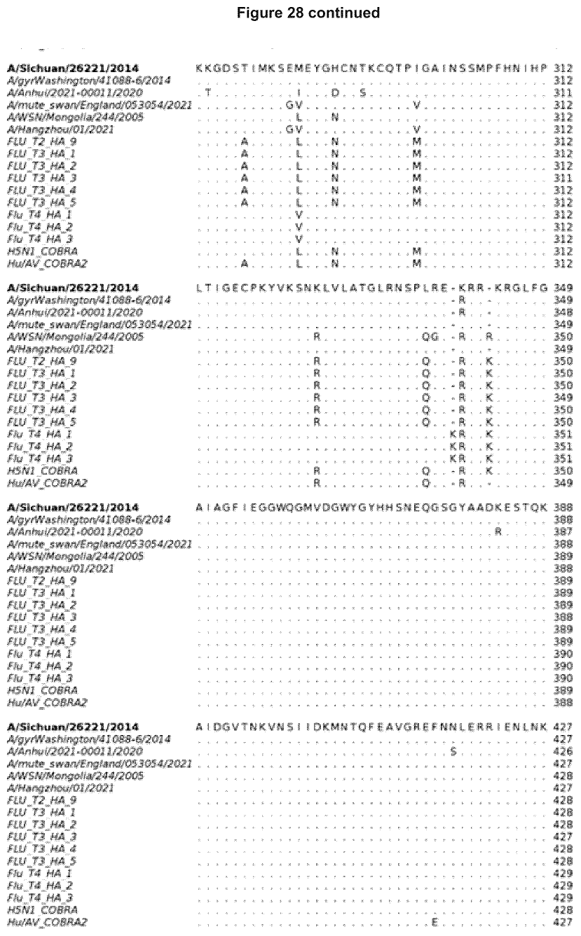

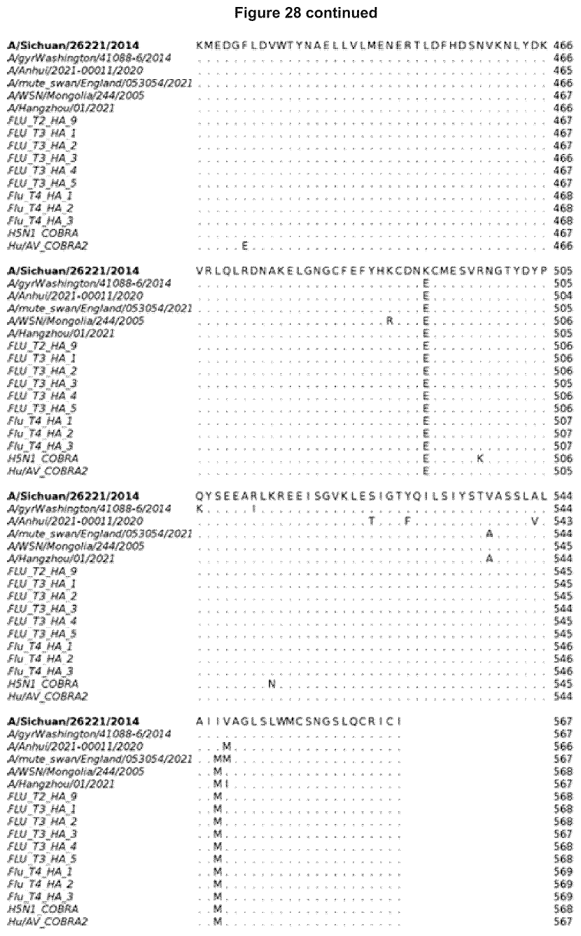

FIG. 28 shows the amino acid sequences of FLU_T4_HA_1 (SEQ ID NO:71), FLU_T4_HA_2 (SEQ ID NO:80), and FLU_T4_HA_3 (SEQ ID NO:89) in alignment with the amino acid sequences of previously designed tier 3 (T3) H5 sequences, and with the H5 amino acid sequence of influenza A H5 strains. The residue positions on the alignment correspond to the residue positions of A/Sichuan/26221/2014.

FIGS. 29-34 show neutralisation assays in mice immunised with Tier 4 (T4) vaccine candidates, previously designed sequences, or WT strains vs challenge strain. Each of FLU_T4_HA_1, FLU_T4_HA_2, and FLU_T4_HA_3 elicits a comparable neutralising response to H5 strains which are homologous to the challenge strain, and a higher response to heterologous strains.

Table 2 below summarises amino acid residue differences between the H5 A/Sichuan/2014 isolate and tier 4 (T4) H5 designs of the invention.

TABLE 2

| | H5 residue of | | | |
|---|---|---|---|---|
| H5 residue | A/Sichuan/2014 | | | |
| position of | (SEQ ID | FLU_T4_HA_1 | FLU_T4_HA_2 | FLU_T4_HA_3 |
| A/Sichuan/2014 | NO: 100) | (SEQ ID NO: 71) | (SEQ ID NO: 80) | (SEQ ID NO: 89) |
| 107 | Y | Y | F | F |
| 142 | E | E | E | N |
| 200 | A | A | A | T |
| 231 | T | T | T | N |
| 238 | Q | Q | E | Q |

### FLU_T4_HA_1 Polypeptides and Encoding Nucleic Acid Molecules.

According to the invention there is provided an isolated polypeptide comprising an amino acid sequence of SEQ ID NO:71 (FLU_T4_HA_1: HA0 amino acid sequence).

According to the invention there is provided an isolated nucleic acid molecule comprising a nucleotide sequence that encodes an amino acid sequence of SEQ ID NO:71 (FLU_T4_HA_1: HA0 amino acid sequence), or the complement thereof.

Optionally the nucleotide sequence that encodes an amino acid sequence of SEQ ID NO:71 (FLU_T4_HA_1: HA0 amino acid sequence) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length with the nucleotide sequence of SEQ ID NO:72 (FLU_T4_HA_1: HA0 nucleic acid sequence), or the complement thereof.

According to the invention there is provided an isolated nucleic acid molecule comprising a nucleotide sequence of SEQ ID NO:72 (FLU_T4_HA_1: HA0 nucleic acid sequence), or the complement thereof.

According to the invention there is also provided an isolated polypeptide comprising an amino acid sequence of SEQ ID NO:73 (FLU_T4_HA_1: head region amino acid sequence).

According to the invention there is provided an isolated nucleic acid molecule comprising a nucleotide sequence that encodes an amino acid sequence of SEQ ID NO:73 (FLU_T4_HA_1: head region amino acid sequence), or the complement thereof.

Optionally the nucleotide sequence that encodes an amino acid sequence of SEQ ID NO:73 (FLU_T4_HA_1: head region amino acid sequence) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length with the nucleotide sequence SEQ ID NO:74 (FLU_T4_HA_1: head region nucleic acid sequence), or the complement thereof.

According to the invention there is provided an isolated nucleic acid molecule comprising a nucleotide sequence of SEQ ID NO:74 (FLU_T4_HA_1: head region nucleic acid sequence), or the complement thereof.

According to the invention there is provided an isolated polypeptide comprising an amino acid sequence of SEQ ID NO:75 (FLU_T4_HA_1: first stem region amino acid sequence).

According to the invention there is provided an isolated nucleic acid molecule comprising a nucleotide sequence that encodes an amino acid sequence of SEQ ID NO:75 (FLU_T4_HA_1: first stem region amino acid sequence), or the complement thereof.

Optionally the nucleotide sequence that encodes an amino acid sequence of SEQ ID NO:75 (FLU_T4_HA_1: first stem region amino acid sequence) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length with the nucleotide sequence SEQ ID NO:76 (FLU_T4_HA_1: first stem region nucleic acid sequence), or the complement thereof.

According to the invention there is provided an isolated nucleic acid molecule comprising a nucleotide sequence of SEQ ID NO:76 (FLU_T4_HA_1: first stem region nucleic acid sequence), or the complement thereof.

According to the invention there is provided an isolated polypeptide comprising an amino acid sequence of SEQ ID NO:77 (FLU_T4_HA_1: second stem region amino acid sequence).

According to the invention there is provided an isolated nucleic acid molecule comprising a nucleotide sequence that encodes an amino acid sequence of SEQ ID NO:77 (FLU_T4_HA_1: second stem region amino acid sequence), or the complement thereof.

Optionally the nucleotide sequence that encodes an amino acid sequence of SEQ ID NO:77 (FLU_T4_HA_1: second stem region amino acid sequence) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length with the nucleotide sequence SEQ ID NO:78 (FLU_T4_HA_1: second stem region nucleic acid sequence), or the complement thereof.

According to the invention there is provided an isolated nucleic acid molecule comprising a nucleotide sequence of SEQ ID NO:78 (FLU_T4_HA_1: second stem region nucleic acid sequence), or the complement thereof.

According to the invention there is provided an isolated nucleic acid molecule comprising a nucleotide sequence of SEQ ID NO:79 (pEVAC-FLU_T4_HA_1), or the complement thereof.

### FLU_T4_HA_2 Polypeptides and Encoding Nucleic Acid Molecules

According to the invention there is provided an isolated polypeptide comprising an amino acid sequence of SEQ ID NO:80 (FLU_T4_HA_2: HA0 amino acid sequence).

According to the invention there is provided an isolated nucleic acid molecule comprising a nucleotide sequence that encodes an amino acid sequence of SEQ ID NO:80 (FLU_T4_HA_2: HA0 amino acid sequence), or the complement thereof.

Optionally the nucleotide sequence that encodes an amino acid sequence of SEQ ID NO:80 (FLU_T4_HA_2: HA0 amino acid sequence) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length with the nucleotide sequence of SEQ ID NO:81 (FLU_T4_HA_2: HA0 nucleic acid sequence), or the complement thereof.

According to the invention there is provided an isolated nucleic acid molecule comprising a nucleotide sequence of SEQ ID NO:81 (FLU_T4_HA_2: HA0 nucleic acid sequence), or the complement thereof.

According to the invention there is also provided an isolated polypeptide which comprises an amino acid sequence of SEQ ID NO:80 (FLU_T4_HA_2: HA0 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:80, and which comprises an amino acid residue F at a position corresponding to amino acid residue 107 of SEQ ID NO:100 (A/Sichuan/2014 H5), or an amino acid residue E at a position corresponding to amino acid residue 238 of SEQ ID NO:100 (A/Sichuan/2014 H5).

Optionally the polypeptide further comprises:
an amino acid residue E at a position corresponding to amino acid residue 142 of SEQ ID NO:100 (A/Sichuan/2014 H5);
an amino acid residue A at a position corresponding to amino acid residue 172 of SEQ ID NO:100 (A/Sichuan/2014 H5);
an amino acid residue A at a position corresponding to amino acid residue 200 of SEQ ID NO:100 (A/Sichuan/2014 H5);
an amino acid residue T at a position corresponding to amino acid residue 231 of SEQ ID NO:100 (A/Sichuan/2014 H5);
an amino acid residue R at a position corresponding to amino acid residue 344 of SEQ ID NO:100 (A/Sichuan/2014 H5); or
an amino acid residue K at a position corresponding to amino acid residue 345 of SEQ ID NO:100 (A/Sichuan/2014 H5).

According to the invention there is provided an isolated nucleic acid molecule comprising a nucleotide sequence that encodes an amino acid sequence of SEQ ID NO:80 (FLU_T4_HA_2: HA0 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:80, and which comprises an amino acid residue F at a position corresponding to amino acid residue 107 of SEQ ID NO:100 (A/Sichuan/2014 H5), or an amino acid residue E at a position corresponding to amino acid residue 238 of SEQ ID NO:100 (A/Sichuan/2014 H5), or the complement thereof.

According to the invention there is also provided an isolated polypeptide which comprises an amino acid sequence of SEQ ID NO:80 (FLU_T4_HA_2: HA0 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:80, and which comprises an amino acid residue F at a position corresponding to amino acid residue 107 of SEQ ID NO:100 (A/Sichuan/2014 H5), and an amino acid residue E at a position corresponding to amino acid residue 238 of SEQ ID NO:100 (A/Sichuan/2014 H5).

Optionally the polypeptide further comprises:
an amino acid residue E at a position corresponding to amino acid residue 142 of SEQ ID NO:100 (A/Sichuan/2014 H5);
an amino acid residue A at a position corresponding to amino acid residue 172 of SEQ ID NO:100 (A/Sichuan/2014 H5);
an amino acid residue A at a position corresponding to amino acid residue 200 of SEQ ID NO:100 (A/Sichuan/2014 H5);
an amino acid residue T at a position corresponding to amino acid residue 231 of SEQ ID NO:100 (A/Sichuan/2014 H5);
an amino acid residue R at a position corresponding to amino acid residue 344 of SEQ ID NO:100 (A/Sichuan/2014 H5); or
an amino acid residue K at a position corresponding to amino acid residue 345 of SEQ ID NO:100 (A/Sichuan/2014 H5).

According to the invention there is provided an isolated nucleic acid molecule comprising a nucleotide sequence that encodes an amino acid sequence of SEQ ID NO:80 (FLU_T4_HA_2: HA0 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:80, and which comprises an amino acid residue F at a position corresponding to amino acid residue 107 of SEQ ID NO:100 (A/Sichuan/2014 H5), and an amino acid residue E at a position corresponding to amino acid residue 238 of SEQ ID NO:100 (A/Sichuan/2014 H5), or the complement thereof.

According to the invention there is also provided an isolated polypeptide comprising an amino acid sequence of SEQ ID NO:82 (FLU_T4_HA_2: head region amino acid sequence).

According to the invention there is provided an isolated nucleic acid molecule comprising a nucleotide sequence that encodes an amino acid sequence of SEQ ID NO:82 (FLU_T4_HA_2: head region amino acid sequence), or the complement thereof.

Optionally the nucleotide sequence that encodes an amino acid sequence of SEQ ID NO:82 (FLU_T4_HA_2: head region amino acid sequence) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length with the nucleotide sequence SEQ ID NO:83 (FLU_T4_HA_2: head region nucleic acid sequence), or the complement thereof.

According to the invention there is provided an isolated nucleic acid molecule comprising a nucleotide sequence of SEQ ID NO:83 (FLU_T4_HA_2: head region nucleic acid sequence), or the complement thereof.

According to the invention there is also provided an isolated polypeptide which comprises an amino acid sequence of SEQ ID NO:82 (FLU_T4_HA_2: head region amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:82, and which comprises an amino acid residue F at a position corresponding to amino acid residue 107 of SEQ ID NO:100 (A/Sichuan/2014 H5), or an amino acid residue E at a position corresponding to amino acid residue 238 of SEQ ID NO:100 (A/Sichuan/2014 H5).

Optionally the polypeptide further comprises:
an amino acid residue E at a position corresponding to amino acid residue 142 of SEQ ID NO:100 (A/Sichuan/2014 H5);
an amino acid residue A at a position corresponding to amino acid residue 172 of SEQ ID NO:100 (A/Sichuan/2014 H5);
an amino acid residue A at a position corresponding to amino acid residue 200 of SEQ ID NO:100 (A/Sichuan/2014 H5); or
an amino acid residue T at a position corresponding to amino acid residue 231 of SEQ ID NO:100 (A/Sichuan/2014 H5).

According to the invention there is provided an isolated nucleic acid molecule comprising a nucleotide sequence that encodes an amino acid sequence of SEQ ID NO:82 (FLU_T4_HA_2: head region amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:82, and which comprises an amino acid residue F at a position corresponding to amino acid residue 107 of SEQ ID NO:100 (A/Sichuan/2014 H5), or an amino acid residue E at a position corresponding to amino acid residue 238 of SEQ ID NO:100 (A/Sichuan/2014 H5), or the complement thereof.

According to the invention there is also provided an isolated polypeptide which comprises an amino acid sequence of SEQ ID NO:82 (FLU_T4_HA_2: head region amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:82, and which comprises an amino acid residue F at a position corresponding to amino acid residue 107 of SEQ ID NO:100 (A/Sichuan/2014 H5), and an amino acid residue E at a position corresponding to amino acid residue 238 of SEQ ID NO:100 (A/Sichuan/2014 H5).

Optionally the polypeptide further comprises:
an amino acid residue E at a position corresponding to amino acid residue 142 of SEQ ID NO:100 (A/Sichuan/2014 H5);
an amino acid residue A at a position corresponding to amino acid residue 172 of SEQ ID NO:100 (A/Sichuan/2014 H5);
an amino acid residue A at a position corresponding to amino acid residue 200 of SEQ ID NO:100 (A/Sichuan/2014 H5); or
an amino acid residue T at a position corresponding to amino acid residue 231 of SEQ ID NO:100 (A/Sichuan/2014 H5).

According to the invention there is provided an isolated nucleic acid molecule comprising a nucleotide sequence that encodes an amino acid sequence of SEQ ID NO:82 (FLU_T4_HA_2: head region amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:82, and which comprises an amino acid residue F at a position corresponding to amino acid residue 107 of SEQ ID NO:100 (A/Sichuan/2014 H5), and an amino acid residue E at a position corresponding to amino acid residue 238 of SEQ ID NO:100 (A/Sichuan/2014 H5), or the complement thereof.

According to the invention there is provided an isolated polypeptide comprising an amino acid sequence of SEQ ID NO:84 (FLU_T4_HA_2: first stem region amino acid sequence).

According to the invention there is provided an isolated nucleic acid molecule comprising a nucleotide sequence that encodes an amino acid sequence of SEQ ID NO:84 (FLU_T4_HA_2: first stem region amino acid sequence), or the complement thereof.

Optionally the nucleotide sequence that encodes an amino acid sequence of SEQ ID NO:84 (FLU_T4_HA_2: first stem region amino acid sequence) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length with the nucleotide sequence SEQ ID NO:85 (FLU_T4_HA_2: first stem region nucleic acid sequence), or the complement thereof.

According to the invention there is provided an isolated nucleic acid molecule comprising a nucleotide sequence of SEQ ID NO:85 (FLU_T4_HA_2: first stem region nucleic acid sequence), or the complement thereof.

According to the invention there is provided an isolated polypeptide comprising an amino acid sequence of SEQ ID NO:86 (FLU_T4_HA_2: second stem region amino acid sequence).

According to the invention there is provided an isolated nucleic acid molecule comprising a nucleotide sequence that encodes an amino acid sequence of SEQ ID NO:86 (FLU_T4_HA_2: second stem region amino acid sequence), or the complement thereof.

Optionally the nucleotide sequence that encodes an amino acid sequence of SEQ ID NO:86 (FLU_T4_HA_2: second stem region amino acid sequence) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length with the nucleotide sequence SEQ ID NO:87 (FLU_T4_HA_2: second stem region nucleic acid sequence), or the complement thereof.

According to the invention there is provided an isolated nucleic acid molecule comprising a nucleotide sequence of SEQ ID NO:87 (FLU_T4_HA_2: second stem region nucleic acid sequence), or the complement thereof.

According to the invention there is provided an isolated nucleic acid molecule comprising a nucleotide sequence of SEQ ID NO:88 (pEVAC-FLU_T4_HA_2), or the complement thereof.

There is also provided according to the invention an isolated nucleic acid molecule comprising a nucleotide sequence that encodes an amino acid sequence of any of the FLU_T4_HA_2 polypeptides of the invention above, or the complement thereof.

### FLU_T4_HA_3 Polypeptides and Encoding Nucleic Molecules

According to the invention there is provided an isolated polypeptide comprising an amino acid sequence of SEQ ID NO:89 (FLU_T4_HA_3: HA0 amino acid sequence).

According to the invention there is provided an isolated nucleic acid molecule comprising a nucleotide sequence that encodes an amino acid sequence of SEQ ID NO:89 (FLU_T4_HA_3: HA0 amino acid sequence), or the complement thereof.

Optionally the nucleotide sequence that encodes an amino acid sequence of SEQ ID NO:89 (FLU_T4_HA_3: HA0 amino acid sequence) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length with the nucleotide sequence of SEQ ID NO:90 (FLU_T4_HA_3: HA0 nucleic acid sequence), or the complement thereof.

According to the invention there is provided an isolated nucleic acid molecule comprising a nucleotide sequence of SEQ ID NO:90 (FLU_T4_HA_3: HA0 nucleic acid sequence), or the complement thereof.

According to the invention there is also provided an isolated polypeptide which comprises an amino acid sequence of SEQ ID NO:89, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:89, and which comprises an amino acid residue F at a position corresponding to amino acid residue 107 of SEQ ID NO:100 (A/Sichuan/2014 H5), an amino acid residue N at a position corresponding to amino acid residue 142 of SEQ ID NO:100 (A/Sichuan/2014 H5), an amino acid residue T at a position corresponding to amino acid residue 200 of SEQ ID NO:100 (A/Sichuan/2014 H5), or an amino acid residue N at a position corresponding to amino acid residue 231 of SEQ ID NO:100 (A/Sichuan/2014 H5).

Optionally the polypeptide further comprises:
an amino acid residue T at a position corresponding to amino acid residue 172 of SEQ ID NO:100 (A/Sichuan/2014 H5);
an amino acid residue Q at a position corresponding to amino acid residue 238 of SEQ ID NO:100 (A/Sichuan/2014 H5);
an amino acid residue R at a position corresponding to amino acid residue 344 of SEQ ID NO:100 (A/Sichuan/2014 H5); or
an amino acid residue K at a position corresponding to amino acid residue 345 of SEQ ID NO:100 (A/Sichuan/2014 H5).

According to the invention there is provided an isolated nucleic acid molecule comprising a nucleotide sequence that encodes an amino acid sequence of SEQ ID NO:89 (FLU_T4_HA_3: HA0 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:89, and which comprises an amino acid residue F at a position corresponding to amino acid residue 107 of SEQ ID NO:100 (A/Sichuan/2014 H5), an amino acid residue N at a position corresponding to amino acid residue 142 of SEQ ID NO:100 (A/Sichuan/2014 H5), an amino acid residue T at a position corresponding to amino acid residue 200 of SEQ ID NO:100 (A/Sichuan/2014 H5), or an amino acid residue N at a position corresponding to amino acid residue 231 of SEQ ID NO:100 (A/Sichuan/2014 H5), or the complement thereof.

According to the invention there is also provided an isolated polypeptide which comprises an amino acid sequence of SEQ ID NO:89, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:89, and which comprises an amino acid residue F at a position corresponding to amino acid residue 107 of SEQ ID NO:100 (A/Sichuan/2014 H5), an amino acid residue N at a position corresponding to amino acid residue 142 of SEQ ID NO:100 (A/Sichuan/2014 H5), an amino acid residue T at a position corresponding to amino acid residue 200 of SEQ ID NO:100 (A/Sichuan/2014 H5), and an amino acid residue N at a position corresponding to amino acid residue 231 of SEQ ID NO:100 (A/Sichuan/2014 H5).

Optionally the polypeptide further comprises:
an amino acid residue T at a position corresponding to amino acid residue 172 of SEQ ID NO:100 (A/Sichuan/2014 H5);
an amino acid residue Q at a position corresponding to amino acid residue 238 of SEQ ID NO:100 (A/Sichuan/2014 H5);
an amino acid residue R at a position corresponding to amino acid residue 344 of SEQ ID NO:100 (A/Sichuan/2014 H5); or
an amino acid residue K at a position corresponding to amino acid residue 345 of SEQ ID NO:100 (A/Sichuan/2014 H5).

According to the invention there is provided an isolated nucleic acid molecule comprising a nucleotide sequence that encodes an amino acid sequence of SEQ ID NO:89 (FLU_T4_HA_3: HA0 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:89, and which comprises an amino acid residue F at a position corresponding to amino acid residue 107 of SEQ ID NO:100 (A/Sichuan/2014 H5), an amino acid residue N at a position corresponding to amino acid residue 142 of SEQ ID NO:100 (A/Sichuan/2014 H5), an amino acid residue T at a position corresponding to amino acid residue 200 of SEQ ID NO:100 (A/Sichuan/2014 H5), and an amino acid residue N at a position corresponding to amino acid residue 231 of SEQ ID NO:100 (A/Sichuan/2014 H5), or the complement thereof.

According to the invention there is also provided an isolated polypeptide comprising an amino acid sequence of SEQ ID NO:91 (FLU_T4_HA_3: head region amino acid sequence).

According to the invention there is provided an isolated nucleic acid molecule comprising a nucleotide sequence that encodes an amino acid sequence of SEQ ID NO:91 (FLU_T4_HA_3: head region amino acid sequence), or the complement thereof.

Optionally the nucleotide sequence that encodes an amino acid sequence of SEQ ID NO:91 (FLU_T4_HA_3: head region amino acid sequence) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length with the nucleotide sequence SEQ ID NO:92 (FLU_T4_HA_3: head region nucleic acid sequence), or the complement thereof.

According to the invention there is provided an isolated nucleic acid molecule comprising a nucleotide sequence of SEQ ID NO:92 (FLU_T4_HA_3: head region nucleic acid sequence), or the complement thereof.

According to the invention there is also provided an isolated polypeptide which comprises an amino acid sequence of SEQ ID NO:91 (FLU_T4_HA_3: head region amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:91, and which comprises an amino acid residue F at a position corresponding to amino acid residue 107 of SEQ ID NO:100 (A/Sichuan/2014 H5), an amino acid residue N at a position corresponding to amino acid residue 142 of SEQ ID NO:100 (A/Sichuan/2014 H5), an amino acid residue T at a position corresponding to amino acid residue 200 of SEQ ID NO:100 (A/Sichuan/2014 H5), or an amino acid residue N at a position corresponding to amino acid residue 231 of SEQ ID NO:100 (A/Sichuan/2014 H5).

Optionally the polypeptide further comprises:
an amino acid residue T at a position corresponding to amino acid residue 172 of SEQ ID NO:100 (A/Sichuan/2014 H5); or
an amino acid residue Q at a position corresponding to amino acid residue 238 of SEQ ID NO:100 (A/Sichuan/2014 H5).

According to the invention there is provided an isolated nucleic acid molecule comprising a nucleotide sequence that encodes an amino acid sequence of SEQ ID NO:91 (FLU_T4_HA_3: head region amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:91, and which comprises an amino acid residue F at a position corresponding to amino acid residue 107 of SEQ ID NO:100 (A/Sichuan/2014 H5), an amino acid residue N at a position corresponding to amino acid residue 142 of SEQ ID NO:100 (A/Sichuan/2014 H5), an amino acid residue T at a position corresponding to amino acid residue 200 of SEQ ID NO:100 (A/Sichuan/2014 H5), or an amino acid residue N at a position corresponding to amino acid residue 231 of SEQ ID NO:100 (A/Sichuan/2014 H5), or the complement thereof.

According to the invention there is also provided an isolated polypeptide which comprises an amino acid sequence of SEQ ID NO:91 (FLU_T4_HA_3: head region amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:91, and which comprises an amino acid residue F at a position corresponding to amino acid residue 107 of SEQ ID NO:100 (A/Sichuan/2014 H5), an amino acid residue N at a position corresponding to amino acid residue 142 of SEQ ID NO:100 (A/Sichuan/2014 H5), an amino acid residue T at a position corresponding to amino acid residue 200 of SEQ ID NO:100 (A/Sichuan/2014 H5), and an amino acid residue N at a position corresponding to amino acid residue 231 of SEQ ID NO:100 (A/Sichuan/2014 H5).

Optionally the polypeptide further comprises:
an amino acid residue T at a position corresponding to amino acid residue 172 of SEQ ID NO:100 (A/Sichuan/2014 H5); or
an amino acid residue Q at a position corresponding to amino acid residue 238 of SEQ ID NO:100 (A/Sichuan/2014 H5).

According to the invention there is provided an isolated nucleic acid molecule comprising a nucleotide sequence that encodes an amino acid sequence of SEQ ID NO:91 (FLU_T4_HA_3: head region amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:91, and which comprises an amino acid residue F at a position corresponding to amino acid residue 107 of SEQ ID NO:100 (A/Sichuan/2014 H5), an amino acid residue N at a position corresponding to amino acid residue 142 of SEQ ID NO:100 (A/Sichuan/2014 H5), an amino acid residue T at a position corresponding to amino acid residue 200 of SEQ ID NO:100 (A/Sichuan/2014 H5), and an amino acid residue N at a position corresponding to amino acid residue 231 of SEQ ID NO:100 (A/Sichuan/2014 H5), or the complement thereof.

According to the invention there is provided an isolated polypeptide comprising an amino acid sequence of SEQ ID NO:93 (FLU_T4_HA_3: first stem region amino acid sequence).

According to the invention there is provided an isolated nucleic acid molecule comprising a nucleotide sequence that encodes an amino acid sequence of SEQ ID NO:93 (FLU_T4_HA_3: first stem region amino acid sequence), or the complement thereof.

Optionally the nucleotide sequence that encodes an amino acid sequence of SEQ ID NO:93 (FLU_T4_HA_3: first stem region amino acid sequence) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length with the nucleotide sequence SEQ ID NO:94 (FLU_T4_HA_3: first stem region nucleic acid sequence), or the complement thereof.

According to the invention there is provided an isolated nucleic acid molecule comprising a nucleotide sequence of SEQ ID NO:94 (FLU_T4_HA_3: first stem region nucleic acid sequence), or the complement thereof.

According to the invention there is provided an isolated polypeptide comprising an amino acid sequence of SEQ ID NO:95 (FLU_T4_HA_3: second stem region amino acid sequence).

According to the invention there is provided an isolated nucleic acid molecule comprising a nucleotide sequence that encodes an amino acid sequence of SEQ ID NO:95 (FLU_T4_HA_3: second stem region amino acid sequence), or the complement thereof.

Optionally the nucleotide sequence that encodes an amino acid sequence of SEQ ID NO:95 (FLU_T4_HA_3: second stem region amino acid sequence) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length with the nucleotide sequence SEQ ID NO:96 (FLU_T4_HA_3: second stem region nucleic acid sequence), or the complement thereof.

According to the invention there is provided an isolated nucleic acid molecule comprising a nucleotide sequence of SEQ ID NO:96 (FLU_T4_HA_3: second stem region nucleic acid sequence), or the complement thereof.

According to the invention there is provided an isolated nucleic acid molecule comprising a nucleotide sequence of SEQ ID NO:97 (pEVAC-FLU_T4_HA_3), or the complement thereof.

There is also provided according to the invention an isolated nucleic acid molecule comprising a nucleotide sequence that encodes an amino acid sequence of any of the FLU_T4_HA_3 polypeptides of the invention above, or the complement thereof.

### M2

The extracellular domain of M2 has been identified as being almost invariant across all influenza A strains. This presents as a potential solution to the problem of creating a universal influenza A vaccine that elicits broad-spectrum protection against all influenza A infections.

The Applicant has identified amino acid sequences and their encoding nucleic acid molecules that induce a broadly neutralising immune response against M2 of influenza A.

M2 embodiments of the invention are described below.

According to the invention there is provided an isolated polypeptide which comprises an amino acid sequence of SEQ ID NO:14, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:14.

There is also provided according to the invention an isolated nucleic acid molecule, comprising a nucleotide sequence of SEQ ID NO:15, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:15, over its entire length, or the complement thereof.

### Neuraminidase

The Applicant has also identified amino acid sequences and their encoding nucleic acid molecules that include epitopes of neuraminidase that are conserved by several different influenza subtypes.

Neuraminidase embodiments of the invention are described below.

According to the invention there is provided an isolated polypeptide which comprises an amino acid sequence of SEQ ID NO:16, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:16.

According to the invention there is provided an isolated polypeptide which comprises an amino acid sequence of SEQ ID NO:18, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:18.

There is also provided according to the invention an isolated nucleic acid molecule, comprising a nucleotide sequence of SEQ ID NO:17, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:17, over its entire length, or the complement thereof.

There is also provided according to the invention an isolated nucleic acid molecule, comprising a nucleotide sequence of SEQ ID NO:19, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:19, over its entire length, or the complement thereof.

According to the invention there is provided an isolated polypeptide comprising an amino acid sequence of SEQ ID NO:98 (FLU_T3_NA_3 amino acid sequence).

According to the invention there is provided an isolated polypeptide which comprises an amino acid sequence of SEQ ID NO:98 (FLU_T3_NA_3 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:98.

According to the invention there is provided an isolated nucleic acid molecule comprising a nucleotide sequence that encodes an amino acid sequence of SEQ ID NO: 98 (FLU_T3_NA_3 amino acid sequence), or the complement thereof.

Optionally the nucleotide sequence that encodes an amino acid sequence of SEQ ID NO: 98 (FLU_T3_NA_3 amino acid sequence) is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical over its entire length with the nucleotide sequence of SEQ ID NO:99 (FLU_T3_NA_3 nucleic acid sequence), or the complement thereof.

According to the invention there is provided an isolated nucleic acid molecule comprising a nucleotide sequence of SEQ ID NO:99 (FLU_T3_NA_3 nucleic acid sequence), or the complement thereof.

### Influenza A H1

The Applicant has also designed amino acid sequences and their encoding nucleic acid molecules that can be used in vaccines to induce broad H1 immunity and protection against divergent strains of influenza A. The designed amino acid sequences are referred to as FLU_T2_HA_3_I3 and FLU_T2_HA_4 below.

H1 embodiments of the invention are described below.

FLU_T2_HA_3_I3:

FLU_T2_HA_3_I3 embodiments of the invention are described below.

There is also provided according to the invention an isolated polypeptide, which comprises an amino acid sequence of SEQ ID NO:22 (FLU_T2_HA_3_I3).

There is also provided according to the invention an isolated polynucleotide, which comprises a nucleotide sequence encoding an amino acid sequence of SEQ ID NO:22 (FLU_T2_HA_3_I3), or the complement thereof.

The nucleotide sequence may comprise a sequence of SEQ ID NO:23, or the complement thereof.

There is also provided according to the invention an isolated polypeptide, which comprises an amino acid sequence of SEQ ID NO:22 (FLU_T2_HA_3_I3), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:22.

There is also provided according to the invention an isolated polynucleotide, which comprises a nucleotide sequence of SEQ ID NO:23, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:23, over its entire length, or the complement thereof.

FLU_T2_HA_4:

FLU_T2_HA_4 embodiments of the invention are described below.

There is also provided according to the invention an isolated polypeptide, which comprises an amino acid sequence of SEQ ID NO:68 (FLU_T2_HA_4).

There is also provided according to the invention an isolated polynucleotide, which comprises a nucleotide sequence encoding an amino acid sequence of SEQ ID NO:69 (FLU_T2_HA_4), or the complement thereof.

The nucleotide sequence may comprise a sequence of SEQ ID NO:69, or the complement thereof.

There is also provided according to the invention an isolated polypeptide, which comprises an amino acid sequence of SEQ ID NO:68 (FLU_T2_HA_4), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:68.

There is also provided according to the invention an isolated polynucleotide, which comprises a nucleotide sequence of SEQ ID NO:69, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:69, over its entire length, or the complement thereof. H5 and H1 embodiments of the invention are referred to collectively as HA embodiments below.

### Combination Vaccines

To prevent vaccine escape more effectively, vaccines with a combination of 2 or more (preferably 3 or more) evolutionarily constrained, computationally designed viral antigen targets are provided, each designed to independently give the maximum breadth of vaccine protection. Vaccines of the invention may comprise ancestral antigen based designs of HA, NA and M2, either alone or in combination. Furthermore, combinations of modified HA and NA antigen structures that are not predominantly found to circulate widely as natural combinations in humans are provided (e.g. a group 1 HA combined with a group 2 NA not found to circulate and to co-evolve together, such as H1N1 or H3N2).

Polypeptides or nucleic acid molecules of the invention may be combined in any suitable combination (for example, HA and/or M2 and/or neuraminidase embodiments of the invention, H5 and/or M2 and/or neuraminidase embodiments of the invention, H1 and/or M2 and/or neuraminidase embodiments of the invention, or FLU_T2_HA_3_I3 and/or Flu_T2_NA_3 and/or Flu_T2_M2_1 embodiments of the invention, or FLU_T2_HA_4 and/or Flu_T2_NA_3 and/or Flu_T2_M2_1 embodiments of the invention) to provide an influenza vaccine that protects against far more influenza strains than current vaccines. In some embodiments such combination vaccines protect against several influenza A and B variants (especially those embodiments that include M2 embodiments, as M2 is better conserved between influenza A and B).

Optionally, one embodiment of each different category of embodiment is used in combination. For example, an HA embodiment (H5 or H1), and/or an M2 embodiment and/or a neuraminidase embodiment.

Optionally a trivalent vaccine combines H5, M2, and neuraminidase embodiments of the invention.

Optionally a trivalent vaccine of the invention combines an H5 embodiment, an M2 embodiment, and a neuraminidase embodiment of the invention.

Optionally a trivalent vaccine combines H1, M2, and neuraminidase embodiments of the invention.

Optionally a trivalent vaccine of the invention combines an H1 embodiment, an M2 embodiment, and a neuraminidase embodiment of the invention.

Optionally a nucleic acid vector of the invention comprises:
i) a nucleic acid molecule encoding a polypeptide which comprises an amino acid sequence of SEQ ID NO:7 or 8, or a nucleic acid molecule encoding a polypeptide which comprises an amino acid sequence of SEQ ID NO:10 or 11, or a nucleic acid molecule encoding a polypeptide which comprises an amino acid sequence of SEQ ID NO:1 or 3, or a nucleic acid molecule encoding a polypeptide which comprises an amino acid sequence of SEQ ID NO:27 or 29, or a nucleic acid molecule encoding a polypeptide which comprises an amino acid sequence of SEQ ID NO:35 or 37, or a nucleic acid molecule encoding a polypeptide which comprises an amino acid sequence of SEQ ID NO:43 or 45 (examples of H5 embodiments); and/or
ii) a nucleic acid molecule encoding a polypeptide which comprises an amino acid sequence of SEQ ID NO:14 (examples of M2 embodiments); and/or
iii) a nucleic acid molecule encoding a polypeptide which comprises an amino acid sequence of SEQ ID NO:16, or a nucleic acid molecule encoding a polypeptide which comprises an amino acid sequence of SEQ ID NO:18 (examples of neuraminidase embodiments).

Optionally a nucleic acid vector of the invention comprises:
i) a nucleic acid molecule encoding a polypeptide which comprises an amino acid sequence of SEQ ID NO:22, or a nucleic acid molecule encoding a polypeptide which comprises an amino acid sequence of SEQ ID NO:68 (examples of H1 embodiments); and/or
ii) a nucleic acid molecule encoding a polypeptide which comprises an amino acid sequence of SEQ ID NO:14 (examples of M2 embodiments); and/or
iii) a nucleic acid molecule encoding a polypeptide which comprises an amino acid sequence of SEQ ID NO:16, or a nucleic acid molecule encoding a polypeptide which comprises an amino acid sequence of SEQ ID NO:18 (examples of neuraminidase embodiments).

Optionally a nucleic acid vector of the invention comprises:
i) a nucleic acid molecule which comprises nucleotide sequence encoding FLU_T2_HA_3_I3 amino acid sequence (SEQ ID NO:22), or the complement thereof; and/or
ii) a nucleic acid molecule which comprises nucleotide sequence encoding FLU_T2_NA_3 amino acid sequence (SEQ ID NO:16), or the complement thereof; and/or
iii) a nucleic acid molecule which comprises nucleotide sequence encoding FLU_T2_M2_1 amino acid sequence (SEQ ID NO:14), or the complement thereof.

Optionally a nucleic acid vector of the invention comprises:
i) a nucleic acid molecule which comprises a nucleotide sequence encoding an amino acid sequence of SEQ ID NO:22 (FLU_T2_HA_3_I3 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:22, or the complement thereof; and/or
ii) a nucleic acid molecule which comprises a nucleotide sequence encoding an amino acid sequence of SEQ ID NO:16 (FLU_T2_NA_3 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:16, or the complement thereof; and/or
iii) a nucleic acid molecule which comprises a nucleotide sequence encoding an amino acid sequence of SEQ ID NO:14 (FLU_T2_M2_1 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:14, or the complement thereof.

Optionally the nucleic acid molecule of (i) comprises a nucleotide sequence encoding an amino acid sequence of SEQ ID NO:22 (FLU_T2_HA_3_I3 amino acid sequence), or the complement thereof.

Optionally the nucleic acid molecule of (ii) comprises a nucleotide sequence encoding an amino acid sequence of SEQ ID NO:16 (FLU_T2_NA_3 amino acid sequence), or the complement thereof.

Optionally the nucleic acid molecule of (iii) comprises a nucleotide sequence encoding an amino acid sequence of SEQ ID NO:14 (FLU_T2_M2_1 amino acid sequence), or the complement thereof.

Optionally the nucleotide sequence encoding FLU_T2_HA_3_I3 amino acid sequence (SEQ ID NO:22), or the complement thereof, comprises the nucleotide sequence of SEQ ID NO:23, or the complement thereof.

Optionally the nucleotide sequence encoding FLU_T2_NA_3 amino acid sequence (SEQ ID NO:16), or the complement thereof, comprises the nucleotide sequence of SEQ ID NO:17, or the complement thereof.

Optionally the nucleotide sequence encoding FLU_T2_M2_1 amino acid sequence (SEQ ID NO:14), or the complement thereof, comprises the nucleotide sequence of SEQ ID NO:15, or the complement thereof.

Optionally a vector of the invention further comprises a promoter operably linked to each nucleic acid molecule.

Optionally a vector of the invention is a pEVAC-based vector.

The immune response may be humoral and/or a cellular immune response. A cellular immune response is a response of a cell of the immune system, such as a B-cell, T-cell, macrophage or polymorphonucleocyte, to a stimulus such as an antigen or vaccine. An immune response can include any cell of the body involved in a host defence response, including for example, an epithelial cell that secretes an interferon or a cytokine. An immune response includes, but is not limited to, an innate immune response or inflammation.

Optionally a polypeptide of the invention induces a protective immune response. A protective immune response refers to an immune response that protects a subject from infection or disease (i.e. prevents infection or prevents the development of disease associated with infection). Methods of measuring immune responses are well known in the art and include, for example, measuring proliferation and/or activity of lymphocytes (such as B or T cells), secretion of cytokines or chemokines, inflammation, or antibody production.

Optionally a polypeptide of the invention is able to induce the production of antibodies and/or a T-cell response in a human or non-human animal to which the polypeptide has been administered (either as a polypeptide or, for example, expressed from an administered nucleic acid expression vector).

The similarity between amino acid or nucleic acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a given gene or protein will possess a relatively high degree of sequence identity when aligned using standard methods. Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237-244, 1988; Higgins and Sharp, CABIOS 5:151-153, 1989; Corpet et al., Nucleic Acids' Research 16:10881-10890, 1988; and Pearson and Lipman, Proc. Natl. Acad. Sci. U.S.A. 85:2444, 1988. Altschul et al., Nature Genet. 6:119-129, 1994. The NCBI Basic Local Alignment Search Tool (BLAST) (Altschul et al., J. Mol. Biol. 215:403-410, 1990) is available from several sources, including the National Center for Biotechnology Information (NCBI, Bethesda, MD) and on the Internet, for use in connection with the sequence analysis programs blastp, blastn, blastx, tblastn and tblastx.

Sequence identity between nucleic acid sequences, or between amino acid sequences, can be determined by comparing an alignment of the sequences. When an equivalent position in the compared sequences is occupied by the same nucleotide, or amino acid, then the molecules are identical at that position. Scoring an alignment as a percentage of identity is a function of the number of identical nucleotides or amino acids at positions shared by the compared sequences. When comparing sequences, optimal alignments may require gaps to be introduced into one or more of the sequences to take into consideration possible insertions and deletions in the sequences. Sequence comparison methods may employ gap penalties so that, for the same number of identical molecules in sequences being compared, a sequence alignment with as few gaps as possible, reflecting higher relatedness between the two compared sequences, will achieve a higher score than one with many gaps. Calculation of maximum percent identity involves the production of an optimal alignment, taking into consideration gap penalties.

Suitable computer programs for carrying out sequence comparisons are widely available in the commercial and public sector. Examples include MatGat (Campanella et al., 2003, BMC Bioinformatics 4: 29; program available from http://bitincka.com/ledion/matgat), Gap (Needleman & Wunsch, 1970, J. Mol. Biol. 48: 443-453), FASTA (Altschul et al., 1990, J. Mol. Biol. 215: 403-410; program available from http://www.ebi.ac.uk/fasta), Clustal W2.0 and X 2.0 (Larkin et al., 2007, Bioinformatics 23: 2947-2948; program available from http://www.ebi.ac.uk/tools/clustalw2) and EMBOSS Pairwise Alignment Algorithms (Needleman & Wunsch, 1970, supra; Kruskal, 1983, In: Time warps, string edits and macromolecules: the theory and practice of sequence comparison, Sankoff & Kruskal (eds), pp 1-44, Addison Wesley; programs available from http://www.ebi.ac.uk/tools/emboss/align). All programs may be run using default parameters.

For example, sequence comparisons may be undertaken using the “needle” method of the EMBOSS Pairwise Alignment Algorithms, which determines an optimum alignment (including gaps) of two sequences when considered over their entire length and provides a percentage identity score. Default parameters for amino acid sequence comparisons (“Protein Molecule” option) may be Gap Extend penalty: 0.5, Gap Open penalty: 10.0, Matrix: Blosum 62.

The sequence comparison may be performed over the full length of the reference sequence.

Sequences described herein include reference to an amino acid sequence comprising amino acid residues “at positions corresponding to positions” of another amino acid sequence. Such corresponding positions may be identified, for example, from an alignment of the sequences using a sequence alignment method described herein, or another sequence alignment method known to the person of ordinary skill in the art.

### Vectors

There is also provided according to the invention a vector comprising a nucleic acid molecule of the invention.

Optionally a vector of the invention comprises a nucleic acid molecule encoding a polypeptide which comprises an amino acid sequence of SEQ ID NO:27 or 29.

Optionally a vector of the invention comprises a nucleic acid molecule encoding a polypeptide which comprises an amino acid sequence of SEQ ID NO:35 or 37.

Optionally a vector of the invention comprises a nucleic acid molecule encoding a polypeptide which comprises an amino acid sequence of SEQ ID NO:43 or 45.

Optionally a vector of the invention comprises a nucleic acid molecule encoding a polypeptide which comprises an amino acid sequence of SEQ ID NO:7 or 8.

Optionally a vector of the invention comprises a nucleic acid molecule encoding a polypeptide which comprises an amino acid sequence of SEQ ID NO:10 or 11.

Optionally a vector of the invention further comprises a promoter operably linked to the nucleic acid.

Optionally the promoter is for expression of a polypeptide encoded by the nucleic acid in mammalian cells.

Optionally the promoter is for expression of a polypeptide encoded by the nucleic acid in yeast or insect cells.

Optionally the vector is a vaccine vector.

Optionally the vector is a viral vaccine vector, a bacterial vaccine vector, an RNA vaccine vector, an mRNA vaccine vector, or a DNA vaccine vector.

Optionally the vector is a DNA vector.

Optionally the vector is a mRNA vector.

A polynucleotide of the invention may comprise a DNA or an RNA molecule. For embodiments in which the polynucleotide comprises an RNA molecule, it will be appreciated that the nucleic acid sequence of the polynucleotide will be the same as that recited in the respective SEQ ID, or the complement thereof, but with each ‘T’ nucleotide replaced by ‘U’.

As discussed in more detail below, a polynucleotide of the invention may include one or more modified nucleosides.

A polynucleotide of the invention may include one or more nucleotide analogs known to those of skill in the art.

A nucleic acid molecule of the invention may comprise a DNA or an RNA molecule. For embodiments in which the nucleic acid molecule comprises an RNA molecule, it will be appreciated that the molecule may comprise an RNA sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with, or identical with, any of SEQ ID NOs: 2, 4, or 6, in which each ‘T’ nucleotide is replaced by ‘U’, or the complement thereof.

For example, it will be appreciated that where an RNA vaccine vector comprising a nucleic acid of the invention is provided, the nucleic acid sequence of the nucleic acid of the invention will be an RNA sequence, so may comprise for example an RNA nucleic acid sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with, or identical with, any of SEQ ID NOs: 2, 4, or 6 in which each ‘T’ nucleotide is replaced by ‘U’, or the complement thereof.

Viral vaccine vectors use viruses to deliver nucleic acid (for example, DNA or RNA) into human or non-human animal cells. The nucleic acid contained in the virus encodes one or more antigens that, once expressed in the infected human or non-human animal cells, elicit an immune response. Both humoral and cell-mediated immune responses can be induced by viral vaccine vectors. Viral vaccine vectors combine many of the positive qualities of nucleic acid vaccines with those of live attenuated vaccines. Like nucleic acid vaccines, viral vaccine vectors carry nucleic acid into a host cell for production of antigenic proteins that can be tailored to stimulate a range of immune responses, including antibody, T helper cell (CD4⁺ T cell), and cytotoxic T lymphocyte (CTL, CD8⁺ T cell) mediated immunity. Viral vaccine vectors, unlike nucleic acid vaccines, also have the potential to actively invade host cells and replicate, much like a live attenuated vaccine, further activating the immune system like an adjuvant. A viral vaccine vector therefore generally comprises a live attenuated virus that is genetically engineered to carry nucleic acid (for example, DNA or RNA) encoding protein antigens from an unrelated organism. Although viral vaccine vectors are generally able to produce stronger immune responses than nucleic acid vaccines, for some diseases viral vectors are used in combination with other vaccine technologies in a strategy called heterologous prime-boost. In this system, one vaccine is given as a priming step, followed by vaccination using an alternative vaccine as a booster. The heterologous prime-boost strategy aims to provide a stronger overall immune response. Viral vaccine vectors may be used as both prime and boost vaccines as part of this strategy. Viral vaccine vectors are reviewed by Ura et al., 2014 (Vaccines 2014, 2, 624-641) and Choi and Chang, 2013 (Clinical and Experimental Vaccine Research 2013; 2:97-105).

Optionally the viral vaccine vector is based on a viral delivery vector, such as a Poxvirus (for example, Modified Vaccinia Ankara (MVA), NYVAC, AVIPOX), herpesvirus (e.g. HSV, CMV, Adenovirus of any host species), Morbillivirus (e.g. measles), Alphavirus (e.g. SFV, Sendai), Flavivirus (e.g. Yellow Fever), or Rhabdovirus (e.g. VSV)-based viral delivery vector, a bacterial delivery vector (for example, Salmonella, E. coli), an RNA expression vector, or a DNA expression vector.

Adenoviruses are by far the most utilised and advanced viral vectors developed for SARS2 vaccines. They are non-enveloped double-stranded DNA (dsDNA) viruses with a packaging capacity of up to 7.5 kb of foreign genes. Recombinant Adenovirus vectors are widely used because of their high transduction efficiency, high level of transgene expression, and broad range of viral tropism. These vaccines are highly cell specific, highly efficient in gene transduction, and efficient at inducing an immune response. Adenovirus vaccines are effective at triggering and priming T cells, leading to long term and high level of antigenic protein expression and therefore long lasting protection.

Where viral vectors are used in accordance with the invention, it may be advantageous that each HA and/or M2 and/or neuraminidase embodiment of the invention, H5 and/or M2 and/or neuraminidase embodiment of the invention, H1 and/or M2 and/or neuraminidase embodiment of the invention, or FLU_T2_HA_3_I3 and/or Flu_T2_NA_3 and/or Flu_T2_M2_1 embodiment of the invention, or FLU_T2_HA_4 and/or Flu_T2_NA_3 and/or Flu_T2_M2_1 embodiment of the invention, is encoded as part of the same viral vaccine vector. For example, it may be easier (and less costly) to make a single vector encoding each of the H5, M2, and neuraminidase embodiments, than several different vectors, each encoding a different H5, M2, or neuraminidase embodiment.

Optionally the nucleic acid expression vector is a nucleic acid expression vector, and a viral pseudotype vector.

Optionally the nucleic acid expression vector is a vaccine vector.

Optionally the nucleic acid expression vector comprises, from a 5′ to 3′ direction: a promoter; a splice donor site (SD); a splice acceptor site (SA); and a terminator signal, wherein the multiple cloning site is located between the splice acceptor site and the terminator signal.

Optionally the promoter comprises a CMV immediate early 1 enhancer/promoter (CMV-IE-E/P) and/or the terminator signal comprises a terminator signal of a bovine growth hormone gene (Tbgh) that lacks a KpnI restriction endonuclease site.

Optionally the nucleic acid expression vector further comprises an origin of replication, and nucleic acid encoding resistance to an antibiotic. Optionally the origin of replication comprises a pUC-plasmid origin of replication and/or the nucleic acid encodes resistance to kanamycin.

Optionally the vector is a pEVAC-based expression vector.

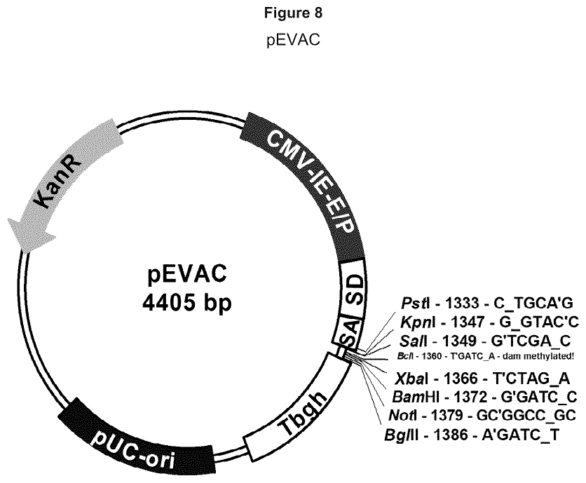

Optionally the nucleic acid expression vector comprises a nucleic acid sequence of SEQ ID NO:21 (pEVAC). The pEVAC vector has proven to be a highly versatile expression vector for generating viral pseudotypes as well as direct DNA vaccination of animals and humans. The pEVAC expression vector is described in more detail in Example 11 below. FIG. 8 shows a plasmid map for pEVAC.

The terms “polynucleotide” and “nucleic acid” are used interchangeably herein.

Optionally the, or each vaccine vector is an RNA vaccine vector.

Optionally the, or each vaccine vector is an mRNA vaccine vector.

A polynucleotide of the invention may comprise a DNA molecule.

The or each polynucleotide of a pharmaceutical composition, a combined preparation, or a vector, of the invention may comprise a DNA molecule.

A vector of the invention may be a DNA vector.

The or each vector of a pharmaceutical composition or a combined preparation of the invention may be a DNA vector.

A polynucleotide of the invention, or a polynucleotide of a pharmaceutical composition, a combined preparation, or a vector, of the invention, may be provided as part of a DNA vaccine.

There is also provided according to the invention a DNA vaccine which comprises a polynucleotide of the invention, a vector of the invention, or a pharmaceutical composition or a combined preparation of the invention which comprises one or more polynucleotides, wherein the or each polynucleotide is a DNA molecule.

A polynucleotide of the invention may comprise an RNA molecule.

The or each polynucleotide of a pharmaceutical composition, a combined preparation, or a vector, of the invention may comprise an RNA molecule.

A vector of the invention may be an RNA vector.

The or each vector of a pharmaceutical composition or a combined preparation of the invention may be an RNA vector.

A polynucleotide of the invention, or a polynucleotide of a pharmaceutical composition, a combined preparation, or a vector, of the invention, may be provided as part of an RNA vaccine.

There is also provided according to the invention an RNA vaccine which comprises a polynucleotide of the invention, a vector of the invention, or a pharmaceutical composition or a combined preparation of the invention which comprises one or more polynucleotides, wherein the or each polynucleotide is an RNA molecule.

A polynucleotide of the invention may comprise an mRNA molecule.

The or each polynucleotide of a pharmaceutical composition, a combined preparation, or a vector, of the invention may comprise an mRNA molecule.

A vector of the invention may be an mRNA vector.

The or each vector of a pharmaceutical composition or a combined preparation of the invention may be an mRNA vector.

Messenger RNA (mRNA) Vaccines

A polynucleotide of the invention, or a polynucleotide of a pharmaceutical composition, a combined preparation, or a vector, of the invention, may be provided as part of an mRNA vaccine.

There is also provided according to the invention an mRNA vaccine which comprises a polynucleotide of the invention, a vector of the invention, or a pharmaceutical composition or a combined preparation of the invention which comprises one or more polynucleotides, wherein the or each polynucleotide comprises an mRNA molecule.

Messenger RNA (mRNA) vaccines are a new form of vaccine (recently reviewed in Pardi et al., Nature Reviews Drug Discovery Volume 17, pages 261-279(2018); Wang et al., Molecular Cancer (2021) 20:33: mRNA vaccine: a potential therapeutic strategy). The first mRNA vaccines to be approved for use were BNT162b2 (BioNTech's vaccine manufactured by Pfizer) and mRNA-1273 (manufactured by Modema) during the COVID-19 pandemic. mRNA vaccines have a unique feature of temporarily promoting the expression of antigen (typically days). The expression of the exogenous antigen is controlled by the lifetime of encoding mRNA, which is regulated by cellular degradation pathways. While this transient nature of protein expression requires repeated administration for the treatment of genetic diseases and cancers, it is extremely beneficial for vaccines, where prime or prime-boost vaccination is sufficient to develop highly specific adaptive immunity without any exposure to the contagion.

mRNA based vaccines trigger an immune response after the synthetic mRNA which encodes viral antigens transfects human cells. The cytosolic mRNA molecules are then translated by the host's own cellular machinery into specific viral antigens. These antigens may then be presented on the cell surface where they can be recognised by immune cells, triggering an immune response.

The structural elements of a vaccine vector mRNA molecule are similar to those of natural mRNA, comprising a 5′ cap, 5′ untranslated region (UTR), coding region (for example, comprising an open reading frame encoding a polypeptide of the invention), 3′ UTR, and a poly(A) tail. The 5′ UTR (also known as a leader sequence, transcript leader, or leader RNA) is the region of an mRNA that is directly upstream from the initiation codon. This region is important for the regulation of translation of a transcript. In many organisms, the 5′ UTR forms complex secondary structure to regulate translation. The 5′ UTR begins at the transcription start site and ends one nucleotide (nt) before the initiation sequence (usually AUG) of the coding region. In eukaryotes, the length of the 5′ UTR tends to be anywhere from 100 to several thousand nucleotides long. The differing sizes are likely due to the complexity of the eukaryotic regulation which the 5′ UTR holds as well as the larger pre-initiation complex that must form to begin translation. The eukaryotic 5′ UTR contains the Kozak consensus sequence (ACCAUG (initiation codon underlined) (SEQ ID NO:36), which contains the initiation codon AUG. The constructs described herein contain an elongated Kozak sequence: GCCACCAUG (initiation codon underlined) (SEQ ID NO:37).

Two major types of RNA are currently studied as vaccines: non-replicating mRNA and virally derived, self-amplifying RNA. While both types of vaccines share a common structure in mRNA constructs, self-amplifying RNA vaccines contain additional sequences in the coding region for RNA replication, including RNA-dependent RNA polymerases.

BNT162b2 vaccine construct comprises a lipid nanoparticle (LNP) encapsulated mRNA molecule encoding trimerised full-length SARS2 S protein with a PP mutation (at residue positions 986-987). The mRNA is encapsulated in 80 nm ionizable cationic lipid nanoparticles. mRNA-1273 vaccine construct is also based on an LNP vector, but the synthetic mRNA encapsulated within the lipid construct encodes the full-length SARS2 S protein.

U.S. Pat. No. 10,702,600 B1 (ModemaTX) describes betacoronavirus mRNA vaccines, including suitable LNPs for use in such vaccines.

A nucleic acid vaccine (for example, a mRNA) of the invention may be formulated in a lipid nanoparticle.

mRNA vaccines have several advantages in comparison with conventional vaccines containing inactivated (or live attenuated) disease-causing organisms. Firstly, mRNA-based vaccines can be rapidly developed due to design flexibility and the ability of the constructs to mimic antigen structure and expression as seen in the course of a natural infection. mRNA vaccines can be developed within days or months based on sequencing information from a target virus, while conventional vaccines often take years and require a deep understanding of the target virus to make the vaccine effective and safe. Secondly, these novel vaccines can be rapidly produced. Due to high yields from in vitro transcription reactions, mRNA production can be rapid, inexpensive and scalable (due to chemical synthesis rather than biological growth of cells or bacteria). Thirdly, vaccine risks are low. mRNA does not contain infectious viral elements or cell debris that pose risks for infection and insertional mutagenesis (as the mRNA is generated synthetically). Anti-vector immunity is also avoided as mRNA is the minimally immunogenic genetic vector, allowing repeated administration of the vaccine. The challenge for effective application of mRNA vaccines lies in cytosolic delivery. mRNA isolates are rapidly degraded by extracellular RNases and cannot penetrate cell membranes to be transcribed in the cytosol. However, efficient in vivo delivery can be achieved by formulating mRNA into carrier molecules, allowing rapid uptake and expression in the cytoplasm. To date, numerous delivery methods have been developed including lipid-, polymer-, or peptide-based delivery, virus-like replicon particle, cationic nanoemulsion, naked mRNAs, and dendritic cell-based delivery (each reviewed in Wang et al., supra). Decationic lipid nanoparticle (LNP) delivery is the most appealing and commonly used mRNA vaccine delivery tool.

Exogenous mRNA may be highly immunostimulatory. Single-stranded RNA (ssRNA) molecules are considered a pathogen associated molecular pattern (PAMP), and are recognised by various Toll-like receptors (TLR) which elicit a pro-inflammatory reaction. Although a strong cellular and humoral immune response is desirable in response to vaccination, the innate immune reaction elicited by exogenous mRNA may cause undesirable side-effects in the subject. The U-rich sequence of mRNA is a key element to activate TLR (Wang et al., supra). Additionally, enzymatically synthesised mRNA preparations contain double stranded RNA (dsRNA) contaminants as aberrant products of the in vitro transcription (IVT) process. dsRNA is a potent PAMP, and elicits downstream reactions resulting in the inhibition of translation and the degradation of cellular mRNA and ribosomal RNA (Pardi et al., supra). Thus, the mRNA may suppress antigen expression and thus reduce vaccine efficacy.

Studies over the past decade have shown that the immunostimulatory effect of mRNA can be shaped by the purification of IVT mRNA, the introduction of modified nucleosides, complexing the mRNA with various carrier molecules (Pardi et al., supra), adding poly(A) tails or optimising mRNA with GC-rich sequence (Wang et al., supra). Chemical modification of uridine is a common approach to minimise the immunogenicity of foreign mRNA. Incorporation of pseudouridine (ψ) and N1-methylpseudouridine (m1ψ) to IVT mRNA prevents TLR activation and other innate immune sensors, thus reducing pro-inflammatory signalling in response to the exogenous mRNA. Such nucleoside modification also suppresses recognition of dsRNA species (Pardi et al., supra) and can reduce innate immune sensing of exogenous mRNA translation (Hou et al. Nature Reviews Materials, 2021, https://doi.org/10.1038/s41578-021-00358-0).

Other nucleoside chemical modifications include, but are not limited to, 5-methylcytidine (m5C), 5-methyluridine (m5U), N1-methyladenosine (m1A), N6-methyladenosine (m6A), 2-thiouridine (s2U), and 5-methoxyuridine (5moU) (Wang et al., supra).

The IVT mRNA molecules used in the mRNA-1273 and BNT162b2 COVID-19 vaccines were prepared by replacing uridine with m1ψ, and their sequences were optimized to encode a stabilized pre-fusion spike protein with two pivotal proline substitutions (Hou et al., supra). However, CureVac's mRNA vaccine candidate, CVnCoV, uses unmodified nucleosides and relies on a combination of mRNA sequence alterations to allow immune evasion without affecting the expressed protein. Firstly, CVnCoV has a higher GC content (63%) than rival vaccines (BNT162b2 has 56%) and the original SARS-CoV-2 virus itself (37%). Secondly, the vaccine comprises C-rich motifs which bind to poly(C)-binding protein, enhancing both the stability and expression of the mRNA. A further modification of CVnCoV is that it contains a histone stem-loop sequence as well as a poly(A) tail, to enhance the longevity and translation of the mRNA (Hubert, B., 2021. The CureVac Vaccine, and a brief tour through some of the wonders of nature. URL https://berthub.eu/articles/posts/curevac-vaccine-and-wonders-of-biology/.(accessed 15.09.21). However, the vaccine had disappointing results from phase III clinical trials, which experts assert are down to the decision not to incorporate chemically modified nucleosides into the mRNA sequence. Nonetheless, CureVac and Acuitas Therapeutics delivered erythropoietin (EPO)-encoding mRNA, which has rich GC codons, to pigs with lipid nanoparticles (LNPs). Their results indicated EPO-related responses were elicited without immunogenicity (Wang et al., supra), suggesting that there is still scope for unmodified mRNA nucleoside-based vaccines.

A polynucleotide of the invention may comprise an mRNA molecule.

The or each polynucleotide of a pharmaceutical composition, a combined preparation, or a vector, of the invention may comprise an mRNA molecule.

A vector of the invention may be an mRNA vector.

The or each vector of a pharmaceutical composition or a combined preparation of the invention may be an mRNA vector.

A polynucleotide of the invention, or a polynucleotide of a pharmaceutical composition, a combined preparation, or a vector, of the invention, may be provided as part of an mRNA vaccine.

There is also provided according to the invention an mRNA vaccine which comprises a polynucleotide of the invention, a vector of the invention, or a pharmaceutical composition or a combined preparation of the invention which comprises one or more polynucleotides, wherein the or each polynucleotide comprises an mRNA molecule.

RNA or mRNA of a polynucleotide of the invention, or of a polynucleotide of a pharmaceutical composition, a combined preparation, a vector, or a vaccine, of the invention may be produced by in vitro transcription (IVT).

A polynucleotide of the invention, or a polynucleotide of a pharmaceutical composition, a combined preparation, a vector, or a vaccine, of the invention may comprise one or more modified nucleosides.

The one or more modified nucleosides may be present in DNA or RNA of a polynucleotide of the invention, or of a polynucleotide of a pharmaceutical composition, a combined preparation, a vector, or a vaccine, of the invention.

Optionally, at least one chemical modification is selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine. In some embodiments, the chemical modification is in the 5-position of the uracil. In some embodiments, the chemical modification is a N1-methylpseudouridine. In some embodiments, the chemical modification is a N1-ethylpseudouridine.

For example, an RNA or an mRNA of a polynucleotide of the invention, or of a polynucleotide of a pharmaceutical composition, a combined preparation, a vector, or a vaccine, of the invention may comprise one or more of the following modified nucleosides:
pseudouridine (ψ);
N1-methylpseudouridine (m1ψ)
5-methylcytidine (m5C)
5-methyluridine (m5U)
N1-methyladenosine (m1A)
N6-methyladenosine (m6A)
2-thiouridine (s2U)
5-methoxyuridine (5moU)

In some embodiments, 100% of the uracil in the open reading frame have a chemical modification. In some embodiments, a chemical modification is in the 5-position of the uracil. In some embodiments, a chemical modification is a N1-methyl pseudouridine. In some embodiments, 100% of the uracil in the open reading frame have a N1-methyl pseudouridine in the 5-position of the uracil.

The polynucleotide may contain from about 1% to about 100% modified nucleotides (or nucleosides) (either in relation to overall nucleotide content, or in relation to one or more types of nucleotide, i.e., any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%). Any remaining percentage is accounted for by the presence of unmodified A, G, U, or C.

Optionally a polynucleotide of the invention, or of a polynucleotide of a pharmaceutical composition, a combined preparation, a vector, or a vaccine, of the invention, comprises an RNA molecule in which the nucleic acid sequence of the polynucleotide is the same as that recited in the respective SEQ ID, or the complement thereof, but with each ‘U’ replaced by m1ψ.

Optionally a polynucleotide of the invention, or of a polynucleotide of a pharmaceutical composition, a combined preparation, a vector, or a vaccine, of the invention, comprises an mRNA molecule in which the nucleic acid sequence of the polynucleotide is the same as that recited in the respective SEQ ID, or the complement thereof, but with each ‘U’ replaced by m1ψ.

Optionally a polynucleotide of the invention, or of a polynucleotide of a pharmaceutical composition, a combined preparation, a vector, or a vaccine, of the invention, comprises an RNA molecule in which the nucleic acid sequence of the polynucleotide is the same as that recited in the respective SEQ ID, or the complement thereof, but with at least 50% of the ‘U’s replaced by m1ψ. The remaining ‘U’s may all be unmodified, or may comprise unmodified and one or more other modified nucleosides.

Optionally a polynucleotide of the invention, or of a polynucleotide of a pharmaceutical composition, a combined preparation, a vector, or a vaccine, of the invention, comprises an mRNA molecule in which the nucleic acid sequence of the polynucleotide is the same as that recited in the respective SEQ ID, or the complement thereof, but with at least 50% of the ‘U’s replaced by m1ψ. The remaining ‘U’s may all be unmodified, or may comprise unmodified and one or more other modified nucleosides.

Optionally a polynucleotide of the invention, or of a polynucleotide of a pharmaceutical composition, a combined preparation, a vector, or a vaccine, of the invention, comprises an RNA molecule in which the nucleic acid sequence of the polynucleotide is the same as that recited in the respective SEQ ID, or the complement thereof, but with at least 90% of the ‘U’s replaced by m1ψ. The remaining ‘U’s may all be unmodified, or may comprise unmodified and one or more other modified nucleosides.

Optionally a polynucleotide of the invention, or of a polynucleotide of a pharmaceutical composition, a combined preparation, a vector, or a vaccine, of the invention, comprises an mRNA molecule in which the nucleic acid sequence of the polynucleotide is the same as that recited in the respective SEQ ID, or the complement thereof, but with at least 90% of the ‘U’s replaced by m1ψ. The remaining ‘U’s may all be unmodified, or may comprise unmodified and one or more other modified nucleosides. mRNA vaccines of the invention may be co-administered with an immunological adjuvant, for example MF59 (Novartis), TrMix, RNActive (CureVac AG), RNAdjuvant (again reviewed in Wang et al., supra).

Thus, in preferred embodiments, each vector of a pharmaceutical composition, or combined preparation, of the invention is an mRNA vaccine vector.

There is also provided according to the invention an isolated cell comprising or transfected with a vector of the invention.

There is also provided according to the invention a fusion protein comprising a polypeptide of the invention.

There is also provided according to the invention a pseudotyped virus comprising a polypeptide of the invention.

### Pharmaceutical Compositions

According to the invention there is also provided a pharmaceutical composition comprising a polypeptide of the invention, and a pharmaceutically acceptable carrier, excipient, or diluent.

A pharmaceutical composition of the invention may include polypeptides of the invention in any suitable combination (for example, HA and/or M2 and/or neuraminidase embodiments of the invention, H5 and/or M2 and/or neuraminidase embodiments of the invention, H1 and/or M2 and/or neuraminidase embodiments of the invention, or FLU_T2_HA_3_I3 and/or Flu_T2_NA_3 and/or Flu_T2_M2_1 embodiments of the invention, or FLU_T2_HA_4 and/or Flu_T2_NA_3 and/or Flu_T2_M2_1 embodiments of the invention).

Optionally, one embodiment of each different category of embodiment is used in combination. For example, an HA embodiment (H5 or H1), and/or an M2 embodiment and/or a neuraminidase embodiment. Optionally a pharmaceutical composition of the invention comprises:
i) a polypeptide which comprises an amino acid sequence of SEQ ID NO:7 or 8, or a polypeptide which comprises an amino acid sequence of SEQ ID NO:10 or 11, or a polypeptide which comprises an amino acid sequence of SEQ ID NO:1 or 3 (examples of H5 embodiments); and/or
ii) a polypeptide which comprises an amino acid sequence of SEQ ID NO:14 (examples of M2 embodiments); and/or
iii) a polypeptide which comprises an amino acid sequence of SEQ ID NO:16, a polypeptide which comprises an amino acid sequence of SEQ ID NO:18 (examples of neuraminidase embodiments).

Optionally a pharmaceutical composition of the invention comprises:
i) a polypeptide which comprises an amino acid sequence of SEQ ID NO:7 or 8, or a polypeptide which comprises an amino acid sequence of SEQ ID NO:10 or 11, or a polypeptide which comprises an amino acid sequence of SEQ ID NO:1 or 3, or a polypeptide which comprises an amino acid sequence of SEQ ID NO:27 or 29, or a polypeptide which comprises an amino acid sequence of SEQ ID NO:35 or 37, or a polypeptide which comprises an amino acid sequence of SEQ ID NO:43 or 45 (examples of H5 embodiments); and/or
ii) a polypeptide which comprises an amino acid sequence of SEQ ID NO:14 (examples of M2 embodiments); and/or
iii) a polypeptide which comprises an amino acid sequence of SEQ ID NO:16, a polypeptide which comprises an amino acid sequence of SEQ ID NO:18 (examples of neuraminidase embodiments).

Optionally a pharmaceutical composition of the invention comprises:
i) a polypeptide which comprises an amino acid sequence of SEQ ID NO:22, or a polypeptide which comprises an amino acid sequence of SEQ ID NO:68 (examples of H1 embodiments); and/or
ii) a polypeptide which comprises an amino acid sequence of SEQ ID NO:14 (examples of M2 embodiments); and/or
iii) a polypeptide which comprises an amino acid sequence of SEQ ID NO:16, or a polypeptide which comprises an amino acid sequence of SEQ ID NO:18 (examples of neuraminidase embodiments).

Optionally a pharmaceutical composition of the invention comprises:
i) a polypeptide which comprises an amino acid sequence of SEQ ID NO:22 (FLU_T2_HA_3_I3 amino acid sequence); and/or
ii) a polypeptide which comprises an amino acid sequence of SEQ ID NO:14 (FLU_T2_M2_1 amino acid sequence); and/or
iii) a polypeptide which comprises an amino acid sequence of SEQ ID NO:16 (FLU_T2_NA_3 amino acid sequence).

Optionally a pharmaceutical composition of the invention comprises:
i) a polypeptide which comprises an amino acid sequence of SEQ ID NO:22 (FLU_T2_HA_3_I3 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:22; and/or
ii) a polypeptide which comprises an amino acid sequence of SEQ ID NO:14 (FLU_T2_M2_1 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:14; and/or
iii) a polypeptide which comprises an amino acid sequence of SEQ ID NO:16 (FLU_T2_NA_3 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:16.

Optionally the polypeptide of (i) comprises an amino acid sequence of SEQ ID NO:22 (FLU_T2_HA_3_I3 amino acid sequence).

Optionally the polypeptide of (ii) comprises an amino acid sequence of SEQ ID NO:14 (FLU_T2_M2_1 amino acid sequence).

Optionally the polypeptide of (iii) comprises an amino acid sequence of SEQ ID NO:16 (FLU_T2_NA_3 amino acid sequence).

According to the invention there is also provided a pharmaceutical composition comprising a nucleic acid of the invention, and a pharmaceutically acceptable carrier, excipient, or diluent.

A pharmaceutical composition of the invention may include nucleic acid molecules of the invention in any suitable combination (for example, HA and/or M2 and/or neuraminidase embodiments of the invention, H5 and/or M2 and/or neuraminidase embodiments of the invention, H1 and/or M2 and/or neuraminidase embodiments of the invention, or FLU_T2_HA_3_I3 and/or Flu_T2_NA_3 and/or Flu_T2_M2_1 embodiments of the invention, or FLU_T2_HA_4 and/or Flu_T2_NA_3 and/or Flu_T2_M2_1 embodiments of the invention).

Optionally, one embodiment of each different category of embodiment is used in combination. For example, an HA embodiment (H5 or H1), and/or an M2 embodiment and/or a neuraminidase embodiment.

Optionally a pharmaceutical composition of the invention comprises:
i) a nucleic acid molecule encoding a polypeptide which comprises an amino acid sequence of SEQ ID NO:7 or 8, or a nucleic acid molecule encoding a polypeptide which comprises an amino acid sequence of SEQ ID NO:10 or 11, or a nucleic acid molecule encoding a polypeptide which comprises an amino acid sequence of SEQ ID NO:1 or 3 (examples of H5 embodiments); and/or
ii) a nucleic acid molecule encoding a polypeptide which comprises an amino acid sequence of SEQ ID NO:14 (examples of M2 embodiments); and/or
iii) a nucleic acid molecule encoding a polypeptide which comprises an amino acid sequence of SEQ ID NO:16, or a nucleic acid molecule encoding a polypeptide which comprises an amino acid sequence of SEQ ID NO:18 (examples of neuraminidase embodiments).

Optionally a pharmaceutical composition of the invention comprises:
i) a nucleic acid molecule encoding a polypeptide which comprises an amino acid sequence of SEQ ID NO:7 or 8, or a nucleic acid molecule encoding a polypeptide which comprises an amino acid sequence of SEQ ID NO:10 or 11, or a nucleic acid molecule encoding a polypeptide which comprises an amino acid sequence of SEQ ID NO:1 or 3, or a nucleic acid molecule encoding a polypeptide which comprises an amino acid sequence of SEQ ID NO:27 or 29, or a nucleic acid molecule encoding a polypeptide which comprises an amino acid sequence of SEQ ID NO:35 or 37, or a nucleic acid molecule encoding a polypeptide which comprises an amino acid sequence of SEQ ID NO:43 or 45 (examples of H5 embodiments); and/or
ii) a nucleic acid molecule encoding a polypeptide which comprises an amino acid sequence of SEQ ID NO:14 (examples of M2 embodiments); and/or
iii) a nucleic acid molecule encoding a polypeptide which comprises an amino acid sequence of SEQ ID NO:16, or a nucleic acid molecule encoding a polypeptide which comprises an amino acid sequence of SEQ ID NO:18 (examples of neuraminidase embodiments).

Optionally a pharmaceutical composition of the invention comprises:
i) a nucleic acid molecule encoding a polypeptide which comprises an amino acid sequence of SEQ ID NO:22, or a nucleic acid molecule encoding a polypeptide which comprises an amino acid sequence of SEQ ID NO:68 (examples of H1 embodiments); and/or
ii) a nucleic acid molecule encoding a polypeptide which comprises an amino acid sequence of SEQ ID NO:14 (examples of M2 embodiments); and/or
iii) a nucleic acid molecule encoding a polypeptide which comprises an amino acid sequence of SEQ ID NO:16, or a nucleic acid molecule encoding a polypeptide which comprises an amino acid sequence of SEQ ID NO:18 (examples of neuraminidase embodiments).

Optionally a pharmaceutical composition of the invention comprises:
i) an isolated polynucleotide which comprises nucleotide sequence encoding FLU_T2_HA_3_I3 amino acid sequence (SEQ ID NO:22), or the complement thereof; and/or
ii) an isolated polynucleotide which comprises nucleotide sequence encoding FLU_T2_NA_3 amino acid sequence (SEQ ID NO:16), or the complement thereof; and/or
iii) an isolated polynucleotide which comprises nucleotide sequence encoding FLU_T2_M2_1 amino acid sequence (SEQ ID NO:14), or the complement thereof.

According to the invention there is also provided a pharmaceutical composition which comprises:
i) an isolated polynucleotide which comprises nucleotide sequence encoding FLU_T2_HA_3_I3 amino acid sequence (SEQ ID NO:22), or the complement thereof; and
ii) an isolated polynucleotide which comprises nucleotide sequence encoding FLU_T2_NA_3 amino acid sequence (SEQ ID NO:16), or the complement thereof.

According to the invention there is also provided a pharmaceutical composition which comprises:
i) an isolated polynucleotide which comprises nucleotide sequence encoding FLU_T2_HA_3_I3 amino acid sequence (SEQ ID NO:22), or the complement thereof; and
ii) an isolated polynucleotide which comprises nucleotide sequence encoding FLU_T2_M2_1 amino acid sequence (SEQ ID NO:14), or the complement thereof.

According to the invention there is also provided a pharmaceutical composition which comprises:
i) an isolated polynucleotide which comprises nucleotide sequence encoding FLU_T2_NA_3 amino acid sequence (SEQ ID NO:16), or the complement thereof; and
ii) an isolated polynucleotide which comprises nucleotide sequence encoding FLU_T2_M2_1 amino acid sequence (SEQ ID NO:14), or the complement thereof.

According to the invention there is also provided a pharmaceutical composition which comprises:
i) an isolated polynucleotide which comprises nucleotide sequence encoding FLU_T2_HA_3_I3 amino acid sequence (SEQ ID NO:22), or the complement thereof;
ii) an isolated polynucleotide which comprises nucleotide sequence encoding FLU_T2_NA_3 amino acid sequence (SEQ ID NO:16), or the complement thereof; and
iii) an isolated polynucleotide which comprises nucleotide sequence encoding FLU_T2_M2_1 amino acid sequence (SEQ ID NO:14), or the complement thereof.

Optionally a pharmaceutical composition of the invention comprises:
i) an isolated polynucleotide which comprises a nucleotide sequence encoding an amino acid sequence of SEQ ID NO:22 (FLU_T2_HA_3_I3 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:22, or the complement thereof; and/or
ii) an isolated polynucleotide which comprises a nucleotide sequence encoding an amino acid sequence of SEQ ID NO:16 (FLU_T2_NA_3 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:16, or the complement thereof; and/or
iii) an isolated polynucleotide which comprises a nucleotide sequence encoding an amino acid sequence of SEQ ID NO:14 (FLU_T2_M2_1 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:14, or the complement thereof.

Optionally the polynucleotide of (i) comprises a nucleotide sequence encoding an amino acid sequence of SEQ ID NO:22 (FLU_T2_HA_3_I3 amino acid sequence), or the complement thereof.

Optionally the polynucleotide of (ii) comprises a nucleotide sequence encoding an amino acid sequence of SEQ ID NO:16 (FLU_T2_NA_3 amino acid sequence), or the complement thereof.

Optionally the polynucleotide of (iii) comprises a nucleotide sequence encoding an amino acid sequence of SEQ ID NO:14 (FLU_T2_M2_1 amino acid sequence), or the complement thereof.

Optionally the nucleotide sequence encoding FLU_T2_HA_3_I3 amino acid sequence (SEQ ID NO:22), or the complement thereof, comprises the nucleotide sequence of SEQ ID NO:23, or the complement thereof.

Optionally the nucleotide sequence encoding FLU_T2_NA_3 amino acid sequence (SEQ ID NO:16), or the complement thereof, comprises the nucleotide sequence of SEQ ID NO:17, or the complement thereof.

Optionally the nucleotide sequence encoding FLU_T2_M2_1 amino acid sequence (SEQ ID NO:14), or the complement thereof, comprises the nucleotide sequence of SEQ ID NO:15, or the complement thereof.

Optionally a pharmaceutical composition of the invention comprises:
i) an isolated polynucleotide which comprises nucleotide sequence encoding FLU_T2_HA_4 amino acid sequence (SEQ ID NO:68), or the complement thereof; and
ii) an isolated polynucleotide which comprises nucleotide sequence encoding FLU_T2_NA_3 amino acid sequence (SEQ ID NO:16), or the complement thereof.

Optionally a pharmaceutical composition of the invention comprises:
i) an isolated polynucleotide which comprises nucleotide sequence encoding FLU_T2_HA_4 amino acid sequence (SEQ ID NO:68), or the complement thereof; and
ii) an isolated polynucleotide which comprises nucleotide sequence encoding FLU_T2_M2_1 amino acid sequence (SEQ ID NO:14), or the complement thereof.

Optionally a pharmaceutical composition of the invention comprises:
i) an isolated polynucleotide which comprises nucleotide sequence encoding FLU_T2_NA_3 amino acid sequence (SEQ ID NO:16), or the complement thereof; and
ii) an isolated polynucleotide which comprises nucleotide sequence encoding FLU_T2_M2_1 amino acid sequence (SEQ ID NO:14), or the complement thereof.

Optionally a pharmaceutical composition of the invention comprises:
i) an isolated polynucleotide which comprises nucleotide sequence encoding FLU_T2_HA_4 amino acid sequence (SEQ ID NO:68), or the complement thereof;
ii) an isolated polynucleotide which comprises nucleotide sequence encoding FLU_T2_NA_3 amino acid sequence (SEQ ID NO:16), or the complement thereof; and
iii) an isolated polynucleotide which comprises nucleotide sequence encoding FLU_T2_M2_1 amino acid sequence (SEQ ID NO:14), or the complement thereof.

Optionally the nucleotide sequence encoding FLU_T2_HA_4 amino acid sequence (SEQ ID NO:68), or the complement thereof, comprises the nucleotide sequence of SEQ ID NO:69, or the complement thereof.

Optionally the nucleotide sequence encoding FLU_T2_NA_3 amino acid sequence (SEQ ID NO:16), or the complement thereof, comprises the nucleotide sequence of SEQ ID NO:17, or the complement thereof.

Optionally the nucleotide sequence encoding FLU_T2_M2_1 amino acid sequence (SEQ ID NO:14), or the complement thereof, comprises the nucleotide sequence of SEQ ID NO:15, or the complement thereof.

Optionally each polynucleotide comprises a DNA molecule.

Optionally each polynucleotide comprises a messenger RNA (mRNA) molecule.

According to the invention there is also provided a pharmaceutical composition comprising a vector of the invention, and a pharmaceutically acceptable carrier, excipient, or diluent.

Optionally a pharmaceutical composition of the invention further comprises an adjuvant for enhancing an immune response in a subject to the polypeptide, or to a polypeptide encoded by the nucleic acid, of the composition.

Each different nucleic acid molecule of a pharmaceutical composition of the invention may be provided as part of a separate vector.

According to the invention there is also provided a pharmaceutical composition comprising a vector of the invention, and a pharmaceutically acceptable carrier, excipient, or diluent.

Optionally a pharmaceutical composition of the invention further comprises an adjuvant for enhancing an immune response in a subject to the polypeptides, or to polypeptides encoded by the nucleic acids, of the composition.

According to the invention there is also provided a pharmaceutical composition comprising a vector of the invention, and a pharmaceutically acceptable carrier, excipient, or diluent.

### Combined Preparations

The term “combined preparation” as used herein refers to a “kit of parts” in the sense that the combination components (i) and (ii), or (i), (ii) and (iii), as defined herein, can be dosed independently or by use of different fixed combinations with distinguished amounts of the combination components (i) and (ii), or (i), (ii) and (iii). The components can be administered simultaneously or one after the other. If the components are administered one after the other, preferably the time interval between administration is chosen such that the therapeutic effect of the combined use of the components is greater than the effect which would be obtained by use of only any one of the combination components (i) and (ii), or (i), (ii) and (iii).

The components of the combined preparation may be present in one combined unit dosage form, or as a first unit dosage form of component (i) and a separate, second unit dosage form of component (ii), or as a first unit dosage form of component (i), a separate, second unit dosage form of component (ii), and a separate, third unit dosage form of component (iii). The ratio of the total amounts of the combination component (i) to the combination component (ii), or of the combination component (i) to the combination component (ii) and to the combination component (iii) to be administered in the combined preparation can be varied, for example in order to cope with the needs of a patient sub-population to be treated, or the needs of the single patient, which can be due, for example, to the particular disease, age, sex, or body weight of the patient.

Preferably, there is at least one beneficial effect, for example an enhancing of the effect of the component (i), or an enhancing of the effect of the component (ii), or a mutual enhancing of the effect of the combination components (i) and (ii), or an enhancing of the effect of the component (i), or an enhancing of the effect of the component (ii), or an enhancing of the effect of the component (iii), or a mutual enhancing of the effect of the combination components (i), (ii), and (iii), for example a more than additive effect, additional advantageous effects, fewer side effects, less toxicity, or a combined therapeutic effect compared with an effective dosage of one or both of the combination components (i) and (ii), or (i), (ii), and (iii), and very preferably a synergism of the combination components (i) and (ii), or (i), (ii), and (iii).

A combined preparation of the invention may be provided as a pharmaceutical combined preparation for administration to a mammal, preferably a human. The component (i) may optionally be provided together with a pharmaceutically acceptable carrier, excipient, or diluent, and/or the component (ii) may optionally be provided together with a pharmaceutically acceptable carrier, excipient, or diluent, or the component (i) may optionally be provided together with a pharmaceutically acceptable carrier, excipient, or diluent, and/or the component (ii) may optionally be provided together with a pharmaceutically acceptable carrier, excipient, or diluent and/or the component (iii) may optionally be provided together with a pharmaceutically acceptable carrier, excipient, or diluent.

A combined preparation of the invention may include polypeptides of the invention in any suitable combination (for example, HA and/or M2 and/or neuraminidase embodiments of the invention, H5 and/or M2 and/or neuraminidase embodiments of the invention, H1 and/or M2 and/or neuraminidase embodiments of the invention, or FLU_T2_HA_3_I3 and/or Flu_T2_NA_3 and/or Flu_T2_M2_1 embodiments of the invention, or FLU_T2_HA_4 and/or Flu_T2_NA_3 and/or Flu_T2_M2_1 embodiments of the invention).

Optionally, one embodiment of each different category of embodiment is used in combination. For example, an HA embodiment (H5 or H1), and/or an M2 embodiment and/or a neuraminidase embodiment.

According to the invention there is provided a combined preparation, which comprises:
i) a polypeptide which comprises an amino acid sequence of SEQ ID NO:7 or 8, or a polypeptide which comprises an amino acid sequence of SEQ ID NO:10 or 11, or a polypeptide which comprises an amino acid sequence of SEQ ID NO:1 or 3 (examples of H5 embodiments); and/or
ii) a polypeptide which comprises an amino acid sequence of SEQ ID NO:14 (examples of M2 embodiments); and/or
iii) a polypeptide which comprises an amino acid sequence of SEQ ID NO:16, a polypeptide which comprises an amino acid sequence of SEQ ID NO:18 (examples of neuraminidase embodiments).

According to the invention there is provided a combined preparation, which comprises:
i) a polypeptide which comprises an amino acid sequence of SEQ ID NO:7 or 8, or a polypeptide which comprises an amino acid sequence of SEQ ID NO:10 or 11, or a polypeptide which comprises an amino acid sequence of SEQ ID NO:1 or 3, or a polypeptide which comprises an amino acid sequence of SEQ ID NO:27 or 29, or a polypeptide which comprises an amino acid sequence of SEQ ID NO:35 or 37, or a polypeptide which comprises an amino acid sequence of SEQ ID NO:43 or 45 (examples of H5 embodiments); and/or
ii) a polypeptide which comprises an amino acid sequence of SEQ ID NO:14 (examples of M2 embodiments); and/or
iii) a polypeptide which comprises an amino acid sequence of SEQ ID NO:16, a polypeptide which comprises an amino acid sequence of SEQ ID NO:18 (examples of neuraminidase embodiments).

According to the invention there is also provided a combined preparation, which comprises:
i) a polypeptide which comprises an amino acid sequence of SEQ ID NO:22, or a polypeptide which comprises an amino acid sequence of SEQ ID NO:68 (examples of H1 embodiments); and/or
ii) a polypeptide which comprises an amino acid sequence of SEQ ID NO:14 (examples of M2 embodiments); and/or
iii) a polypeptide which comprises an amino acid sequence of SEQ ID NO:16, or a polypeptide which comprises an amino acid sequence of SEQ ID NO:18 (examples of neuraminidase embodiments).

Optionally a combined preparation of the invention comprises:
i) a polypeptide which comprises an amino acid sequence of SEQ ID NO:22 (FLU_T2_HA_3_I3 amino acid sequence); and/or
ii) a polypeptide which comprises an amino acid sequence of SEQ ID NO:14 (FLU_T2_M2_1 amino acid sequence); and/or
iii) a polypeptide which comprises an amino acid sequence of SEQ ID NO:16 (FLU_T2_NA_3 amino acid sequence).

Optionally a combined preparation of the invention comprises:
i) a polypeptide which comprises an amino acid sequence of SEQ ID NO:22 (FLU_T2_HA_3_I3 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:22; and/or
ii) a polypeptide which comprises an amino acid sequence of SEQ ID NO:14 (FLU_T2_M2_1 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:14; and/or
iii) a polypeptide which comprises an amino acid sequence of SEQ ID NO:16 (FLU_T2_NA_3 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:16.

Optionally the polypeptide of (i) comprises an amino acid sequence of SEQ ID NO:22 (FLU_T2_HA_3_I3 amino acid sequence); Optionally the polypeptide of (ii) comprises an amino acid sequence of SEQ ID NO:14 (FLU_T2_M2_1 amino acid sequence).

Optionally the polypeptide of (iii) comprises an amino acid sequence of SEQ ID NO:16 (FLU_T2_NA_3 amino acid sequence).

A combined preparation of the invention may include nucleic acid molecules of the invention in any suitable combination (for example, HA and/or M2 and/or neuraminidase embodiments of the invention, H5 and/or M2 and/or neuraminidase embodiments of the invention, H1 and/or M2 and/or neuraminidase embodiments of the invention, or FLU_T2_HA_3_I3 and/or Flu_T2_NA_3 and/or Flu_T2_M2_1 embodiments of the invention, or FLU_T2_HA_4 and/or Flu_T2_NA_3 and/or Flu_T2_M2_1 embodiments of the invention).

Optionally, one embodiment of each different category of embodiment is used in combination. For example, an HA embodiment (H5 or H1), and/or an M2 embodiment and/or a neuraminidase embodiment.

Optionally a combined preparation of the invention comprises:
i) a nucleic acid molecule encoding a polypeptide which comprises an amino acid sequence of SEQ ID NO:7 or 8, or a nucleic acid molecule encoding a polypeptide which comprises an amino acid sequence of SEQ ID NO:10 or 11, or a nucleic acid molecule encoding a polypeptide which comprises an amino acid sequence of SEQ ID NO:1 or 3 (examples of H5 embodiments); and/or
ii) a nucleic acid molecule encoding a polypeptide which comprises an amino acid sequence of SEQ ID NO:14 (examples of M2 embodiments); and/or
iii) a nucleic acid molecule encoding a polypeptide which comprises an amino acid sequence of SEQ ID NO:16, or a nucleic acid molecule encoding a polypeptide which comprises an amino acid sequence of SEQ ID NO:18 (examples of neuraminidase embodiments).

Optionally a combined preparation of the invention comprises:
i) a nucleic acid molecule encoding a polypeptide which comprises an amino acid sequence of SEQ ID NO:7 or 8, or a nucleic acid molecule encoding a polypeptide which comprises an amino acid sequence of SEQ ID NO:10 or 11, or a nucleic acid molecule encoding a polypeptide which comprises an amino acid sequence of SEQ ID NO:1 or 3, or a nucleic acid molecule encoding a polypeptide which comprises an amino acid sequence of SEQ ID NO:27 or 29, or a nucleic acid molecule encoding a polypeptide which comprises an amino acid sequence of SEQ ID NO:35 or 37, or a nucleic acid molecule encoding a polypeptide which comprises an amino acid sequence of SEQ ID NO:43 or 45 (examples of H5 embodiments); and/or
ii) a nucleic acid molecule encoding a polypeptide which comprises an amino acid sequence of SEQ ID NO:14 (examples of M2 embodiments); and/or
iii) a nucleic acid molecule encoding a polypeptide which comprises an amino acid sequence of SEQ ID NO:16, or a nucleic acid molecule encoding a polypeptide which comprises an amino acid sequence of SEQ ID NO:18 (examples of neuraminidase embodiments).

Optionally a combined preparation of the invention comprises:
i) a nucleic acid molecule encoding a polypeptide which comprises an amino acid sequence of SEQ ID NO:22, or a nucleic acid molecule encoding a polypeptide which comprises an amino acid sequence of SEQ ID NO:68 (examples of H1 embodiments); and/or
ii) a nucleic acid molecule encoding a polypeptide which comprises an amino acid sequence of SEQ ID NO:14 (examples of M2 embodiments); and/or
iii) a nucleic acid molecule encoding a polypeptide which comprises an amino acid sequence of SEQ ID NO:16, or a nucleic acid molecule encoding a polypeptide which comprises an amino acid sequence of SEQ ID NO:18 (examples of neuraminidase embodiments).

According to the invention there is also provided a combined preparation which comprises:
i) an isolated polynucleotide which comprises nucleotide sequence encoding FLU_T2_HA_3_I3 amino acid sequence (SEQ ID NO:22), or the complement thereof; and/or
ii) an isolated polynucleotide which comprises nucleotide sequence encoding FLU_T2_NA_3 amino acid sequence (SEQ ID NO:16), or the complement thereof; and/or
iii) an isolated polynucleotide which comprises nucleotide sequence encoding FLU_T2_M2_1 amino acid sequence (SEQ ID NO:14), or the complement thereof.

According to the invention there is also provided a combined preparation which comprises:
i) an isolated polynucleotide which comprises a nucleotide sequence encoding an amino acid sequence of SEQ ID NO:22 (FLU_T2_HA_3_I3 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:22, or the complement thereof; and/or
ii) an isolated polynucleotide which comprises a nucleotide sequence encoding an amino acid sequence of SEQ ID NO:16 (FLU_T2_NA_3 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:16, or the complement thereof; and/or
iii) an isolated polynucleotide which comprises a nucleotide sequence encoding an amino acid sequence of SEQ ID NO:14 (FLU_T2_M2_1 amino acid sequence), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:14, or the complement thereof.

Optionally the polynucleotide of (i) comprises a nucleotide sequence encoding an amino acid sequence of SEQ ID NO:22 (FLU_T2_HA_3_I3 amino acid sequence), or the complement thereof.

Optionally the polynucleotide of (ii) comprises a nucleotide sequence encoding an amino acid sequence of SEQ ID NO:16 (FLU_T2_NA_3 amino acid sequence), or the complement thereof.

Optionally the polynucleotide of (iii) comprises a nucleotide sequence encoding an amino acid sequence of SEQ ID NO:14 (FLU_T2_M2_1 amino acid sequence), or the complement thereof.

According to the invention there is also provided a combined preparation which comprises:
i) an isolated polynucleotide which comprises nucleotide sequence encoding FLU_T2_HA_3_I3 amino acid sequence (SEQ ID NO:22), or the complement thereof; and
ii) an isolated polynucleotide which comprises nucleotide sequence encoding FLU_T2_NA_3 amino acid sequence (SEQ ID NO:16), or the complement thereof.

According to the invention there is also provided a combined preparation which comprises:
i) an isolated polynucleotide which comprises nucleotide sequence encoding FLU_T2_HA_3_I3 amino acid sequence (SEQ ID NO:22), or the complement thereof; and
ii) an isolated polynucleotide which comprises nucleotide sequence encoding FLU_T2_M2_1 amino acid sequence (SEQ ID NO:14), or the complement thereof.

According to the invention there is also provided a combined preparation which comprises:
i) an isolated polynucleotide which comprises nucleotide sequence encoding FLU_T2_NA_3 amino acid sequence (SEQ ID NO:16), or the complement thereof; and
ii) an isolated polynucleotide which comprises nucleotide sequence encoding FLU_T2_M2_1 amino acid sequence (SEQ ID NO:14), or the complement thereof.

According to the invention there is also provided a combined preparation which comprises:
i) an isolated polynucleotide which comprises nucleotide sequence encoding FLU_T2_HA_3_I3 amino acid sequence (SEQ ID NO:22), or the complement thereof;
ii) an isolated polynucleotide which comprises nucleotide sequence encoding FLU_T2_NA_3 amino acid sequence (SEQ ID NO:16), or the complement thereof; and
iii) an isolated polynucleotide which comprises nucleotide sequence encoding FLU_T2_M2_1 amino acid sequence (SEQ ID NO:14), or the complement thereof.

Optionally the nucleotide sequence encoding FLU_T2_HA_3_I3 amino acid sequence (SEQ ID NO:22), or the complement thereof, comprises the nucleotide sequence of SEQ ID NO:23, or the complement thereof.

Optionally the nucleotide sequence encoding FLU_T2_NA_3 amino acid sequence (SEQ ID NO:16), or the complement thereof, comprises the nucleotide sequence of SEQ ID NO:17, or the complement thereof.

Optionally the nucleotide sequence encoding FLU_T2_M2_1 amino acid sequence (SEQ ID NO:14), or the complement thereof, comprises the nucleotide sequence of SEQ ID NO:15, or the complement thereof.

According to the invention there is also provided a combined preparation which comprises:
i) an isolated polynucleotide which comprises nucleotide sequence encoding FLU_T2_HA_4 amino acid sequence (SEQ ID NO:68), or the complement thereof; and
ii) an isolated polynucleotide which comprises nucleotide sequence encoding FLU_T2_NA_3 amino acid sequence (SEQ ID NO:16), or the complement thereof.

According to the invention there is also provided a combined preparation which comprises:
i) an isolated polynucleotide which comprises nucleotide sequence encoding FLU_T2_HA_4 amino acid sequence (SEQ ID NO:68), or the complement thereof; and
ii) an isolated polynucleotide which comprises nucleotide sequence encoding FLU_T2_M2_1 amino acid sequence (SEQ ID NO:14), or the complement thereof.

According to the invention there is also provided a combined preparation which comprises:
i) an isolated polynucleotide which comprises nucleotide sequence encoding FLU_T2_NA_3 amino acid sequence (SEQ ID NO:16), or the complement thereof; and
ii) an isolated polynucleotide which comprises nucleotide sequence encoding FLU_T2_M2_1 amino acid sequence (SEQ ID NO:14), or the complement thereof.

According to the invention there is also provided a combined preparation which comprises:
i) an isolated polynucleotide which comprises nucleotide sequence encoding FLU_T2_HA_4 amino acid sequence (SEQ ID NO:68), or the complement thereof;
ii) an isolated polynucleotide which comprises nucleotide sequence encoding FLU_T2_NA_3 amino acid sequence (SEQ ID NO:16), or the complement thereof; and
iii) an isolated polynucleotide which comprises nucleotide sequence encoding FLU_T2_M2_1 amino acid sequence (SEQ ID NO:14), or the complement thereof.

Optionally the nucleotide sequence encoding FLU_T2_HA_4 amino acid sequence (SEQ ID NO:68), or the complement thereof, comprises the nucleotide sequence of SEQ ID NO:69, or the complement thereof.

Optionally the nucleotide sequence encoding FLU_T2_NA_3 amino acid sequence (SEQ ID NO:16), or the complement thereof, comprises the nucleotide sequence of SEQ ID NO:17, or the complement thereof.

Optionally the nucleotide sequence encoding FLU_T2_M2_1 amino acid sequence (SEQ ID NO:14), or the complement thereof, comprises the nucleotide sequence of SEQ ID NO:15, or the complement thereof.

Optionally each polynucleotide comprises a DNA molecule.

Optionally each polynucleotide comprises a messenger RNA (mRNA) molecule.

Each different nucleic acid molecule of a combined preparation of the invention may be provided as part of a separate vector.

Optionally a combined preparation of the invention further comprises an adjuvant for enhancing an immune response in a subject to the polypeptides, or to the polypeptides encoded by the nucleic acids, of the combined preparation.

### Strings

Embodiments of the invention in which different polypeptides of the invention are encoded as part of the same polynucleotide (or nucleic acid), or are provided in the same polypeptide (i.e. as “strings” of different subunits, for example, HA and/or M2 and/or neuraminidase embodiments of the invention, H5 and/or M2 and/or neuraminidase embodiments of the invention, H1 and/or M2 and/or neuraminidase embodiments of the invention, or FLU_T2_HA_3_I3 and/or Flu_T2_NA_3 and/or Flu_T2_M2_1 embodiments of the invention, or FLU_T2_HA_4 and/or Flu_T2_NA_3 and/or Flu_T2_M2_1 embodiments of the invention), are particularly advantageous since use of such a “string” as part of a vaccine requires testing only of the single product containing the “string” for safety and efficacy, rather than testing each different subunit individually. This dramatically reduces the time and cost of developing the vaccine compared with individual subunits. In some embodiments, a combination of different strings (polynucleotide and/or polypeptide), or a combination of one or more strings and one or more single subunits (polypeptide or encoded subunit) may be used.

Optionally, one embodiment of each different category of embodiment is used in combination. For example, an HA embodiment (H5 or H1), and/or an M2 embodiment and/or a neuraminidase embodiment.

Strategies for multigene co-expression include introduction of multiple vectors, use of multiple promoters in a single vector, fusion proteins, proteolytic cleavage sites between genes, internal ribosome entry sites (IRES), and “self-cleaving” 2A peptides. Multicistronic vectors based on IRES nucleotide sequence and self-cleaving 2A peptides are reviewed in Shaimardanova et al. (Pharmaceutics 2019, 11, 580; doi:10.3390/pharmaceutics11110580).

In one embodiment of the invention, known as panH1N1 (described below in Example 15 below), a polypeptide comprising a string of the following subunits joined by self-cleaving 2A peptides is provided:

FLU_T2_HA_3_I3 (amino acid SEQ ID NO:22), FLU_T2_NA_3 (amino acid SEQ ID NO:16), and FLU_T2_M2_1 (amino acid SEQ ID NO:14).

The amino acid sequence of panH1N1 is provided as SEQ ID NO:63.

According to the invention there is also provided an isolated polypeptide which comprises an amino acid sequence of SEQ ID NO:63.

According to the invention there is provided an isolated polypeptide which comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:63.

### Vaccines

Vaccines of the invention may be provided, for example, as nucleic acid vaccines, either as separate polynucleotides, each encoding a different subunit (HA and/or M2 and/or neuraminidase embodiment of the invention, for example a H5 and/or M2 and/or neuraminidase embodiment of the invention, a H1 and/or M2 and/or neuraminidase embodiment of the invention, or a FLU_T2_HA_3_I3 and/or Flu_T2_NA_3 and/or Flu_T2_M2_1 embodiment of the invention, or a FLU_T2_HA_4 and/or Flu_T2_NA_3 and/or Flu_T2_M2_1 embodiment of the invention) (for administration together or separately) or pieced together in a string as a single polynucleotide encoding all of the subunits. The separate polynucleotides may be administered as a mixture together (for example, as a pharmaceutical composition comprising the separate polynucleotides), or co-administered or administered sequentially in any order (in which case, the separate polynucleotides may be provided as a combined preparation for co-administration or sequential administration). Nucleic acid vaccines may be provided as DNA, RNA, or mRNA vaccines. Production and application of multicistronic constructs (for example, where the subunits are provided in a string as a single polynucleotide) is reviewed by Shaimardanova et al. (Pharmaceutics 2019, 11, 580; doi:10.3390/pharmaceutics11110580).

Vaccine constructs of the invention may also be provided, for example, either as separate polypeptides, each comprising a different subunit (for example, HA, M2, or neuraminidase embodiments of the invention, H5, M2, or neuraminidase embodiments of the invention, H1, M2, or neuraminidase embodiments of the invention, or FLU_T2_HA_3_I3, or Flu_T2_NA_3, or Flu_T2_M2_1 embodiments of the invention, or FLU_T2_HA_4, or Flu_T2_NA 3, or Flu_T2_M2_1 embodiments of the invention) or pieced together in a string as a single polypeptide comprising all of the subunits (for example, HA and M2 and neuraminidase embodiments of the invention, H5 and M2 and neuraminidase embodiments of the invention, H1 and M2 and neuraminidase embodiments of the invention, or FLU_T2_HA_3_I3 and Flu_T2_NA_3 and Flu_T2_M2_1 embodiments of the invention, or FLU_T2_HA_4 and Flu_T2_NA_3 and Flu_T2_M2_1 embodiments of the invention). The separate polypeptides may be administered as a mixture together (for example, as a pharmaceutical composition comprising the separate polypeptides), or co-administered or administered sequentially in any order (in which case, the separate polypeptides may be provided as a combined preparation for co-administration or sequential administration).

Optionally, one embodiment of each different category of embodiment is used in combination. For example, an HA embodiment (H5 or H1), and/or an M2 embodiment and/or a neuraminidase embodiment.

Strategies for multigene co-expression include introduction of multiple vectors, use of multiple promoters in a single vector, fusion proteins, proteolytic cleavage sites between genes, internal ribosome entry sites (IRES), and “self-cleaving” 2A peptides. Multicistronic vectors based on IRES nucleotide sequence and self-cleaving 2A peptides are reviewed in Shaimardanova et al. (Pharmaceutics 2019, 11, 580; doi:10.3390/pharmaceutics11110580).

In one embodiment of the invention (described below in Example 15), a nucleic acid molecule with a nucleotide sequence of SEQ ID NO:25 encoding a string of the following subunits joined by self-cleaving 2A peptides (known as panH1N1) is provided:

FLU_T2_HA_3_I3 (amino acid SEQ ID NO:22), FLU_T2_NA_3 (amino acid SEQ ID NO:16), and FLU_T2_M2_1 (amino acid SEQ ID NO:14).

There is also provided according to the invention an isolated polynucleotide comprising nucleotide sequence encoding FLU_T2_HA_3_I3 (amino acid SEQ ID NO:22), nucleotide sequence encoding FLU_T2_NA_3 (amino acid SEQ ID NO:16), and nucleotide sequence encoding FLU_T2_M2_1 (amino acid SEQ ID NO:14).

There is also provided according to the invention an isolated polynucleotide comprising nucleotide sequence of SEQ ID NO:23, nucleotide sequence of SEQ ID NO:17, and nucleotide sequence of SEQ ID NO:15.

According to the invention there is provided an isolated nucleic acid molecule, which comprises a nucleotide sequence encoding a polypeptide which comprises an amino acid sequence of SEQ ID NO:63, or the complement thereof.

There is also provided according to the invention an isolated nucleic acid molecule which comprises a nucleotide sequence of SEQ ID NO:25, or the complement thereof.

According to the invention there is also provided an isolated nucleic acid molecule, which comprises a nucleotide sequence encoding a polypeptide which comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:63, or the complement thereof.

There is also provided according to the invention an isolated nucleic acid molecule which comprises a nucleotide sequence of SEQ ID NO:25, or a nucleotide sequence which has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity along its entire length with the nucleotide sequence of SEQ ID NO:25, which encodes a polypeptide which comprises an amino acid sequence of SEQ ID NO:63, or the complement thereof.

There is also provided according to the invention an isolated nucleic acid molecule which comprises a nucleotide sequence encoding a polypeptide which comprises an amino acid sequence of SEQ ID NO:63, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:63, wherein the nucleic acid molecule comprises a nucleotide sequence of SEQ ID NO:25, or a nucleotide sequence which has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity along its entire length with the nucleotide sequence of SEQ ID NO:25, or the complement thereof.

There is also provided according to the invention an isolated nucleic acid molecule which comprises a nucleotide sequence of SEQ ID NO:25, or a nucleotide sequence which has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity along its entire length with the nucleotide sequence of SEQ ID NO:25, or the complement thereof.

Optionally, an isolated nucleic acid molecule of the invention comprises a DNA molecule, an RNA molecule, or an mRNA molecule.

Where mRNA vaccines are used in accordance with the invention, it is preferred that each designed subunit of a string of the invention is encoded as part of a separate mRNA vaccine vector.

### Methods of Treatment and Uses

There is also provided according to the invention a method of inducing an immune response to an influenza virus in a subject, which comprises administering to the subject an effective amount of a polypeptide of the invention, a nucleic acid of the invention, a vector of the invention, a pharmaceutical composition, or a combined preparation, of the invention.

There is also provided according to the invention a method of immunising a subject against an influenza virus, which comprises administering to the subject an effective amount of a polypeptide of the invention, a nucleic acid of the invention, a vector of the invention, a pharmaceutical composition, or a combined preparation, of the invention.

An effective amount is an amount to produce an antigen-specific immune response in a subject.

There is further provided according to the invention a polypeptide of the invention, a nucleic acid of the invention, a vector of the invention, a pharmaceutical composition of the invention, or a combined preparation, for use as a medicament.

There is further provided according to the invention a polypeptide of the invention, a nucleic acid of the invention, a vector of the invention, a pharmaceutical composition of the invention, or a combined preparation, for use in the prevention, treatment, or amelioration of an influenza viral infection.

There is also provided according to the invention use of a polypeptide of the invention, a nucleic acid of the invention, a vector of the invention, or a pharmaceutical composition of the invention, or a combined preparation, in the manufacture of a medicament for the prevention, treatment, or amelioration of an influenza viral infection.

### Administration

Any suitable route of administration may be used. Methods of administration include, but are not limited to, intradermal, intramuscular, intraperitoneal, parenteral, intravenous, subcutaneous, vaginal, rectal, intranasal, inhalation or oral. Parenteral administration, such as subcutaneous, intravenous or intramuscular administration, is generally achieved by injection. Injectables can be prepared in conventional forms, either as liquid solutions or suspensions, solid forms suitable for solution or suspension in liquid prior to injection, or as emulsions. Injection solutions and suspensions can be prepared from sterile powders, granules, and tablets of the kind previously described. Administration can be systemic or local. Routes for systemic administration in general include, for example, transdermal, oral, parenteral routes, including subcutaneous, intravenous, intramuscular, intraarterial, intradermal and intraperitoneal injections and/or intranasal administration routes. Routes for local administration in general include, for example, topical administration mutes but also intradermal, transdermal, subcutaneous, or intramuscular injections or intralesional, intracranial, intrapulmonal, intracardial, and sublingual injections.

Compositions may be administered in any suitable manner, such as with pharmaceutically acceptable carriers. Pharmaceutically acceptable carriers are determined in part by the particular composition being administered, as well as by the particular method used to administer the composition. Preparations for parenteral administration include sterile aqueous or nonaqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Administration can be accomplished by single or multiple doses. The dose administered to a subject in the context of the present disclosure should be sufficient to induce a beneficial therapeutic response in a subject over time, or to inhibit or prevent infection. The dose required will vary from subject to subject depending on the species, age, weight and general condition of the subject, the severity of the infection being treated, the particular composition being used and its mode of administration. An appropriate dose can be determined by one of ordinary skill in the art using only routine experimentation.

The present disclosure includes methods comprising administering an RNA vaccine, an mRNA vaccine, or a DNA vaccine to a subject in need thereof. The exact amount required will vary from subject to subject, depending on the species, age, and general condition of the subject, the severity of the disease, the particular composition, its mode of administration, its mode of activity, and the like.

The RNA or DNA is typically formulated in dosage unit form for ease of administration and uniformity of dosage. It will be understood, however, that the total daily usage of the RNA may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of the RNA or DNA, as provided herein, may be as low as 20 pg, administered for example as a single dose or as two 10 pg doses. In some embodiments, the effective amount is a total dose of 20 μg-300 μg or 25 μg-300 μg. For example, the effective amount may be a total dose of 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg, 200 μg, 250 μg, or 300 μg. In some embodiments, the effective amount is a total dose of 20 μg. In some embodiments, the effective amount is a total dose of 25 μg. In some embodiments, the effective amount is a total dose of 50 μg. In some embodiments, the effective amount is a total dose of 75 μg. In some embodiments, the effective amount is a total dose of 100 μg. In some embodiments, the effective amount is a total dose of 150 μg. In some embodiments, the effective amount is a total dose of 200 μg. In some embodiments, the effective amount is a total dose of 250 μg. In some embodiments, the effective amount is a total dose of 300 μg.

The RNA or DNA described herein can be formulated into a dosage form described herein, such as an intranasal, intratracheal, or injectable (e.g., intravenous, intraocular, intravitreal, intramuscular, intradermal, intracardiac, intraperitoneal, and subcutaneous).

Optionally, an RNA (e.g., mRNA) or DNA vaccine is formulated in an effective amount to produce an antigen specific immune response in a subject.

In some embodiments, the effective amount is a total dose of 25 μg to 1000 μg, or 50 μg to 1000 μg. In some embodiments, the effective amount is a total dose of 100 μg. In some embodiments, the effective amount is a dose of 25 μg administered to the subject a total of two times. In some embodiments, the effective amount is a dose of 100 μg administered to the subject a total of two times. In some embodiments, the effective amount is a dose of 400 μg administered to the subject a total of two times. In some embodiments, the effective amount is a dose of 500 μg administered to the subject a total of two times.

Optionally a dosage of between 10 μg/kg and 400 μg/kg of the nucleic acid vaccine is administered to the subject. In some embodiments the dosage of the RNA or DNA polynucleotide (or nucleic acid) is 1-5 μg, 5-10 μg, 10-15 μg, 15-20 μg, 10-25 μg, 20-25 μg, 20-50 μg, 30-50 μg, 40-50 μg, 40-60 μg, 60-80 μg, 60-100 μg, 50-100 μg, 80-120 μg, 40-120 μg, 40-150 μg, 50-150 μg, 50-200 μg, 80-200 μg, 100-200 μg, 120-250 μg, 150-250 μg, 180-280 μg, 200-300 μg, 50-300 μg, 80-300 μg, 100-300 μg, 40-300 μg, 50-350 μg, 100-350 μg, 200-350 μg, 300-350 μg, 320-400 μg, 40-380 μg, 40-100 μg, 100-400 μg, 200-400 μg, or 300-400 μg per dose. In some embodiments, the nucleic acid vaccine is administered to the subject by intradermal or intramuscular injection. In some embodiments, the nucleic acid vaccine is administered to the subject on day zero. In some embodiments, a second dose of the nucleic acid vaccine is administered to the subject on day twenty one.

### Pharmaceutically Acceptable Carriers

Pharmaceutically acceptable carriers include, but are not limited to, saline, buffered saline, dextrose, water, glycerol, ethanol, and combinations thereof. The carrier and composition can be sterile, and the formulation suits the mode of administration. The composition can also contain minor amounts of wetting or emulsifying agents, or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, and magnesium carbonate. Any of the common pharmaceutical carriers, such as sterile saline solution or sesame oil, can be used. The medium can also contain conventional pharmaceutical adjunct materials such as, for example, pharmaceutically acceptable salts to adjust the osmotic pressure, buffers, preservatives and the like. Other media that can be used with the compositions and methods provided herein are normal saline and sesame oil.

In some embodiments, the compositions comprise a pharmaceutically acceptable carrier and/or an adjuvant. For example, the adjuvant can be alum, Freund's complete adjuvant, a biological adjuvant or immunostimulatory oligonucleotides (such as CpG oligonucleotides). The pharmaceutically acceptable carriers (vehicles) useful in this disclosure are conventional. Remington's Pharmaceutical Sciences, by E. W. Martin, Mack Publishing Co., Easton, PA, 15^{th} Edition (1975), describes compositions and formulations suitable for pharmaceutical delivery of one or more therapeutic compositions, such as one or more influenza vaccines, and additional pharmaceutical agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Optionally a composition of the invention is administered intramuscularly.

Optionally the composition is administered intramuscularly, intradermally, subcutaneously by needle or by gene gun, or electroporation.

Aspects of the invention are defined in the following numbered paragraphs:

1. An isolated polypeptide comprising a haemagluttinin subtype 5 (H5) globular head domain, and optionally a haemagluttinin stem domain, with the following amino acid residues at positions 156, 157, 171, 172, and 205 of the head domain:
156: R;
157: P or S, preferably P;
171: D or N;
172: T or A, preferably T; and
205: K or R, preferably K

2. An isolated polypeptide according to paragraph 1, with the following amino acid residues at positions 156, 157, 171, 172, and 205 of the head domain:
156: R;
157: P;
171: D;
172: T; and
205: K

3. An isolated polypeptide according to paragraph 1 or 2, which comprises an amino acid sequence of SEQ ID NO:7 or 8, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:7 or 8 and which has the following amino acid residues at positions corresponding to positions 156, 157, 171, 172, and 205 of SEQ ID NO:7 or 8:
156: R;
157: P;
171: D;
172: T; and
205: K

4. An isolated polypeptide according to paragraph 1, with the following amino acid residues at positions 156, 157, 171, 172, and 205 of the head domain:
156: R;
157: P;
171: N;
172: T; and
205: K

5. An isolated polypeptide according to paragraph 1 or 4, which comprises an amino acid sequence of SEQ ID NO:10 or 11, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:10 or 11 and which has the following amino acid residues at positions corresponding to positions 156, 157, 171, 172, and 205 of SEQ ID NO:10 or 11:
156: R;
157: P;
171: N;
172: T; and
205: K

6. An isolated polypeptide according to paragraph 1, with the following amino acid residues at positions 156, 157, 171, 172, and 205 of the head domain:
156: R;
157: S;
171: N;
172: A; and
205: R

7. An isolated polypeptide according to paragraph 1 or 6, which comprises an amino acid sequence of SEQ ID NO:1 or 3, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:1 or 3 and which has the following amino acid residues at positions corresponding to positions 156, 157, 171, 172, and 205 of SEQ ID NO:1 or 3:
156: R;
157: S;
171: N;
172: A; and
205: R

8. An isolated polypeptide according to any preceding paragraph, with the following amino acid residues at positions 416 and 434 of the stem domain:
416: F; and
434: F

9. An isolated polypeptide which comprises the following amino acid sequence: R(P/S)SFFRNVWLIKKN(D/N)(T/A)YPTIKRSYNNTNQEDLLVLWGIHHPNDAAEQT(K/R) (SEQ ID NO:13), or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:13 and which has the following amino acid residues at positions corresponding to positions 1, 2, 16, 17, and 50 of SEQ ID NO:13:
1: R;
2: P or S, preferably P;
16: D or N;
17: T or A, preferably T; and
50: K or R, preferably K.

10. An isolated polypeptide according to paragraph 9, with the following amino acid residues at positions 1, 2, 16, 17, and 50 of the amino acid sequence, or at positions corresponding to positions 1, 2, 16, 17, and 50 of SEQ ID NO:13:
1: R;
2: P;
16: D;
17: T; and
50: K

11. An isolated polypeptide according to paragraph 9, with the following amino acid residues at positions 1, 2, 16, 17, and 50 of the amino acid sequence, or at positions corresponding to positions 1, 2, 16, 17, and 50 of SEQ ID NO:13:
1: R;
2: P;
16: N;
17: T; and
50: K

12. An isolated polypeptide according to paragraph 9, with the following amino acid residues at positions 1, 2, 16, 17, and 50 of the amino acid sequence, or at positions corresponding to positions 1, 2, 16, 17, and 50 of SEQ ID NO:13:
1: R;
2: S;
16: N;
17: A; and
50: R

13. An isolated polypeptide which comprises an amino acid sequence of any of SEQ ID NOs:5, 9, or 12, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of any of SEQ ID NO:5, 9, or 12 and which has the following amino acid residues at positions corresponding to positions 148 and 166 of SEQ ID NO:5, 9, or 12:
148: F; and
166: F

14. An isolated polypeptide which comprises an amino acid sequence of SEQ ID NO:14, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:14.

15. An isolated polypeptide which comprises an amino acid sequence of SEQ ID NO:16, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:16.

16. An isolated polypeptide which comprises an amino acid sequence of SEQ ID NO:18, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:18.

17. An isolated nucleic acid molecule encoding a polypeptide according to any of paragraphs 1 to 16, or an isolated nucleic acid molecule comprising a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with the nucleic acid molecule over its entire length, or the complement thereof.

18. An isolated nucleic acid molecule according to paragraph 17, comprising a nucleotide sequence of SEQ ID NO:2, 4, or 6, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:2, 4, or 6, over its entire length, or the complement thereof.

19. An isolated nucleic acid molecule according to paragraph 17, comprising a nucleotide sequence of SEQ ID NO:15, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:15, over its entire length, or the complement thereof.

20. An isolated nucleic acid molecule according to paragraph 17, comprising a nucleotide sequence of SEQ ID NO:17, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:17, over its entire length, or the complement thereof.

21. An isolated nucleic acid molecule according to paragraph 17, comprising a nucleotide sequence of SEQ ID NO:19, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO:19, over its entire length, or the complement thereof.

22. A vector comprising a nucleic acid molecule of any of paragraphs 17 to 21.

23. A vector according to paragraph 22, comprising a nucleic acid molecule encoding a polypeptide of any of paragraphs 1 to 12.

24. A vector according to paragraph 22 or 23, comprising a nucleic acid molecule encoding a polypeptide of paragraph 14.

25. A vector according to any of paragraphs 22 to 24, comprising a nucleic acid molecule encoding a polypeptide of paragraph 15 or 16.

26. A vector according to any of paragraphs 22 to 25, comprising a nucleic acid molecule encoding a polypeptide which comprises an amino acid sequence of SEQ ID NO:7 or 8.

27. A vector according to any of paragraphs 22 to 26, comprising a nucleic acid molecule encoding a polypeptide which comprises an amino acid sequence of SEQ ID NO:10 or 11.

28. A vector according to any of paragraphs 22 to 27, comprising a nucleic acid molecule encoding a polypeptide which comprises an amino acid sequence of SEQ ID NO:1 or 3.

29. A vector according to any of paragraphs 22 to 28, comprising a nucleic acid molecule encoding a polypeptide which comprises an amino acid sequence of SEQ ID NO:14.

30. A vector according to any of paragraphs 22 to 29, comprising a nucleic acid molecule encoding a polypeptide which comprises an amino acid sequence of SEQ ID NO:16.

31. A vector according to any of paragraphs 22 to 30, comprising a nucleic acid molecule encoding a polypeptide which comprises an amino acid sequence of SEQ ID NO:18.

32. A vector according to any of paragraphs 22 to 31, which further comprises a promoter operably linked to the, or each nucleic acid molecule.

33. A vector according to paragraph 32, wherein the, or each promoter is for expression of a polypeptide encoded by the nucleic acid in mammalian cells.

34. A vector according to paragraph 33, wherein the, or each promoter is for expression of a polypeptide encoded by the nucleic acid in yeast or insect cells.

35. A vector according to any of paragraphs 22 to 34, which is a vaccine vector.

36. A vector according to paragraph 35, which is a viral vaccine vector, a bacterial vaccine vector, an RNA vaccine vector, or a DNA vaccine vector.

37. An isolated cell comprising a vector of any of paragraphs 22 to 36.

38. A fusion protein comprising a polypeptide according to any of paragraphs 1 to 16.

39. A pharmaceutical composition comprising a polypeptide according to any of paragraphs 1 to 16, and a pharmaceutically acceptable carrier, excipient, or diluent.

40. A pharmaceutical composition according to paragraph 39, comprising a polypeptide of any of paragraphs 1 to 12.

41. A pharmaceutical composition according to paragraph 39 or 40, comprising a polypeptide of paragraph 14.

42. A pharmaceutical composition according to any of paragraphs 39 to 41, comprising a polypeptide of paragraph 15 or 16.

43. A pharmaceutical composition according to any of paragraphs 39 to 42, comprising a polypeptide which comprises an amino acid sequence of SEQ ID NO:7 or 8.

44. A pharmaceutical composition according to any of paragraphs 39 to 43, comprising a polypeptide which comprises an amino acid sequence of SEQ ID NO:10 or 11.

45. A pharmaceutical composition according to any of paragraphs 39 to 44, comprising a polypeptide which comprises an amino acid sequence of SEQ ID NO:1 or 3.

46. A pharmaceutical composition according to any of paragraphs 39 to 45, comprising a polypeptide which comprises an amino acid sequence of SEQ ID NO:14.

47. A pharmaceutical composition according to any of paragraphs 39 to 46, comprising a polypeptide which comprises an amino acid sequence of SEQ ID NO:16.

48. A pharmaceutical composition according to any of paragraphs 39 to 47, comprising a polypeptide which comprises an amino acid sequence of SEQ ID NO:18.

49. A pharmaceutical composition comprising a nucleic acid according to any of paragraphs 17 to 21, and a pharmaceutically acceptable carrier, excipient, or diluent.

50. A pharmaceutical composition according to paragraph 49, comprising a nucleic acid molecule encoding a polypeptide of any of paragraphs 1 to 12.

51. A pharmaceutical composition according to paragraph 49 or 50, comprising a nucleic acid molecule encoding a polypeptide of paragraph 14.

52. A pharmaceutical composition according to any of paragraphs 49 to 51, comprising a nucleic acid molecule encoding a polypeptide of paragraph 15 or 16.

53. A pharmaceutical composition according to any of paragraphs 49 to 52, comprising a nucleic acid molecule encoding a polypeptide which comprises an amino acid sequence of SEQ ID NO:7 or 8.

54. A pharmaceutical composition according to any of paragraphs 49 to 53, comprising a nucleic acid molecule encoding a polypeptide which comprises an amino acid sequence of SEQ ID NO:10 or 11.

55. A pharmaceutical composition according to any of paragraphs 49 to 54, comprising a nucleic acid molecule encoding a polypeptide which comprises an amino acid sequence of SEQ ID NO:1 or 3.

56. A pharmaceutical composition according to any of paragraphs 49 to 55, comprising a nucleic acid molecule encoding a polypeptide which comprises an amino acid sequence of SEQ ID NO:14.

57. A pharmaceutical composition according to any of paragraphs 49 to 56, comprising a nucleic acid molecule encoding a polypeptide which comprises an amino acid sequence of SEQ ID NO:16.

58. A pharmaceutical composition according to any of paragraphs 49 to 57, comprising a nucleic acid molecule encoding a polypeptide which comprises an amino acid sequence of SEQ ID NO:18.

59. A pharmaceutical composition comprising a vector according to any of paragraphs 22 to 36, and a pharmaceutically acceptable carrier, excipient, or diluent.

60. A pharmaceutical composition according to any of paragraphs 39 to 59, which further comprises an adjuvant for enhancing an immune response in a subject to the polypeptide, or to a polypeptide encoded by the nucleic acid, of the composition.

61. A method of inducing an immune response to an influenza virus in a subject, which comprises administering to the subject an effective amount of a polypeptide according to any of paragraphs 1 to 16, a nucleic acid according to any of paragraphs 17 to 21, a vector according to any of paragraphs 22 to 36, or a pharmaceutical composition according to any of paragraphs 39 to 60.

62. A method of immunising a subject against an influenza virus, which comprises administering to the subject an effective amount of a polypeptide according to any of paragraphs 1 to 16, a nucleic acid according to any of paragraphs 17 to 21, a vector according to any of paragraphs 22 to 36, or a pharmaceutical composition according to any of paragraphs 39 to 60.

63. A polypeptide according to any of paragraphs 1 to 16, a nucleic acid according to any of paragraphs 17 to 21, a vector according to any of paragraphs 22 to 36, or a pharmaceutical composition according to any of paragraphs 39 to 60, for use as a medicament.

64. A polypeptide according to any of paragraphs 1 to 16, a nucleic acid according to any of paragraphs 17 to 21, a vector according to any of paragraphs 22 to 36, or a pharmaceutical composition according to any of paragraphs 39 to 60, for use in the prevention, treatment, or amelioration of an influenza viral infection.

65. Use of a polypeptide according to any of paragraphs 1 to 16, a nucleic acid according to any of paragraphs 17 to 21, a vector according to any of paragraphs 22 to 36, or a pharmaceutical composition according to any of paragraphs 39 to 60, in the manufacture of a medicament for the prevention, treatment, or amelioration of an influenza viral infection.

66. An isolated polypeptide, which comprises an amino acid sequence of SEQ ID NO:22 (FLU_T2_HA_3_I3).

67. An isolated polypeptide, which comprises an amino acid sequence of SEQ ID NO:16 (FLU_T2_NA_3).

68. An isolated polypeptide, which comprises an amino acid sequence of SEQ ID NO:14 (FLU_T2_M2_1).

69. An isolated polynucleotide, which comprises a nucleotide sequence encoding an amino acid sequence of SEQ ID NO:22 (FLU_T2_HA_3_I3), or the complement thereof.

70. A polynucleotide according to paragraph 69, wherein the nucleotide sequence comprises a sequence of SEQ ID NO:23, or the complement thereof.

71. An isolated polynucleotide, which comprises a nucleotide sequence encoding an amino acid sequence of SEQ ID NO:16 (FLU_T2_NA_3), or the complement thereof.

72. A polynucleotide according to paragraph 71, wherein the nucleotide sequence comprises a sequence of SEQ ID NO:17, or the complement thereof.

73. An isolated polynucleotide, which comprises a nucleotide sequence encoding an amino acid sequence of SEQ ID NO:14 (FLU_T2_M2_1), or the complement thereof.

74. A polynucleotide according to paragraph 73, wherein the nucleotide sequence comprises a sequence of SEQ ID NO:15, or the complement thereof.

75. An isolated polynucleotide which comprises nucleotide sequence encoding FLU_T2_HA_3_I3 amino acid sequence (SEQ ID NO:22), and FLU_T2_NA_3 amino acid sequence (SEQ ID NO:16), or the complement thereof.

76. An isolated polynucleotide which comprises nucleotide sequence encoding FLU_T2_HA_3_I3 amino acid sequence (SEQ ID NO:22), and FLU_T2_M2_1 amino acid sequence (SEQ ID NO:14), or the complement thereof.

77. An isolated polynucleotide which comprises nucleotide sequence encoding FLU_T2_NA_3 amino acid sequence (SEQ ID NO:16), and FLU_T2_M2_1 amino acid sequence (SEQ ID NO:14), or the complement thereof.

78. An isolated polynucleotide which comprises nucleotide sequence encoding FLU_T2_HA_3_I3 amino acid sequence (SEQ ID NO:22), FLU_T2_NA_3 amino acid sequence (SEQ ID NO:16), and FLU_T2_M2_1 amino acid sequence (SEQ ID NO:14), or the complement thereof.

79. A polynucleotide according to any of paragraphs 69 to 78, which comprises a DNA molecule.

80. A polynucleotide according to any of paragraphs 69 to 78, which comprises a messenger RNA (mRNA) molecule.

81. A pharmaceutical composition which comprises:
i) an isolated polynucleotide which comprises nucleotide sequence encoding FLU_T2_HA_3_I3 amino acid sequence (SEQ ID NO:22), or the complement thereof; and
ii) an isolated polynucleotide which comprises nucleotide sequence encoding FLU_T2_NA_3 amino acid sequence (SEQ ID NO:16), or the complement thereof.

82. A pharmaceutical composition which comprises:
i) an isolated polynucleotide which comprises nucleotide sequence encoding FLU_T2_HA_3_I3 amino acid sequence (SEQ ID NO:22), or the complement thereof; and
ii) an isolated polynucleotide which comprises nucleotide sequence encoding FLU_T2_M2_1 amino acid sequence (SEQ ID NO:14), or the complement thereof.

83. A pharmaceutical composition which comprises:
i) an isolated polynucleotide which comprises nucleotide sequence encoding FLU_T2_NA_3 amino acid sequence (SEQ ID NO:16), or the complement thereof; and
ii) an isolated polynucleotide which comprises nucleotide sequence encoding FLU_T2_M2_1 amino acid sequence (SEQ ID NO:14), or the complement thereof.

84. A pharmaceutical composition which comprises:
i) an isolated polynucleotide which comprises nucleotide sequence encoding FLU_T2_HA_3_I3 amino acid sequence (SEQ ID NO:22), or the complement thereof;
ii) an isolated polynucleotide which comprises nucleotide sequence encoding FLU_T2_NA_3 amino acid sequence (SEQ ID NO:16), or the complement thereof; and
iii) an isolated polynucleotide which comprises nucleotide sequence encoding FLU_T2_M2_1 amino acid sequence (SEQ ID NO:14), or the complement thereof.

85. A pharmaceutical composition according to any of paragraphs 81, 82, or 84, wherein the nucleotide sequence encoding FLU_T2_HA_3_I3 amino acid sequence (SEQ ID NO:22), or the complement thereof, comprises the nucleotide sequence of SEQ ID NO:23, or the complement thereof.

86. A pharmaceutical composition according to any of paragraphs 81, 83, or 84, wherein the nucleotide sequence encoding FLU_T2_NA_3 amino acid sequence (SEQ ID NO:16), or the complement thereof, comprises the nucleotide sequence of SEQ ID NO:17, or the complement thereof.

87. A pharmaceutical composition according to any of paragraphs 82, 83, or 84, wherein the nucleotide sequence encoding FLU_T2_M2_1 amino acid sequence (SEQ ID NO:14), or the complement thereof, comprises the nucleotide sequence of SEQ ID NO:15, or the complement thereof.

88. A pharmaceutical composition according to any of paragraphs 81 to 87, wherein each polynucleotide comprises a DNA molecule.

89. A pharmaceutical composition according to any of paragraphs 81 to 87, wherein each polynucleotide comprises a messenger RNA (mRNA) molecule.

90. A combined preparation which comprises:
i) an isolated polynucleotide which comprises nucleotide sequence encoding FLU_T2_HA_3_I3 amino acid sequence (SEQ ID NO:22), or the complement thereof; and
ii) an isolated polynucleotide which comprises nucleotide sequence encoding FLU_T2_NA_3 amino acid sequence (SEQ ID NO:16), or the complement thereof.

91. A combined preparation which comprises:
i) an isolated polynucleotide which comprises nucleotide sequence encoding FLU_T2_HA_3_I3 amino acid sequence (SEQ ID NO:22), or the complement thereof; and
ii) an isolated polynucleotide which comprises nucleotide sequence encoding FLU_T2_M2_1 amino acid sequence (SEQ ID NO:14), or the complement thereof.

92. A combined preparation which comprises:
i) an isolated polynucleotide which comprises nucleotide sequence encoding FLU_T2_NA_3 amino acid sequence (SEQ ID NO:16), or the complement thereof; and
ii) an isolated polynucleotide which comprises nucleotide sequence encoding FLU_T2_M2_1 amino acid sequence (SEQ ID NO:14), or the complement thereof.

93. A combined preparation which comprises:
i) an isolated polynucleotide which comprises nucleotide sequence encoding FLU_T2_HA_3_I3 amino acid sequence (SEQ ID NO:22), or the complement thereof;
ii) an isolated polynucleotide which comprises nucleotide sequence encoding FLU_T2_NA_3 amino acid sequence (SEQ ID NO:16), or the complement thereof; and
iii) an isolated polynucleotide which comprises nucleotide sequence encoding FLU_T2_M2_1 amino acid sequence (SEQ ID NO:14), or the complement thereof.

94. A combined preparation according to any of paragraphs 90, 91, or 93, wherein the nucleotide sequence encoding FLU_T2_HA_3_I3 amino acid sequence (SEQ ID NO:22), or the complement thereof, comprises the nucleotide sequence of SEQ ID NO:23, or the complement thereof.

95. A combined preparation according to any of paragraphs 90, 92, or 93, wherein the nucleotide sequence encoding FLU_T2_NA_3 amino acid sequence (SEQ ID NO:16), or the complement thereof, comprises the nucleotide sequence of SEQ ID NO:17, or the complement thereof.

96. A combined preparation according to any of paragraphs 91, 92, or 93, wherein the nucleotide sequence encoding FLU_T2_M2_1 amino acid sequence (SEQ ID NO:14), or the complement thereof, comprises the nucleotide sequence of SEQ ID NO:15, or the complement thereof.

97. A combined preparation according to any of paragraphs 90 to 96, wherein each polynucleotide comprises a DNA molecule.

98. A combined preparation according to any of paragraphs 90 to 96, wherein each polynucleotide comprises a messenger RNA (mRNA) molecule.

99. A vector comprising a polynucleotide of any of paragraphs 69 to 80.

100. A vector according to paragraph 99, which further comprises a promoter operably linked to the nucleotide sequence.

101. A vector according to paragraph 99, which further comprises, for each nucleotide sequence of the vector encoding a separate polypeptide, a separate promoter operably linked to that nucleotide sequence.

102. A vector according to paragraph 99, which is a DNA vector.

103. A vector according to paragraph 99, which is a messenger (mRNA) vector.

104. A pharmaceutical composition which comprises:
i) a vector comprising a polynucleotide which comprises nucleotide sequence encoding FLU_T2_HA_3_I3 amino acid sequence (SEQ ID NO:22), or the complement thereof; and
ii) a vector comprising a polynucleotide which comprises nucleotide sequence encoding FLU_T2_NA_3 amino acid sequence (SEQ ID NO:16), or the complement thereof.

105. A pharmaceutical composition which comprises:
i) a vector comprising a polynucleotide which comprises nucleotide sequence encoding FLU_T2_HA_3_I3 amino acid sequence (SEQ ID NO:22), or the complement thereof; and
ii) a vector comprising a polynucleotide which comprises nucleotide sequence encoding FLU_T2_M2_1 amino acid sequence (SEQ ID NO:14), or the complement thereof.

106. A pharmaceutical composition which comprises:
i) a vector comprising a polynucleotide which comprises nucleotide sequence encoding FLU_T2_NA_3 amino acid sequence (SEQ ID NO:16), or the complement thereof; and
ii) a vector comprising a polynucleotide which comprises nucleotide sequence encoding FLU_T2_M2_1 amino acid sequence (SEQ ID NO:14), or the complement thereof.

107. A pharmaceutical composition which comprises:
i) a vector comprising a polynucleotide which comprises nucleotide sequence encoding FLU_T2_HA_3_I3 amino acid sequence (SEQ ID NO:22), or the complement thereof;
ii) a vector comprising a polynucleotide which comprises nucleotide sequence encoding FLU_T2_NA_3 amino acid sequence (SEQ ID NO:16), or the complement thereof; and
iii) a vector comprising a polynucleotide which comprises nucleotide sequence encoding FLU_T2_M2_1 amino acid sequence (SEQ ID NO:14), or the complement thereof.

108. A pharmaceutical composition according to any of paragraphs 104, 105, or 107, wherein the nucleotide sequence encoding FLU_T2_HA_3_I3 amino acid sequence (SEQ ID NO:22), or the complement thereof, comprises the nucleotide sequence of SEQ ID NO:23, or the complement thereof.

109. A pharmaceutical composition according to any of paragraphs 104, 106, or 107, wherein the nucleotide sequence encoding FLU_T2_NA_3 amino acid sequence (SEQ ID NO:16), or the complement thereof, comprises the nucleotide sequence of SEQ ID NO:17, or the complement thereof.

110. A pharmaceutical composition according to any of paragraphs 105, 106, or 107, wherein the nucleotide sequence encoding FLU_T2_M2_1 amino acid sequence (SEQ ID NO:14), or the complement thereof, comprises the nucleotide sequence of SEQ ID NO:15, or the complement thereof.

111. A pharmaceutical composition according to any of paragraphs 104 to 110, wherein each vector comprises a promoter operably linked to the encoding nucleotide sequence.

112. A pharmaceutical composition according to any of paragraphs 104 to 110, wherein each vector is a DNA vector.

113. A pharmaceutical composition according to any of paragraphs 104 to 110, wherein each vector is a messenger (mRNA) vector.

114. A combined preparation which comprises:
i) a vector comprising a polynucleotide which comprises nucleotide sequence encoding FLU_T2_HA_3_I3 amino acid sequence (SEQ ID NO:22), or the complement thereof; and
ii) a vector comprising a polynucleotide which comprises nucleotide sequence encoding FLU_T2_NA_3 amino acid sequence (SEQ ID NO:16), or the complement thereof.

115. A combined preparation which comprises:
i) a vector comprising a polynucleotide which comprises nucleotide sequence encoding FLU_T2_HA_3_I3 amino acid sequence (SEQ ID NO:22), or the complement thereof; and
ii) a vector comprising a polynucleotide which comprises nucleotide sequence encoding FLU_T2_M2_1 amino acid sequence (SEQ ID NO:14), or the complement thereof.

116. A combined preparation which comprises:
i) a vector comprising a polynucleotide which comprises nucleotide sequence encoding FLU_T2_NA_3 amino acid sequence (SEQ ID NO:16), or the complement thereof; and
ii) a vector comprising a polynucleotide which comprises nucleotide sequence encoding FLU_T2_M2_1 amino acid sequence (SEQ ID NO:14), or the complement thereof.

117. A combined preparation which comprises:
i) a vector comprising a polynucleotide which comprises nucleotide sequence encoding FLU_T2_HA_3_I3 amino acid sequence (SEQ ID NO:22), or the complement thereof;
ii) a vector comprising a polynucleotide which comprises nucleotide sequence encoding FLU_T2_NA_3 amino acid sequence (SEQ ID NO:16), or the complement thereof; and
iii) a vector comprising a polynucleotide which comprises nucleotide sequence encoding FLU_T2_M2_1 amino acid sequence (SEQ ID NO:14), or the complement thereof.

118. A combined preparation according to any of paragraphs 114, 115, or 117, wherein the nucleotide sequence encoding FLU_T2_HA_3_I3 amino acid sequence (SEQ ID NO:22), or the complement thereof, comprises the nucleotide sequence of SEQ ID NO:23, or the complement thereof.

119. A combined preparation according to any of paragraphs 114, 116, or 117, wherein the nucleotide sequence encoding FLU_T2_NA_3 amino acid sequence (SEQ ID NO:16), or the complement thereof, comprises the nucleotide sequence of SEQ ID NO:17, or the complement thereof.

120. A combined preparation according to any of paragraphs 115, 116, or 117, wherein the nucleotide sequence encoding FLU_T2_M2_1 amino acid sequence (SEQ ID NO:14), or the complement thereof, comprises the nucleotide sequence of SEQ ID NO:15, or the complement thereof.

121. A combined preparation according to any of paragraphs 114 to 120, wherein each vector comprises a promoter operably linked to the encoding nucleotide sequence.

122. A combined preparation according to any of paragraphs 114 to 120, wherein each vector is a DNA vector.

123. A combined preparation according to any of paragraphs 114 to 120, wherein each vector is a messenger (mRNA) vector.

124. A vector according to paragraph 100 or 101, a pharmaceutical composition according to paragraph 111, or a combined preparation according to paragraph 121, wherein the, or each promoter is for expression of a polypeptide encoded by the polynucleotide in mammalian cells.

125. A vector according to paragraph 100 or 101, a pharmaceutical composition according to paragraph 111, or a combined preparation according to paragraph 121, wherein the, or each promoter is for expression of a polypeptide encoded by the polynucleotide in yeast or insect cells.

126. A vector according to any of paragraphs 99-103, a pharmaceutical composition according to any of paragraphs 104-113, or a combined preparation according to any of paragraphs 114-123, wherein the, or each vector is a vaccine vector.

127. A vector, pharmaceutical composition, or combined preparation, according to paragraph 126, wherein the, or each vaccine vector is a viral vaccine vector, a bacterial vaccine vector, an RNA vaccine vector, an mRNA vaccine vector, or a DNA vaccine vector.

128. A vector according to any of paragraphs 99-102, 124, 126, or 127, a pharmaceutical composition according to any of paragraphs 104, 105, 107-112, 124, 126, or 127, or a combined preparation according to any of paragraphs 114, 115, 117-122, 124, 126, or 127, wherein the vector comprising a polynucleotide which comprises nucleotide sequence encoding FLU_T2_HA_3_I3 amino acid sequence (SEQ ID NO:22) comprises the nucleotide sequence of SEQ ID NO:24, or the complement thereof,

129. An isolated cell comprising a vector of any of paragraphs 99-103, 124, 126, or 127.

130. An isolated polypeptide which comprises FLU_T2_HA_3_I3 amino acid sequence (SEQ ID NO:22), FLU_T2_NA_3 amino acid sequence (SEQ ID NO:16), and FLU_T2_M2_1 amino acid sequence (SEQ ID NO: 14).

131. An isolated polypeptide which comprises FLU_T2_HA_3_I3 amino acid sequence (SEQ ID NO:22), and FLU_T2_NA_3 amino acid sequence (SEQ ID NO:16).

132. An isolated polypeptide which comprises FLU_T2_HA_3_I3 amino acid sequence (SEQ ID NO:22), and FLU_T2_M2_1 amino acid sequence (SEQ ID NO: 14).

133. An isolated polypeptide which comprises FLU_T2_NA_3 amino acid sequence (SEQ ID NO:16), and FLU_T2_M2_1 amino acid sequence (SEQ ID NO: 14).

134. A pharmaceutical composition which comprises:
i) a polypeptide which comprises FLU_T2_HA_3_I3 amino acid sequence (SEQ ID NO:22); and
ii) a polypeptide which comprises FLU_T2_NA_3 amino acid sequence (SEQ ID NO:16).

135. A pharmaceutical composition which comprises:
i) a polypeptide which comprises FLU_T2_HA_3_I3 amino acid sequence (SEQ ID NO:22); and
ii) a polypeptide which comprises FLU_T2_M2_1 amino acid sequence (SEQ ID NO:14).

136. A pharmaceutical composition which comprises:
i) a polypeptide which comprises FLU_T2_NA_3 amino acid sequence (SEQ ID NO:16); and
ii) a polypeptide which comprises FLU_T2_M2_1 amino acid sequence (SEQ ID NO:14).

137. A pharmaceutical composition which comprises:
i) a polypeptide which comprises FLU_T2_HA_3_I3 amino acid sequence (SEQ ID NO:22);
ii) a polypeptide which comprises FLU_T2_NA_3 amino acid sequence (SEQ ID NO:16); and
iii) a polypeptide which comprises FLU_T2_M2_1 amino acid sequence (SEQ ID NO:14).

138. A combined preparation which comprises:
i) a polypeptide which comprises FLU_T2_HA_3_I3 amino acid sequence (SEQ ID NO:22); and
ii) a polypeptide which comprises FLU_T2_NA_3 amino acid sequence (SEQ ID NO:16).

139. A combined preparation which comprises:
i) a polypeptide which comprises FLU_T2_HA_3_I3 amino acid sequence (SEQ ID NO:22); and
ii) a polypeptide which comprises FLU_T2_M2_1 amino acid sequence (SEQ ID NO:14).

140. A combined preparation which comprises:
i) a polypeptide which comprises FLU_T2_NA_3 amino acid sequence (SEQ ID NO:16); and
ii) a polypeptide which comprises FLU_T2_M2_1 amino acid sequence (SEQ ID NO:14).

141. A combined preparation which comprises:
i) a polypeptide which comprises FLU_T2_HA_3_I3 amino acid sequence (SEQ ID NO:22);
ii) a polypeptide which comprises FLU_T2_NA_3 amino acid sequence (SEQ ID NO:16); and
iii) a polypeptide which comprises FLU_T2_M2_1 amino acid sequence (SEQ ID NO:14).

142. A pharmaceutical composition, which comprises an isolated polynucleotide according to any of paragraphs 69-80, and a pharmaceutically acceptable carrier, excipient, or diluent.

143 A pharmaceutical composition, which comprises a vector according to any of paragraphs 99-103, and a pharmaceutically acceptable carrier, excipient, or diluent.

144. A pharmaceutical composition, which comprises an isolated polypeptide according to any of paragraphs 66-68, or 130-133, and a pharmaceutically acceptable carrier, excipient, or diluent.

145. A pharmaceutical composition according to any of paragraphs 81-89, 104-113, 124-128, 134-137, or 142-144, which further comprises an adjuvant for enhancing an immune response in a subject to a polypeptide, or to a polypeptide encoded by a nucleotide, of the composition.

146. A polynucleotide according to any of paragraphs 182-188, which comprises one or more modified nucleosides.

147. A vector according to any of paragraphs 99-103, or 124-128, wherein the polynucleotide of the vector comprises one or more modified nucleosides.

148. A pharmaceutical composition according to any of paragraphs 81-89, 104-113, 124-128, 134-137, or 142-145, wherein the or each polynucleotide of the composition comprises one or more modified nucleosides.

149. A combined preparation according to any of paragraphs 90-98, 114-128, or 138-141, wherein each nucleic acid of the combined preparation comprises one or more modified nucleosides.

150. A polynucleotide according to paragraph 146, a vector according to paragraph 147, a pharmaceutical composition according to paragraph 148, or a combined preparation according to paragraph 149, wherein the or each polynucleotide comprises a messenger RNA (mRNA).

151. A polynucleotide according to paragraph 146 or 150, a vector according to paragraph 147 or 150, a pharmaceutical composition according to paragraph 148 or 150, or a combined preparation according to paragraph 149 or 150, wherein the one or more modified nucleosides comprise a 1-methylpseudouridine modification.

152. A polynucleotide according to paragraph 146 or 150 or 151, a vector according to paragraph 147 or 150 or 151, a pharmaceutical composition according to paragraph 148 or 150 or 151, or a combined preparation according to paragraph 149 or 150 or 151, wherein the one or more modified nucleosides comprise a 1-methylpseudouridine modification.

153. A polynucleotide according to any of paragraphs 146 or 150-152, a vector according to any of paragraphs 147, or 150-152, a pharmaceutical composition according to any of paragraphs 148, or 150-152, or a combined preparation according to any of paragraphs 149-152, wherein at least 80% of the uridines in the open reading frame have been modified.

154. A fusion protein comprising a polypeptide according to any of paragraphs 66-68, or 130-133.

155. A pseudotyped virus particle comprising a polypeptide according to any of paragraphs 66-68, or 130-133.

156. A method of inducing an immune response to an influenza virus in a subject, which comprises administering to the subject an effective amount of a polypeptide according to any of paragraphs 66-68, or 130-133, a polynucleotide according to any of paragraphs 69-80, 146, or 150-153, a vector according to any of paragraphs 99-103, 124-128, 147, or 150-153, a pharmaceutical composition according to any of paragraphs 81-89, 104-113,124-128,134-137, 142-145, 148, or 150-153, or a combined preparation according to any of paragraphs 90-98, 114-128, 138-141, or 149-153.

157. A method of immunising a subject against an influenza virus, which comprises administering to the subject an effective amount of a polypeptide according to any of paragraphs 66-68, or 130-133, a polynucleotide according to any of paragraphs 69-80, 146, or 150-153, a vector according to any of paragraphs 99-103, 124-128, 147, or 150-153, a pharmaceutical composition according to any of paragraphs 81-89, 104-113, 124-128, 134-137, 142-145, 148, or 150-153, or a combined preparation according to any of paragraphs 90-98, 114-128, 138-141, or 149-153.

158. A polypeptide according to any of paragraphs 66-68, or 130-133, a polynucleotide according to any of paragraphs 69-80, 146, or 150-153, a vector according to any of paragraphs 99-103, 124-128, 147, or 150-153, a pharmaceutical composition according to any of paragraphs 81-89, 104-113, 124-128, 134-137, 142-145, 148, or 150-153, or a combined preparation according to any of paragraphs 90-98, 114-128, 138-141, or 149-153, for use as a medicament

159. A polypeptide according to any of paragraphs 66-68, or 130-133, a polynucleotide according to any of paragraphs 69-80, 146, or 150-153, a vector according to any of paragraphs 99-103, 124-128, 147, or 150-153, a pharmaceutical composition according to any of paragraphs 81-89, 104-113, 124-128, 134-137, 142-145, 148, or 150-153, or a combined preparation according to any of paragraphs 90-98, 114-128, 138-141, or 149-153, for use in the prevention, treatment, or amelioration of an influenza viral infection.

160. Use of a polypeptide according to any of paragraphs 66-68, or 130-133, a polynucleotide according to any of paragraphs 69-80, 146, or 150-153, a vector according to any of paragraphs 99-103, 124-128, 147, or 150-153, a pharmaceutical composition according to any of paragraphs 81-89, 104-113, 124-128, 134-137, 142-145, 148, or 150-153, or a combined preparation according to any of paragraphs 90-98, 114-128, 138-141, or 149-153, in the manufacture of a medicament for the prevention, treatment, or amelioration of an influenza viral infection.

161. A combined preparation, which comprises:
i) a polypeptide of any of paragraphs 1 to 12;
ii) a polypeptide of paragraph 14; and
iii) a polypeptide of paragraph 15 or 16.

162. A combined preparation, which comprises:
i) a polypeptide of any of paragraphs 1 to 12; and
ii) a polypeptide of paragraph 14.

163. A combined preparation, which comprises:
i) a polypeptide of any of paragraphs 1 to 12; and
ii) a polypeptide of paragraph 15 or 16.

164. A combined preparation, which comprises:
i) a polypeptide of paragraph 14; and
ii) a polypeptide of paragraph 15 or 16.

165. A combined preparation, which comprises:
i) a polynucleotide encoding a polypeptide of any of paragraphs 1 to 12;
ii) a polynucleotide encoding a polypeptide of paragraph 14; and
iii) a polynucleotide encoding a polypeptide of paragraph 15 or 16.

166. A combined preparation, which comprises:
i) a polynucleotide encoding a polypeptide of any of paragraphs 1 to 12; and
ii) a polynucleotide encoding a polypeptide of paragraph 14.

167. A combined preparation, which comprises:
i) a polynucleotide encoding a polypeptide of any of paragraphs 1 to 12; and
ii) a polynucleotide encoding a polypeptide of paragraph 15 or 16.

168. A combined preparation, which comprises:
i) a polynucleotide encoding a polypeptide of paragraph 14; and
ii) a polynucleotide encoding a polypeptide of paragraph 15 or 16.

169. A combined preparation according to any of paragraphs 161, 162, or 163, which comprises a polypeptide which comprises an amino acid sequence of SEQ ID NO:7 or 8.

170. A combined preparation according to any of paragraphs 161, 162, or 163, comprising a polypeptide which comprises an amino acid sequence of SEQ ID NO:10 or 11.

171. A combined preparation according to any of paragraphs 161, 162, or 163, comprising a polypeptide which comprises an amino acid sequence of SEQ ID NO:1 or 3.

172. A combined preparation according to any of paragraphs 161, 162, or 164, comprising a polypeptide which comprises an amino acid sequence of SEQ ID NO:14.

173. A combined preparation according to any of paragraphs 161, 163, or 164, comprising a polypeptide which comprises an amino acid sequence of SEQ ID NO:16.

174. A combined preparation according to any of paragraphs 161, 163, or 164, comprising a polypeptide which comprises an amino acid sequence of SEQ ID NO:18.

175. A combined preparation according to any of paragraphs 165, 166, or 167, which comprises a polynucleotide encoding a polypeptide which comprises an amino acid sequence of SEQ ID NO:7 or 8.

176. A combined preparation according to any of paragraphs 165, 166, or 167, comprising a polynucleotide encoding a polypeptide which comprises an amino acid sequence of SEQ ID NO:10 or 11.

177. A combined preparation according to any of paragraphs 165, 166, or 167, comprising a polynucleotide encoding a polypeptide which comprises an amino acid sequence of SEQ ID NO:1 or 3.

178. A combined preparation according to any of paragraphs 165, 166, or 168, comprising a polynucleotide encoding a polypeptide which comprises an amino acid sequence of SEQ ID NO:14.

179. A combined preparation according to any of paragraphs 165, 167, or 168, comprising a polynucleotide encoding a polypeptide which comprises an amino acid sequence of SEQ ID NO:16.

180. A combined preparation according to any of paragraphs 165, 167, or 168, comprising a polynucleotide encoding a polypeptide which comprises an amino acid sequence of SEQ ID NO:18.

181. A combined preparation according to any of paragraphs 165-168, or 175-180, wherein each polynucleotide comprises a DNA molecule.

182. A combined preparation according to any of paragraphs 165-168, or 175-180, wherein each polynucleotide comprises a messenger RNA (mRNA) molecule.

183. A combined preparation according to any of paragraphs 165-168, or 175-180, wherein each polynucleotide is provided by a vector.

184. A combined preparation according to paragraph 183, wherein each vector comprises a promoter operably linked to the encoding nucleotide sequence.

185. A combined preparation according to paragraph 183 or 184, wherein each vector is a DNA vector.

186. A combined preparation according to paragraph 183 or 184, wherein each vector is a messenger (mRNA) vector.

187. A combined preparation according to paragraph 183, wherein each promoter is for expression of a polypeptide encoded by the polynucleotide in mammalian cells.

188. A combined preparation according to paragraph 183, wherein each promoter is for expression of a polypeptide encoded by the polynucleotide in yeast or insect cells.

189. A combined preparation according to any of paragraphs 183-188, wherein each vector is a vaccine vector.

190. A combined preparation according to paragraph 189, wherein each vaccine vector is a viral vaccine vector, a bacterial vaccine vector, an RNA vaccine vector, an mRNA vaccine vector, or a DNA vaccine vector.

191. A combined preparation according to any of paragraphs 165-168, 175-190, wherein each nucleic acid of the combined preparation comprises one or more modified nucleosides.

192. A combined preparation according to paragraph 191, wherein each polynucleotide comprises a messenger RNA (mRNA).

193. A combined preparation according to paragraph 191 or 192, wherein the one or more modified nucleosides comprise a 1-methylpseudouridine modification.

194. A combined preparation according to paragraph 191 or 192 or 193, wherein the one or more modified nucleosides comprise a 1-methylpseudouridine modification.

195. A combined preparation according to any of paragraphs 191-194, wherein at least 80% of the uridines in the open reading frame have been modified.

Embodiments of the invention are now described, by way of example only, with reference to the accompanying drawings, in which:

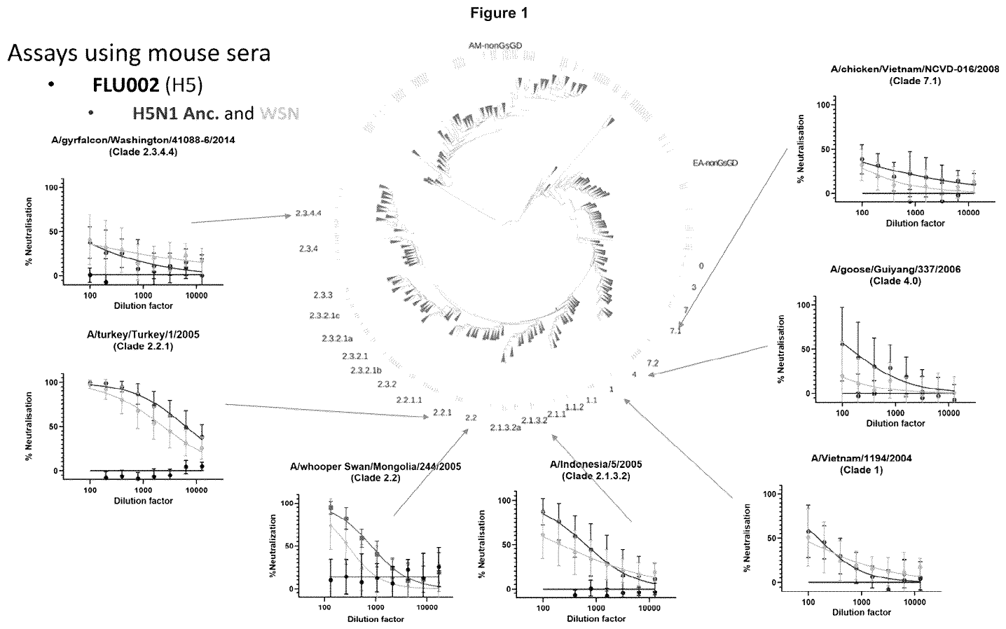

FIG. 1 shows the results of a neutralisation assay illustrating the strength of neutralising antibody responses to various pseudotyped viruses with H5 from different clades and sub-clades;

FIG. 2 shows an amino acid sequence comparison of different embodiments of polypeptides of the invention;

FIG. 3 shows an amino acid sequence comparison of different embodiments of polypeptides of the invention and prior art COBRA sequences;

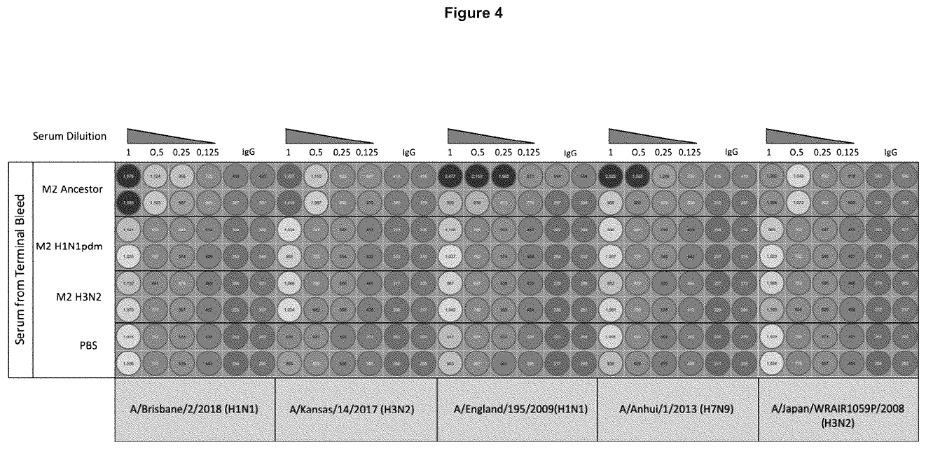

FIG. 4 shows the results of a flow cytometry-based immunofluorescence assay to test the ability of mouse sera, obtained following immunisation of mice with an embodiment of the invention, to target M2 molecules from various influenza A isolates;

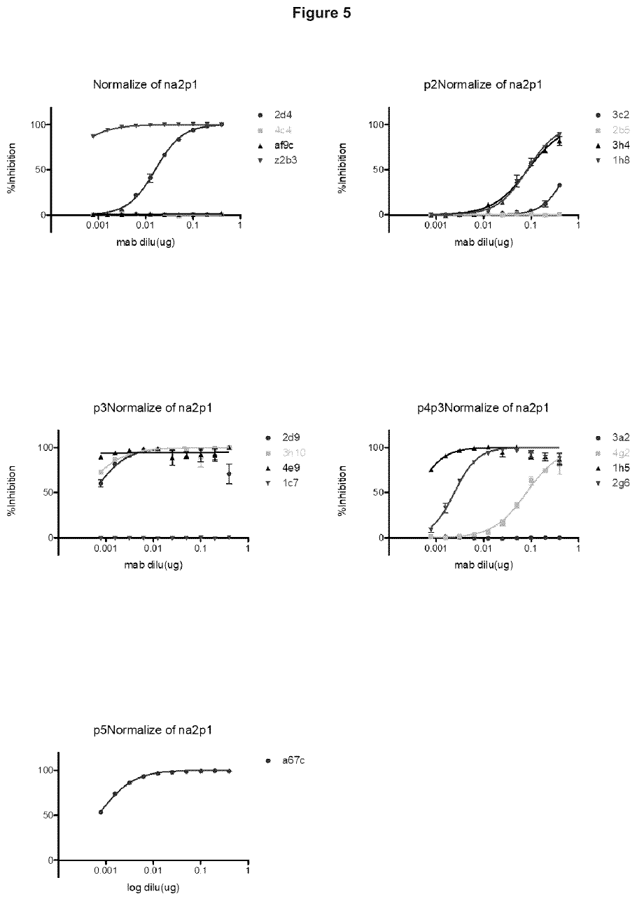

FIG. 5 shows the results of a Pseudotype-based Enzyme-Linked Lectin Assay (pELLA) using FLU_T2_NA_3;

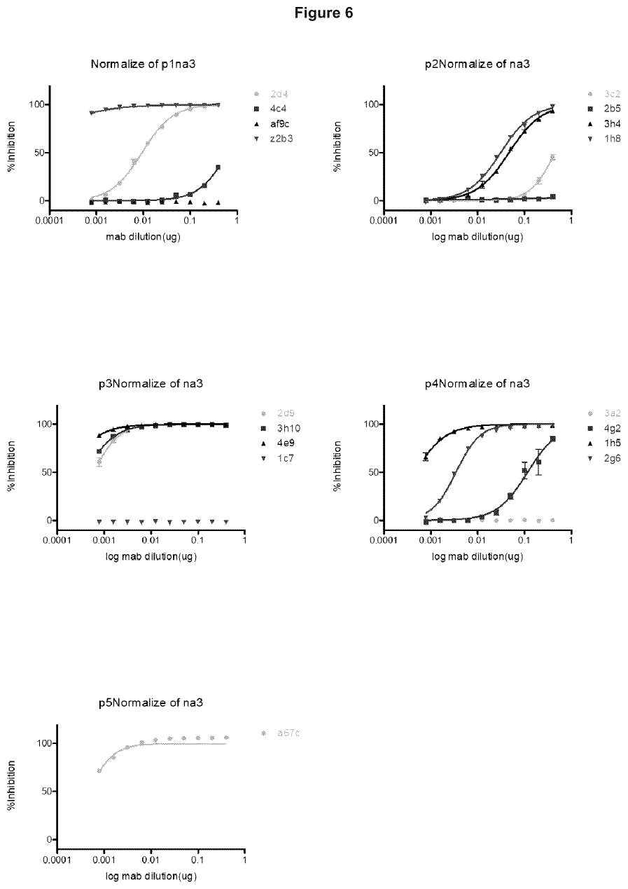

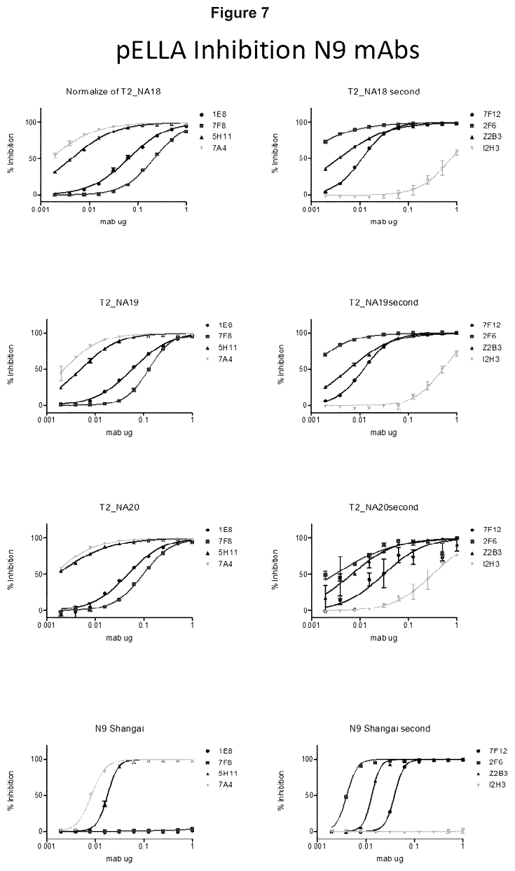

FIG. 6 shows the results of a pELLA using FLU_T2_NA_4;

FIG. 7 shows the results of a pELLA with N9 mAbs;

FIG. 8 shows a plasmid map for pEVAC vector;

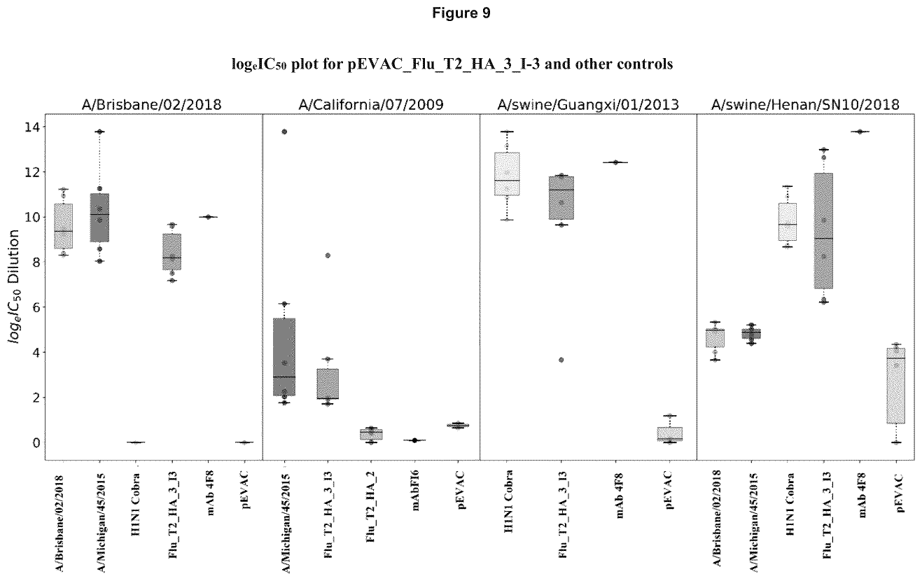

FIG. 9 shows logₑIC₅₀ plot for pEVAC_Flu_T2_HA_3_-3 and other controls;

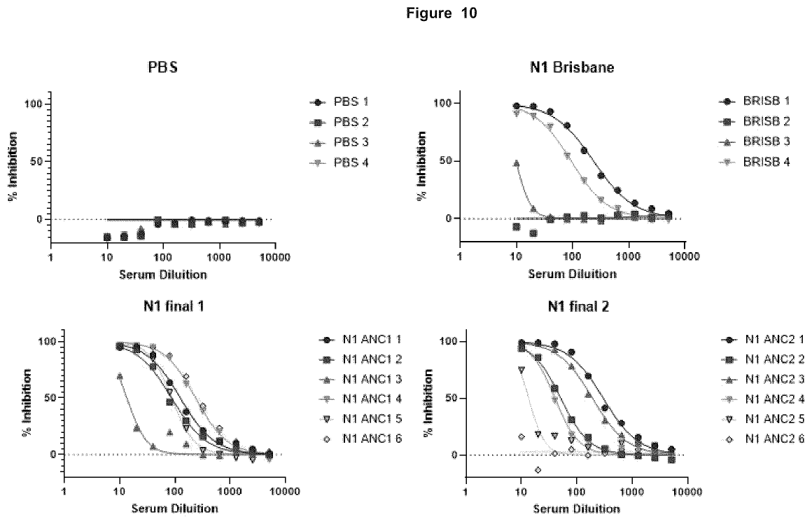

FIG. 10 shows inhibition of enzymatic activity of A/Brisbane/02/2018 neuraminidase by sera from mouse vaccinated by (A) PBS, (B) Primary strain—A/Brisbane/02/2018, (C) N1_Final_1, (D) N1_Final_2 (Flu_T2_NA_3);

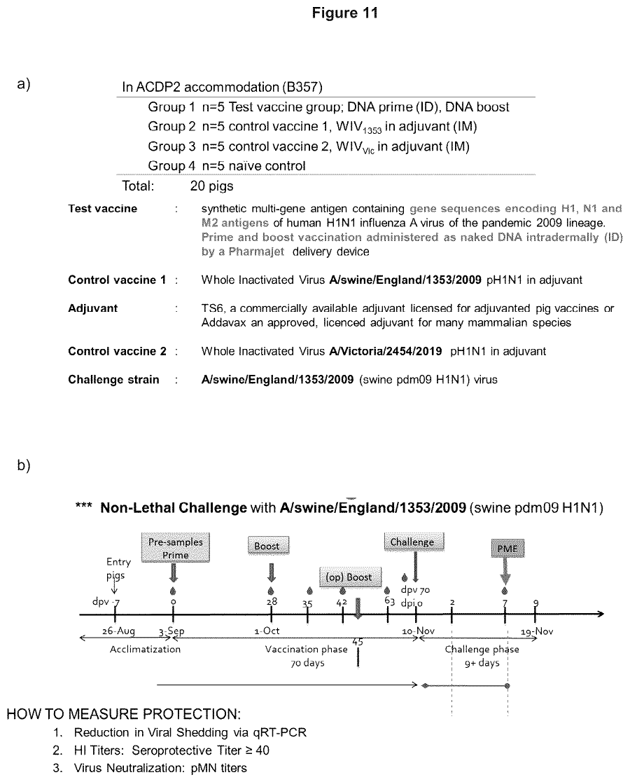

FIGS. 11a and 11b show a vaccination protocol for panH1N1 in pigs;

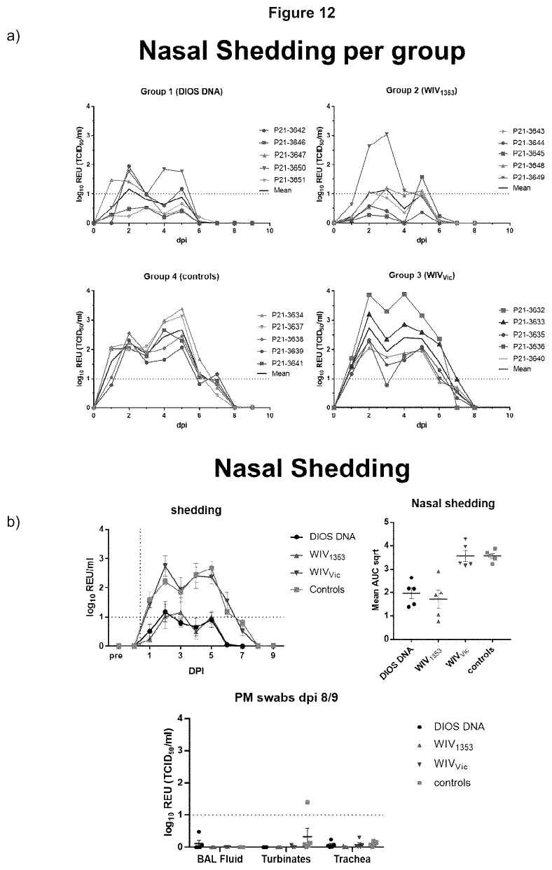

FIG. 12 shows nasal shedding of viral RNA in pigs post infection in four different vaccination groups, monitored daily by RRT-qPCR. FIG. 12b illustrates virus titration measurements from bronchoalveolar lavage (BAL) fluid, turbinates, and trachea samples from pigs in each group;

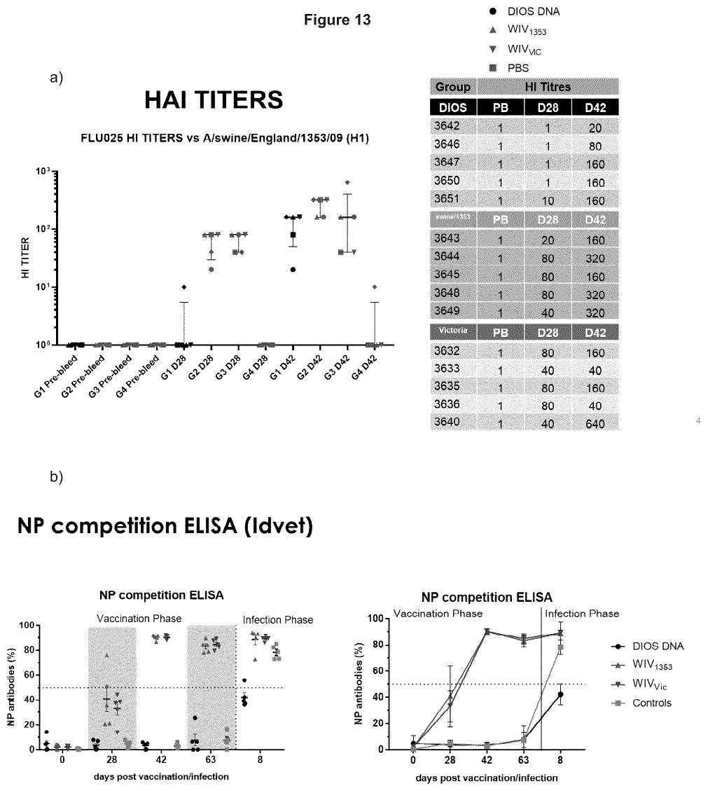

FIG. 13a shows the results of a HAI assay across four vaccination groups vs SW/EN/09 at different time points. FIG. 13b shows the results of an NP competition ELISA (Idvet);

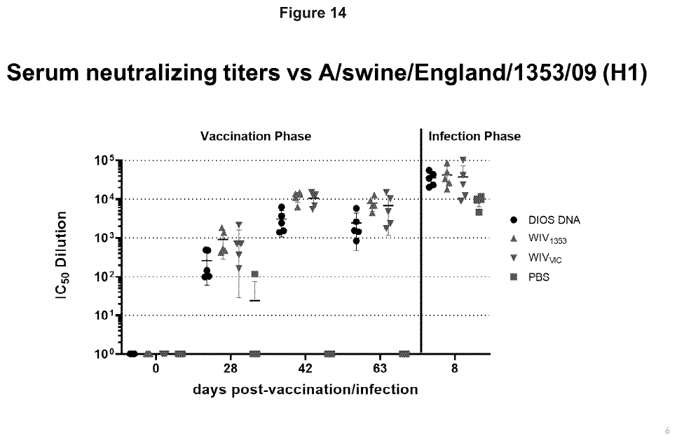

FIG. 14 shows serum neutralising titers at different days post vaccination/infection vs SW/EN/09;

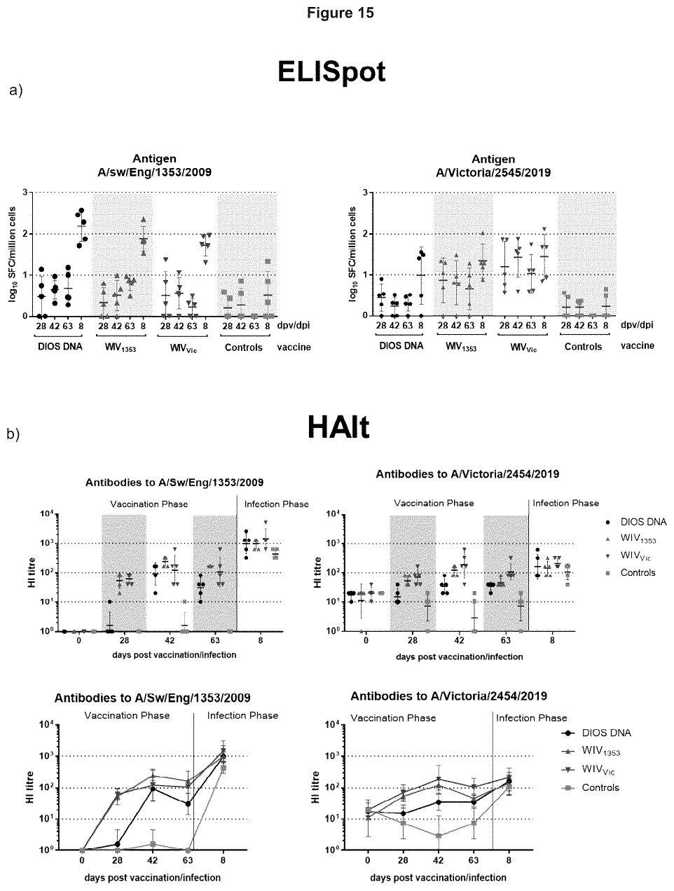

FIG. 15a shows the results of a T-cell peptide stimulation assay; splenocytes were stimulated with the peptides spanning A/swine/England/1353/2009 strain and a/Victoria/2454/HA. FIG. 15b shows a HAI assay. The top panel shows distribution of the hemagglutinin inhibition titre 0 days, 28 days, 42 days and 63 days post vaccination and 8 days post infection. The titres were checked against A/swine/England/1353/2009 strain and a/Victoria/2454/2019 strain. The lower panel illustrates the mean values for each group;

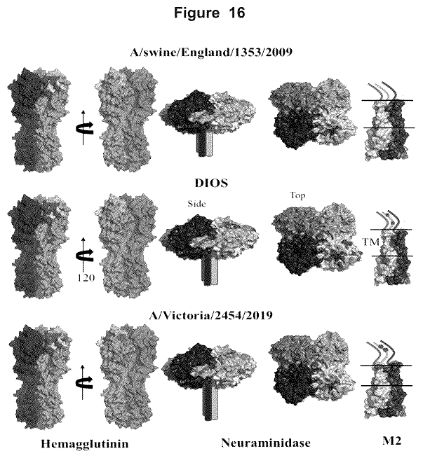

FIG. 16 shows a 3D model of DIOS panH1N1 designed vaccine, comprising HA, NA, and M2 polypeptides;

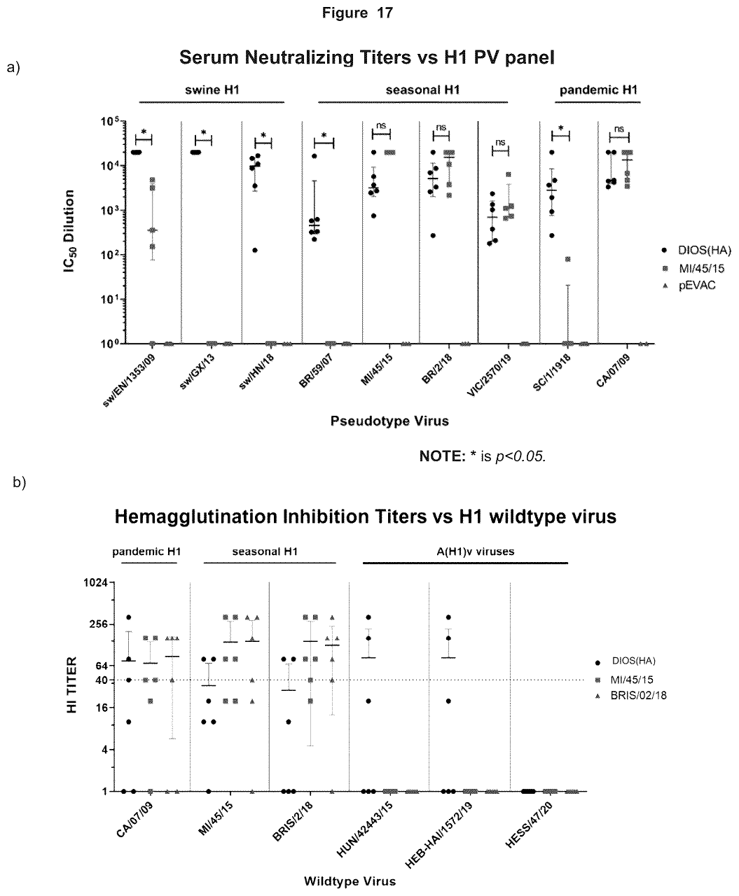

FIG. 17a shows the results of a serum neutralisation assay in mice vs H1 pseudovirus panel using FLU_T2_HA_3_I3. FIG. 17b shows the results of a HAI assay vs a panel of H1 wildtype viruses in mice;

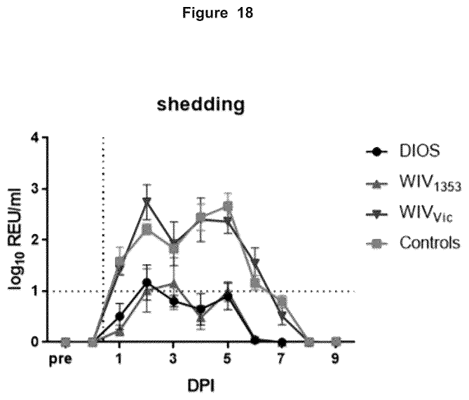

FIG. 18 shows viral RNA shedding in pigs vaccinated with panH1N1 and controls at a number of time points post infection with A/swine/EN/1353/09 10 weeks post-prime;

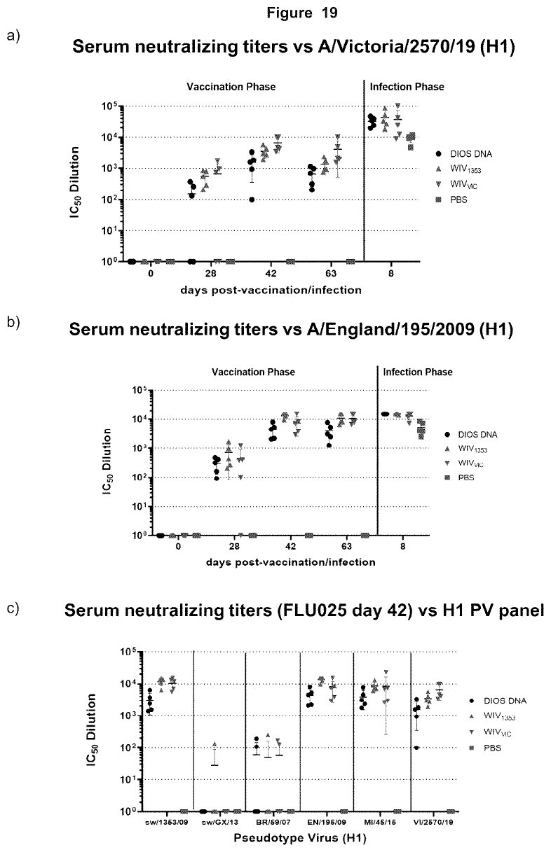

FIGS. 19a and 19b show the results of a serum neutralisation assay in pigs using panH1N1 vs H1 clades at various time points. FIG. 19c shows the results of a neutralisation assay v a panel of H1 pseudoviruses using panH1N1 in pigs;

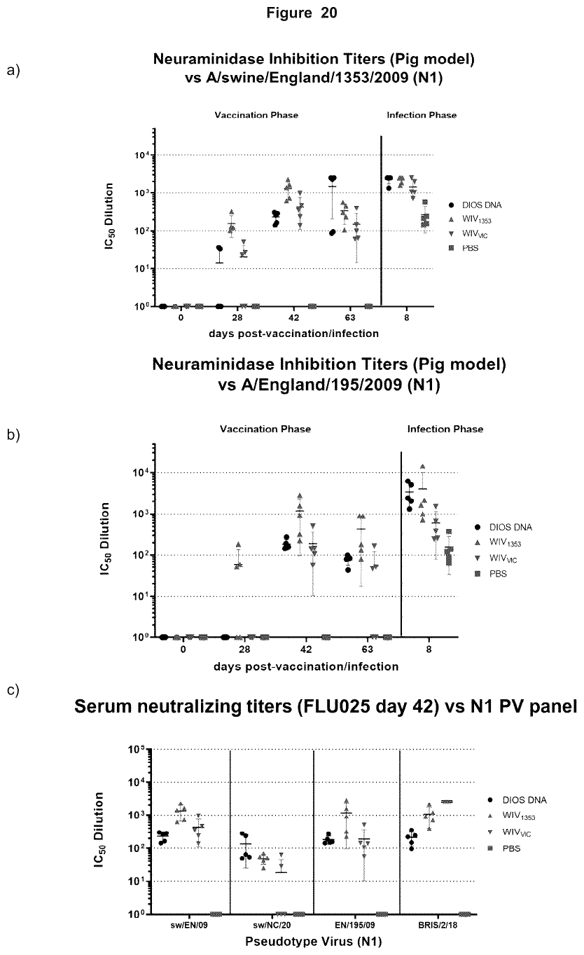

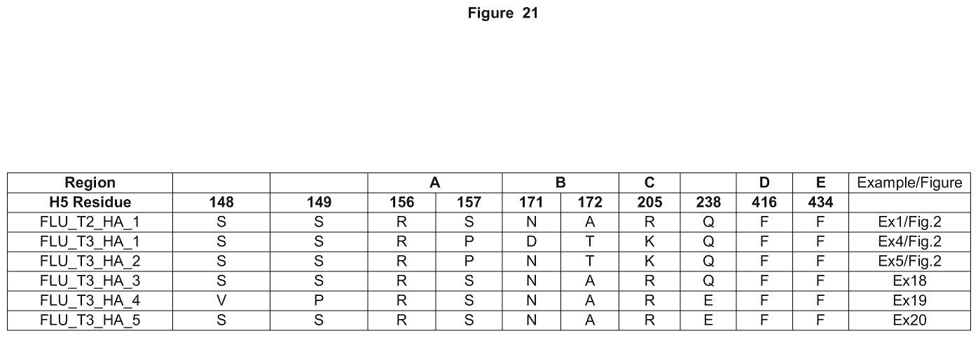

FIGS. 20a and 20b show an ELLA (Enzyme-Linked Lectin Assay) to assess the inhibition activity of the NA component of panH1N1 against A/swine/England/1353/2009 (FIG. 20a) and A/England/195/2009 (FIG. 20b) at a series of time points post-vaccination/infection.

FIG. 20c shows an ELLA against a panel of NA expressing pseudoviruses at 42 days post vaccination;

FIG. 21 summarises differences in amino acid sequence of the influenza haemagluttinin H5 for different embodiments of the invention, including differences at positions A-E of H5 for the embodiments;

FIG. 22 shows a multiple sequence alignment comparing the amino acid sequence of embodiments of the invention with two influenza isolates. In the figure, differences in amino acid residues are shown underlined, with amino acid differences across designed sequences FLU_T2_HA_1 and FLU_T3_HA_1/2/3/4/5 shown highlighted; and

FIG. 23 shows serum neutralisation data for T3 H5 vaccine designs against a panel of 9 antigenically different H5Nx.

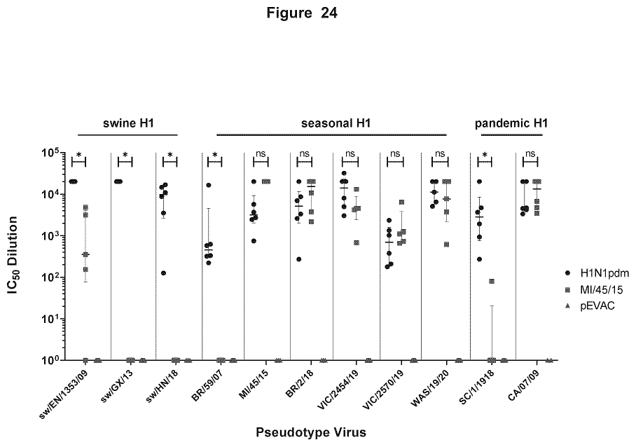

FIG. 24 shows an updated FIG. 17a, wherein two additional seasonal H1 wildtype strains are used as challenges against the designed panH1N1 vaccine.

FIG. 25 summarises novel amino acid residue changes in FLU_T4_HA_1, FLU_T4_HA_2, and FLU_T4_HA_3 designed sequences. These novel amino acid residue changes are shown in bold and underline.

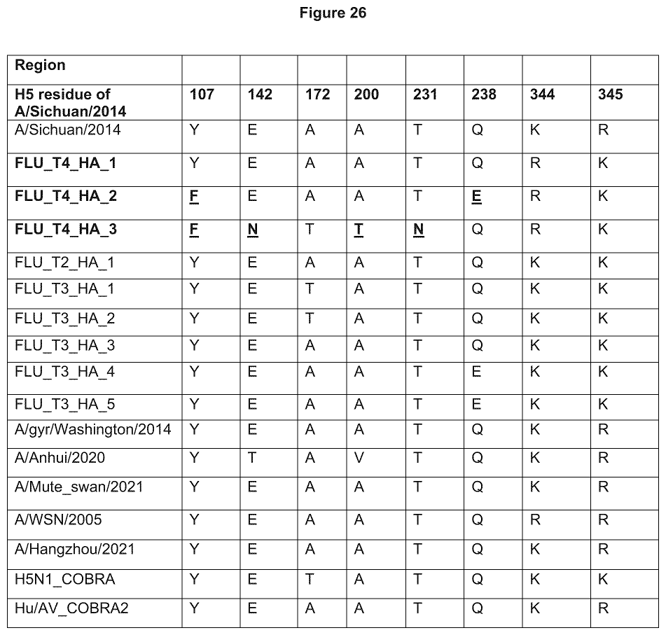

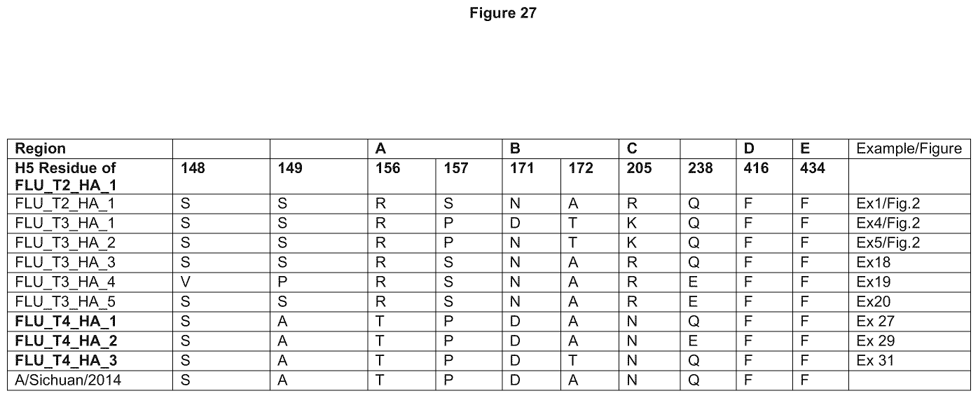

FIG. 26 shows important amino acid residue positions of influenza H5. The residues shown in bold and underline format are novel amino acid residues in the H5 Tier 4 designs.

FIG. 27 summarises amino acid residues of H5 FLU_T4_HA_1, FLU_T4_HA_2, and FLU_T4_HA_3, at further important residue positions of H5.

FIG. 28 shows a multiple sequence alignment of H5 amino acid sequence for FLU_T4_HA_1, FLU_T4_HA_2, and FLU_T4_HA_3, known wild-type influenza H5 strains, and previously designed H5 sequences. The amino acid residue positions in the figure correspond to the amino acid residue positions of A/Sichuan/2014.

FIG. 29A-I shows the neutralising activity of the candidate H5 vaccine antigens, previous designed sequences, and WT sequences, against a panel of clade 2.3.4.4 H5 viruses.

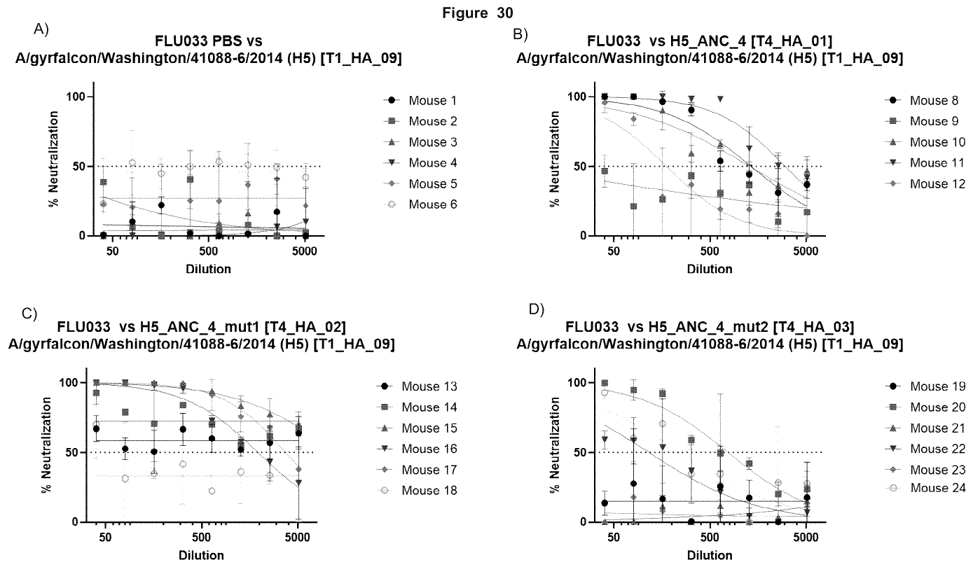

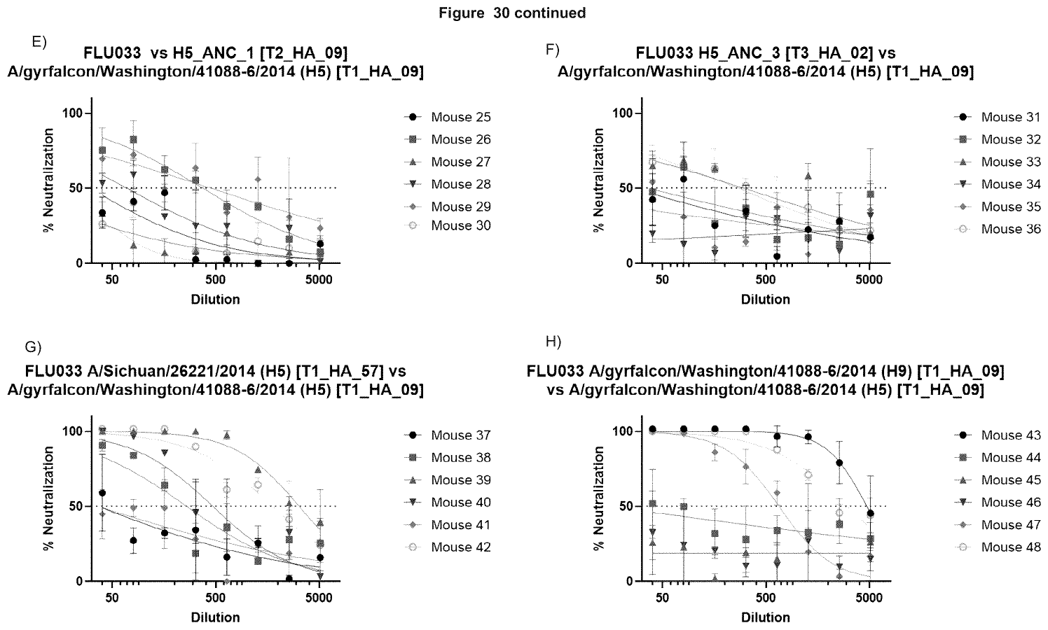

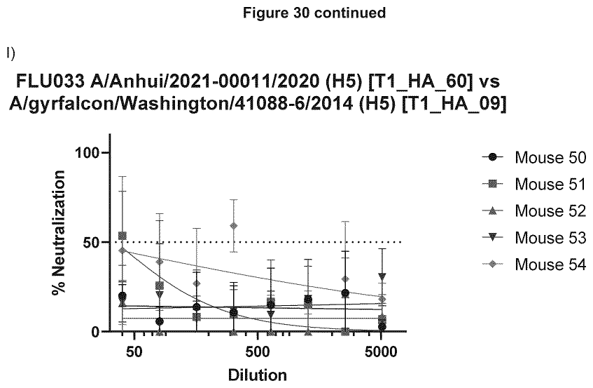

FIGS. 30A-I show individual neutralisation curves for mice immunised with designed sequences or WT sequences, vs A/gyrfalcon/Washington/41088-6/2014) clade 2.3.4.4c. challenge strain.

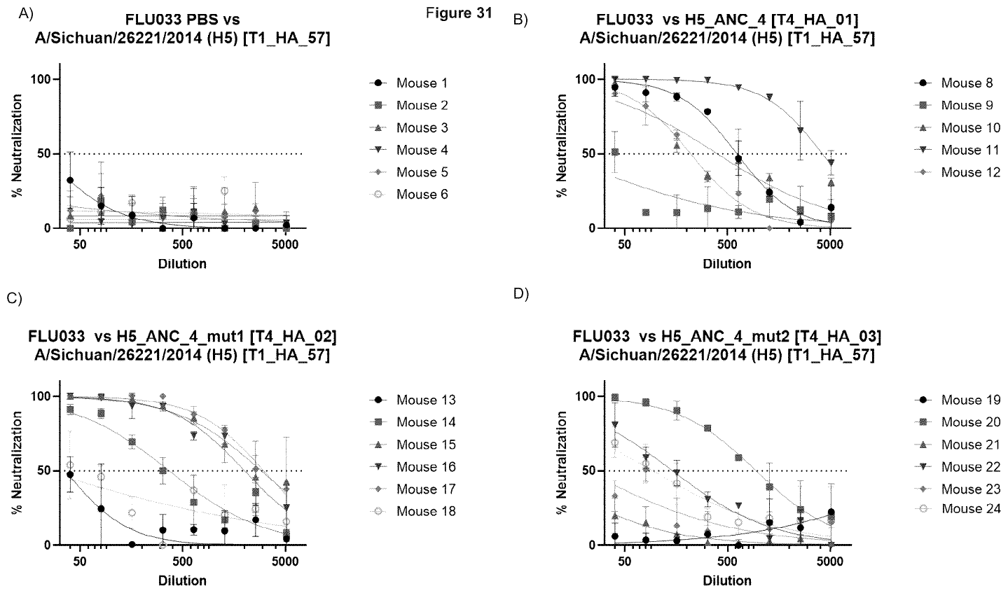

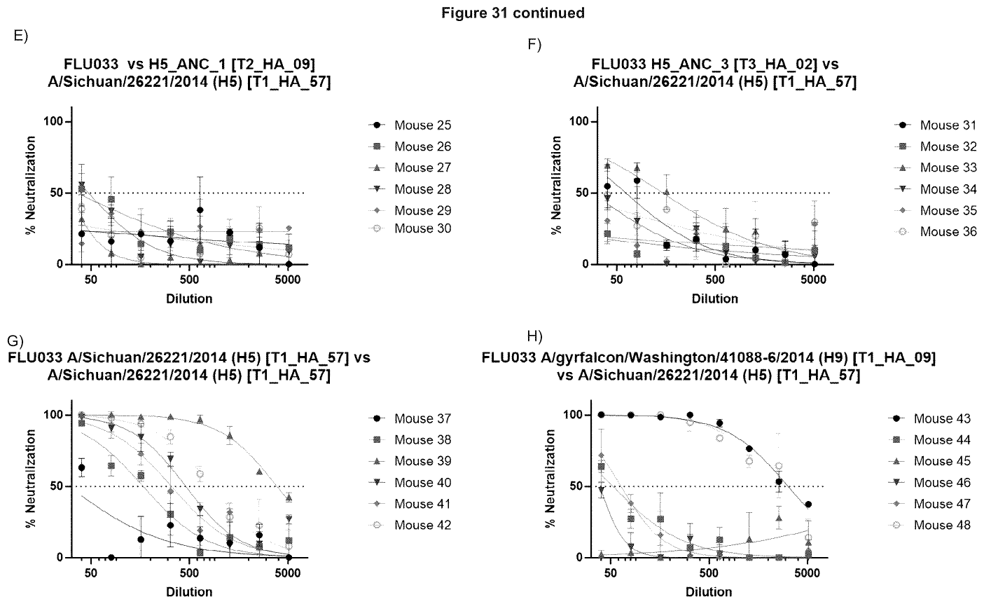

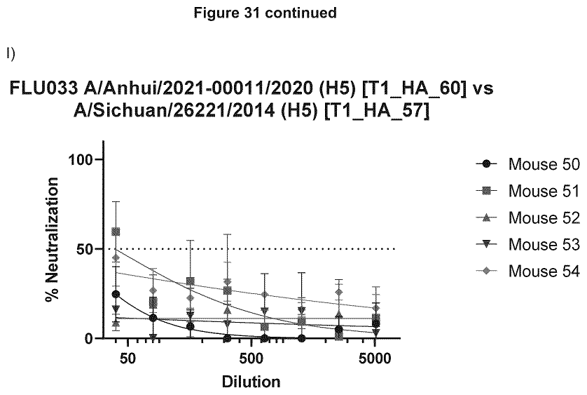

FIGS. 31A-I show individual neutralisation curves for mice immunised with designed sequences or WT sequences, vs A/Sichuan/26221/2014 clade 2.3.4.4a challenge strain.

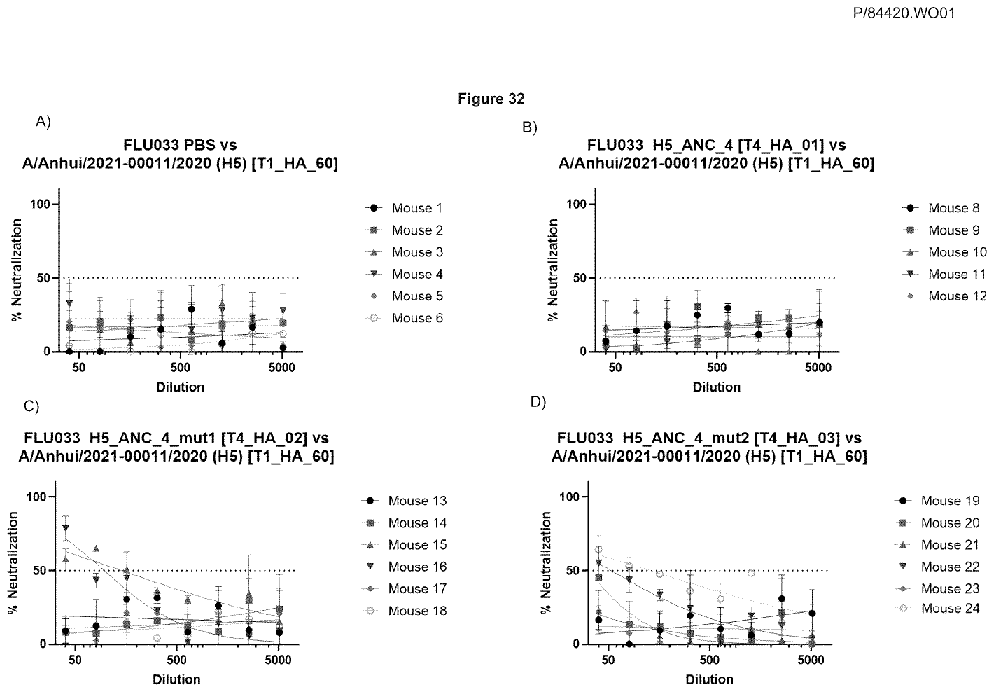

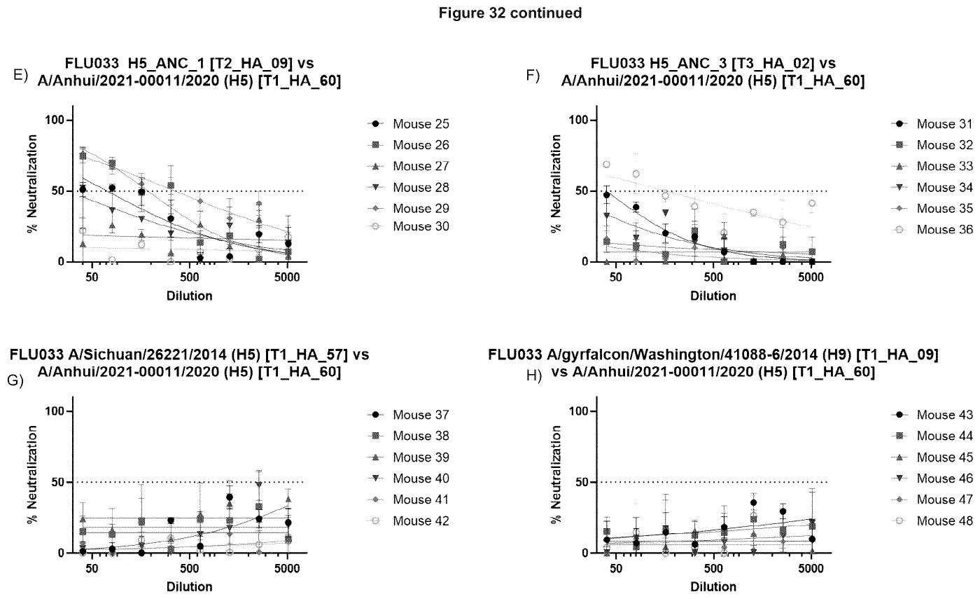

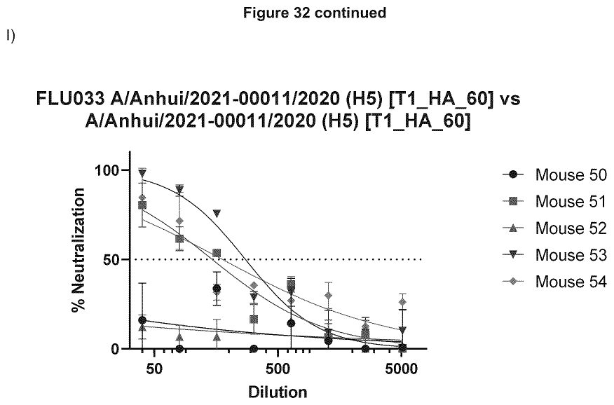

FIGS. 32A-I show individual neutralisation curves for mice immunised with designed sequences or WT sequences, vs A/Anhui/2021-00011/2020 clade 2.3.4.4h challenge strain.

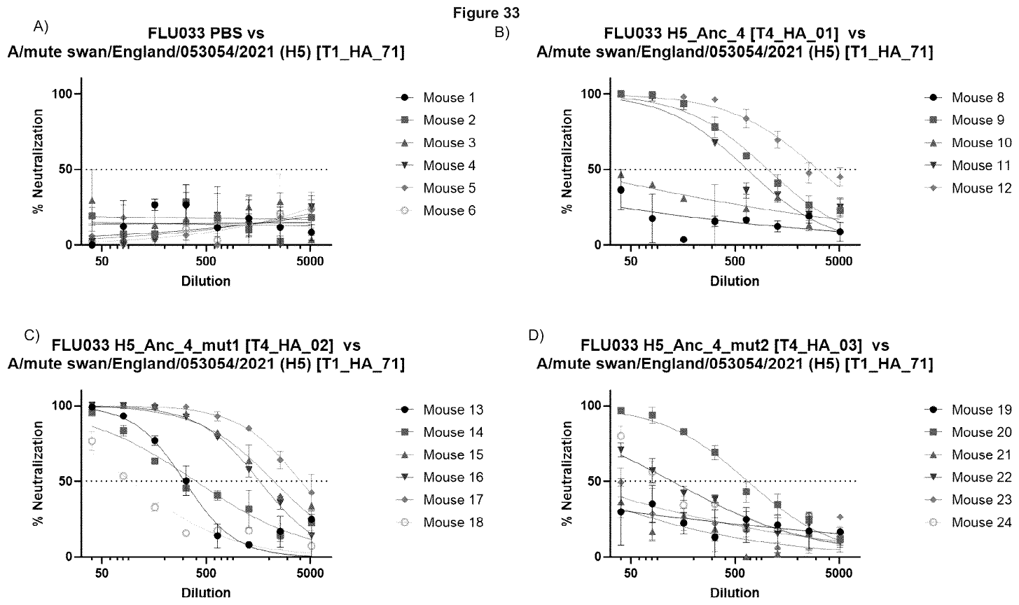

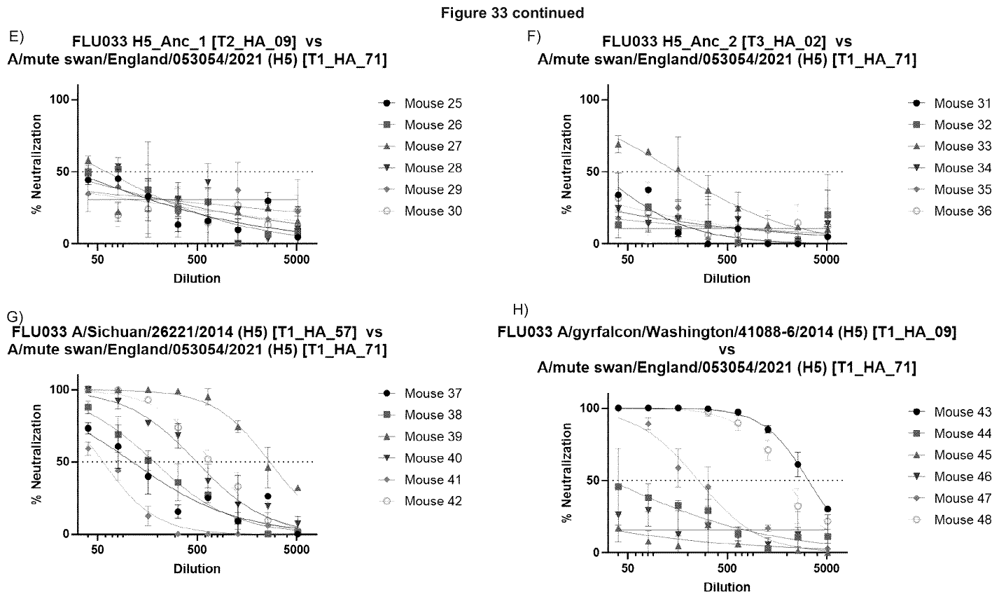

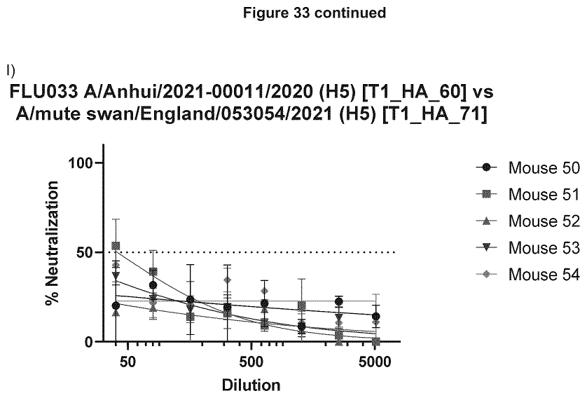

FIGS. 33A-I show individual neutralisation curves for mice immunised with designed sequences or WT sequences, vs A/mute swan/England/053054/2021 clade 2.3.4.4b. challenge strain.

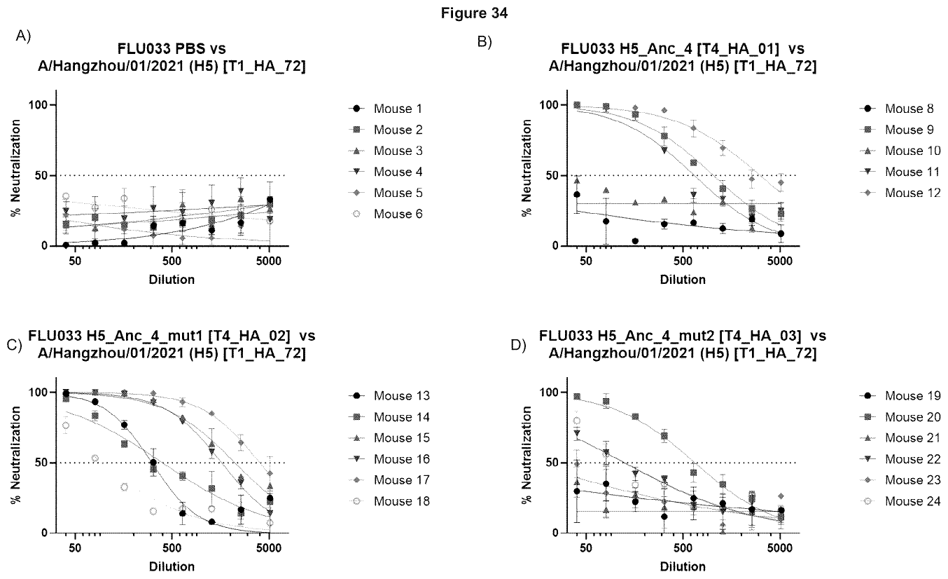

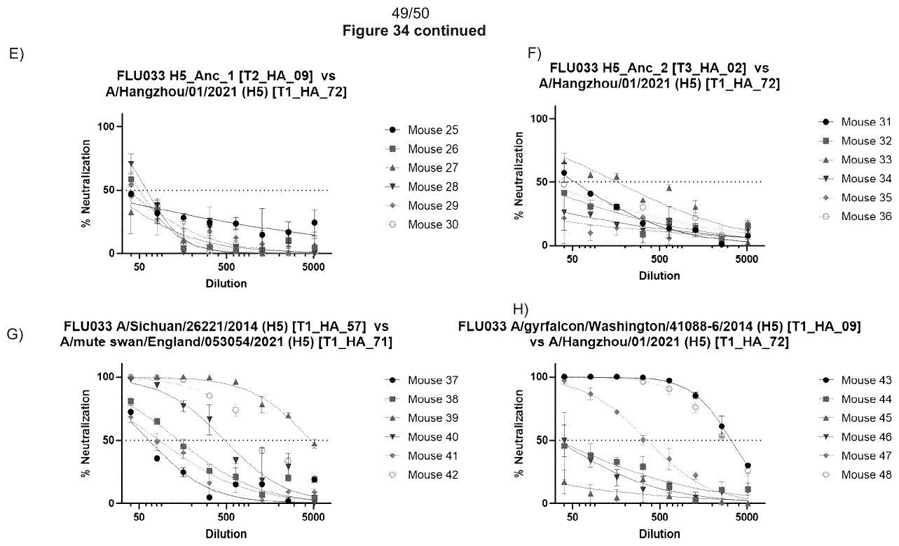

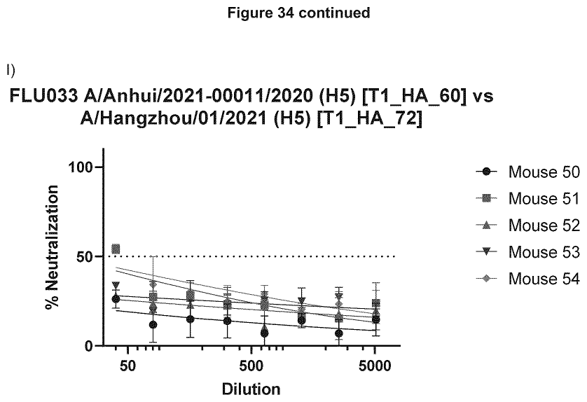

FIGS. 34A-I show individual neutralisation curves for mice immunised with designed sequences or WT sequences, vs A/Hangzhou/01/2021 clade 2.3.4.4b. challenge strain.

Table of SEQ ID NOs:

| SEQ ID NO: | Description |
|---|---|
| 1 | FLU_T2_HA_1: HA0 amino acid sequence |
| 2 | FLU_T2_HA_1: HA0 nucleic acid sequence |
| 3 | FLU_T2_HA_1: head region amino acid sequence |
| 4 | FLU_T2_HA_1: head region nucleic acid sequence |
| 5 | FLU_T2_HA_1: stem region amino acid sequence |
| 6 | FLU_T2_HA_1: stem region nucleic acid sequence |
| 7 | FLU_T3_HA_1: HA0 amino acid sequence |
| 8 | FLU_T3_HA_1: head region amino acid sequence |
| 9 | FLU_T3_HA_1: stem region amino acid sequence |
| 10 | FLU_T3_HA_2: HA0 amino acid sequence |
| 11 | FLU_T3_HA_2: head region amino acid sequence |
| 12 | FLU_T3_HA_2: stem region amino acid sequence |
| 13 | Fragment of H5 globular head domain |
| 14 | FLU_T2_M2_1: amino acid sequence |
| 15 | FLU_T2_M2_1: nucleic acid sequence |
| 16 | FLU_T2_NA_3 (N1_FINAL_2): amino acid sequence |
| 17 | FLU_T2_NA_3 (N1_FINAL_2): nucleic acid sequence |
| 18 | FLU_T2_NA_4 (N1_FINAL_3): amino acid sequence |
| 19 | FLU_T2_NA_4 (N1_FINAL_3): nucleic acid sequence |
| 20 | pEVAC multiple cloning site sequence |
| 21 | Entire pEVAC sequence |
| 22 | FLU_T2_HA_3_13: amino acid sequence |
| 23 | FLU_T2_HA_3_13: nucleic acid sequence |
| 24 | pEVAC- FLU_T2_HA_3_I3: nucleic acid sequence |
| 25 | panH1N1: nucleic acid sequence |
| 26 | pEVAC_panH1N1: nucleic acid sequence |
| 27 | FLU_T3_HA_3: HA0 amino acid sequence |
| 28 | FLU_T3_HA_3: HA0 nucleic acid sequence |
| 29 | FLU_T3_HA_3: head region amino acid sequence |
| 30 | FLU_T3_HA_3: head region nucleic acid sequence |
| 31 | FLU_T3_HA_3: first stem region amino acid sequence |
| 32 | FLU_T3_HA_3: first stem region nucleic acid sequence |
| 33 | FLU_T3_HA_3: second stem region amino acid sequence |
| 34 | FLU_T3_HA_3: second stem region nucleic acid sequence |
| 35 | FLU_T3_HA_4: HA0 amino acid sequence |
| 36 | FLU_T3_HA_4: HA0 nucleic acid sequence |
| 37 | FLU_T3_HA_4: head region amino acid sequence |
| 38 | FLU_T3_HA_4: head region nucleic acid sequence |
| 39 | FLU_T3_HA_4: first stem region amino acid sequence |
| 40 | FLU_T3_HA_4: first stem region nucleic acid sequence |
| 41 | FLU_T3_HA_4: second stem region amino acid sequence |
| 42 | FLU_T3_HA_4: second stem region nucleic acid sequence |
| 43 | FLU_T3_HA_5: HA0 amino acid sequence |
| 44 | FLU_T3_HA_5: HA0 nucleic acid sequence |
| 45 | FLU_T3_HA_5: head region amino acid sequence |
| 46 | FLU_T3_HA_5: head region nucleic acid sequence |
| 47 | FLU_T3_HA_5: first stem region amino acid sequence |
| 48 | FLU_T3_HA_5: first stem region nucleic acid sequence |
| 49 | FLU_T3_HA_5: second stem region amino acid sequence |
| 50 | FLU_T3_HA_5: second stem region nucleic acid sequence |
| 51 | FLU_T3_HA_1: first stem region amino acid sequence |
| 52 | FLU_T3_HA_1: first stem region nucleic acid sequence |
| 53 | FLU_T3_HA_1: second stem region amino acid sequence |
| 54 | FLU_T3_HA_1: second stem region nucleic acid sequence |
| 55 | FLU_T3_HA_1: HA0 nucleic acid sequence |
| 56 | FLU_T3_HA_1: head region nucleic acid sequence |
| 57 | FLU_T3_HA_2: first stem region amino acid sequence |
| 58 | FLU_T3_HA_2: first stem region nucleic acid sequence |
| 59 | FLU_T3_HA_2: second stem region amino acid sequence |
| 60 | FLU_T3_HA_2: second stem region nucleic acid sequence |
| 61 | FLU_T3_HA_2: HA0 nucleic acid sequence |
| 62 | FLU_T3_HA_2: head region nucleic acid sequence |
| 63 | panH1N1: amino acid sequence |
| 64 | A/whooper swan/Mongolia/244/2005 H5 (H5_WSN) |
| 65 | A/gyrfalcon/Washington/41088-6/2014 (H5_GYR) |
| 66 | First 2A self-cleaving peptide sequence: GSGEGRGSLLTCGDVEENPGP |
| 67 | Second 2A self-cleaving peptide sequence: |
| | GSGATNFSLLKQAGDVEENPGP |
| 68 | FLU_T2_HA_4 - amino acid sequence |
| 69 | FLU_T2_HA_4 - nucleic acid sequence |
| 70 | pEVAC-FLU_T2_HA_4 nucleic acid sequence |
| 71 | FLU_T4_HA_1: HA0 amino acid sequence |
| 72 | FLU_T4_HA_1: HA0 nucleic acid sequence |
| 73 | FLU_T4_HA_1: head region amino acid sequence |
| 74 | FLU_T4_HA_1: head region nucleic acid sequence |
| 75 | FLU_T4_HA_1: first stem region amino acid sequence |
| 76 | FLU_T4_HA_1: first stem region nucleic acid sequence |
| 77 | FLU_T4_HA_1: second stem region amino acid sequence |
| 78 | FLU_T4_HA_1: second stem region nucleic acid sequence |
| 79 | pEVAC-FLU_T4_HA_1 - nucleic acid sequence |
| 80 | FLU_T4_HA_2: HA0 amino acid sequence |
| 81 | FLU_T4_HA_2: HA0 nucleic acid sequence |
| 82 | FLU_T4_HA_2: head region amino acid sequence |
| 83 | FLU_T4_HA_2: head region nucleic acid sequence |
| 84 | FLU_T4_HA_2: first stem region amino acid sequence |
| 85 | FLU_T4_HA_2: first stem region nucleic acid sequence |
| 86 | FLU_T4_HA_2: second stem region amino acid sequence |
| 87 | FLU_T4_HA_2: second stem region nucleic acid sequence |
| 88 | pEVAC-FLU_T4_HA_2 - nucleic acid sequence |
| 89 | FLU_T4_HA_3: HA0 amino acid sequence |
| 90 | FLU_T4_HA_3: HA0 nucleic acid sequence |
| 91 | FLU_T4_HA_3: head region amino acid sequence |
| 92 | FLU_T4_HA_3: head region nucleic acid sequence |
| 93 | FLU_T4_HA_3: first stem region amino acid sequence |
| 94 | FLU_T4_HA_3: first stem region nucleic acid sequence |
| 95 | FLU_T4_HA_3: second stem region amino acid sequence |
| 96 | FLU_T4_HA_3: second stem region nucleic acid sequence |
| 97 | pEVAC-FLU_T4_HA_3 - nucleic acid sequence |
| 98 | FLU_T3_NA_3 amino acid sequence |
| 99 | FLU_T3_NA_3 nucleic acid sequence |
| 100 | A/Sichuan/2014 H5 amino acid sequence |

### EXAMPLE 1—FLU_T2_HA_1

This example provides amino acid sequences of the influenza haemagluttinin H5 head and stem regions for an embodiment of the invention known as FLU_T2_HA_1. In SEQ ID NO:1 below, the amino acid residues of the stem region are shown underlined. The amino acid residues of the head region are the remaining residues.

FLU_T2_HA_1-HAO amino acid sequence
(SEQ ID NO: 1):
MEKIVLLLAIVSLVKSDQICIGYHANNSTEQVDTIMEKNVTVTHA
QDILEKTHNGKLCDLDGVKPLILRDCSVAGWLLGNPMCDEFINVP
EWSYIVEKANPANDLCYPGNFNDYEELKHLLSRINHFEKIQIIPK
SSWSDHEASSGVSSACPYQGRSSFFRNVVWLIKKNNAYPTIKRSY
NNTNQEDLLVLWGIHHPNDAAEQTRLYQNPTTYISVGTSTLNQRL
VPKIATRSKVNGQSGRMEFFWTILKPNDAINFESNGNFIAPEYAY
KIVKKGDSAIMKSELEYGNCNTKCQTPMGAINSSMPFHNIHPLTI
GECPKYVKSNRLVLATGLRNSPQRERRRKKRGLFGAIAGFIEGG
WQGMVDGWYGYHHSNEQGSGYAADKESTQKAIDGVTNKVNSIIDK
MNTQFEAVGREFNNLERRIENLNKKMEDGFLDVWTYNAELLVLME
NERTLDFHDSNVKNLYDKVRLQLRDNAKELGNGCFEFYHKCDNEC
MESVRNGTYDYPQYSEEARLKREEISGVKLESIGTYQILSIYSTV
ASSLALAIMVAGLSLWMCSNGSLQCRICI
FLU_T2_HA_1-HAO nucleic acid sequence
(SEQ ID NO: 2):
atggaaaagattgtgctgctgctggccatcgtgtccctggtcaag
agcgatcaaatctgcatcggctaccacgccaacaacagcaccgaa
caggtggacaccattatggaaaagaacgtgaccgtgacacacgcc
caggacatcctggaaaagacccacaacggcaagctgtgcgacctg
gatggcgtgaagcctctgatcctgagagattgctctgtggccggc
tggctgctgggcaatcctatgtgcgacgagttcatcaacgtgccc
gagtggtcctatatcgtggaaaaggccaatcctgccaacgacctg
tgctaccccggcaacttcaacgactacgaggaactgaaacatctg
ctgagccggatcaaccacttcgagaagatccagatcatccccaag
tcctcttggagcgatcacgaggcctctagcggagtgtctagcgcc
tgtccttaccaaggcagaagcagcttcttccggaacgtcgtgtgg
ctgatcaagaagaacaacgcttaccccaccatcaagcggagctac
aacaacaccaatcaagaggacctgctggtgctgtggggcatccac
catcctaatgatgccgccgagcagacccggctgtaccagaatcct
acaacctacatcagcgtgggcaccagcacactgaaccagagactg
gtgcctaagatcgccaccagatccaaagtgaacggccagagcggc
cggatggaattcttctggaccatcctgaagcctaacgacgccatc
aacttcgagagcaacggcaactttatcgcccctgagtacgcctac
aagatcgtgaagaagggcgacagcgccatcatgaagtccgagctg
gaatacggcaactgcaacaccaagtgtcagacccctatgggcgcc
atcaatagcagcatgcccttccacaacattcaccctctgaccatc
ggcgagtgccccaaatacgtgaagtccaacagactggtcctggcc
accggcctgagaaattctccacagagagagcggcgcagaaagaag
agaggcctgtttggagccattgccggctttatcgaaggcggctgg
caaggcatggttgacggatggtacggctatcaccacagcaatgag
caaggctctggctacgccgccgacaaagagagcacacagaaagcc
atcgacggcgtgaccaacaaagtgaatagcatcatcgacaagatg
aacacccagttcgaggccgtgggcagagagttcaacaacctggaa
agacggatcgagaacctgaacaagaagatggaggacggcttcctg
gacgtgtggacctataatgccgagctgctggtcctgatggaaaac
gagagaaccctggacttccacgacagcaacgtgaagaacctgtac
gacaaagtgcggctccagctgcgggacaatgccaaagaactcggc
aacggctgcttcgagttctaccacaagtgcgacaacgagtgcatg
gaaagcgtgcggaacgqcacctacgactaccctcagtactctgag
gaagcccggctgaagagagaagagatcagcggagtgaagctggaa
tccatcggcacataccagatcctgagcatctacagcaccgtggcc
tcttctctggccctggctattatggtggctggcctgagcctgtgg
atgtgctctaatggcagcctccagtgccggatctgcatc
FLU_T2_HA_1-head region amino acid sequence
(SEQ ID NO: 3):
THNGKLCDLDGVKPLILRDCSVAGWLLGNPMCDEFINVPEWSYIV
EKANPANDLCYPGNFNDYEELKHLLSRINHFEKIQIIPKSSWSDH
EASSGVSSACPYQGRSSFFRNVVWLIKKNNAYPTIKRSYNNTNQE
DLLVLWGIHHPNDAAEQTRLYQNPTTYISVGTSTLNQRLVPKIAT
RSKVNGQSGRMEFFWTILKPNDAINFESNGNFIAPEYAYKIVKKG
DSAIMKSELEYGNCNTKCQTPMGAINSSMPFHNIHPLTIGECP

The amino acid residues at positions 156, 157, 171, 172, and 205 are shown underlined in the above sequence (and are R, S, N, A, and R, respectively).

FLU_T2_HA_1-head region nucleic acid sequence
(SEQ ID NO: 4):
acccacaacggcaagctgtgcgacctggatggcgtgaagcctctg
atcctgagagattgctctgtggccggctggctgctgggcaatcct
atgtgcgacgagttcatcaacgtgcccgagtggtcctatatcgtg
gaaaaggccaatcctgccaacgacctgtgctaccccggcaacttc
aacgactacgaggaactgaaacatctgctgagccggatcaaccac
ttcgagaagatccagatcatccccaagtcctcttggagcgatcac
gaggcctctagcggagtgtctagcgcctgtccttaccaaggcaga
agcagcttcttccggaacgtcgtgtggctgatcaagaagaacaac
gcttaccccaccatcaagcggagctacaacaacaccaatcaagag
gacctgctggtgctgtggggcatccaccatcctaatgatgccgcc
gagcagacccggctgtaccagaatcctacaacctacatcagcgtg
ggcaccagcacactgaaccagagactggtgcctaagatcgccacc
agatccaaagtgaacggccagagcggccggatggaattcttctgg
accatcctgaagcctaacgacgccatcaacttcgagagcaacggc
aactttatcgcccctgagtacgcctacaagatcgtgaagaagggc
gacagcgccatcatgaagtccgagctggaatacggcaactgcaac
accaagtgtcagacccctatgggcgccatcaatagcagcatgccc
ttccacaacattcaccctctgaccatcggcgagtgcccc
FLU_T2_HA_1-stem region amino acid sequence
(SEQ ID NO: 5):
MEKIVLLLAIVSLVKSDQICIGYHANNSTEQVDTIMEKNVTVTHA
QDILEKKYVKSNRLVLATGLRNSPQRERRRKKRGLFGAIAGFIEG
GWQGMVDGWYGYHHSNEQGSGYAADKESTQKAIDGVTNKVNSIID
KMNTQFEAVGREFNNLERRIENLNKKMEDGFLDVWTYNAELLVLM
ENERTLDFHDSNVKNLYDKVRLQLRDNAKELGNGCFEFYHKCDNE
CMESVRNGTYDYPQYSEEARLKREEISGVKLESIGTYQILSIYST
VASSLALAIMVAGLSLWMCSNGSLQCRICI

The amino acid residues at positions 416 and 434 (or at positions 148 and 166 if counting from the beginning of the stem region) are shown underlined in the above sequence (and are F and F, respectively).

FLU_T2_HA_1-stem region nucleic
acid sequence (SEQ ID NO: 6):
atggaaaagattgtgctgctgctggccatcgtgtccctggtcaaga
gcgatcaaatctgcatcggctaccacgccaacaacagcaccgaac
aggtggacaccattatggaaaagaacgtgaccgtgacacacgccc
aggacatcctggaaaagaaatacgtgaagtccaacagactggtcc
tggccaccggcctgagaaattctccacagagagagcggcgcagaa
agaagagaggcctgtttggagccattgccggctttatcgaaggcg
gctggcaaggcatggttgacggatggtacggctatcaccacagca
atgagcaaggctctggctacgccgccgacaaagagagcacacaga
aagccatcgacggcgtgaccaacaaagtgaatagcatcatcgaca
agatgaacacccagttcgaggccgtgggcagagagttcaacaacc
tggaaagacggatcgagaacctgaacaagaagatggaggacggct
tcctggacgtgtggacctataatgccgagctgctggtcctgatgg
aaaacgagagaaccctggacttccacgacagcaacgtgaagaacc
tgtacgacaaagtgcggctccagctgcgggacaatgccaaagaac
tcggcaacggctgcttcgagttctaccacaagtgcgacaacgagt
gcatggaaagcgtgcggaacggcacctacgactaccctcagtact
ctgaggaagcccggctgaagagagaagagatcagcggagtgaagc
tggaatccatcggcacataccagatcctgagcatctacagcaccg
tggcctcttctctggccctggctattatggtggctggcctgagcc
tgtggatgtgctctaatggcagcctccagtgccggatctgcatc

### EXAMPLE 2

FLU_T2_HA_1 was tested for its ability to elicit a broadly neutralising antibody response to pseudotyped viruses with H5 from different clades and sub-clades.

Immunisation of Mice with DNA Vaccine:

Female BALB/c mice, 8-10 weeks old, were immunised 4 times (week 0, week 2, week 4, week 6) and bled 6-7 times (week 0, week 2, week 4, week 6, week 8, week 10, week 12) with:
50 μg FLU_T2_HA_1 DNA in pEVAC vector (see ‘H5N1 Anc.’ in FIG. 1);
50 μg A/whooper swan/Mongolia/244/2005 (H5) DNA in pEVAC vector (see ‘WSN’ in FIG. 1), which is a primary isolate strain sequenced in 2005 from a whooper swan (i.e. an H5 control); or
50 μl PBS.

DNA was injected subcutaneously into the rear flank of the mice. The DNA and the PBS are endotoxin free.

Ability of Mouse Sera to Neutralise Pseudotyped Viruses with H5 from Different Clades and Sub-Clades:

Mouse sera collected following the immunisations was tested against the following pseudotyped viruses (with H5 from different clades and sub-clades):
A/gyrfalcon/Washington/41088-6/2014 (H5, clade 2.3.4.4);
A/turkey/Turkey/1/2005 (H5, clade 2.2.1);
A/whooper Swan/Mongolia/244/2005 (H5, clade 2.2)—homologous to the H5 control;
A/Indonesia/5/2005 (H5, clade 2.1.3.2);
A/Vietnam/1194/2004 (H5, clade 1);
A/goose/Guiyang/337/2006 (H5, clade 4);
A/chicken/Vietnam/NCVD-016/2008 (H5, clade 7.1)

FIG. 1 shows the results of a neutralisation assay illustrating the strength of neutralising antibody responses to the various pseudotyped viruses. The results illustrate the ability of each vaccine to elicit broadly neutralising antibody responses to a diverse panel of pseudotyped viruses with H5 from different clades and sub-clades.

The results show that administering mice the FLU_T2_HA_1 DNA vaccine gave a significantly greater cross-clade immune response than immunisation with the A/whooper swan/Mongolia/244/2005 H5 control vaccine, and the naïve mouse serum.

### EXAMPLE 3—DESIGN OF FLU_T3_HA_1 AND FLU_T3_HA_2

This example describes the design of amino acid sequences of two further embodiments of the invention, FLU_T3_HA_1 and FLU_T3_HA_2.

As described in Example 2 above, mouse sera obtained following immunisation with FLU_T2_HA_1 DNA vaccine neutralised many clades of H5 but was less effective against clades 2.3.4 and 7.1. These two clades are currently in circulation in birds, and are among the most dominant co-circulating H5N1 viruses in poultry in Asia, with sporadic cases of infection occurring regularly in humans and other mammals.

Epitope regions in the H5 head region important for neutralisation of clade 2.3.4 and clade 7.1 were identified using available protein structural data. The amino acid sequences of these epitopes were compared with FLU_T2_HA_1 to identify amino acid positions that may have abrogated the neutralisation of these two clades by the mouse sera.

Amino acid positions within FLU_T2_HA_1 were identified that, when changed to particular amino acid residues, can elicit an antibody response that is able to neutralise clades 2.3.4 and 7.1 without abrogating the neutralisation of other clades. These positions are at amino acid residues 157, 171, 172, and 205 of the H5 protein (see positions A, B and C in FIG. 2). The influence of these mutations on the stability of the HA protein, as well as its interaction with known antibodies against clade 2.3.4 and clade 7, were checked by energetics calculations. The mutations that stabilised the protein and its interaction with such antibodies, while minimally altering the neutralisation of other clades, were selected for. The resulting new HA sequences are termed FLU_T3_HA_1 and FLU_T3_HA_2.

FIG. 2 shows an amino acid sequence comparison of FLU_T2_HA_1 with FLU_T3_HA_1 and FLU_T3_HA_2. FLU_T3_HA_1 is described in more detail in Example 4, and FLU_T3_HA_2 is described in more detail in Example 5, below.

### EXAMPLE 4—FLU_T3_HA_1

This example provides amino acid sequences of the influenza haemagluttinin H5 head and stem regions for an embodiment of the invention known as FLU_T3_HA_1. In SEQ ID NO:7 below, the amino acid residues of the stem region are shown underlined. The amino acid residues of the head region are the remaining residues.

FLU_T3_HA_1-HAO amino acid sequence
(SEQ ID NO: 7):
MEKIVLLLAIVSLVKSDQICIGYHANNSTEQVDTIMEKNVTVTHA
QDILEKTHNGKLCDLDGVKPLILRDCSVAGWLLGNPMCDEFINVP
EWSYIVEKANPANDLCYPGNFNDYEELKHLLSRINHFEKIQIIPK
SSWSDHEASSGVSSACPYQGRPSFFRNVVWLIKKNDTYPTIKRSY
NNTNQEDLLVLWGIHHPNDAAEQTKLYQNPTTYISVGTSTLNQRL
VPKIATRSKVNGQSGRMEFFWTILKPNDAINFESNGNFIAPEYAY
KIVKKGDSAIMKSELEYGNCNTKCQTPMGAINSSMPFHNIHPLTI
GECPKYVKSNRLVLATGLRNSPQRERRRKKRGLFGAIAGFIEGGW
QGMVDGWYGYHHSNEQGSGYAADKESTQKAIDGVTNKVNSIIDKM
NTQFEAVGREFNNLERRIENLNKKMEDGFLDVWTYNAELLVLMEN
ERTLDFHDSNVKNLYDKVRLQLRDNAKELGNGCFEFYHKCDNECM
ESVRNGTYDYPQYSEEARLKREEISGVKLESIGTYQILSIYSTVA
SSLALAIMVAGLSLWMCSNGSLQCRICI
FLU_T3_HA_1-head region amino acid
sequence (SEQ ID NO: 8):
THNGKLCDLDGVKPLILRDCSVAGWLLGNPMCDEFINVPEWSYIV
EKANPANDLCYPGNFNDYEELKHLLSRINHFEKIQIIPKSSWSDH
EASSGVSSACPYQGRPSFFRNVVWLIKKNDTYPTIKRSYNNTNQE
DLLVLWGIHHPNDAAEQTKLYQNPTTYISVGTSTLNQRLVPKIAT
RSKVNGQSGRMEFFWTILKPNDAINFESNGNFIAPEYAYKIVKKG
DSAIMKSELEYGNCNTKCQTPMGAINSSMPFHNIHPLTIGECP

The amino acid residues at positions 156, 157, 171, 172, and 205 are shown underlined in the above sequence (and are R, P, D, T, and K, respectively).

FLU_T3_HA_1-stem region amino acid
sequence (SEQ ID NO: 9):
MEKIVLLLAIVSLVKSDQICIGYHANNSTEQVDTIMEKNVTVTHA
QDILEKKYVKSNRLVLATGLRNSPQRERRRKKRGLFGAIAGFIEG
GWQGMVDGWYGYHHSNEQGSGYAADKESTQKAIDGVTNKVNSIID
KMNTQFEAVGREFNNLERRIENLNKKMEDGFLDVWTYNAELLVLM
ENERTLDFHDSNVKNLYDKVRLQLRDNAKELGNGCFEFYHKCDNE
CMESVRNGTYDYPQYSEEARLKREEISGVKLESIGTYQILSIYST
VASSLALAIMVAGLSLWMCSNGSLQCRICI

The amino acid residues at positions 416 and 434 are shown underlined in the above sequence (and are F and F, respectively).

### EXAMPLE 5—INFLUENZA H5 T3_HA_2

This example provides amino acid sequences of the influenza H5 head and stem regions for an embodiment of the invention known as FLU_T3_HA_2. In SEQ ID NO:4 below, the amino acid residues of the stem region are shown underlined. The amino acid residues of the head region are the remaining residues.

FLU_T3_HA_2-HAO amino acid sequence
(SEQ ID NO: 10):
MEKIVLLLAIVSLVKSDQICIGYHANNSTEQVDTIMEKNVTVTHA
QDILEKTHNGKLCDLDGVKPLILRDCSVAGWLLGNPMCDEFINVP
EWSYIVEKANPANDLCYPGNFNDYEELKHLLSRINHFEKIQIIPK
SSWSDHEASSGVSSACPYQGRPSFFRNVVWLIKKNNTYPTIKRSY
NNTNQEDLLVLWGIHHPNDAAEQTKLYQNPTTYISVGTSTLNQRL
VPKIATRSKVNGQSGRMEFFWTILKPNDAINFESNGNFIAPEYAY
KIVKKGDSAIMKSELEYGNCNTKCQTPMGAINSSMPFHNIHPLTI
GECPKYVKSNRLVLATGLRNSPQRERRRKKRGLFGAIAGFIEGGW
QGMVDGWYGYHHSNEQGSGYAADKESTQKAIDGVTNKVNSIIDKM
NTQFEAVGREFNNLERRIENLNKKMEDGFLDVWTYNAELLVLMEN
ERTLDFHDSNVKNLYDKVRLQLRDNAKELGNGCFEFYHKCDNECM
ESVRNGTYDYPQYSEEARLKREEISGVKLESIGTYQILSIYSTVA
SSLALAIMVAGLSLWMCSNGSLQCRICI
FLU_T3_HA_2-head region amino acid sequence
(SEQ ID NO: 11):
THNGKLCDLDGVKPLILRDCSVAGWLLGNPMCDEFINVPEWSYIV
EKANPANDLCYPGNFNDYEELKHLLSRINHFEKIQIIPKSSWSDH
EASSGVSSACPYQGRPSFFRNVVWLIKKNNTYPTIKRSYNNTNQE
DLLVLWGIHHPNDAAEQTKLYQNPTTYISVGTSTLNQRLVPKIAT
RSKVNGQSGRMEFFWTILKPNDAINFESNGNFIAPEYAYKIVKKG
DSAIMKSELEYGNCNTKCQTPMGAINSSMPFHNIHPLTIGECP

The amino acid residues at positions 156, 157, 171, 172, and 205 are shown underlined in the above sequence (and are R, P, N, T, and K, respectively).

FLU_T3_HA_2-stem region amino
acid sequence (SEQ ID NO: 12):
MEKIVLLLAIVSLVKSDQICIGYHANNSTEQVDTIMEKNVTVTHA
QDILEKKYVKSNRLVLATGLRNSPQRERRRKKRGLFGAIAGFIEG
GWQGMVDGWYGYHHSNEQGSGYAADKESTQKAIDGVTNKVNSIID
KMNTQFEAVGREFNNLERRIENLNKKMEDGELDVWTYNAELLVLM
ENERTLDFHDSNVKNLYDKVRLQLRDNAKELGNGCFEFYHKCDNE
CMESVRNGTYDYPQYSEEARLKREEISGVKLESIGTYQILSIYST
VASSLALAIMVAGLSLWMCSNGSLQCRICI

The amino acid residues at positions 416 and 434 are shown underlined in the above sequence (and are F and F, respectively).

### EXAMPLE 6—COMPARISON OF FLU_T3_HA_1 AND FLU_T3_HA_2 WITH PRIOR ART COBRA H5 TIER 2 DESIGN

FIG. 3 shows an amino acid comparison of FLU_T3_HA_1 and FLU_T3_HA_2 with a prior art COBRA H5 Tier 2 design. There are amino acid differences at three positions (A, B, and C) in the head region which have been introduced in FLU_T3_HA_1 and FLU_T3_HA_2 to increase the affinity of the antigen towards antibodies of important clades. The amino acid differences are at residue numbers 156, 157, 171, 172, and 205 of the head region. There are additional amino acid differences at two positions (C and D) in the stem region which have been introduced in FLU_T3_HA_1 and FLU_T3_HA_2 to stabilise the stem region in both the pre- and post-fusion state. The amino acid differences are at residue numbers 416 and 434 of the stem region.

### EXAMPLE 7—FLU_T2_M2_1

This example provides the amino acid and nucleic acid sequences of the influenza M2 region for an embodiment of the invention known as FLU_T2_M2_1.

FLU_T2_M2_1-amino acid sequence
(SEQ ID NO: 14):
MSLLTEVETPTRNGWECRCSDSSDPLVIAASIIGILHLILWILDR
LFFKCIYRRLKYGLKRGPSTEGVPESMREEYRQKQQSAVDVDDGH
FVNIELE
FLU_T2_M2_1-nucleic acid sequence
(SEQ ID NO: 15):
ATGTCTCTGCTGACCGAGGTGGAAACCCCTACCAGAAATGGCTGG
GAGTGCAGATGCAGCGACAGCAGCGATCCTCTGGTTATCGCCGCC
AGCATCATCGGCATCCTGCACCTGATCCTGTGGATCCTGGACCGG
CTGTTCTTCAAGTGCATCTACCGGCGGCTGAAGTACGGCCTGAAG
AGAGGCCCTTCTACAGAGGGCGTGCCCGAGAGCATGCGGGAAGAG
TACAGACAGAAACAGCAGAGCGCCGTGGACGTGGACGATGGCCAC
TTCGTGAACATCGAGCTGGAA

### EXAMPLE 8—IMMUNE RESPONSE ELICITED BY FLU_T2_M2_1

This example describes a flow cytometry-based immunofluorescence assay to test the ability of mouse sera, obtained following immunisation of mice with FLU_T2_M2_1 DNA vaccine, to target M2 molecules from influenza A isolates of different subtypes.

Immunisation of Mice with DNA Vaccine:

4 groups of 6 Balb/c mice, 8-10 weeks old, were immunised 4 times (week 0, week 2, week 4, week 6) and bled 6 times (week 0, week 2, week 4, week 6, week 8, week 10) with:
50 μg FLU_T2_M2_1 DNA in pEVAC vector (see ‘M2 ancestor.’ in FIG. 5);
50 μg FLU_T1_M2_1 DNA in pEVAC vector (M2 from H1N1pdm, see ‘M2 H1N1’ in FIG. 5);
50 μg FLU_T1_M2_2 DNA in pEVAC vector (M2 from H3N2, see ‘M2 H3N2’ in FIG. 5); or
50 μl PBS.

DNA was injected subcutaneously into the rear flank of the mice. The DNA and the PBS are endotoxin free.

Ability of Mouse Sera to Target M2 from Influenza Isolates of Different Subtypes:

HEK293T cells were transfected with pEVAC vector expressing M2 DNA from the following isolates:
A/Brisbane/2/2018 (H1N1);
A/Kansas/14/2017 (H3N2);
A/England/195/2009 (H1N1);
A/Anhui/1/2013 (H7N9); and
A/Japan/WRAIR1059P/2008 (H3N2)

Serum was pooled for each group (six mice per group), serially diluted and incubated with cells for 30 minutes at room temperature. Mouse IgG isotype antibody was used as negative control staining. After incubation, cells were washed twice in PBS, and then incubated with Goat anti-mouse AF647 secondary antibody for 30 minutes at room temperature, in the dark. Before FACS analysis, cells were washed with PBS another two times. Analysis was performed using Attune NxT FACS (Thermo Fisher).

FIG. 4 shows the results of a flow cytometry-based immunofluorescence assay illustrating the ability of the mouse serum antibodies to target M2s from the different influenza isolates. The results illustrate the ability of each vaccine to target M2 from influenza isolates of different subtypes.

The results show that administering mice the FLU_T2_M2_1 DNA vaccine (M2 ancestor) elicited a significantly greater immune response against M2 across different influenza sub-types than immunisation with M2 from H1N1 or H3N2 isolates, and the naïve mouse serum.

### EXAMPLE 9—FLU_T2_NA_3 AND FLU_T2_NA_4

This example provides the amino acid and nucleic acid sequences of the influenza neuraminidase region for embodiments of the invention known as FLU_T2_NA_3 and FLU_T2_NA_4.

FLU_T2_NA_3 (N1_FINAL_2)-amino acid
sequence (SEQ ID NO: 16):
MNPNQKIITIGSICMVVGIISLILQIGNIISIWVSHSIQTGNQNQ
PETCNQSIITYENNTWVNQTYVNISNTNFVAEQAVASVALAGNSS
LCPISGWAIYSKDNGIRIGSKGDVFVIREPFISCSHLECRTFFLT
QGALLNDKHSNGTVKDRSPYRTLMSCPVGEAPSPYNSRFESVAWS
ASACHDGISWLTIGISGPDNGAVAVLKYNGIITDTIKSWRNNILR
TQESECACINGSCFTIMTDGPSNGQASYKIFKIEKGKVVKSVELN
APNYHYEECSCYPDAGEVMCVCRDNWHGSNRPWVSFNQNLEYQIG
YICSGVFGDNPRPNDGTGSCGPVSSNGAYGVKGFSFKYGKGVWIG
RTKSTSSRSGFEMIWDPNGWTETDSSFSVKQDIVAITDWSGYSGS
FVQHPELTGLDCMRPCFWVELIRGRPKENTIWTSGSSISFCGVNS
DTVGWSWPDGAELPFTIDK
FLU_T2_NA_3 (N1_FINAL_2)-nucleic acid
sequence (SEQ ID NO: 17):
atgaatccaaatcagaaaataataaccattgggtcaatctgtatg
gtagttggaataatcagcctaatattacaaattgggaacataatc
tcaatatgggttagccattcaattcagactggaaatcaaaaccaa
cctgaaacatgcaaccaaagcatcattacttatgaaaacaacact
tgggtgaatcaaacatatgttaacatcagcaataccaattttgtt
gctgaacaggctgtagcttcagtggcattagcgggcaattcctct
ctctgccccattagtggggggctatatacagcaaggacaatggca
taaggattggttccaagggagatgtatttgtcataagagagccat
tcatttcatgctcccacttggaatgcaggaccttttttctgactc
aaggagccttgttgaatgacaaacattccaatggaaccgttaaag
acagaagcccctacagaaccttaatgagctgtcctgttggtgagg
ctccctctccatacaattcaaggtttgagtcggttgcttggtcag
caagtgcttgccatgatggcattagctggttgacaattggaattt
ccgggccagacaatggggcagtggctgtattgaaatacaatggca
taataacagacactatcaaaagttggagaaacaacatattgagga
cacaagagtctgaatgtgcctgcataaatggttcttgctttacta
taatgaccgatggaccaagtaatgggcaggcctcatacaagattt
tcaagatagagaaggggaaggtagtcaaatcagtcgagttgaatg
cccctaattaccactacgaggaatgttcctgttatcctgatgctg
gcgaagtaatgtgtgtgtgcagggataattggcatggttcgaatc
gaccatgggtgtctttcaatcaaaatctggagtatcaaataggat
acatatgcagtggggttttcggagacaatccacgccccaatgatg
gaacaggcagctgtggtccagtgtcttctaatggagcatatggag
taaagggattttcatttaagtacggcaagggtgtttggataggga
gaactaagagcactagttccaggagtggatttgagatgatttggg
atcccaatggatggacagagacagatagtagtttctcagtgaagc
aagatattgtagcaataactgattggtcaggatatagcgggagtt
ttgtccaacatccagaattaacagggctggactgcatgaggcctt
gcttctgggttgaactaatcagaggacggcctaaggagaacacaa
tctggactagtgggagcagcatttccttctgtggtgtaaatagcg
acactgtgggttggtcttggccagacggtgctgagttgccattca
ccattgacaag
FLU_T2_NA_4 (N1_FINAL_3)-amino acid
sequence (SEQ ID NO: 18):
MNPNQKIITIGSICMVVGIISLILQIGNIISIWVSHSIQTGNQNH
PETCNQSIITYENNTWVNQTYVNISNTNVVAGQDATSVILAGNSS
LCPISGWAIYSKDNGIRIGSKGDVFVIREPFISCSHLECRTFFLT
QGALLNDKHSNGTVKDRSPYRTLMSCPVGEAPSPYNSRFESVAWS
ASACHDGMGWLTIGISGPDNGAVAVLKYNGIITDTIKSWRNNILR
TQESECACVNGSCFTIMTDGPSNGQASYKIFKIEKGKVIKSIELN
APNYHYEECSCYPDTGKVMCVCRDNWHGSNRPWVSFDQNLDYQIG
YICSGVFGDNPRPNDGTGSCGPVSSNGANGVKGFSFRYGNGVWIG
RTKSTSSRSGFEMIWDPNGWTETDSSFSVKQDIVAITDWSGYSGS
FVQHPELTGLDCMRPCFWVELIRGQPKENTIWTSGSSISFCGVNS
DTVGWSWPDGAELPFTIDK
FLU_T2_NA_4 (N1_FINAL_3)-nucleic acid
sequence (SEQ ID NO: 19):
atgaatccaaatcaaaaaataataaccattgggtcaatctgtatg
gtagttggaataattagcctaatattgcaaatagggaatataatc
tcaatatgggttagccattcaattcaaactggaaatcaaaaccat
cctgaaacatgcaaccaaagcatcattacctatgaaaataacacc
tgggtgaatcaaacatatgttaacattagcaatactaacgttgtt
gctggacaggatgcaacttcagtgatattagccggcaattcctct
ctttgccccatcagtggggggctatatacagcaaagacaatggca
taagaattggttccaaaggagacgtttttgtcataagagagccat
ttatttcatgctctcacttggaatgcaggaccttttttctgactc
aaggcgccttgctgaatgacaagcattcaaatgggaccgtcaagg
acagaagcccctatagaaccttaatgagctgccctgttggtgaag
ctccgtctccgtacaattcaaggttcgaatcggttgcttggtcag
caagtgcatgccatgatggcatgggctggctaacaatcggaattt
ccggtccagataatggagcagtggctgtattaaaatacaatggta
taataacagacaccatcaaaagttggaggaacaacatattgagaa
cgcaagagtctgaatgtgcctgtgtaaatggttcatgttttacta
taatgaccgatggcccaagtaatgggcaggcctcgtacaaaattt
tcaagatagagaaggggaaggttattaaatcaattgagttgaatg
cacctaattaccactacgaggaatgttcctgttaccctgatacag
gtaaagtgatgtgtgtgtgcagagacaattggcatggttcgaatc
gaccatgggtgtctttcgatcaaaatctggattatcaaataggat
acatctgcagtggggttttcggtgacaatccgcgtcccaatgatg
gaacaggcagctgtggtccagtgtcttctaatggagcaaatggag
taaagggattttcatttaggtatggtaatggtgtttggataggaa
gaactaaaagtaccagttccagaagcgggtttgagatgatttggg
atcctaatggatggacagagactgatagtagtttctctgtgaaac
aagatattgtagcaataactgattggtcagggtacagcgggagtt
tcgttcaacatcctgagctaacagggctggactgcatgaggcctt
gcttctgggttgaattaatcaggggacaacctaaagagaacacaa
tctggactagtgggagcagcatttccttttgtggcgtaaatagtg
atactgtaggttggtcttggccagacggtgctgagttgccattca
ccattgacaag

### EXAMPLE 10—ANTIBODY INHIBITION OF NEURAMINIDASE ACTIVITY OF FLU_T2_NA_3 AND FLU_T2_NA_4

This example describes screening of neuraminidase polypeptides according to embodiments of the invention (FLU_T2_NA_3 and FLU_T2_NA_4) against a panel of monoclonal antibodies that recognise different neuraminidase epitopes.

Neuraminidase vaccines elicit binding antibodies or antibodies that inhibit the activity of the neuraminidase enzyme. This has been shown to correlate with reduction of severity of disease, but not necessarily protection from infection. They also reduce transmission from infected vaccinated people, as the viruses require the NA activity to exit from infected cells.

Pseudotype Based Enzyme-Linked Lectin Assay (pELLA)

Lentiviral pseudotypes are produced bearing the neuraminidase of selected influenza virus strains (e.g. the N9 from A/Shanghai/02/2013 (H7N9) or of a polypeptide according to an embodiment of the invention (e.g. T2_NA_3).

These pseudotypes bearing NA are used to digest the carbohydrate fetuin from pre-coated ELISA plates in a dilution series. The resulting product from the digested fetuin contains terminal galactose residues that can be recognised by the peanut lectin (conjugated to horseradish peroxidase).

The more the NA digests the fetuin, the more galactose is exposed, so more peanut lectin (HRPO) attaches to the galactose. An ELISA-based readout proportional to the enzymatic activity of the NA is obtained (Couzens et al., J Virol Methods. 2014 Dec. 15; 210:7-14.)

The NA-pseudotypes are first titrated, then an inhibition assay is performed with antibodies or serum to ‘knock down’ the activity of the enzyme with antibodies. As this is a functional assay, it will only detect antibodies interfering with the enzymatic activity of the NA.

FIG. 5:

Panel of monoclonal antibodies tested against FLU_T2_NA_3 (N1_FINAL_2):
Strong inhibition of NA activity by: 2D4, Z2B3, 3H4, 1H8, 2D9, 3H10, 4E9, 4G2, 1H5, 2G6, A67C
Weak inhibition by: 3C2
No inhibition by: AF9C, 4C4, 2B5, 1C7, 3A2

FLU_T2_NA_3 (=N1_FINAL_2=na2=na2p1 in FIG. 5)

FIG. 6:

Panel of monoclonal antibodies tested against FLU_T2_NA_4 (N1_FINAL_3):
Strong inhibition of NA activity by: Z2B3, 2D4, 1H8, 3H4, 2D9, 3H10, 4E9, 1H5, 2G6, 4G2, A67C
Weak inhibition by: 4C4, 3C2
No inhibition by: AF9C, 2B5, 1C7, 3A2

FLU_T2_NA_4 (=N1_FINAL_3=p1na3=na3 in FIG. 6)

FIG. 7:

Panel of monoclonal antibodies tested against FLU_T2_NA_18 (N9_FINAL_1), FLU_T2_NA_19 (N9_FINAL_2), FLU_T2_NA_20 (N9_FINAL_3):
Strong inhibition of NA activity by: 1E8, 7F8, 5H11, 7A4, 7F12, 2F6, Z2B3, 1E8
Weak inhibition by: 12H3
No inhibition by: N/A

For the wild type N9 (A/Shanghai/02/2013):
Strong inhibition by: 1E8, 5H11, 7A4, 2F6, 7F12, Z2B3
No inhibition by: 7F8 and 12H3

It was concluded from the results described above, and shown in FIGS. 5-7, that neuraminidase polypeptides according to embodiments of the invention (FLU_T2_NA_3 and FLU_T2_NA_4) contain epitopes conserved between N1 from seasonal H1N1, pandemic H1N1 and N1 from avian H5N1, as well as conserved epitope (Z2B3 mAb) between N1 and N9.

### Monoclonal Antibody Panel:

mAbs from Hongguan Wan, FDA:

| | | |
|---|---|---|
| mAb_1E8 | N9 | Wan et al., Journal of Virology, 2013, Vol. 87(16): 9290-9300; |
| mAb_7F8 | N9 | Wan et al., Journal of Virology, 2018, Vol. 92(4): 1-17; |
| mAb_11B2 | N9 | Wan et al., Nat Commun., 2015, Feb 10; 6: 6114; |
| mAb_5H11 | N9 | |
| mAb_7A4 | N9 | |
| mAb_7F12 | N9 | |
| mAb_2F6 | N9 | |
| mAb_3A2 | N1 | |
| mAb_4G2 | N1 | |
| mAb_1H5 | N1 | |
| mAb_2G6 | N1 | |
| mAb_2D9 | N1 | |
| mAb_3H10 | N1 | |
| mAb_4E9 | N1 | |
| mAb_1C7 | N1 | |
| mAb_3C2 | N1 | |
| mAb_2B5 | N1 | |
| mAb_3H4 | N1 | |
| mAb_1H8 | N1 | |
| mAb_2D4 | N1 | |
| mAb_4C4 | N1 | |

mAbs from Alain Townsend, Oxford:
mAb_AF9C N1 from seasonal and pandemic H1N1 Rijal et al., Journal of Virology, February 2020 Volume 94 Issue 4, 1-17;
mAb_Z2B3 N1 and N9 Rijal et al., Journal of Virology, February 2020 Volume 94 Issue 4, 1-17

### FACS Binding Assay:

The NA is expressed on the cell surface of HEK293T/17 cells and serum/mAbs are allowed to bind to it. Binding is detected with a secondary antibody directed to the mouse or human serum antibodies. The cells are passed through a Fluorescent activated cell sampler (FACS cytometer) and the amount of binding present in a sample is measured. This binding is irrespective of whether the antibodies interfere with the enzymatic activity. These may be antibodies that act through ADCC mechanisms through immune cells.

### EXAMPLE 11—PEVAC EXPRESSION VECTOR

FIG. 8 shows a map of the pEVAC expression vector. The sequence of the multiple cloning site of the vector is given below, followed by its entire nucleotide sequence.

```
Sequence of pEVAC Multiple Cloning Site (MCS) (SEQ ID NO: 20):
                                                                                  PstI [symbol]  SalI
pEVAC 1301 ACAGACTGTT CCTTTCCATG GGTCTTTTCT GCAGTCACCG TC [symbol] GT
                                        BclI XbaI BamHI [symbol]   BglII
pEVAC 1351 CGACACGTGT GATCATCTAG AGGATCC [symbol] AGATC T
Entire Sequence of pEVAC (SEQ ID NO: 21):
```

| | | |
|---|---|---|
| CMV-IE-E/P: | 248-989 | CMV immediate early 1 enhancer/promoter |
| KanR: | 3445-4098 | Kanamycin resistance |
| SD: | 990-1220 | Splice donor |
| SA: | 1221-1343 | Splice acceptor |
| Tbgh: | 1392-1942 | Terminator signal from bovine growth hormone |
| pUC-ori: | 2096-2769 | pUC-plasmid origin of replication |

```
      1 TCGCGCGTTT CGGTGATGAC GGTGAAAACC TCTGACACAT GCAGCTCCCG
    51 GAGACGGTCA CAGCTTGTCT GTAAGCGGAT GCCGGGAGCA GACAAGCCCG
  101 TCAGGGCGCG TCAGCGGGTG TTGGCGGGTG TCGGGGCTGG CTTAACTATG
  151 CGGCATCAGA GCAGATTGTA CTGAGAGTGC ACCATATGCG GTGTGAAATA
  201 CCGCACAGAT GCGTAAGGAG AAAATACCGC ATCAGATTGG CTATTGGCCA
  251 TTGCATACGT TGTATCCATA TCATAATATG TACATTTATA TTGGCTCATG
  301 TCCAACATTA CCGCCATGTT GACATTGATT ATTGACTAGT TATTAATAGT
  351 AATCAATTAC GGGGTCATTA GTTCATAGCC CATATATGGA GTTCCGCGTT
  401 ACATAACTTA CGGTAAATGG CCCGCCTGGC TGACCGCCCA ACGACCCCCG
  451 CCCATTGACG TCAATAATGA CGTATGTTCC CATAGTAACG CCAATAGGGA
  501 CTTTCCATTG ACGTCAATGG GTGGAGTATT TACGGTAAAC TGCCCACTTG
  551 GCAGTACATC AAGTGTATCA TATGCCAAGT ACGCCCCCTA TTGACGTCAA
  601 TGACGGTAAA TGGCCCGCCT GGCATTATGC CCAGTACATG ACCTTATGGG
  651 ACTTTCCTAC TTGGCAGTAC ATCTACGTAT TAGTCATCGC TATTACCATG
  701 GTGATGCGGT TTTGGCAGTA CATCAATGGG CGTGGATAGC GGTTTGACTC
  751 ACGGGGATTT CCAAGTCTCC ACCCCATTGA CGTCAATGGG AGTTTGTTTT
  801 GGCACCAAAA TCAACGGGAC TTTCCAAAAT GTCGTAACAA CTCCGCCCCA
  851 TTGACGCAAA TGGGCGGTAG GCGTGTACGG TGGGAGGTCT ATATAAGCAG
  901 AGCTCGTTTA GTGAACCGTC AGATCGCCTG GAGACGCCAT CCACGCTGTT
  951 TTGACCTCCA TAGAAGACAC CGGGACCGAT CCAGCCTCCA TCGGCTCGCA
1001 TCTCTCCTTC ACGCGCCCGC CGCCCTACCT GAGGCCGCCA TCCACGCCGG
1051 TTGAGTCGCG TTCTGCCGCC TCCCGCCTGT GGTGCCTCCT GAACTGCGTC
1101 CGCCGTCTAG GTAAGTTTAA AGCTCAGGTC GAGACCGGGC CTTTGTCCGG
1151 CGCTCCCTTG GAGCCTACCT AGACTCAGCC GGCTCTCCAC GCTTTGCCTG
1201 ACCCTGCTTG CTCAACTCTA GTTAACGGTG GAGGGCAGTG TAGTCTGAGC
1251 AGTACTCGTT GCTGCCGCGC GCGCCACCAG ACATAATAGC TGACAGACTA
1301 ACAGACTGTT CCTTTCCATG GGTCTTTTCT GCAGTCACCG TCGGTACCGT
1351 CGACACGTGT GATCATCTAG AGGATCCGCG GCCGCAGATC TGCTGTGCCT
1401 TCTAGTTGCC AGCCATCTGT TGTTTGCCCC TCCCCCGTGC CTTCCTTGAC
1451 CCTGGAAGGT GCCACTCCCA CTGTCCTTTC CTAATAAAAT GAGGAAATTG
1501 CATCGCATTG TCTGAGTAGG TGTCATTCTA TTCTGGGGGG TGGGGTGGGG
1551 CAGGACAGCA AGGGGGAGGA TTGGGAAGAC AATAGCAGGC ATGCTGGGGA
1601 TGCGGTGGGC TCTATGGCTA CCCAGGTGCT GAAGAATTGA CCCGGTTCCT
1651 CCTGGGCCAG AAAGAAGCAG GCACATCCCC TTCTCTGTGA CACACCCTGT
1701 CCACGCCCCT GGTTCTTAGT TCCAGCCCCA CTCATAGGAC ACTCATAGCT
1751 CAGGAGGGCT CCGCCTTCAA TCCCACCCGC TAAAGTACTT GGAGCGGTCT
1801 CTCCCTCCCT CATCAGCCCA CCAAACCAAA CCTAGCCTCC AAGAGTGGGA
1851 AGAAATTAAA GCAAGATAGG CTATTAAGTG CAGAGGGAGA GAAAATGCCT
1901 CCAACATGTG AGGAAGTAAT GAGAGAAATC ATAGAATTTT AAGGCCATGA
1951 TTTAAGGCCA TCATGGCCTT AATCTTCCGC TTCCTCGCTC ACTGACTCGC
2001 TGCGCTCGGT CGTTCGGCTG CGGCGAGCGG TATCAGCTCA CTCAAAGGCG
2051 GTAATACGGT TATCCACAGA ATCAGGGGAT AACGCAGGAA AGAACATGTG
2101 AGCAAAAGGC CAGCAAAAGG CCAGGAACCG TAAAAAGGCC GCGTTGCTGG
2151 CGTTTTTCCA TAGGCTCCGC CCCCCTGACG AGCATCACAA AAATCGACGC
2201 TCAAGTCAGA GGTGGCGAAA CCCGACAGGA CTATAAAGAT ACCAGGCGTT
2251 TCCCCCTGGA AGCTCCCTCG TGCGCTCTCC TGTTCCGACC CTGCCGCTTA
2301 CCGGATACCT GTCCGCCTTT CTCCCTTCGG GAAGCGTGGC GCTTTCTCAT
2351 AGCTCACGCT GTAGGTATCT CAGTTCGGTG TAGGTCGTTC GCTCCAAGCT
2401 GGGCTGTGTG CACGAACCCC CCGTTCAGCC CGACCGCTGC GCCTTATCCG
2451 GTAACTATCG TCTTGAGTCC AACCCGGTAA GACACGACTT ATCGCCACTG
2501 GCAGCAGCCA CTGGTAACAG GATTAGCAGA GCGAGGTATG TAGGCGGTGC
2551 TACAGAGTTC TTGAAGTGGT GGCCTAACTA CGGCTACACT AGAAGAACAG
2601 TATTTGGTAT CTGCGCTCTG CTGAAGCCAG TTACCTTCGG AAAAAGAGTT
2651 GGTAGCTCTT GATCCGGCAA ACAAACCACC GCTGGTAGCG GTGGTTTTTT
2701 TGTTTGCAAG CAGCAGATTA CGCGCAGAAA AAAAGGATCT CAAGAAGATC
2751 CTTTGATCTT TTCTACGGGG TCTGACGCTC AGTGGAACGA AAACTCACGT
2801 TAAGGGATTT TGGTCATGAG ATTATCAAAA AGGATCTTCA CCTAGATCCT
2851 TTTAAATTAA AAATGAAGTT TTAAATCAAT CTAAAGTATA TATGAGTAAA
2901 CTTGGTCTGA CAGTTACCAA TGCTTAATCA GTGAGGCACC TATCTCAGCG
2951 ATCTGTCTAT TTCGTTCATC CATAGTTGCC TGACTCGGGG GGGGGGGGCG
3001 CTGAGGTCTG CCTCGTGAAG AAGGTGTTGC TGACTCATAC CAGGCCTGAA
3051 TCGCCCCATC ATCCAGCCAG AAAGTGAGGG AGCCACGGTT GATGAGAGCT
3101 TTGTTGTAGG TGGACCAGTT GGTGATTTTG AACTTTTGCT TTGCCACGGA
3151 ACGGTCTGCG TTGTCGGGAA GATGCGTGAT CTGATCCTTC AACTCAGCAA
3201 AAGTTCGATT TATTCAACAA AGCCGCCGTC CCGTCAAGTC AGCGTAATGC
3251 TCTGCCAGTG TTACAACCAA TTAACCAATT CTGATTAGAA AAACTCATCG
3301 AGCATCAAAT GAAACTGCAA TTTATTCATA TCAGGATTAT CAATACCATA
3351 TTTTTGAAAA AGCCGTTTCT GTAATGAAGG AGAAAACTCA CCGAGGCAGT
3401 TCCATAGGAT GGCAAGATCC TGGTATCGGT CTGCGATTCC GACTCGTCCA
3451 ACATCAATAC AACCTATTAA TTTCCCCTCG TCAAAAATAA GGTTATCAAG
3501 TGAGAAATCA CCATGAGTGA CGACTGAATC CGGTGAGAAT GGCAAAAGCT
3551 TATGCATTTC TTTCCAGACT TGTTCAACAG GCCAGCCATT ACGCTCGTCA
3601 TCAAAATCAC TCGCATCAAC CAAACCGTTA TTCATTCGTG ATTGCGCCTG
3651 AGCGAGACGA AATACGCGAT CGCTGTTAAA AGGACAATTA CAAACAGGAA
3701 TCGAATGCAA CCGGCGCAGG AACACTGCCA GCGCATCAAC AATATTTTCA
3751 CCTGAATCAG GATATTCTTC TAATACCTGG AATGCTGTTT TCCCGGGGAT
3801 CGCAGTGGTG AGTAACCATG CATCATCAGG AGTACGGATA AAATGCTTGA
3851 TGGTCGGAAG AGGCATAAAT TCCGTCAGCC AGTTTAGTCT GACCATCTCA
3901 TCTGTAACAT CATTGGCAAC GCTACCTTTG CCATGTTTCA GAAACAACTC
3951 TGGCGCATCG GGCTTCCCAT ACAATCGATA GATTGTCGCA CCTGATTGCC
4001 CGACATTATC GCGAGCCCAT TTATACCCAT ATAAATCAGC ATCCATGTTG
4051 GAATTTAATC GCGGCCTCGA GCAAGACGTT TCCCGTTGAA TATGGCTCAT
4101 AACACCCCTT GTATTACTGT TTATGTAAGC AGACAGTTTT ATTGTTCATG
4151 ATGATATATT TTTATCTTGT GCAATGTAAC ATCAGAGATT TTGAGACACA
4201 ACGTGGCTTT CCCCCCCCCC CCATTATTGA AGCATTTATC AGGGTTATTG
4251 TCTCATGAGC GGATACATAT TTGAATGTAT TTAGAAAAAT AAACAAATAG
4301 GGGTTCCGCG CACATTTCCC CGAAAAGTGC CACCTGACGT CTAAGAAACC
4351 ATTATTATCA TGACATTAAC CTATAAAAAT AGGCGTATCA CGAGGCCCTT
4401 TCGTC
```

### EXAMPLE 12—FLU_T2_HA_3_I3

This example provides the amino acid and nucleic acid sequences of the influenza H1 region for an embodiment of the invention known as FLU_T2_HA_3_I3.

FLU_T2_HA_3_I3-amino acid sequence
(SEQ ID NO: 22):
MKAILVVLLYTFATANADTLCIGYHANNSTDTVDTVLEKNVTVTH
SVNLLEDKHNGKLCKLRGVAPLHLGKCNIAGWILGNPECESLSTA
SSWSYIVETSSSDNGTCYPGDFINYEELREQLSSVSSFERFEIFP
KTSSWPNHDSNKGVTAACPHAGAKSFYKNLIWLVKKGNSYPKLSK
SYINDKGKEVLVLWGIHHPSTTADQQSLYQNADAYVFVGTSRYSK
KFKPEIAIRPKVRDQEGRMNYYWTLVEPGDKITFEATGNLVVPRY
AFAMERNAGSGIIISDTPVHDCNTTCQTPEGAINTSLPFQNIHPI
TIGKCPKYVKSTKLRLATGLRNVPSIQSRGLFGAIAGFIEGGWTG
MVDGWYGYHHQNEQGSGYAADLKSTQNAIDKITNKVNSVIEKMNT
QFTAVGKEFNHLEKRIENLNKKVDDGELDIWTYNAELLVLLENER
TLDYHDSNVKNLYEKVRNQLKNNAKEIGNGCFEFYHKCDNTCMES
VKNGTYDYPKYSEEAKLNREKIDGVKLESTRIYQILAIYSTVASS
LVLVVSLGAISFWMCSNGSLQCRICI
FLU_T2_HA_3_I3-nucleic acid sequence
(SEQ ID NO: 23)
ATGAAGGCTATTCTGGTGGTGCTGCTGTACACCTTCGCCACCGCC
AATGCCGATACACTGTGTATTGGCTACCACGCCAACAACAGCACC
GACACCGTGGATACCGTGCTGGAAAAGAACGTGACCGTGACACAC
AGCGTGAACCTGCTGGAAGATAAGCACAACGGCAAGCTGTGCAAG
CTGAGAGGCGTTGCACCTCTGCACCTGGGCAAGTGTAATATCGCC
GGCTGGATCCTGGGCAACCCTGAGTGTGAAAGCCTGAGCACAGCC
AGCAGCTGGTCCTACATCGTGGAAACCAGCAGCAGCGACAACGGC
ACATGCTACCCCGGCGACTTCATCAACTACGAGGAACTGAGAGAG
CAGCTGAGCAGCGTCAGCAGCTTCGAGAGATTCGAGATTTTCCCC
AAGACCTCCAGCTGGCCCAACCACGATTCTAACAAGGGCGTGACA
GCCGCCTGTCCTCATGCCGGCGCTAAGAGCTTCTACAAGAACCTG
ATCTGGCTGGTCAAGAAGGGCAACAGCTACCCCAAGCTGAGCAAG
AGCTACATCAACGACAAGGGCAAAGAGGTGCTGGTCCTCTGGGGC
ATCCACCATCCTTCTACAACAGCCGACCAGCAGAGCCTGTACCAG
AATGCCGATGCCTACGTGTTCGTGGGCACCAGCAGATACAGCAAG
AAGTTCAAGCCCGAGATCGCCATCAGACCCAAAGTGCGGGATCAA
GAGGGCAGAATGAACTACTACTGGACCCTGGTGGAACCCGGCGAC
AAGATCACATTTGAGGCCACAGGCAACCTGGTGGTCCCTAGATAC
GCCTTCGCCATGGAAAGAAATGCCGGCAGCGGCATCATCATCAGC
GACACACCTGTGCACGACTGCAACACCACCTGTCAGACACCTGAG
GGCGCCATCAATACCAGCCTGCCTTTCCAGAACATTCACCCCATC
ACCATCGGCAAGTGCCCCAAATACGTGAAGTCCACAAAGCTGAGA
CTGGCCACCGGCCTGAGAAATGTGCCTAGCATCCAGAGCAGAGGC
CTGTTTGGAGCCATTGCCGGCTTTATCGAAGGCGGCTGGACAGGC
ATGGTTGACGGATGGTACGGCTACCACCATCAGAATGAGCAAGGC
AGCGGATACGCCGCCGATCTGAAGTCTACACAGAACGCCATCGAT
AAGATCACCAACAAAGTGAACAGCGTGATCGAGAAGATGAACACC
CAGTTCACCGCCGTGGGAAAAGAGTTCAACCACCTGGAAAAGCGC
ATCGAGAACCTGAACAAGAAGGTGGACGACGGCTTCCTGGACATC
TGGACCTATAATGCCGAGCTGCTCGTGCTGCTCGAGAACGAGAGA
ACCCTGGACTACCACGACAGCAACGTGAAGAACCTGTACGAGAAA
GTGCGGAACCAGCTGAAGAACAACGCCAAAGAGATCGGCAACGGC
TGCTTCGAGTTCTACCACAAGTGCGACAATACCTGCATGGAAAGC
GTGAAGAATGGCACCTACGACTACCCTAAGTACAGCGAGGAAGCC
AAGCTGAACCGCGAGAAGATTGACGGCGTGAAGCTGGAAAGCACC
CGGATCTATCAGATCCTGGCCATCTACAGCACAGTGGCCTCTAGC
CTGGTGCTGGTGGTGTCTCTGGGAGCCATCAGCTTTTGGATGTGC
AGCAATGGCAGCCTCCAGTGCCGGATCTGCATC

### EXAMPLE 13—PEVAC-FLU_T2_HA-3-I-3

This example provides the nucleic acid sequence of pEVAC-FLU_T2_HA-3-I-3.

| pEVAC-FLU_T2_HA-3-1-3-nucleic acid sequence (SEQ ID NO: 24): | |
|---|---|
| LOCUS 17ADKK4C_I-3_pVRC8400EVAC_Ar 6083 bp DNA circular | |
| FEATURES | Location/Qualifiers |
| promoter | complement(5925 . . . 5953) |
| | /label = “Amp_Rpromoter” |
| promoter | 868 . . . 987 |
| | /label = “CMV2_promoter” |
| CDS | complement(4963 . . . 5778) |
| | /label = “Kana(R)” |
| rep_origin | complement(3818 . . . 4437) |
| | /label = “pBR322_origin” |
| primer | complement(29 . . . 51) |
| | /label = “pGEX3_primer” |
| primer | 855 . . . 875 |
| | /label = “CMV_fwd_primer” |
| primer | 899 . . . 918 |
| | /label = “pCEP_fwd_primer” |
| primer | 901 . . . 925 |
| | /label = “LNCX_primer” |
| polyA_site | 3071 . . . 3295 |
| | /label = “BGH\pA” |
| promoter | 394 . . . 904 |
| | /label = “CMV_Promoter” |
| CDS | 1343 . . . 3063 |
| | /label = “I-3” |

TCGCGCGTTTCGGTGATGACGGTGAAAACCTCTGACACATGCAGCTCCCGGAGACGGTCACAGCTTG
TCTGTAAGCGGATGCCGGGAGCAGACAAGCCCGTCAGGGCGCGTCAGCGGGTGTTGGCGGGTGTCGG
GGCTGGCTTAACTATGCGGCATCAGAGCAGATTGTACTGAGAGTGCACCATATGCGGTGTGAAATAC
CGCACAGATGCGTAAGGAGAAAATACCGCATCAGATTGGCTATTGGCCATTGCATACGTTGTATCCA
TATCATAATATGTACATTTATATTGGCTCATGTCCAACATTACCGCCATGTTGACATTGATTATTGA
CTAGTTATTAATAGTAATCAATTACGGGGTCATTAGTTCATAGCCCATATATGGAGTTCCGCGTTAC
ATAACTTACGGTAAATGGCCCGCCTGGCTGACCGCCCAACGACCCCCGCCCATTGACGTCAATAATG
ACGTATGTTCCCATAGTAACGCCAATAGGGACTTTCCATTGACGTCAATGGGTGGAGTATTTACGGT
AAACTGCCCACTTGGCAGTACATCAAGTGTATCATATGCCAAGTACGCCCCCTATTGACGTCAATGA
CGGTAAATGGCCCGCCTGGCATTATGCCCAGTACATGACCTTATGGGACTTTCCTACTTGGCAGTAC
ATCTACGTATTAGTCATCGCTATTACCATGGTGATGCGGTTTTGGCAGTACATCAATGGGCGTGGAT
AGCGGTTTGACTCACGGGGATTTCCAAGTCTCCACCCCATTGACGTCAATGGGAGTTTGTTTTGGCA
CCAAAATCAACGGGACTTTCCAAAATGTCGTAACAACTCCGCCCCATTGACGCAAATGGGCGGTAGG
CGTGTACGGTGGGAGGTCTATATAAGCAGAGCTCGTTTAGTGAACCGTCAGATCGCCTGGAGACGCC
ATCCACGCTGTTTTGACCTCCATAGAAGACACCGGGACCGATCCAGCCTCCATCGGCTCGCATCTCT
CCTTCACGCGCCCGCCGCCCTACCTGAGGCCGCCATCCACGCCGGTTGAGTCGCGTTCTGCCGCCTC
CCGCCTGTGGTGCCTCCTGAACTGCGTCCGCCGTCTAGGTAAGTTTAAAGCTCAGGTCGAGACCGGG
CCTTTGTCCGGCGCTCCCTTGGAGCCTACCTAGACTCAGCCGGCTCTCCACGCTTTGCCTGACCCTG
CTTGCTCAACTCTAGTTAACGGTGGAGGGCAGTGTAGTCTGAGCAGTACTCGTTGCTGCCGCGCGCG
CCACCAGACATAATAGCTGACAGACTAACAGACTGTTCCTTTCCATGGGTCTTTTCTGCAGTCACCG
TCGGTACCGCCACCATGAAGGCTATTCTGGTGGTGCTGCTGTACACCTTCGCCACCGCCAATGCCGA
TACACTGTGTATTGGCTACCACGCCAACAACAGCACCGACACCGTGGATACCGTGCTGGAAAAGAAC
GTGACCGTGACACACAGCGTGAACCTGCTGGAAGATAAGCACAACGGCAAGCTGTGCAAGCTGAGAG
GCGTTGCACCTCTGCACCTGGGCAAGTGTAATATCGCCGGCTGGATCCTGGGCAACCCTGAGTGTGA
AAGCCTGAGCACAGCCAGCAGCTGGTCCTACATCGTGGAAACCAGCAGCAGCGACAACGGCACATGC
TACCCCGGCGACTTCATCAACTACGAGGAACTGAGAGAGCAGCTGAGCAGCGTCAGCAGCTTCGAGA
GATTCGAGATTTTCCCCAAGACCTCCAGCTGGCCCAACCACGATTCTAACAAGGGCGTGACAGCCGC
CTGTCCTCATGCCGGCGCTAAGAGCTTCTACAAGAACCTGATCTGGCTGGTCAAGAAGGGCAACAGC
TACCCCAAGCTGAGCAAGAGCTACATCAACGACAAGGGCAAAGAGGTGCTGGTCCTCTGGGGCATCC
ACCATCCTTCTACAACAGCCGACCAGCAGAGCCTGTACCAGAATGCCGATGCCTACGTGTTCGTGGG
CACCAGCAGATACAGCAAGAAGTTCAAGCCCGAGATCGCCATCAGACCCAAAGTGCGGGATCAAGAG
GGCAGAATGAACTACTACTGGACCCTGGTGGAACCCGGCGACAAGATCACATTTGAGGCCACAGGCA
ACCTGGTGGTCCCTAGATACGCCTTCGCCATGGAAAGAAATGCCGGCAGCGGCATCATCATCAGCGA
CACACCTGTGCACGACTGCAACACCACCTGTCAGACACCTGAGGGCGCCATCAATACCAGCCTGCCT
TTCCAGAACATTCACCCCATCACCATCGGCAAGTGCCCCAAATACGTGAAGTCCACAAAGCTGAGAC
TGGCCACCGGCCTGAGAAATGTGCCTAGCATCCAGAGCAGAGGCCTGTTTGGAGCCATTGCCGGCTT
TATCGAAGGCGGCTGGACAGGCATGGTTGACGGATGGTACGGCTACCACCATCAGAATGAGCAAGGC
AGCGGATACGCCGCCGATCTGAAGTCTACACAGAACGCCATCGATAAGATCACCAACAAAGTGAACA
GCGTGATCGAGAAGATGAACACCCAGTTCACCGCCGTGGGAAAAGAGTTCAACCACCTGGAAAAGCG
CATCGAGAACCTGAACAAGAAGGTGGACGACGGCTTCCTGGACATCTGGACCTATAATGCCGAGCTG
CTCGTGCTGCTCGAGAACGAGAGAACCCTGGACTACCACGACAGCAACGTGAAGAACCTGTACGAGA
AAGTGCGGAACCAGCTGAAGAACAACGCCAAAGAGATCGGCAACGGCTGCTTCGAGTTCTACCACAA
GTGCGACAATACCTGCATGGAAAGCGTGAAGAATGGCACCTACGACTACCCTAAGTACAGCGAGGAA
GCCAAGCTGAACCGCGAGAAGATTGACGGCGTGAAGCTGGAAAGCACCCGGATCTATCAGATCCTGG
CCATCTACAGCACAGTGGCCTCTAGCCTGGTGCTGGTGGTGTCTCTGGGAGCCATCAGCTTTTGGAT
GTGCAGCAATGGCAGCCTCCAGTGCCGGATCTGCATCTGAGCGGCCGCAGATCTGCTGTGCCTTCTA
GTTGCCAGCCATCTGTTGTTTGCCCCTCCCCCGTGCCTTCCTTGACCCTGGAAGGTGCCACTCCCAC
TGTCCTTTCCTAATAAAATGAGGAAATTGCATCGCATTGTCTGAGTAGGTGTCATTCTATTCTGGGG
GGTGGGGTGGGGCAGGACAGCAAGGGGGAGGATTGGGAAGACAATAGCAGGCATGCTGGGGATGCGG
TGGGCTCTATGGCTACCCAGGTGCTGAAGAATTGACCCGGTTCCTCCTGGGCCAGAAAGAAGCAGGC
ACATCCCCTTCTCTGTGACACACCCTGTCCACGCCCCTGGTTCTTAGTTCCAGCCCCACTCATAGGA
CACTCATAGCTCAGGAGGGCTCCGCCTTCAATCCCACCCGCTAAAGTACTTGGAGCGGTCTCTCCCT
CCCTCATCAGCCCACCAAACCAAACCTAGCCTCCAAGAGTGGGAAGAAATTAAAGCAAGATAGGCTA
TTAAGTGCAGAGGGAGAGAAAATGCCTCCAACATGTGAGGAAGTAATGAGAGAAATCATAGAATTTT
AAGGCCATGATTTAAGGCCATCATGGCCTTAATCTTCCGCTTCCTCGCTCACTGACTCGCTGCGCTC
GGTCGTTCGGCTGCGGCGAGCGGTATCAGCTCACTCAAAGGCGGTAATACGGTTATCCACAGAATCA
GGGGATAACGCAGGAAAGAACATGTGAGCAAAAGGCCAGCAAAAGGCCAGGAACCGTAAAAAGGCCG
CGTTGCTGGCGTTTTTCCATAGGCTCCGCCCCCCTGACGAGCATCACAAAAATCGACGCTCAAGTCA
GAGGTGGCGAAACCCGACAGGACTATAAAGATACCAGGCGTTTCCCCCTGGAAGCTCCCTCGTGCGC
TCTCCTGTTCCGACCCTGCCGCTTACCGGATACCTGTCCGCCTTTCTCCCTTCGGGAAGCGTGGCGC
TTTCTCATAGCTCACGCTGTAGGTATCTCAGTTCGGTGTAGGTCGTTCGCTCCAAGCTGGGCTGTGT
GCACGAACCCCCCGTTCAGCCCGACCGCTGCGCCTTATCCGGTAACTATCGTCTTGAGTCCAACCCG
GTAAGACACGACTTATCGCCACTGGCAGCAGCCACTGGTAACAGGATTAGCAGAGCGAGGTATGTAG
GCGGTGCTACAGAGTTCTTGAAGTGGTGGCCTAACTACGGCTACACTAGAAGAACAGTATTTGGTAT
CTGCGCTCTGCTGAAGCCAGTTACCTTCGGAAAAAGAGTTGGTAGCTCTTGATCCGGCAAACAAACC
ACCGCTGGTAGCGGTGGTTTTTTTGTTTGCAAGCAGCAGATTACGCGCAGAAAAAAAGGATCTCAAG
AAGATCCTTTGATCTTTTCTACGGGGTCTGACGCTCAGTGGAACGAAAACTCACGTTAAGGGATTTT
GGTCATGAGATTATCAAAAAGGATCTTCACCTAGATCCTTTTAAATTAAAAATGAAGTTTTAAATCA
ATCTAAAGTATATATGAGTAAACTTGGTCTGACAGTTACCAATGCTTAATCAGTGAGGCACCTATCT
CAGCGATCTGTCTATTTCGTTCATCCATAGTTGCCTGACTCGGGGGGGGGGGGCGCTGAGGTCTGCC
TCGTGAAGAAGGTGTTGCTGACTCATACCAGGCCTGAATCGCCCCATCATCCAGCCAGAAAGTGAGG
GAGCCACGGTTGATGAGAGCTTTGTTGTAGGTGGACCAGTTGGTGATTTTGAACTTTTGCTTTGCCA
CGGAACGGTCTGCGTTGTCGGGAAGATGCGTGATCTGATCCTTCAACTCAGCAAAAGTTCGATTTAT
TCAACAAAGCCGCCGTCCCGTCAAGTCAGCGTAATGCTCTGCCAGTGTTACAACCAATTAACCAATT
CTGATTAGAAAAACTCATCGAGCATCAAATGAAACTGCAATTTATTCATATCAGGATTATCAATACC
ATATTTTTGAAAAAGCCGTTTCTGTAATGAAGGAGAAAACTCACCGAGGCAGTTCCATAGGATGGCA
AGATCCTGGTATCGGTCTGCGATTCCGACTCGTCCAACATCAATACAACCTATTAATTTCCCCTCGT
CAAAAATAAGGTTATCAAGTGAGAAATCACCATGAGTGACGACTGAATCCGGTGAGAATGGCAAAAG
CTTATGCATTTCTTTCCAGACTTGTTCAACAGGCCAGCCATTACGCTCGTCATCAAAATCACTCGCA
TCAACCAAACCGTTATTCATTCGTGATTGCGCCTGAGCGAGACGAAATACGCGATCGCTGTTAAAAG
GACAATTACAAACAGGAATCGAATGCAACCGGCGCAGGAACACTGCCAGCGCATCAACAATATTTTC
ACCTGAATCAGGATATTCTTCTAATACCTGGAATGCTGTTTTCCCGGGGATCGCAGTGGTGAGTAAC
CATGCATCATCAGGAGTACGGATAAAATGCTTGATGGTCGGAAGAGGCATAAATTCCGTCAGCCAGT
TTAGTCTGACCATCTCATCTGTAACATCATTGGCAACGCTACCTTTGCCATGTTTCAGAAACAACTC
TGGCGCATCGGGCTTCCCATACAATCGATAGATTGTCGCACCTGATTGCCCGACATTATCGCGAGCC
CATTTATACCCATATAAATCAGCATCCATGTTGGAATTTAATCGCGGCCTCGAGCAAGACGTTTCCC
GTTGAATATGGCTCATAACACCCCTTGTATTACTGTTTATGTAAGCAGACAGTTTTATTGTTCATGA
TGATATATTTTTATCTTGTGCAATGTAACATCAGAGATTTTGAGACACAACGTGGCTTTCCCCCCCC
CCCCATTATTGAAGCATTTATCAGGGTTATTGTCTCATGAGCGGATACATATTTGAATGTATTTAGA
AAAATAAACAAATAGGGGTTCCGCGCACATTTCCCCGAAAAGTGCCACCTGACGTCTAAGAAACCAT
TATTATCATGACATTAACCTATAAAAATAGGCGTATCACGAGGCCCTTTCGTC
//

### EXAMPLE 14—BROAD COVERAGE H1N1 VACCINE CANDIDATE

This example provides a broad coverage H1N1 string-based vaccine construct (panH1N1 vaccine candidate). panH1N1 comprises an isolated polynucleotide comprising nucleotide sequence encoding FLU_T2_HA_3_I3 (SEQ ID NO:23), FLU_T2_NA_3 (SEQ ID NO:17), and FLU_T2_M2_1 (SEQ ID NO:15) designed subunits, covalently linked.

FIG. 9 shows logₑIC₅₀ plot for pEVAC_Flu_T2_HA_3_1-3 and other controls.

Elicitation of neutralising antibodies by our vaccine candidate—Flu_T2_HA_3_1-3 against A/Brisbane/02/2018, A/Califomia/07/2009, A/swine/Guangxi/2013, and A/swine/Henan/SN10/2018 was confirmed using pMN assay. Various controls used are: primary strains viz. A/Brisbane/02/2018, A/Michigan/45/2015, cobra design: H1N1 cobra, our seasonal H1N1 vaccine candidate: Flu_T2_HA_2 and monoclonal antibodies—mAb 4F8 and mAb F16.

FIG. 10 shows inhibition of enzymatic activity of A/Brisbane/02/2018 neuraminidase by sera from mouse vaccinated by (A) PBS, (B) Primary strain—A/Brisbane/02/2018, (C) N1_Final_1, (D) N1_Final_2 (Flu_T2_NA_3).

This data shows superiority in neutralisation breadth to some isolates, or equivalence in breadth to others, compared to the Cobra candidate.

### EXAMPLE 15—PANH1N1 VACCINE CANDIDATE

This example provides the nucleic acid sequence of the broad coverage H1N1 vaccine candidate of the invention known as panH1N1. panH1N1 comprises an isolated polynucleotide comprising nucleotide sequence encoding FLU_T2_HA_3_I3 (SEQ ID NO:23), FLU_T2_NA_3 (SEQ ID NO:17), and FLU_T2_M2_1 (SEQ ID NO:15) designed subunits, covalently linked. The amino acid sequence of panH1N1 (SEQ ID NO:63) is also provided.

panH1N1-nucleic acid sequence
(SEQ ID NO: 25)
ATGAAGGCTATTCTGGTGGTGCTGCTGTACACCTTCGCCACCGCCAATGCCGATACACTGTGTATTGGCTACCA
CGCCAACAACAGCACCGACACCGTGGATACCGTGCTGGAAAAGAACGTGACCGTGACACACAGCGTGAACCTGC
TGGAAGATAAGCACAACGGCAAGCTGTGCAAGCTGAGAGGCGTTGCACCTCTGCACCTGGGCAAGTGTAATATC
GCCGGCTGGATCCTGGGCAACCCTGAGTGTGAAAGCCTGAGCACAGCCAGCAGCTGGTCCTACATCGTGGAAAC
CAGCAGCAGCGACAACGGCACATGCTACCCCGGCGACTTCATCAACTACGAGGAACTGAGAGAGCAGCTGAGCA
GCGTCAGCAGCTTCGAGAGATTCGAGATTTTCCCCAAGACCTCCAGCTGGCCCAACCACGATTCTAACAAGGGC
GTGACAGCCGCCTGTCCTCATGCCGGCGCTAAGAGCTTCTACAAGAACCTGATCTGGCTGGTCAAGAAGGGCAA
CAGCTACCCCAAGCTGAGCAAGAGCTACATCAACGACAAGGGCAAAGAGGTGCTGGTCCTCTGGGGCATCCACC
ATCCTTCTACAACAGCCGACCAGCAGAGCCTGTACCAGAATGCCGATGCCTACGTGTTCGTGGGCACCAGCAGA
TACAGCAAGAAGTTCAAGCCCGAGATCGCCATCAGACCCAAAGTGCGGGATCAAGAGGGCAGAATGAACTACTA
CTGGACCCTGGTGGAACCCGGCGACAAGATCACATTTGAGGCCACAGGCAACCTGGTGGTCCCTAGATACGCCT
TCGCCATGGAAAGAAATGCCGGCAGCGGCATCATCATCAGCGACACACCTGTGCACGACTGCAACACCACCTGT
CAGACACCTGAGGGCGCCATCAATACCAGCCTGCCTTTCCAGAACATTCACCCCATCACCATCGGCAAGTGCCC
CAAATACGTGAAGTCCACAAAGCTGAGACTGGCCACCGGCCTGAGAAATGTGCCTAGCATCCAGAGCAGAGGCC
TGTTTGGAGCCATTGCCGGCTTTATCGAAGGCGGCTGGACAGGCATGGTTGACGGATGGTACGGCTACCACCAT
CAGAATGAGCAAGGCAGCGGATACGCCGCCGATCTGAAGTCTACACAGAACGCCATCGATAAGATCACCAACAA
AGTGAACAGCGTGATCGAGAAGATGAACACCCAGTTCACCGCCGTGGGAAAAGAGTTCAACCACCTGGAAAAGC
GCATCGAGAACCTGAACAAGAAGGTGGACGACGGCTTCCTGGACATCTGGACCTATAATGCCGAGCTGCTCGTG
CTGCTCGAGAACGAGAGAACCCTGGACTACCACGACAGCAACGTGAAGAACCTGTACGAGAAAGTGCGGAACCA
GCTGAAGAACAACGCCAAAGAGATCGGCAACGGCTGCTTCGAGTTCTACCACAAGTGCGACAATACCTGCATGG
AAAGCGTGAAGAATGGCACCTACGACTACCCTAAGTACAGCGAGGAAGCCAAGCTGAACCGCGAGAAGATTGAC
GGCGTGAAGCTGGAAAGCACCCGGATCTATCAGATCCTGGCCATCTACAGCACAGTGGCCTCTAGCCTGGTGCT
GGTGGTGTCTCTGGGAGCCATCAGCTTTTGGATGTGCAGCAATGGCAGCCTCCAGTGCCGGATCTGCATCGGAA
GCGGAGAAGGCAGAGGCAGCCTGCTGACATGCGGAGATGTGGAAGAGAATCCCGGACCTATGAATCCCAACCAG
AAGATCATCACCATCGGCAGCATCTGCATGGTCGTGGGCATCATCAGCCTGATCCTCCAGATCGGCAACATCAT
CTCCATCTGGGTGTCCCACAGCATCCAGACCGGCAATCAGAACCAGCCTGAGACATGCAACCAGTCCATCATCA
CCTACGAGAACAACACCTGGGTCAACCAGACCTACGTGAACATCAGCAACACCAACTTCGTGGCCGAACAGGCC
GTGGCTTCTGTTGCCCTGGCCGGAAATAGCTCTCTGTGCCCTATTAGCGGCTGGGCCATCTACAGCAAGGACAA
CGGCATCCGGATCGGCTCTAAGGGCGACGTGTTCGTGATCAGAGAGCCCTTCATCAGCTGCTCCCACCTGGAAT
GCCGGACATTCTTTCTGACCCAAGGCGCCCTGCTGAACGACAAGCACAGCAATGGCACCGTGAAGGACAGAAGC
CCCTACAGAACCCTGATGAGCTGCCCTGTGGGAGAAGCCCCATCTCCTTACAACAGCAGATTCGAGTCCGTGGC
TTGGAGCGCCTCTGCCTGTCACGATGGAATCAGCTGGCTGACAATCGGCATCAGCGGCCCTGATAATGGCGCTG
TGGCCGTGCTGAAGTACAACGGAATCATCACCGACACCATCAAGAGCTGGCGGAACAACATCCTGCGGACCCAA
GAGTCCGAGTGCGCCTGTATCAATGGCAGCTGCTTCACCATCATGACAGACGGCCCTAGCAATGGCCAGGCCAG
CTACAAGATTTTCAAGATCGAGAAGGGCAAAGTGGTCAAGAGCGTGGAACTGAACGCCCCTAACTACCACTACG
AGGAATGCAGCTGCTACCCCGATGCCGGCGAAGTGATGTGCGTGTGCAGAGACAATTGGCACGGCAGCAACAGA
CCTTGGGTGTCCTTCAACCAGAACCTGGAATATCAGATCGGCTATATCTGCTCCGGCGTGTTCGGCGACAACCC
CAGACCTAATGATGGCACAGGCAGCTGTGGCCCCGTGTCATCTAATGGCGCCTATGGCGTGAAGGGCTTCAGCT
TTAAGTACGGCAAAGGCGTGTGGATCGGCCGGACCAAGAGCACCTCTAGCAGATCCGGCTTCGAGATGATCTGG
GACCCCAACGGCTGGACCGAGACAGATAGCAGCTTCAGCGTGAAGCAGGACATCGTGGCCATCACCGATTGGAG
CGGCTACAGCGGAAGCTTCGTGCAGCACCCTGAACTGACAGGCCTGGACTGCATGAGGCCCTGCTTTTGGGTCG
AGCTGATCCGGGGCAGACCCAAAGAGAACACCATCTGGACAAGCGGCAGCAGCATCAGCTTTTGCGGCGTGAAC
AGCGATACCGTCGGCTGGTCTTGGCCTGATGGTGCCGAGCTGCCTTTCACCATCGACAAAGGATCCGGCGCCAC
CAACTTTAGTCTGCTGAAACAGGCCGGCGACGTCGAAGAGAACCCAGGTCCTATGTCTCTGCTGACCGAGGTGG
AAACCCCTACCAGAAATGGCTGGGAGTGCAGATGCAGCGACAGCAGCGATCCTCTGGTTATCGCCGCCAGCATC
ATCGGCATCCTGCACCTGATCCTGTGGATCCTGGACCGGCTGTTCTTCAAGTGCATCTACCGGCGGCTGAAGTA
CGGCCTGAAGAGAGGCCCTTCTACAGAGGGCGTGCCCGAGAGCATGCGGGAAGAGTACAGACAGAAACAGCAGA
GCGCCGTGGACGTGGACGATGGCCACTTCGTGAACATCGAGCTGGAATGA
pEVAC_panH1N1-nucleic acid sequence
(SEQ ID NO: 26)
TCGCGCGTTTCGGTGATGACGGTGAAAACCTCTGACACATGCAGCTCCCGGAGACGGTCACAGCTTGTCTGTAA
GCGGATGCCGGGAGCAGACAAGCCCGTCAGGGCGCGTCAGCGGGTGTTGGCGGGTGTCGGGGCTGGCTTAACTA
TGCGGCATCAGAGCAGATTGTACTGAGAGTGCACCATATGCGGTGTGAAATACCGCACAGATGCGTAAGGAGAA
AATACCGCATCAGATTGGCTATTGGCCATTGCATACGTTGTATCCATATCATAATATGTACATTTATATTGGCT
CATGTCCAACATTACCGCCATGTTGACATTGATTATTGACTAGTTATTAATAGTAATCAATTACGGGGTCATTA
GTTCATAGCCCATATATGGAGTTCCGCGTTACATAACTTACGGTAAATGGCCCGCCTGGCTGACCGCCCAACGA
CCCCCGCCCATTGACGTCAATAATGACGTATGTTCCCATAGTAACGCCAATAGGGACTTTCCATTGACGTCAAT
GGGTGGAGTATTTACGGTAAACTGCCCACTTGGCAGTACATCAAGTGTATCATATGCCAAGTACGCCCCCTATT
GACGTCAATGACGGTAAATGGCCCGCCTGGCATTATGCCCAGTACATGACCTTATGGGACTTTCCTACTTGGCA
GTACATCTACGTATTAGTCATCGCTATTACCATGGTGATGCGGTTTTGGCAGTACATCAATGGGCGTGGATAGC
GGTTTGACTCACGGGGATTTCCAAGTCTCCACCCCATTGACGTCAATGGGAGTTTGTTTTGGCACCAAAATCAA
CGGGACTTTCCAAAATGTCGTAACAACTCCGCCCCATTGACGCAAATGGGCGGTAGGCGTGTACGGTGGGAGGT
CTATATAAGCAGAGCTCGTTTAGTGAACCGTCAGATCGCCTGGAGACGCCATCCACGCTGTTTTGACCTCCATA
GAAGACACCGGGACCGATCCAGCCTCCATCGGCTCGCATCTCTCCTTCACGCGCCCGCCGCCCTACCTGAGGCC
GCCATCCACGCCGGTTGAGTCGCGTTCTGCCGCCTCCCGCCTGTGGTGCCTCCTGAACTGCGTCCGCCGTCTAG
GTAAGTTTAAAGCTCAGGTCGAGACCGGGCCTTTGTCCGGCGCTCCCTTGGAGCCTACCTAGACTCAGCCGGCT
CTCCACGCTTTGCCTGACCCTGCTTGCTCAACTCTAGTTAACGGTGGAGGGCAGTGTAGTCTGAGCAGTACTCG
TTGCTGCCGCGCGCGCCACCAGACATAATAGCTGACAGACTAACAGACTGTTCCTTTCCATGGGTCTTTTCTGC
AGTCACCGTCGGTACCGCCACCATGAAGGCTATTCTGGTGGTGCTGCTGTACACCTTCGCCACCGCCAATGCCG
ATACACTGTGTATTGGCTACCACGCCAACAACAGCACCGACACCGTGGATACCGTGCTGGAAAAGAACGTGACC
GTGACACACAGCGTGAACCTGCTGGAAGATAAGCACAACGGCAAGCTGTGCAAGCTGAGAGGCGTTGCACCTCT
GCACCTGGGCAAGTGTAATATCGCCGGCTGGATCCTGGGCAACCCTGAGTGTGAAAGCCTGAGCACAGCCAGCA
GCTGGTCCTACATCGTGGAAACCAGCAGCAGCGACAACGGCACATGCTACCCCGGCGACTTCATCAACTACGAG
GAACTGAGAGAGCAGCTGAGCAGCGTCAGCAGCTTCGAGAGATTCGAGATTTTCCCCAAGACCTCCAGCTGGCC
CAACCACGATTCTAACAAGGGCGTGACAGCCGCCTGTCCTCATGCCGGCGCTAAGAGCTTCTACAAGAACCTGA
TCTGGCTGGTCAAGAAGGGCAACAGCTACCCCAAGCTGAGCAAGAGCTACATCAACGACAAGGGCAAAGAGGTG
CTGGTCCTCTGGGGCATCCACCATCCTTCTACAACAGCCGACCAGCAGAGCCTGTACCAGAATGCCGATGCCTA
CGTGTTCGTGGGCACCAGCAGATACAGCAAGAAGTTCAAGCCCGAGATCGCCATCAGACCCAAAGTGCGGGATC
AAGAGGGCAGAATGAACTACTACTGGACCCTGGTGGAACCCGGCGACAAGATCACATTTGAGGCCACAGGCAAC
CTGGTGGTCCCTAGATACGCCTTCGCCATGGAAAGAAATGCCGGCAGCGGCATCATCATCAGCGACACACCTGT
GCACGACTGCAACACCACCTGTCAGACACCTGAGGGCGCCATCAATACCAGCCTGCCTTTCCAGAACATTCACC
CCATCACCATCGGCAAGTGCCCCAAATACGTGAAGTCCACAAAGCTGAGACTGGCCACCGGCCTGAGAAATGTG
CCTAGCATCCAGAGCAGAGGCCTGTTTGGAGCCATTGCCGGCTTTATCGAAGGCGGCTGGACAGGCATGGTTGA
CGGATGGTACGGCTACCACCATCAGAATGAGCAAGGCAGCGGATACGCCGCCGATCTGAAGTCTACACAGAACG
CCATCGATAAGATCACCAACAAAGTGAACAGCGTGATCGAGAAGATGAACACCCAGTTCACCGCCGTGGGAAAA
GAGTTCAACCACCTGGAAAAGCGCATCGAGAACCTGAACAAGAAGGTGGACGACGGCTTCCTGGACATCTGGAC
CTATAATGCCGAGCTGCTCGTGCTGCTCGAGAACGAGAGAACCCTGGACTACCACGACAGCAACGTGAAGAACC
TGTACGAGAAAGTGCGGAACCAGCTGAAGAACAACGCCAAAGAGATCGGCAACGGCTGCTTCGAGTTCTACCAC
AAGTGCGACAATACCTGCATGGAAAGCGTGAAGAATGGCACCTACGACTACCCTAAGTACAGCGAGGAAGCCAA
GCTGAACCGCGAGAAGATTGACGGCGTGAAGCTGGAAAGCACCCGGATCTATCAGATCCTGGCCATCTACAGCA
CAGTGGCCTCTAGCCTGGTGCTGGTGGTGTCTCTGGGAGCCATCAGCTTTTGGATGTGCAGCAATGGCAGCCTC
CAGTGCCGGATCTGCATCGGAAGCGGAGAAGGCAGAGGCAGCCTGCTGACATGCGGAGATGTGGAAGAGAATCC
CGGACCTATGAATCCCAACCAGAAGATCATCACCATCGGCAGCATCTGCATGGTCGTGGGCATCATCAGCCTGA
TCCTCCAGATCGGCAACATCATCTCCATCTGGGTGTCCCACAGCATCCAGACCGGCAATCAGAACCAGCCTGAG
ACATGCAACCAGTCCATCATCACCTACGAGAACAACACCTGGGTCAACCAGACCTACGTGAACATCAGCAACAC
CAACTTCGTGGCCGAACAGGCCGTGGCTTCTGTTGCCCTGGCCGGAAATAGCTCTCTGTGCCCTATTAGCGGCT
GGGCCATCTACAGCAAGGACAACGGCATCCGGATCGGCTCTAAGGGCGACGTGTTCGTGATCAGAGAGCCCTTC
ATCAGCTGCTCCCACCTGGAATGCCGGACATTCTTTCTGACCCAAGGCGCCCTGCTGAACGACAAGCACAGCAA
TGGCACCGTGAAGGACAGAAGCCCCTACAGAACCCTGATGAGCTGCCCTGTGGGAGAAGCCCCATCTCCTTACA
ACAGCAGATTCGAGTCCGTGGCTTGGAGCGCCTCTGCCTGTCACGATGGAATCAGCTGGCTGACAATCGGCATC
AGCGGCCCTGATAATGGCGCTGTGGCCGTGCTGAAGTACAACGGAATCATCACCGACACCATCAAGAGCTGGCG
GAACAACATCCTGCGGACCCAAGAGTCCGAGTGCGCCTGTATCAATGGCAGCTGCTTCACCATCATGACAGACG
GCCCTAGCAATGGCCAGGCCAGCTACAAGATTTTCAAGATCGAGAAGGGCAAAGTGGTCAAGAGCGTGGAACTG
AACGCCCCTAACTACCACTACGAGGAATGCAGCTGCTACCCCGATGCCGGCGAAGTGATGTGCGTGTGCAGAGA
CAATTGGCACGGCAGCAACAGACCTTGGGTGTCCTTCAACCAGAACCTGGAATATCAGATCGGCTATATCTGCT
CCGGCGTGTTCGGCGACAACCCCAGACCTAATGATGGCACAGGCAGCTGTGGCCCCGTGTCATCTAATGGCGCC
TATGGCGTGAAGGGCTTCAGCTTTAAGTACGGCAAAGGCGTGTGGATCGGCCGGACCAAGAGCACCTCTAGCAG
ATCCGGCTTCGAGATGATCTGGGACCCCAACGGCTGGACCGAGACAGATAGCAGCTTCAGCGTGAAGCAGGACA
TCGTGGCCATCACCGATTGGAGCGGCTACAGCGGAAGCTTCGTGCAGCACCCTGAACTGACAGGCCTGGACTGC
ATGAGGCCCTGCTTTTGGGTCGAGCTGATCCGGGGCAGACCCAAAGAGAACACCATCTGGACAAGCGGCAGCAG
CATCAGCTTTTGCGGCGTGAACAGCGATACCGTCGGCTGGTCTTGGCCTGATGGTGCCGAGCTGCCTTTCACCA
TCGACAAAGGATCCGGCGCCACCAACTTTAGTCTGCTGAAACAGGCCGGCGACGTCGAAGAGAACCCAGGTCCT
ATGTCTCTGCTGACCGAGGTGGAAACCCCTACCAGAAATGGCTGGGAGTGCAGATGCAGCGACAGCAGCGATCC
TCTGGTTATCGCCGCCAGCATCATCGGCATCCTGCACCTGATCCTGTGGATCCTGGACCGGCTGTTCTTCAAGT
GCATCTACCGGCGGCTGAAGTACGGCCTGAAGAGAGGCCCTTCTACAGAGGGCGTGCCCGAGAGCATGCGGGAA
GAGTACAGACAGAAACAGCAGAGCGCCGTGGACGTGGACGATGGCCACTTCGTGAACATCGAGCTGGAATGAGC
GGCCGCAGATCTGCTGTGCCTTCTAGTTGCCAGCCATCTGTTGTTTGCCCCTCCCCCGTGCCTTCCTTGACCCT
GGAAGGTGCCACTCCCACTGTCCTTTCCTAATAAAATGAGGAAATTGCATCGCATTGTCTGAGTAGGTGTCATT
CTATTCTGGGGGGTGGGGTGGGGCAGGACAGCAAGGGGGAGGATTGGGAAGACAATAGCAGGCATGCTGGGGAT
GCGGTGGGCTCTATGGCTACCCAGGTGCTGAAGAATTGACCCGGTTCCTCCTGGGCCAGAAAGAAGCAGGCACA
TCCCCTTCTCTGTGACACACCCTGTCCACGCCCCTGGTTCTTAGTTCCAGCCCCACTCATAGGACACTCATAGC
TCAGGAGGGCTCCGCCTTCAATCCCACCCGCTAAAGTACTTGGAGCGGTCTCTCCCTCCCTCATCAGCCCACCA
AACCAAACCTAGCCTCCAAGAGTGGGAAGAAATTAAAGCAAGATAGGCTATTAAGTGCAGAGGGAGAGAAAATG
CCTCCAACATGTGAGGAAGTAATGAGAGAAATCATAGAATTTTAAGGCCATGATTTAAGGCCATCATGGCCTTA
ATCTTCCGCTTCCTCGCTCACTGACTCGCTGCGCTCGGTCGTTCGGCTGCGGCGAGCGGTATCAGCTCACTCAA
AGGCGGTAATACGGTTATCCACAGAATCAGGGGATAACGCAGGAAAGAACATGTGAGCAAAAGGCCAGCAAAAG
GCCAGGAACCGTAAAAAGGCCGCGTTGCTGGCGTTTTTCCATAGGCTCCGCCCCCCTGACGAGCATCACAAAAA
TCGACGCTCAAGTCAGAGGTGGCGAAACCCGACAGGACTATAAAGATACCAGGCGTTTCCCCCTGGAAGCTCCC
TCGTGCGCTCTCCTGTTCCGACCCTGCCGCTTACCGGATACCTGTCCGCCTTTCTCCCTTCGGGAAGCGTGGCG
CTTTCTCATAGCTCACGCTGTAGGTATCTCAGTTCGGTGTAGGTCGTTCGCTCCAAGCTGGGCTGTGTGCACGA
ACCCCCCGTTCAGCCCGACCGCTGCGCCTTATCCGGTAACTATCGTCTTGAGTCCAACCCGGTAAGACACGACT
TATCGCCACTGGCAGCAGCCACTGGTAACAGGATTAGCAGAGCGAGGTATGTAGGCGGTGCTACAGAGTTCTTG
AAGTGGTGGCCTAACTACGGCTACACTAGAAGAACAGTATTTGGTATCTGCGCTCTGCTGAAGCCAGTTACCTT
CGGAAAAAGAGTTGGTAGCTCTTGATCCGGCAAACAAACCACCGCTGGTAGCGGTGGTTTTTTTGTTTGCAAGC
AGCAGATTACGCGCAGAAAAAAAGGATCTCAAGAAGATCCTTTGATCTTTTCTACGGGGTCTGACGCTCAGTGG
AACGAAAACTCACGTTAAGGGATTTTGGTCATGAGATTATCAAAAAGGATCTTCACCTAGATCCTTTTAAATTA
AAAATGAAGTTTTAAATCAATCTAAAGTATATATGAGTAAACTTGGTCTGACAGTTACCAATGCTTAATCAGTG
AGGCACCTATCTCAGCGATCTGTCTATTTCGTTCATCCATAGTTGCCTGACTCGGGGGGGGGGGGCGCTGAGGT
CTGCCTCGTGAAGAAGGTGTTGCTGACTCATACCAGGCCTGAATCGCCCCATCATCCAGCCAGAAAGTGAGGGA
GCCACGGTTGATGAGAGCTTTGTTGTAGGTGGACCAGTTGGTGATTTTGAACTTTTGCTTTGCCACGGAACGGT
CTGCGTTGTCGGGAAGATGCGTGATCTGATCCTTCAACTCAGCAAAAGTTCGATTTATTCAACAAAGCCGCCGT
CCCGTCAAGTCAGCGTAATGCTCTGCCAGTGTTACAACCAATTAACCAATTCTGATTAGAAAAACTCATCGAGC
ATCAAATGAAACTGCAATTTATTCATATCAGGATTATCAATACCATATTTTTGAAAAAGCCGTTTCTGTAATGA
AGGAGAAAACTCACCGAGGCAGTTCCATAGGATGGCAAGATCCTGGTATCGGTCTGCGATTCCGACTCGTCCAA
CATCAATACAACCTATTAATTTCCCCTCGTCAAAAATAAGGTTATCAAGTGAGAAATCACCATGAGTGACGACT
GAATCCGGTGAGAATGGCAAAAGCTTATGCATTTCTTTCCAGACTTGTTCAACAGGCCAGCCATTACGCTCGTC
ATCAAAATCACTCGCATCAACCAAACCGTTATTCATTCGTGATTGCGCCTGAGCGAGACGAAATACGCGATCGC
TGTTAAAAGGACAATTACAAACAGGAATCGAATGCAACCGGCGCAGGAACACTGCCAGCGCATCAACAATATTT
TCACCTGAATCAGGATATTCTTCTAATACCTGGAATGCTGTTTTCCCGGGGATCGCAGTGGTGAGTAACCATGC
ATCATCAGGAGTACGGATAAAATGCTTGATGGTCGGAAGAGGCATAAATTCCGTCAGCCAGTTTAGTCTGACCA
TCTCATCTGTAACATCATTGGCAACGCTACCTTTGCCATGTTTCAGAAACAACTCTGGCGCATCGGGCTTCCCA
TACAATCGATAGATTGTCGCACCTGATTGCCCGACATTATCGCGAGCCCATTTATACCCATATAAATCAGCATC
CATGTTGGAATTTAATCGCGGCCTCGAGCAAGACGTTTCCCGTTGAATATGGCTCATAACACCCCTTGTATTAC
TGTTTATGTAAGCAGACAGTTTTATTGTTCATGATGATATATTTTTATCTTGTGCAATGTAACATCAGAGATTT
TGAGACACAACGTGGCTTTCCCCCCCCCCCCATTATTGAAGCATTTATCAGGGTTATTGTCTCATGAGCGGATA
CATATTTGAATGTATTTAGAAAAATAAACAAATAGGGGTTCCGCGCACATTTCCCCGAAAAGTGCCACCTGACG
TCTAAGAAACCATTATTATCATGACATTAACCTATAAAAATAGGCGTATCACGAGGCCCTTTCGTC
panH1N1-amino acid sequence
(SEQ ID NO: 63)
MKAILVVLLYTFATANADTLCIGYHANNSTDTVDTVLEKNVTVTHSVNLLEDKHNGKLCKLRGVAPLHLGKCNI
AGWILGNPECESLSTASSWSYIVETSSSDNGTCYPGDFINYEELREQLSSVSSFERFEIFPKTSSWPNHDSNKG
VTAACPHAGAKSFYKNLIWLVKKGNSYPKLSKSYINDKGKEVLVLWGIHHPSTTADQQSLYQNADAYVFVGTSR
YSKKFKPEIAIRPKVRDQEGRMNYYWTLVEPGDKITFEATGNLVVPRYAFAMERNAGSGIIISDTPVHDCNTTC
QTPEGAINTSLPFQNIHPITIGKCPKYVKSTKLRLATGLRNVPSIQSRGLFGAIAGFIEGGWTGMVDGWYGYHH
QNEQGSGYAADLKSTQNAIDKITNKVNSVIEKMNTQFTAVGKEFNHLEKRIENLNKKVDDGELDIWTYNAELLV
LLENERTLDYHDSNVKNLYEKVRNQLKNNAKEIGNGCFEFYHKCDNTCMESVKNGTYDYPKYSEEAKLNREKID
GVKLESTRIYQILAIYSTVASSLVLVVSLGAISFWMCSNGSLQCRICI [symbol] MNPNQ
KIITIGSICMVVGIISLILQIGNIISIWVSHSIQTGNQNQPETCNQSIITYENNTWVNQTYVNISNTNFVAEQA
VASVALAGNSSLCPISGWAIYSKDNGIRIGSKGDVFVIREPFISCSHLECRTFFLTQGALLNDKHSNGTVKDRS
PYRTLMSCPVGEAPSPYNSRFESVAWSASACHDGISWLTIGISGPDNGAVAVLKYNGIITDTIKSWRNNILRTQ
ESECACINGSCFTIMTDGPSNGQASYKIFKIEKGKVVKSVELNAPNYHYEECSCYPDAGEVMCVCRDNWHGSNR
PWVSFNQNLEYQIGYICSGVFGDNPRPNDGTGSCGPVSSNGAYGVKGFSFKYGKGVWIGRTKSTSSRSGFEMIW
DPNGWTETDSSFSVKQDIVAITDWSGYSGSFVQHPELTGLDCMRPCFWVELIRGRPKENTIWTSGSSISFCGVN
SDTVGWSWPDGAELPFTIDK [symbol] PMSLLTEVETPTRNGWECRCSDSSDPLVIAASI
IGILHLILWILDRLFFKCIYRRLKYGLKRGPSTEGVPESMREEYRQKQQSAVDVDDGHFVNIELE

The panH1N1 amino acid sequence (SEQ ID NO:63) shown above includes a first 2A self-cleaving peptide sequence (GSGEGRGSLLTCGDVEENPGP; SEQ ID NO:66), shown highlighted in bold, between the amino acid sequences of the FLU_T2_HA_3_I3 and FLU_T2_NA_3 subunits, and a second 2A self-cleaving peptide sequence (GSGATNFSLLKQAGDVEENPGP; SEQ ID NO:67), shown highlighted in bold, between the amino acid sequences of the FLU_T2_NA_3 and FLU_T2_M2_1 subunits.

Strategies for multigene co-expression include introduction of multiple vectors, use of multiple promoters in a single vector, fusion proteins, proteolytic cleavage sites between genes, internal ribosome entry sites (IRES), and “self-cleaving” 2A peptides. Multicistronic vectors based on IRES nucleotide sequence and self-cleaving 2A peptides are reviewed in Shaimardanova et al. (Pharmaceutics 2019, 11, 580; doi:10.3390/pharmaceutics11110580).

2A self-cleaving peptides are 18-22 amino-acid-long viral oligopeptides that mediate “cleavage” of polypeptides during translation in eukaryotic cells (Liu et al., Scientific Reports 7, Article number: 2193 (2017)). The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified. The mechanism of 2A-mediated “self-cleavage” is ribosome skipping the formation of a glycyl-prolyl peptide bond at the C-terminus of the 2A. A highly conserved sequence GDVEXNPGP is shared by different 2As at the C-terminus, and is essential for the creation of steric hindrance and ribosome skipping. There are three possibilities for a 2A-mediated skipping event: (1) Successful skipping and recommencement of translation results in two “cleaved” proteins: the protein upstream of the 2A is attached to the complete 2A peptide except for the C-terminal proline, and the protein downstream of the 2A is attached to one proline at the N-terminus; (2) Successful skipping but ribosome fall-off and discontinued translation results in only the protein upstream of 2A; (3) Unsuccessful skipping and continued translation resulting in a fusion protein. Overall, 2A peptides lead to relatively high levels of downstream protein expression compared to other strategies for multi-gene co-expression, and they are small in size thus bearing a lower risk of interfering with the function of co-expressed genes.

### EXAMPLE 16—IMMUNOGENICITY AND EFFICACY OF A BROADLY REACTIVE H1N1 INFLUENZA VACCINE IN PIGS

### Background

The continued antigenic change (drift) of influenza A virus strains over time in the human population necessitates twice-yearly updates to the human seasonal vaccine composition. Development of a broadly cross-reactive ‘universal’ vaccine that does not require such frequent updates would be a considerable advantage. The aim of this study was to assess a novel broadly cross-reactive vaccine technology in the pig model of influenza.

### Methods:

The test vaccine in this study was a structure-based computational synthetic multi-gene antigen of human-origin H1N1 influenza A virus, panH1N1 (also referred to as DIOSynVax-H1N1). It was administered needle-free to 5 pigs as DNA intradermally (ID) using the PharmaJet® Tropis® system. Two control whole, inactivated virus (WV) vaccines of the same pandemic lineage, A/swine/England/1353/2009 (WIV₁₃₅₃) and A/Victoria/2454/2019 (WIV_{Vic}) in oil-in-water adjuvant were administered intramuscularly to 5 pigs each at the same 4-week interval. Six weeks following the second immunisation all groups were challenged with the swine-origin pH1N1 strain A/swine/England/1353/2009 (1.7×10⁶ TCID₅₀ per pig intranasally). Pigs were monitored daily post-inoculation (dpi) until end of experiment on 8 or 9 dpi. The immunisation and bleed protocol of pigs tested is illustrated in FIGS. 11a and b.

### Results:

Nasal shedding of viral RNA was monitored daily by RRT-qPCR (FIG. 12). All challenged animals shed viral RNA, reaching a peak between 2 to 5 dpi by RRT-qPCR (FIG. 12a). Area under the curve analysis showed significant reduction in the nasal shedding of viral RNA in the animal groups vaccinated with the broadly reactive panH1N1 (P=0.0012) or WIV₁₃ (P=0.0003) vaccines when compared to the naïve control or WIV_{Vic} vaccinated groups (FIG. 12b). All pigs resolved the infection by 8-9 dpi, as shown in the lower graph of FIG. 12b which illustrates virus titration measurements from bronchoalveolar lavage (BAL) fluid, turbinates, and trachea samples from pigs of each group.

Influenza virus-specific serum antibody levels were monitored longitudinally by Haemagluttinin inhibition Assay (HAI; FIG. 13a), and NP ELISA (FIG. 13b). Both assays revealed significant antibody levels in the WV-vaccinated groups, even after a single vaccination. The panH1N1 immunised pigs mounted an equivalent HA antibody response to the WV vaccines after the boost vaccination (i.e. after D28). Antibodies were found to be neutralising, as shown in the serum neutralisation assay in FIG. 14. Further evidence that the panH1N1 vaccine provides similar protection to WIV₁s is shown in the ELISopt and HAI assays of FIG. 15. In particular, FIG. 15a shows a T-cell peptide stimulation assay, wherein splenocytes were stimulated with the peptides spanning A/Swine/england/1353/2009 HA and A/Victoria/2545/2019 H A. Higher the values on the y-axis indicate a higher T-cell response. Each point corresponds to one pig. panH1N1 vaccinated group has better T-cell response than the controls post infection. FIG. 15b shows a HAI assay. The top panel shows distribution of the hemagglutinin inhibition titre 0 days, 28 days, 42 days and 63 days post vaccination and 8 days post infection. The titres were checked against A/swine/England/1353/2009 strain and a/Victoria/2454/2019 strain. The lower panel illustrates the mean values for each group.

### Conclusion:

Importantly this study demonstrated proof-of-concept that pigs immunised with the broadly neutralising panH1N1 vaccine were protected as well as a whole virion, inactivated adjuvanted vaccine homologous to the challenge strain (WIV₁₃₅₃). In contrast, a WV vaccine made from a human-origin strain from the same pH1N1 1A.3.3.2 lineage (WIVvic), failed to show any protection in the presence of significant antibody levels.

### EXAMPLE 17—OPTIMISED VACCINE GENERATES NEUTRALISING IMMUNE RESPONSES AND PROTECTS AGAINST HUMAN AND SWINE H1N1 INFLUENZA IN MICE AND PIGS

### Background

Influenza A's (IAV) zoonotic transmission and constant evolution in multiple species especially birds and pigs heighten the potential emergence of novel strains at the human-animal interface. The cornerstone of influenza prevention and control is still strain-specific vaccination, however pitfalls associated with this have decreased vaccine effectiveness. To cover seasonal, zoonotic, and pandemic threats, we present an elegant Digitally designed, Immune Optimised, Synthetic (DIOS) vaccine to induce broad H1, N1 and M2 subtype-specific immunity and protection against divergent strains in mouse and pig models.

### Methods

For mouse immunogenicity studies, individual immunogen, FLU_T2_HA_3_I3, was injected subcutaneously 4 times in 2-week intervals and terminal bleeds taken 10 weeks post-first immunization. For the pig challenge, a prime-boost regimen (4 week interval) was employed and the panH1N1 vaccine candidate administered intradermally via PharmaJet® Tropis. Controls delivered intramuscularly included whole inactivated virus (WV) representing swine and human influenza. Pigs were challenged with A/swine/EN/1353/09 10 weeks post-prime. Efficacy was measured as reduced viral shedding. Serum neutralizing titers were monitored using pseudotype neutralization (pMN), enzyme-linked lectin assay (ELLA) and hemagglutination inhibition (HAI).

### Results

FIG. 16 shows surface representations of hemagglutinin (HA), neuraminidase (NA) and M2 from A/swine/EN/1353/09, A/Victoria/2454/2019 H1N1 strains and our DIOS vaccine candidate, panH1N1. Coloured residues show defined antigenic sites with non-conserved residues between swine/EN/09 and panH1N1 highlighted in red, and between swine/EN/09 and Victoria/19 in magenta.

As shown in FIG. 17a and FIG. 24, we observed excellent immune responses in all mice (n=6) vaccinated with FLU_T2_HA_3_I3 (referred to as DIOS(HA) in the FIG. 17, and H1N1dpm in FIG. 24) against all H1N1 strains tested that are comparable or superior to the control A/Michigan/45/15(H1) (*p<0.05). Values are calculated as the fold dilution of sera that resulted in 50% neutralization of virus via pMN. FIG. 17b shows administration of FLU_T2_HA_3_I3 in mice elicits effective antibody binding responses to six H1 wild-type influenza viruses tested.

In pigs, reduced viral shedding in nasal swabs (expressed as mean log Relative equivalent units (REU) of viral RNA) was observed post-challenge in the panH1N1 (n=5) and WIV₁₃₅₃ (homologous to the challenge strain) (n=5) groups (FIG. 18). Serum neutralising antibodies against HA and NA of A/swine/England/1353/2009 and other relevant H1N1 viruses were also detected in groups given panH1N1 and WIV₁₃₅₃. FIG. 19a and FIG. 19b show serum neutralising titers vs VI/2570119 and EN/195/09 as monitored at specific times, FIG. 19c shows serum neutralisation with panH1N1 vaccination against a panel of H1 expressing pseudoviruses at 42 days post vaccination.

FIGS. 20a and 20b show an ELLA (Enzyme-Linked Lectin Assay) to assess the inhibition activity of the NA component of panH1N1 against A/swine/England/1353/2009 (FIG. 20a) and A/England/195/2009 (FIG. 20b) at a series of time points post-vaccination/infection. FIG. 20c shows an ELLA against a panel of NA expressing pseudoviruses at 42 days post vaccination.

PanH1N1 is referred to as DIOS in FIGS. 16 and 18, 19, and 20.

### Conclusion

We have shown the immunogenicity and efficacy of the DIOS (panH1N1 and individual FLU_T2_HA3_I3) vaccine against relevant IAV H1N1 strains in vitro and in vivo in mice and pigs. This approach may target different aspects of influenza leading to broadened protection within the same subtype. This can support pandemic preparedness whilst protecting against circulating human influenza. This platform can be translated into other subtypes with the goal of producing a universal influenza vaccine.

### EXAMPLE 18—FLU_T3_HA_3

This example provides amino acid sequences of the influenza haemagluttinin H5 head and stem regions for an embodiment of the invention known as FLU_T3_HA_3. In SEQ ID NO:27 below, the amino acid residues of the stem region are shown underlined. The amino acid residues of the head region are the remaining residues. Similarly, in SEQ ID NO:28 below, the nucleic acid residues of the stem region are shown underlined. The nucleic acid residues of the head region are the remaining residues.

FLU_T3_HA_3-HAO amino acid sequence (SEQ ID NO: 27):
MEKIVLLLAIVSLVKSDQICIGYHANNSTEQVDTIMEKNVTVTHAQDILEKTHNGKLCDL
DGVKPLILRDCSVAGWLLGNPMCDEFINVPEWSYIVEKANPANDLCYPGNENDYEELKHL
LSRINHFEKIQIIPKSSWSDHEAS/GVSSACPYQGRSSFFRNVVWLIKKNNAYPTIKRSY
NNTNQEDLLVLWGIHHPNDAAEQTRLYQNPTTYISVGTSTLNQRLVPKIATRSKVNGQSG
RMEFFWTILKPNDAINFESNGNFIAPEYAYKIVKKGDSAIMKSELEYGNCNTKCQTPMGA
INSSMPFHNIHPLTIGECPKYVKSNRLVLATGLRNSPQRERRRKKRGLFGAIAGFIEGGW
QGMVDGWYGYHHSNEQGSGYAADKESTQKAIDGVTNKVNSIIDKMNTQFEAVGREENNLE
RRIENLNKKMEDGELDVWTYNAELLVLMENERTLDFHDSNVKNLYDKVRLQLRDNAKELG
NGCFEFYHKCDNECMESVRNGTYDYPQYSEEARLKREEISGVKLESIGTYQILSIYSTVA
SSLALAIMVAGLSLWMCSNGSLQCRICI
FLU_T3_HA_3-HAO nucleic acid sequence (SEQ ID NO: 28)
ATGGAAAAGATTGTGCTGCTGCTGGCCATCGTGTCCCTGGTCAAGAGCGATCAAATCTGC
ATCGGCTACCACGCCAACAACAGCACCGAACAGGTGGACACCATTATGGAAAAGAACGTG
ACCGTGACACACGCCCAGGACATCCTGGAAAAGACCCACAACGGCAAGCTGTGCGACCTG
GATGGCGTGAAGCCTCTGATCCTGAGAGATTGCTCTGTGGCCGGCTGGCTGCTGGGCAAT
CCTATGTGCGACGAGTTCATCAACGTGCCCGAGTGGTCCTATATCGTGGAAAAGGCCAAT
CCTGCCAACGACCTGTGCTACCCCGGCAACTTCAACGACTACGAGGAACTGAAACATCTG
CTGAGCCGGATCAACCACTTCGAGAAGATCCAGATCATCCCCAAGTCCTCTTGGAGCGAT
CACGAGGCCTCTGGAGTGTCTAGCGCCTGTCCTTACCAAGGCAGAAGCAGCTTCTTCCGG
AACGTCGTGTGGCTGATCAAGAAGAACAACGCTTACCCCACCATCAAGCGGAGCTACAAC
AACACCAATCAAGAGGACCTGCTGGTGCTGTGGGGCATCCACCATCCTAATGATGCCGCC
GAGCAGACCCGGCTGTACCAGAATCCTACAACCTACATCAGCGTGGGCACCAGCACACTG
AACCAGAGACTGGTGCCTAAGATCGCCACCAGATCCAAAGTGAACGGCCAGAGCGGCCGG
ATGGAATTCTTCTGGACCATCCTGAAGCCTAACGACGCCATCAACTTCGAGAGCAACGGC
AACTTTATCGCCCCTGAGTACGCCTACAAGATCGTGAAGAAGGGCGACAGCGCCATCATG
AAGTCCGAGCTGGAATACGGCAACTGCAACACCAAGTGTCAGACCCCTATGGGCGCCATC
AATAGCAGCATGCCCTTCCACAACATTCACCCTCTGACCATCGGCGAGTGCCCCAAATAC
GTGAAGTCCAACAGACTGGTCCTGGCCACCGGCCTGAGAAATTCTCCACAGAGAGAGCGG
CGCAGAAAGAAGAGAGGCCTGTTTGGAGCCATTGCCGGCTTTATCGAAGGCGGCTGGCAA
GGCATGGTTGACGGATGGTACGGCTATCACCACAGCAATGAGCAAGGCTCTGGCTACGCC
GCCGACAAAGAGAGCACACAGAAAGCCATCGACGGCGTGACCAACAAAGTGAATAGCATC
ATCGACAAGATGAACACCCAGTTCGAGGCCGTGGGCAGAGAGTTCAACAACCTGGAAAGA
CGGATCGAGAACCTGAACAAGAAGATGGAGGACGGCTTCCTGGACGTGTGGACCTATAAT
GCCGAGCTGCTGGTCCTGATGGAAAACGAGAGAACCCTGGACTTCCACGACAGCAACGTG
AAGAACCTGTACGACAAAGTGCGGCTCCAGCTGCGGGACAATGCCAAAGAACTCGGCAAC
GGCTGCTTCGAGTTCTACCACAAGTGCGACAACGAGTGCATGGAAAGCGTGCGGAACGGC
ACCTACGACTACCCTCAGTACTCTGAGGAAGCCCGGCTGAAGAGAGAAGAGATCAGCGGA
GTGAAGCTGGAATCCATCGGCACATACCAGATCCTGAGCATCTACAGCACCGTGGCCTCT
TCTCTGGCCCTGGCTATTATGGTGGCTGGCCTGAGCCTGTGGATGTGCTCTAATGGCAGC
CTCCAGTGCCGGATCTGCATCTGA
FLU_T3_HA_3-head region amino acid sequence (SEQ ID NO: 29)
THNGKLCDLDGVKPLILRDCSVAGWLLGNPMCDEFINVPEWSYIVEKANPANDLCYPGNENDYEELK
HLLSRINHFEKIQIIPKSSWSDHEAS [symbol] GVSSACPYQG [symbol] SFFRNVVWLIKKN [symbol] YPTIKRSYNNTNQ
EDLLVLWGIHHPNDAAEQT [symbol] LYQNPTTYISVGTSTLNQRLVPKIATRSKVNGQSGRMEFFWTILKPN
DAINFESNGNFIAPEYAYKIVKKGDSAIMKSELEYGNCNTKCQTPMGAINSSMPFHNIHPLTIGECP

The amino acid residues at positions 156, 157, 171, 172, and 205 are shown underlined and highlighted in grey in the above sequence (and are R, S, N, A, and R, respectively). The deleted amino acid residue at residue 144 or 145 is shown as “/”, and highlighted in greyscale.

FLU_T3_HA_3-head region nucleic acid sequence
(SEQ ID NO: 30)
ACCCACAACGGCAAGCTGTGCGACCTGGATGGCGTGAAGCCTCTGATCCTGAGAGATTGCTCTGTGG
CCGGCTGGCTGCTGGGCAATCCTATGTGCGACGAGTTCATCAACGTGCCCGAGTGGTCCTATATCGT
GGAAAAGGCCAATCCTGCCAACGACCTGTGCTACCCCGGCAACTTCAACGACTACGAGGAACTGAAA
CATCTGCTGAGCCGGATCAACCACTTCGAGAAGATCCAGATCATCCCCAAGTCCTCTTGGAGCGATC
ACGAGGCCTCTGGAGTGTCTAGCGCCTGTCCTTACCAAGGCAGAAGCAGCTTCTTCCGGAACGTCGT
GTGGCTGATCAAGAAGAACAACGCTTACCCCACCATCAAGCGGAGCTACAACAACACCAATCAAGAG
GACCTGCTGGTGCTGTGGGGCATCCACCATCCTAATGATGCCGCCGAGCAGACCCGGCTGTACCAGA
ATCCTACAACCTACATCAGCGTGGGCACCAGCACACTGAACCAGAGACTGGTGCCTAAGATCGCCAC
CAGATCCAAAGTGAACGGCCAGAGCGGCCGGATGGAATTCTTCTGGACCATCCTGAAGCCTAACGAC
GCCATCAACTTCGAGAGCAACGGCAACTTTATCGCCCCTGAGTACGCCTACAAGATCGTGAAGAAGG
GCGACAGCGCCATCATGAAGTCCGAGCTGGAATACGGCAACTGCAACACCAAGTGTCAGACCCCTAT
GGGCGCCATCAATAGCAGCATGCCCTTCCACAACATTCACCCTCTGACCATCGGCGAGTGCCCC
FLU_T3_HA_3-first stem region amino acid sequence
(SEQ ID NO: 31)
MEKIVLLLAIVSLVKSDQICIGYHANNSTEQVDTIMEKNVTVTHAQDILEK
FLU_T3_HA_3-first stem region nucleic acid sequence
(SEQ ID NO: 32)
ATGGAAAAGATTGTGCTGCTGCTGGCCATCGTGTCCCTGGTCAAGAGCGATCAAATCTGCATCGGCT
ACCACGCCAACAACAGCACCGAACAGGTGGACACCATTATGGAAAAGAACGTGACCGTGACACACGC
CCAGGACATCCTGGAAAAG
FLU_T3_HA_3-second stem region amino acid sequence
(SEQ ID NO: 33)
KYVKSNRLVLATGLRNSPQRERRRKKRGLFGAIAGFIEGGWQGMVDGWYGYHHSNEQGSGYAADKES
TQKAIDGVTNKVNSIIDKMNTQFEAVGRE [symbol] NNLERRIENLNKKMEDG [symbol] LDVWTYNAELLVLMENERT
LDFHDSNVKNLYDKVRLQLRDNAKELGNGCFEFYHKCDNECMESVRNGTYDYPQYSEEARLKREEIS
GVKLESIGTYQILSIYSTVASSLALAIMVAGLSLWMCSNGSLQCRICI

The amino acid residues at positions 416 and 434 (or at positions 148 and 166 if counting from the beginning of the stem region) are shown underlined in the above sequence (and are F and F, respectively).

FLU_T3_HA_3-second stem region nucleic
acid sequence
(SEQ ID NO: 34)
AAATACGTGAAGTCCAACAGACTGGTCCTGGCCACCGGCCTGAGAA
ATTCTCCACAGAGAGAGCGGCGCAGAAAGAAGAGAGGCCTGTTTG
GAGCCATTGCCGGCTTTATCGAAGGCGGCTGGCAAGGCATGGTTG
ACGGATGGTACGGCTATCACCACAGCAATGAGCAAGGCTCTGGCT
ACGCCGCCGACAAAGAGAGCACACAGAAAGCCATCGACGGCGTGA
CCAACAAAGTGAATAGCATCATCGACAAGATGAACACCCAGTTCG
AGGCCGTGGGCAGAGAGTTCAACAACCTGGAAAGACGGATCGAGA
ACCTGAACAAGAAGATGGAGGACGGCTTCCTGGACGTGTGGACCT
ATAATGCCGAGCTGCTGGTCCTGATGGAAAACGAGAGAACCCTGG
ACTTCCACGACAGCAACGTGAAGAACCTGTACGACAAAGTGCGGC
TCCAGCTGCGGGACAATGCCAAAGAACTCGGCAACGGCTGCTTCG
AGTTCTACCACAAGTGCGACAACGAGTGCATGGAAAGCGTGCGGA
ACGGCACCTACGACTACCCTCAGTACTCTGAGGAAGCCCGGCTGA
AGAGAGAAGAGATCAGCGGAGTGAAGCTGGAATCCATCGGCACAT
ACCAGATCCTGAGCATCTACAGCACCGTGGCCTCTCTCTGGCCCT
GGCTATTATGGTGGCTGGCCTGAGCCTGTGGATGTGCTCTAATGG
CAGCCTCCAGTGCCGGATCTGCATCTGA

### EXAMPLE 19—FLU_T3_HA_4

This example provides amino acid sequences of the influenza haemagluttinin H5 head and stem regions for an embodiment of the invention known as FLU_T3_HA_4. In SEQ ID NO:35 below, the amino acid residues of the stem region are shown underlined. The amino acid residues of the head region are the remaining residues. Similarly, in SEQ ID NO:36 below, the nucleic acid residues of the stem region are shown underlined. The nucleic acid residues of the head region are the remaining residues.

FLU_T3_HA_4-HAO amino acid sequence (SEQ ID NO: 35):
MEKIVLLLAIVSLVKSDQICIGYHANNSTEQVDTIMEKNVTVTHAQDILEKTHNGKLCDLDGVKPLI
LRDCSVAGWLLGNPMCDEFINVPEWSYIVEKANPANDLCYPGNENDYEELKHLLSRINHFEKIQIIP
KSSWSDHEASSGVVPACPYQGRSSFFRNVVWLIKKNNAYPTIKRSYNNTNQEDLLVLWGIHHPNDAA
EQTRLYQNPTTYISVGTSTLNQRLVPKIATRSKVNGESGRMEFFWTILKPNDAINFESNGNFIAPEY
AYKIVKKGDSAIMKSELEYGNCNTKCQTPMGAINSSMPFHNIHPLTIGECPKYVKSNRLVLATGLRN
SPQRERRRKKRGLFGAIAGFIEGGWQGMVDGWYGYHHSNEQGSGYAADKESTQKAIDGVTNKVNSII
DKMNTQFEAVGREENNLERRIENLNKKMEDGELDVWTYNAELLVLMENERTLDFHDSNVKNLYDKVR
LQLRDNAKELGNGCFEFYHKCDNECMESVRNGTYDYPQYSEEARLKREEISGVKLESIGTYQILSIY
STVASSLALAIMVAGLSLWMCSNGSLQCRICI
FLU_T3_HA_4-HAO nucleic acid sequence (SEQ ID NO: 36)
ATGGAAAAGATTGTGCTGCTGCTGGCCATCGTGTCCCTGGTCAAGAGCGATCAAATCTGCATCGGCT
ACCACGCCAACAACAGCACCGAACAGGTGGACACCATTATGGAAAAGAACGTGACCGTGACACACGC
CCAGGACATCCTGGAAAAGACCCACAACGGCAAGCTGTGCGACCTGGATGGCGTGAAGCCTCTGATC
CTGAGAGATTGCTCTGTGGCCGGCTGGCTGCTGGGCAATCCTATGTGCGACGAGTTCATCAACGTGC
CCGAGTGGTCCTATATCGTGGAAAAGGCCAATCCTGCCAACGACCTGTGCTACCCCGGCAACTTCAA
CGACTACGAGGAACTGAAACATCTGCTGAGCCGGATCAACCACTTCGAGAAGATCCAGATCATCCCC
AAGTCCTCTTGGAGCGATCACGAGGCCTCTAGCGGAGTGGTGCCGGCCTGTCCTTACCAAGGCAGAA
GCAGCTTCTTCCGGAACGTCGTGTGGCTGATCAAGAAGAACAACGCTTACCCCACCATCAAGCGGAG
CTACAACAACACCAATCAAGAGGACCTGCTGGTGCTGTGGGGCATCCACCATCCTAATGATGCCGCC
GAGCAGACCCGGCTGTACCAGAATCCTACAACCTACATCAGCGTGGGCACCAGCACACTGAACCAGA
GACTGGTGCCTAAGATCGCCACCAGATCCAAAGTGAACGGCGAAAGCGGCCGGATGGAATTCTTCTG
GACCATCCTGAAGCCTAACGACGCCATCAACTTCGAGAGCAACGGCAACTTTATCGCCCCTGAGTAC
GCCTACAAGATCGTGAAGAAGGGCGACAGCGCCATCATGAAGTCCGAGCTGGAATACGGCAACTGCA
ACACCAAGTGTCAGACCCCTATGGGCGCCATCAATAGCAGCATGCCCTTCCACAACATTCACCCTCT
GACCATCGGCGAGTGCCCCAAATACGTGAAGTCCAACAGACTGGTCCTGGCCACCGGCCTGAGAAAT
TCTCCACAGAGAGAGCGGCGCAGAAAGAAGAGAGGCCTGTTTGGAGCCATTGCCGGCTTTATCGAAG
GCGGCTGGCAAGGCATGGTTGACGGATGGTACGGCTATCACCACAGCAATGAGCAAGGCTCTGGCTA
CGCCGCCGACAAAGAGAGCACACAGAAAGCCATCGACGGCGTGACCAACAAAGTGAATAGCATCATC
GACAAGATGAACACCCAGTTCGAGGCCGTGGGCAGAGAGTTCAACAACCTGGAAAGACGGATCGAGA
ACCTGAACAAGAAGATGGAGGACGGCTTCCTGGACGTGTGGACCTATAATGCCGAGCTGCTGGTCCT
GATGGAAAACGAGAGAACCCTGGACTTCCACGACAGCAACGTGAAGAACCTGTACGACAAAGTGCGG
CTCCAGCTGCGGGACAATGCCAAAGAACTCGGCAACGGCTGCTTCGAGTTCTACCACAAGTGCGACA
ACGAGTGCATGGAAAGCGTGCGGAACGGCACCTACGACTACCCTCAGTACTCTGAGGAAGCCCGGCT
GAAGAGAGAAGAGATCAGCGGAGTGAAGCTGGAATCCATCGGCACATACCAGATCCTGAGCATCTAC
AGCACCGTGGCCTCTTCTCTGGCCCTGGCTATTATGGTGGCTGGCCTGAGCCTGTGGATGTGCTCTA
ATGGCAGCCTCCAGTGCCGGATCTGCATCTGA
FLU_T3_HA_4-head region amino acid sequence (SEQ ID NO: 37)
THNGKLCDLDGVKPLILRDCSVAGWLLGNPMCDEFINVPEWSYIVEKANPANDLCYPGNENDYEELK
HLLSRINHFEKIQIIPKSSWSDHEASSGV [symbol] ACPYQG [symbol] SFERNVVWLIKKN [symbol] YPTIKRSYNNTNQ
EDLLVLWGIHHPNDAAEQT [symbol] LYQNPTTYISVGTSTLNQRLVPKIATRSKVNG [symbol] SGRMEFEWTILKPN
DAINFESNGNFIAPEYAYKIVKKGDSAIMKSELEYGNCNTKCQTPMGAINSSMPFHNIHPLTIGECP

The amino acid residues at positions 156, 157, 171, 172, and 205 are highlighted in the above sequence (and are R, S, N, A, and R, respectively). The amino acid residues at positions 148, 149, and 238 are also highlighted, and are V, P, and E, respectively.

FLU_T3_HA_4-head region nucleic acid sequence
(SEQ ID NO: 38)
ACCCACAACGGCAAGCTGTGCGACCTGGATGGCGTGAAGCCTCTGATCCTGAGAGATTGCTCTGTGG
CCGGCTGGCTGCTGGGCAATCCTATGTGCGACGAGTTCATCAACGTGCCCGAGTGGTCCTATATCGT
GGAAAAGGCCAATCCTGCCAACGACCTGTGCTACCCCGGCAACTTCAACGACTACGAGGAACTGAAA
CATCTGCTGAGCCGGATCAACCACTTCGAGAAGATCCAGATCATCCCCAAGTCCTCTTGGAGCGATC
ACGAGGCCTCTAGCGGAGTGGTGCCGGCCTGTCCTTACCAAGGCAGAAGCAGCTTCTTCCGGAACGT
CGTGTGGCTGATCAAGAAGAACAACGCTTACCCCACCATCAAGCGGAGCTACAACAACACCAATCAA
GAGGACCTGCTGGTGCTGTGGGGCATCCACCATCCTAATGATGCCGCCGAGCAGACCCGGCTGTACC
AGAATCCTACAACCTACATCAGCGTGGGCACCAGCACACTGAACCAGAGACTGGTGCCTAAGATCGC
CACCAGATCCAAAGTGAACGGCGAAAGCGGCCGGATGGAATTCTTCTGGACCATCCTGAAGCCTAAC
GACGCCATCAACTTCGAGAGCAACGGCAACTTTATCGCCCCTGAGTACGCCTACAAGATCGTGAAGA
AGGGCGACAGCGCCATCATGAAGTCCGAGCTGGAATACGGCAACTGCAACACCAAGTGTCAGACCCC
TATGGGCGCCATCAATAGCAGCATGCCCTTCCACAACATTCACCCTCTGACCATCGGCGAGTGCCCC
FLU_T3_HA_4-first stem region amino acid sequence
(SEQ ID NO: 39)
MEKIVLLLAIVSLVKSDQICIGYHANNSTEQVDTIMEKNVTVTHAQDILEK
FLU_T3_HA_4-first stem region nucleic acid sequence
(SEQ ID NO: 40)
ATGGAAAAGATTGTGCTGCTGCTGGCCATCGTGTCCCTGGTCAAGAGCGATCAAATCTGCATCGGCT
ACCACGCCAACAACAGCACCGAACAGGTGGACACCATTATGGAAAAGAACGTGACCGTGACACACGC
CCAGGACATCCTGGAAAAG
FLU_T3_HA_4-second stem region amino acid sequence
(SEQ ID NO: 41)
KYVKSNRLVLATGLRNSPQRERRRKKRGLFGAIAGFIEGGWQGMVDGWYGYHHSNEQGSGYAADKES
TQKAIDGVTNKVNSIIDKMNTQFEAVGRE [symbol] NNLERRIENLNKKMEDG [symbol] LDVWTYNAELLVLMENERT
LDFHDSNVKNLYDKVRLQLRDNAKELGNGCFEFYHKCDNECMESVRNGTYDYPQYSEEARLKREEIS
GVKLESIGTYQILSIYSTVASSLALAIMVAGLSLWMCSNGSLQCRICI

The amino acid residues at positions 416 and 434 (or at positions 148 and 166 if counting from the beginning of the stem region) are shown underlined in the above sequence (and are F and F, respectively).

FLU_T3_HA_4-second stem region nucleic
acid sequence
(SEQ ID NO: 42)
AAATACGTGAAGTCCAACAGACTGGTCCTGGCCACCGGCCTGAGAA
ATTCTCCACAGAGAGAGCGGCGCAGAAAGAAGAGAGGCCTGTTTG
GAGCCATTGCCGGCTTTATCGAAGGCGGCTGGCAAGGCATGGTTG
ACGGATGGTACGGCTATCACCACAGCAATGAGCAAGGCTCTGGCT
ACGCCGCCGACAAAGAGAGCACACAGAAAGCCATCGACGGCGTGA
CCAACAAAGTGAATAGCATCATCGACAAGATGAACACCCAGTTCG
AGGCCGTGGGCAGAGAGTTCAACAACCTGGAAAGACGGATCGAGA
ACCTGAACAAGAAGATGGAGGACGGCTTCCTGGACGTGTGGACCT
ATAATGCCGAGCTGCTGGTCCTGATGGAAAACGAGAGAACCCTGG
ACTTCCACGACAGCAACGTGAAGAACCTGTACGACAAAGTGCGGC
TCCAGCTGCGGGACAATGCCAAAGAACTCGGCAACGGCTGCTTCG
AGTTCTACCACAAGTGCGACAACGAGTGCATGGAAAGCGTGCGGA
ACGGCACCTACGACTACCCTCAGTACTCTGAGGAAGCCCGGCTGA
AGAGAGAAGAGATCAGCGGAGTGAAGCTGGAATCCATCGGCACAT
ACCAGATCCTGAGCATCTACAGCACCGTGGCCTCTTCTCTGGCCC
TGGCTATTATGGTGGCTGGCCTGAGCCTGTGGATGTGCTCTAATG
GCAGCCTCCAGTGCCGGATCTGCATCTGA

### EXAMPLE 20—FLU_T3_HA_5

This example provides amino acid sequences of the influenza haemagluttinin H5 head and stem regions for an embodiment of the invention known as FLU_T3_HA_5. In SEQ ID NO:43 below, the amino acid residues of the stem region are shown underlined. The amino acid residues of the head region are the remaining residues. Similarly, in SEQ ID NO:44 below, the nucleic acid residues of the stem region are shown underlined. The nucleic acid residues of the head region are the remaining residues.

FLU_T3_HA_5-HAO amino acid sequence (SEQ ID NO: 43):
MEKIVLLLAIVSLVKSDQICIGYHANNSTEQVDTIMEKNVTVTHAQDILEKTHNGKLCDLDGVKPLI
LRDCSVAGWLLGNPMCDEFINVPEWSYIVEKANPANDLCYPGNENDYEELKHLLSRINHFEKIQIIP
KSSWSDHEASSGVSSACPYQGRSSFERNVVWLIKKNNAYPTIKRSYNNTNQEDLLVLWGIHHPNDAA
EQTRLYQNPTTYISVGTSTLNQRLVPKIATRSKVNGESGRMEFFWTILKPNDAINFESNGNFIAPEY
AYKIVKKGDSAIMKSELEYGNCNTKCQTPMGAINSSMPFHNIHPLTIGECPKYVKSNRLVLATGLRN
SPQRERRRKKRGLFGAIAGFIEGGWQGMVDGWYGYHHSNEQGSGYAADKESTQKAIDGVTNKVNSII
DKMNTQFEAVGREENNLERRIENLNKKMEDGELDVWTYNAELLVLMENERTLDFHDSNVKNLYDKVR
LQLRDNAKELGNGCFEFYHKCDNECMESVRNGTYDYPQYSEEARLKREEISGVKLESIGTYQILSIY
STVASSLALAIMVAGLSLWMCSNGSLQCRICI
FLU_T3_HA_5-HAO nucleic acid sequence (SEQ ID NO: 44)
ATGGAAAAGATTGTGCTGCTGCTGGCCATCGTGTCCCTGGTCAAGAGCGATCAAATCTGCATCGGCT
ACCACGCCAACAACAGCACCGAACAGGTGGACACCATTATGGAAAAGAACGTGACCGTGACACACGC
CCAGGACATCCTGGAAAAGACCCACAACGGCAAGCTGTGCGACCTGGATGGCGTGAAGCCTCTGATC
CTGAGAGATTGCTCTGTGGCCGGCTGGCTGCTGGGCAATCCTATGTGCGACGAGTTCATCAACGTGC
CCGAGTGGTCCTATATCGTGGAAAAGGCCAATCCTGCCAACGACCTGTGCTACCCCGGCAACTTCAA
CGACTACGAGGAACTGAAACATCTGCTGAGCCGGATCAACCACTTCGAGAAGATCCAGATCATCCCC
AAGTCCTCTTGGAGCGATCACGAGGCCTCTAGCGGAGTGTCTAGCGCCTGTCCTTACCAAGGCAGAA
GCAGCTTCTTCCGGAACGTCGTGTGGCTGATCAAGAAGAACAACGCTTACCCCACCATCAAGCGGAG
CTACAACAACACCAATCAAGAGGACCTGCTGGTGCTGTGGGGCATCCACCATCCTAATGATGCCGCC
GAGCAGACCCGGCTGTACCAGAATCCTACAACCTACATCAGCGTGGGCACCAGCACACTGAACCAGA
GACTGGTGCCTAAGATCGCCACCAGATCCAAAGTGAACGGCGAAAGCGGCCGGATGGAATTCTTCTG
GACCATCCTGAAGCCTAACGACGCCATCAACTTCGAGAGCAACGGCAACTTTATCGCCCCTGAGTAC
GCCTACAAGATCGTGAAGAAGGGCGACAGCGCCATCATGAAGTCCGAGCTGGAATACGGCAACTGCA
ACACCAAGTGTCAGACCCCTATGGGCGCCATCAATAGCAGCATGCCCTTCCACAACATTCACCCTCT
GACCATCGGCGAGTGCCCCAAATACGTGAAGTCCAACAGACTGGTCCTGGCCACCGGCCTGAGAAAT
TCTCCACAGAGAGAGCGGCGCAGAAAGAAGAGAGGCCTGTTTGGAGCCATTGCCGGCTTTATCGAAG
GCGGCTGGCAAGGCATGGTTGACGGATGGTACGGCTATCACCACAGCAATGAGCAAGGCTCTGGCTA
CGCCGCCGACAAAGAGAGCACACAGAAAGCCATCGACGGCGTGACCAACAAAGTGAATAGCATCATC
GACAAGATGAACACCCAGTTCGAGGCCGTGGGCAGAGAGTTCAACAACCTGGAAAGACGGATCGAGA
ACCTGAACAAGAAGATGGAGGACGGCTTCCTGGACGTGTGGACCTATAATGCCGAGCTGCTGGTCCT
GATGGAAAACGAGAGAACCCTGGACTTCCACGACAGCAACGTGAAGAACCTGTACGACAAAGTGCGG
CTCCAGCTGCGGGACAATGCCAAAGAACTCGGCAACGGCTGCTTCGAGTTCTACCACAAGTGCGACA
ACGAGTGCATGGAAAGCGTGCGGAACGGCACCTACGACTACCCTCAGTACTCTGAGGAAGCCCGGCT
GAAGAGAGAAGAGATCAGCGGAGTGAAGCTGGAATCCATCGGCACATACCAGATCCTGAGCATCTAC
AGCACCGTGGCCTCTTCTCTGGCCCTGGCTATTATGGTGGCTGGCCTGAGCCTGTGGATGTGCTCTA
ATGGCAGCCTCCAGTGCCGGATCTGCATCTGA
FLU_T3_HA_5-head region amino acid sequence (SEQ ID NO: 45)
THNGKLCDLDGVKPLILRDCSVAGWLLGNPMCDEFINVPEWSYIVEKANPANDLCYPGNENDYEELK
HLLSRINHFEKIQIIPKSSWSDHEASSGV [symbol] ACPYQG [symbol] SFERNVVWLIKKN [symbol] YPTIKRSYNNTNQ
EDLLVLWGIHHPNDAAEQT [symbol] LYQNPTTYISVGTSTLNQRLVPKIATRSKVNG [symbol] SGRMEFFWTILKPN
DAINFESNGNFIAPEYAYKIVKKGDSAIMKSELEYGNCNTKCQTPMGAINSSMPFHNIHPLTIGECP

The amino acid residues at positions 156, 157, 171, 172, and 205 are highlighted in the above sequence (and are R, S, N, A, and R, respectively). The amino acid residues at positions 148, 149, and 238 are also highlighted, and are S, S, and E, respectively.

FLU_T3_HA_5-head region nucleic acid sequence
(SEQ ID NO: 46)
ACCCACAACGGCAAGCTGTGCGACCTGGATGGCGTGAAGCCTCTGATCCTGAGAGATTGCTCTGTGG
CCGGCTGGCTGCTGGGCAATCCTATGTGCGACGAGTTCATCAACGTGCCCGAGTGGTCCTATATCGT
GGAAAAGGCCAATCCTGCCAACGACCTGTGCTACCCCGGCAACTTCAACGACTACGAGGAACTGAAA
CATCTGCTGAGCCGGATCAACCACTTCGAGAAGATCCAGATCATCCCCAAGTCCTCTTGGAGCGATC
ACGAGGCCTCTAGCGGAGTGTCTAGCGCCTGTCCTTACCAAGGCAGAAGCAGCTTCTTCCGGAACGT
CGTGTGGCTGATCAAGAAGAACAACGCTTACCCCACCATCAAGCGGAGCTACAACAACACCAATCAA
GAGGACCTGCTGGTGCTGTGGGGCATCCACCATCCTAATGATGCCGCCGAGCAGACCCGGCTGTACC
AGAATCCTACAACCTACATCAGCGTGGGCACCAGCACACTGAACCAGAGACTGGTGCCTAAGATCGC
CACCAGATCCAAAGTGAACGGCGAAAGCGGCCGGATGGAATTCTTCTGGACCATCCTGAAGCCTAAC
GACGCCATCAACTTCGAGAGCAACGGCAACTTTATCGCCCCTGAGTACGCCTACAAGATCGTGAAGA
AGGGCGACAGCGCCATCATGAAGTCCGAGCTGGAATACGGCAACTGCAACACCAAGTGTCAGACCCC
TATGGGCGCCATCAATAGCAGCATGCCCTTCCACAACATTCACCCTCTGACCATCGGCGAGTGCCCC
FLU_T3_HA_5-first stem region amino acid sequence
(SEQ ID NO: 47)
MEKIVLLLAIVSLVKSDQICIGYHANNSTEQVDTIMEKNVTVTHAQDILEK
FLU_T3_HA_5-first stem region nucleic acid sequence
(SEQ ID NO: 48)
ATGGAAAAGATTGTGCTGCTGCTGGCCATCGTGTCCCTGGTCAAGAGCGATCAAATCTGCATCGGCT
ACCACGCCAACAACAGCACCGAACAGGTGGACACCATTATGGAAAAGAACGTGACCGTGACACACGC
CCAGGACATCCTGGAAAAG
FLU_T3_HA_5-second stem region amino acid sequence
(SEQ ID NO: 49)
KYVKSNRLVLATGLRNSPQRERRRKKRGLFGAIAGFIEGGWQGMVDGWYGYHHSNEQGSGYAADKES
TQKAIDGVTNKVNSIIDKMNTQFEAVGRE [symbol] NNLERRIENLNKKMEDG [symbol] LDVWTYNAELLVLMENERT
LDFHDSNVKNLYDKVRLQLRDNAKELGNGCFEFYHKCDNECMESVRNGTYDYPQYSEEARLKREEIS
GVKLESIGTYQILSIYSTVASSLALAIMVAGLSLWMCSNGSLQCRICI

The amino acid residues at positions 416 and 434 (or at positions 148 and 166 if counting from the beginning of the stem region) are shown underlined in the above sequence (and are F and F, respectively).

FLU_T3_HA_5-second stem region nucleic
acid sequence
(SEQ ID NO: 50)
AAATACGTGAAGTCCAACAGACTGGTCCTGGCCACCGGCCTGAGAA
ATTCTCCACAGAGAGAGCGGCGCAGAAAGAAGAGAGGCCTGTTTG
GAGCCATTGCCGGCTTTATCGAAGGCGGCTGGCAAGGCATGGTTG
ACGGATGGTACGGCTATCACCACAGCAATGAGCAAGGCTCTGGCT
ACGCCGCCGACAAAGAGAGCACACAGAAAGCCATCGACGGCGTGA
CCAACAAAGTGAATAGCATCATCGACAAGATGAACACCCAGTTCG
AGGCCGTGGGCAGAGAGTTCAACAACCTGGAAAGACGGATCGAGA
ACCTGAACAAGAAGATGGAGGACGGCTTCCTGGACGTGTGGACCT
ATAATGCCGAGCTGCTGGTCCTGATGGAAAACGAGAGAACCCTGG
ACTTCCACGACAGCAACGTGAAGAACCTGTACGACAAAGTGCGGC
TCCAGCTGCGGGACAATGCCAAAGAACTCGGCAACGGCTGCTTCG
AGTTCTACCACAAGTGCGACAACGAGTGCATGGAAAGCGTGCGGA
ACGGCACCTACGACTACCCTCAGTACTCTGAGGAAGCCCGGCTGA
AGAGAGAAGAGATCAGCGGAGTGAAGCTGGAATCCATCGGCACAT
ACCAGATCCTGAGCATCTACAGCACCGTGGCCTCTTCTCTGGCCC
TGGCTATTATGGTGGCTGGCCTGAGCCTGTGGATGTGCTCTAATG
GCAGCCTCCAGTGCCGGATCTGCATCTGA

### EXAMPLE 21—FLU_T3_HA_1

This example provides amino acid and nucleic acid sequences of the influenza haemagluttinin H5 stem regions for an embodiment of the invention known as FLU_T3_HA_1. Example 4 above provides the amino acid and nucleic acid sequences for the composite stem region of FLU_T3_HA_1, however the stem regions are separated by a head region. This example also provides the nucleic acid sequence of the H5 head and stem regions, with the stem regions underlined.

FLU_T3_HA_1-first stem region amino acid sequence
(SEQ ID NO: 51)
MEKIVLLLAIVSLVKSDQICIGYHANNSTEQVDTIMEKNVTVTHAQDILEK
FLU_T3_HA_1-first stem region nucleic acid sequence
(SEQ ID NO: 52)
ATGGAAAAGATCGTGCTGCTGCTGGCCATCGTGTCCCTGGTCAAGAGCGACCAAATCTGCATCGGCT
ACCACGCCAACAACAGCACCGAACAGGTGGACACCATTATGGAAAAGAACGTCACCGTGACACACGC
CCAGGACATCCTGGAAAAG
FLU_T3_HA_1-second stem region amino acid sequence
(SEQ ID NO: 53)
KYVKSNRLVLATGLRNSPQRERRRKKRGLFGAIAGFIEGGWQGMVDGWYGYHHSNEQGSGYAADKES
TQKAIDGVTNKVNSIIDKMNTQFEAVGREENNLERRIENLNKKMEDGELDVWTYNAELLVLMENERT
LDFHDSNVKNLYDKVRLQLRDNAKELGNGCFEFYHKCDNECMESVRNGTYDYPQYSEEARLKREEIS
GVKLESIGTYQILSIYSTVASSLALAIMVAGLSLWMCSNGSLQCRICI
FLU_T3_HA_1-second stem region nucleic acid sequence
(SEQ ID NO: 54)
AAATACGTGAAGTCCAACAGACTGGTCCTGGCCACCGGCCTGAGAAACTCTCCCCAGCGCGAGCGGA
GAAGAAAGAAGAGAGGCCTGTTTGGCGCCATTGCCGGCTTTATCGAAGGCGGCTGGCAAGGCATGGT
GGACGGATGGTACGGCTATCACCACAGCAACGAGCAAGGCTCTGGATACGCCGCCGACAAAGAGAGC
ACCCAGAAAGCCATTGACGGCGTGACCAACAAAGTCAACAGCATCATCGACAAGATGAACACCCAGT
TCGAGGCCGTGGGCAGAGAGTTCAACAACCTGGAACGGCGGATCGAGAACCTGAACAAGAAGATGGA
GGACGGCTTCCTGGACGTGTGGACCTACAATGCCGAGCTGCTGGTCCTGATGGAAAACGAGAGAACC
CTGGACTTCCACGACAGCAACGTGAAGAACCTGTACGACAAAGTGCGGCTCCAGCTGCGGGACAACG
CCAAAGAACTCGGCAACGGCTGCTTCGAGTTCTACCACAAGTGCGACAACGAGTGCATGGAAAGCGT
GCGGAACGGCACCTACGACTACCCTCAGTACAGCGAGGAAGCCCGGCTGAAGAGGGAAGAGATCAGC
GGAGTGAAGCTGGAATCCATCGGCACATACCAGATCCTGAGCATCTACAGCACCGTGGCCTCTTCTC
TGGCCCTGGCCATTATGGTGGCTGGCCTGTCTCTGTGGATGTGCAGCAATGGCAGCCTCCAGTGCCG
GATCTGCATCTGA
FLU_T3_HA_1-HAO nucleic acid sequence
(SEQ ID NO: 55)
ATGGAAAAGATCGTGCTGCTGCTGGCCATCGTGTCCCTGGTCAAGAGCGACCAAATCTGCATCGGCT
ACCACGCCAACAACAGCACCGAACAGGTGGACACCATTATGGAAAAGAACGTCACCGTGACACACGC
CCAGGACATCCTGGAAAAGACCCACAACGGCAAGCTGTGCGACCTGGATGGCGTGAAGCCTCTGATC
CTGAGAGATTGCTCTGTGGCCGGATGGCTGCTGGGCAATCCCATGTGCGACGAGTTCATCAACGTGC
CCGAGTGGTCCTATATCGTGGAAAAGGCCAATCCTGCCAACGACCTGTGCTACCCCGGCAACTTCAA
CGACTACGAGGAACTGAAGCACCTCCTGAGCCGGATCAACCACTTCGAGAAGATCCAGATCATCCCC
AAGTCCTCTTGGAGCGACCACGAAGCCTCTAGCGGAGTGTCTAGCGCCTGTCCTTACCAAGGCAGAC
CCAGCTTCTTCCGGAACGTCGTGTGGCTGATCAAGAAGAACGACACATACCCCACCATCAAGCGGAG
CTACAACAACACCAATCAAGAGGACCTGCTGGTGCTGTGGGGCATCCACCATCCTAATGATGCCGCC
GAGCAGACCAAGCTGTATCAGAACCCCACCACCTACATCAGCGTGGGCACCAGCACACTGAACCAGA
GACTGGTGCCTAAGATCGCCACCAGATCCAAAGTGAACGGCCAGAGCGGCAGAATGGAATTCTTCTG
GACCATCCTGAAGCCTAACGACGCCATCAACTTCGAGAGCAACGGCAACTTTATCGCCCCTGAGTAC
GCCTACAAGATCGTGAAGAAGGGCGACAGCGCCATCATGAAGTCCGAGCTGGAATACGGCAACTGCA
ACACCAAGTGTCAGACCCCTATGGGCGCCATCAATAGCAGCATGCCCTTCCACAACATTCACCCTCT
GACCATCGGCGAGTGCCCCAAATACGTGAAGTCCAACAGACTGGTCCTGGCCACCGGCCTGAGAAAC
TCTCCCCAGCGCGAGCGGAGAAGAAAGAAGAGAGGCCTGTTTGGCGCCATTGCCGGCTTTATCGAAG
GCGGCTGGCAAGGCATGGTGGACGGATGGTACGGCTATCACCACAGCAACGAGCAAGGCTCTGGATA
CGCCGCCGACAAAGAGAGCACCCAGAAAGCCATTGACGGCGTGACCAACAAAGTCAACAGCATCATC
GACAAGATGAACACCCAGTTCGAGGCCGTGGGCAGAGAGTTCAACAACCTGGAACGGCGGATCGAGA
ACCTGAACAAGAAGATGGAGGACGGCTTCCTGGACGTGTGGACCTACAATGCCGAGCTGCTGGTCCT
GATGGAAAACGAGAGAACCCTGGACTTCCACGACAGCAACGTGAAGAACCTGTACGACAAAGTGCGG
CTCCAGCTGCGGGACAACGCCAAAGAACTCGGCAACGGCTGCTTCGAGTTCTACCACAAGTGCGACA
ACGAGTGCATGGAAAGCGTGCGGAACGGCACCTACGACTACCCTCAGTACAGCGAGGAAGCCCGGCT
GAAGAGGGAAGAGATCAGCGGAGTGAAGCTGGAATCCATCGGCACATACCAGATCCTGAGCATCTAC
AGCACCGTGGCCTCTTCTCTGGCCCTGGCCATTATGGTGGCTGGCCTGTCTCTGTGGATGTGCAGCA
ATGGCAGCCTCCAGTGCCGGATCTGCATCTGA
FLU_T3_HA_1-head region nucleic acid sequence
(SEQ ID NO: 56)
ACCCACAACGGCAAGCTGTGCGACCTGGATGGCGTGAAGCCTCTGATCCTGAGAGATTGCTCTGTGG
CCGGATGGCTGCTGGGCAATCCCATGTGCGACGAGTTCATCAACGTGCCCGAGTGGTCCTATATCGT
GGAAAAGGCCAATCCTGCCAACGACCTGTGCTACCCCGGCAACTTCAACGACTACGAGGAACTGAAG
CACCTCCTGAGCCGGATCAACCACTTCGAGAAGATCCAGATCATCCCCAAGTCCTCTTGGAGCGAC
CACGAAGCCTCTAGCGGAGTGTCTAGCGCCTGTCCTTACCAAGGCAGACCCAGCTTCTTCCGGAACG
TCGTGTGGCTGATCAAGAAGAACGACACATACCCCACCATCAAGCGGAGCTACAACAACACCAATCA
AGAGGACCTGCTGGTGCTGTGGGGCATCCACCATCCTAATGATGCCGCCGAGCAGACCAAGCTGTAT
CAGAACCCCACCACCTACATCAGCGTGGGCACCAGCACACTGAACCAGAGACTGGTGCCTAAGATCG
CCACCAGATCCAAAGTGAACGGCCAGAGCGGCAGAATGGAATTCTTCTGGACCATCCTGAAGCCTAA
CGACGCCATCAACTTCGAGAGCAACGGCAACTTTATCGCCCCTGAGTACGCCTACAAGATCGTGAAG
AAGGGCGACAGCGCCATCATGAAGTCCGAGCTGGAATACGGCAACTGCAACACCAAGTGTCAGACCC
CTATGGGCGCCATCAATAGCAGCATGCCCTTCCACAACATTCACCCTCTGACCATCGGCGAGTGCCC
C

### EXAMPLE 22—FLU_T3_HA_2

This example provides amino acid and nucleic acid sequences of the influenza haemagluttinin H5 stem regions for an embodiment of the invention known as FLU_T3_HA_2. Example 5 above provides the amino acid and nucleic acid sequences for the composite stem region of FLU_T3_HA_2, however the stem regions are separated by a head region. This example also provides the nucleic acid sequence of the H5 head and stem regions, with the stem regions underlined.

FLU_T3_HA_2-first stem region amino acid sequence
(SEQ ID NO: 57)
MEKIVLLLAIVSLVKSDQICIGYHANNSTEQVDTIMEKNVTVTHAQDILEK
FLU_T3_HA_2-first stem region nucleic acid sequence
(SEQ ID NO: 58)
ATGGAAAAGATCGTGCTGCTGCTGGCCATCGTGTCCCTGGTCAAGAGCGACCAAATCTGCATCGGCT
ACCACGCCAACAACAGCACCGAACAGGTGGACACCATTATGGAAAAGAACGTCACCGTGACACACGC
CCAGGACATCCTGGAAAAG
FLU_T3_HA_2-second stem region amino acid sequence
(SEQ ID NO: 59)
KYVKSNRLVLATGLRNSPQRERRRKKRGLFGAIAGFIEGGWQGMVDGWYGYHHSNEQGSGYAADKES
TQKAIDGVTNKVNSIIDKMNTQFEAVGREENNLERRIENLNKKMEDGELDVWTYNAELLVLMENERT
LDFHDSNVKNLYDKVRLQLRDNAKELGNGCFEFYHKCDNECMESVRNGTYDYPQYSEEARLKREEIS
GVKLESIGTYQILSIYSTVASSLALAIMVAGLSLWMCSNGSLQCRICI
FLU_T3_HA_2-second stem region nucleic acid sequence
(SEQ ID NO: 60)
AAATACGTGAAGTCCAACAGACTGGTCCTGGCCACCGGCCTGAGAAACTCTCCCCAGCGCGAGCGGA
GAAGAAAGAAGAGAGGCCTGTTTGGCGCCATTGCCGGCTTTATCGAAGGCGGCTGGCAAGGCATGGT
GGACGGATGGTACGGCTATCACCACAGCAACGAGCAAGGCTCTGGATACGCCGCCGACAAAGAGAGC
ACCCAGAAAGCCATTGACGGCGTGACCAACAAAGTCAACAGCATCATCGACAAGATGAACACCCAGT
TCGAGGCCGTGGGCAGAGAGTTCAACAACCTGGAACGGCGGATCGAGAACCTGAACAAGAAGATGGA
GGACGGCTTCCTGGACGTGTGGACCTACAATGCCGAGCTGCTGGTCCTGATGGAAAACGAGAGAACC
CTGGACTTCCACGACAGCAACGTGAAGAACCTGTACGACAAAGTGCGGCTCCAGCTGCGGGACAACG
CCAAAGAACTCGGCAACGGCTGCTTCGAGTTCTACCACAAGTGCGACAACGAGTGCATGGAAAGCGT
GCGGAACGGCACCTACGACTACCCTCAGTACAGCGAGGAAGCCCGGCTGAAGAGGGAAGAGATCAGC
GGAGTGAAGCTGGAATCCATCGGCACATACCAGATCCTGAGCATCTACAGCACCGTGGCCTCTTCTC
TGGCCCTGGCCATTATGGTGGCTGGCCTGTCTCTGTGGATGTGCAGCAATGGCAGCCTCCAGTGCCG
GATCTGCATCTGA
FLU_T3_HA_2-HAO nucleic acid sequence
(SEQ ID NO: 61)
ATGGAAAAGATCGTGCTGCTGCTGGCCATCGTGTCCCTGGTCAAGAGCGACCAAATCTGCATCGGCT
ACCACGCCAACAACAGCACCGAACAGGTGGACACCATTATGGAAAAGAACGTCACCGTGACACACGC
CCAGGACATCCTGGAAAAGACCCACAACGGCAAGCTGTGCGACCTGGATGGCGTGAAGCCTCTGATC
CTGAGAGATTGCTCTGTGGCCGGATGGCTGCTGGGCAATCCCATGTGCGACGAGTTCATCAACGTGC
CCGAGTGGTCCTATATCGTGGAAAAGGCCAATCCTGCCAACGACCTGTGCTACCCCGGCAACTTCAA
CGACTACGAGGAACTGAAGCACCTCCTGAGCCGGATCAACCACTTCGAGAAGATCCAGATCATCCCC
AAGTCCTCTTGGAGCGACCACGAAGCCTCTAGCGGAGTGTCTAGCGCCTGTCCTTACCAAGGCAGAC
CCAGCTTCTTCCGGAACGTCGTGTGGCTGATCAAGAAGAACAACACATACCCCACCATCAAGCGGAG
CTACAACAACACCAATCAAGAGGACCTGCTGGTGCTGTGGGGCATCCACCATCCTAATGATGCCGCC
GAGCAGACCAAGCTGTATCAGAACCCCACCACCTACATCAGCGTGGGCACCAGCACACTGAACCAGA
GACTGGTGCCTAAGATCGCCACCAGATCCAAAGTGAACGGCCAGAGCGGCAGAATGGAATTCTTCTG
GACCATCCTGAAGCCTAACGACGCCATCAACTTCGAGAGCAACGGCAACTTTATCGCCCCTGAGTAC
GCCTACAAGATCGTGAAGAAGGGCGACAGCGCCATCATGAAGTCCGAGCTGGAATACGGCAACTGCA
ACACCAAGTGTCAGACCCCTATGGGCGCCATCAATAGCAGCATGCCCTTCCACAACATTCACCCTCT
GACCATCGGCGAGTGCCCCAAATACGTGAAGTCCAACAGACTGGTCCTGGCCACCGGCCTGAGAAAC
TCTCCCCAGCGCGAGCGGAGAAGAAAGAAGAGAGGCCTGTTTGGCGCCATTGCCGGCTTTATCGAAG
GCGGCTGGCAAGGCATGGTGGACGGATGGTACGGCTATCACCACAGCAACGAGCAAGGCTCTGGATA
CGCCGCCGACAAAGAGAGCACCCAGAAAGCCATTGACGGCGTGACCAACAAAGTCAACAGCATCATC
GACAAGATGAACACCCAGTTCGAGGCCGTGGGCAGAGAGTTCAACAACCTGGAACGGCGGATCGAGA
ACCTGAACAAGAAGATGGAGGACGGCTTCCTGGACGTGTGGACCTACAATGCCGAGCTGCTGGTCCT
GATGGAAAACGAGAGAACCCTGGACTTCCACGACAGCAACGTGAAGAACCTGTACGACAAAGTGCGG
CTCCAGCTGCGGGACAACGCCAAAGAACTCGGCAACGGCTGCTTCGAGTTCTACCACAAGTGCGACA
ACGAGTGCATGGAAAGCGTGCGGAACGGCACCTACGACTACCCTCAGTACAGCGAGGAAGCCCGGCT
GAAGAGGGAAGAGATCAGCGGAGTGAAGCTGGAATCCATCGGCACATACCAGATCCTGAGCATCTAC
AGCACCGTGGCCTCTTCTCTGGCCCTGGCCATTATGGTGGCTGGCCTGTCTCTGTGGATGTGCAGCA
ATGGCAGCCTCCAGTGCCGGATCTGCATCTGA
FLU_T3_HA_2-head region nucleic acid sequence
(SEQ ID NO: 62)
ACCCACAACGGCAAGCTGTGCGACCTGGATGGCGTGAAGCCTCTGATCCTGAGAGATTGCTCTGTGG
CCGGATGGCTGCTGGGCAATCCCATGTGCGACGAGTTCATCAACGTGCCCGAGTGGTCCTATATCGT
GGAAAAGGCCAATCCTGCCAACGACCTGTGCTACCCCGGCAACTTCAACGACTACGAGGAACTGAAG
CACCTCCTGAGCCGGATCAACCACTTCGAGAAGATCCAGATCATCCCCAAGTCCTCTTGGAGCGACC
ACGAAGCCTCTAGCGGAGTGTCTAGCGCCTGTCCTTACCAAGGCAGACCCAGCTTCTTCCGGAACGT
CGTGTGGCTGATCAAGAAGAACAACACATACCCCACCATCAAGCGGAGCTACAACAACACCAATCAA
GAGGACCTGCTGGTGCTGTGGGGCATCCACCATCCTAATGATGCCGCCGAGCAGACCAAGCTGTATC
AGAACCCCACCACCTACATCAGCGTGGGCACCAGCACACTGAACCAGAGACTGGTGCCTAAGATCGC
CACCAGATCCAAAGTGAACGGCCAGAGCGGCAGAATGGAATTCTTCTGGACCATCCTGAAGCCTAAC
GACGCCATCAACTTCGAGAGCAACGGCAACTTTATCGCCCCTGAGTACGCCTACAAGATCGTGAAGA
AGGGCGACAGCGCCATCATGAAGTCCGAGCTGGAATACGGCAACTGCAACACCAAGTGTCAGACCCC
TATGGGCGCCATCAATAGCAGCATGCCCTTCCACAACATTCACCCTCTGACCATCGGCGAGTGCCCC

### EXAMPLE 23—RESIDUE DIFFERENCES IN AMINO ACID SEQUENCE OF INFLUENZA TIER 3 H5 VACCINE CANDIDATES FLU_T3_HA_1 TO FLU_T3_HA_5, AND INFLUENZA TIER 2 H5 DESIGN FLU_T2_HA_1

FIG. 21 summarises differences in amino acid sequence of the influenza haemagluttinin H5 for different embodiments of the invention, including differences at positions A-E of H5 for the embodiments. Positions A, B, and C of H5 are at epitope regions in the head region, and the mutations shown in the figure at these positions increase the affinity of H5 towards binding antibodies. Positions D and E are in the H5 stem region, and the mutations at these positions increase the stability of the stem region both in the pre-fusion and post-fusion state. The amino acid residue mutations at positions 148, 149, and 238 of FLU_T3_HA_4, and at position 238 of FLU_T3_HA_5, are at receptor binding sites. These residue mutations reduce the affinity of HA to its receptor (sialic acid) on the surface of target cells, thus increasing the bioavailability of HA for antigen presentation.

FIG. 22 shows a multiple sequence alignment of HA amino acid sequence for FLU_T2_HA_1, FLU_T3_HA_1 to FLU_T3_HA_5, and two influenza isolates H5_WSN (SEQ ID NO:64) and H5 GYR (SEQ ID NO:65). In the figure, differences in amino acid residues are shown underlined, with amino acid differences across designed sequences FLU_T2_HA_1 and FLU_T3_HA_1/2/3/4/5 shown highlighted. The amino acid residues at positions A, B, and C of the head region, and D and E of the stem region, are shown in boxes. These amino acid residues are at residue positions 156, 157, 171, 172, and 205 of the head region, and at residue positions 416 and 434 of the stem region.

>A/WSN/Mongolia/244/2005
(SEQ ID NO: 64)
Amino acid sequence
MEKIVLLLAIVSLVKSDQICIGYHANNSTEQVDTIMEKNVTVTHAQDILEKTHNGKLCDL
DGVKPLILRDCSVAGWLLGNPMCDEFLNVPEWSYIVEKINPANDLCYPGNENDYEELKHL
LSRINHFEKIQIIPKSSWSDHEASSGVSSACPYQGRSSFERNVVWLIKKDNAYPTIKRSY
NNTNQEDLLVLWGIHHPNDAAEQTRLYQNPTTYISVGTSTLNQRLVPKIATRSKVNGQSG
RMEFFWTILKPNDAINFESNGNFIAPENAYKIVKKGDSTIMKSELEYGNCNTKCQTPIGA
INSSMPFHNIHPLTIGECPKYVKSNRLVLATGLRNSPQGE-RRRRKRGLFGAIAGFIEGG
WQGMVDGWYGYHHSNEQGSGYAADKESTQKAIDGVINKVNSIIDKMNTQFEAVGREENNL
ERRIENLNKKMEDGELDVWTYNAELLVLMENERTLDFHDSNVKNLYDKVRLQLRDNAKEL
GNGCFEFYHRCDNECMESVRNGTYDYPQYSEEARLKREEISGVKLESIGTYQILSIYSTV
ASSLALAIMVAGLSLWMCSNGSLQCRICI
>A/gyrfalcon/Washington/41088-6/2014 H5N8
Amino acid sequence
(FLU T1 HA 9; SEQ ID NO: 65))
MEKIVLLLAVISLVKSDQICIGYHANNSTKQVDTIMEKNVTVTHAQDILEKTHNGKLCDL
NGVKPLILKDCSVAGWLLGNPMCDEFIRVPEWSYIVERANPANDLCYPGTLNDYEELKHL
LSRINHFEKTLIIPRSSWPNHETSLGVSAACPYQGASSFERNVVWLIKKNDAYPTIKISY
NNTNREDLLILWGIHHSNNAAEQTNLYKNPDTYVSVGTSTLNQRLVPKIATRSQVNGQSG
RMDFFWTILKPNDAIHFESNGNFIAPEYAYKIVKKGDSTIMKSEMEYGHCNTKCQTPIGA
INSSMPFHNIHPLTIGECPKYVKSNKLVLATGLRNSPLRERRRKRGLFGAIAGFIEGGWQ
GMVDGWYGYHHSNEQGSGYAADKESTQKAIDGVTNKVNSIIDKMNTQFEAVGREENNLER
RIENLNKKMEDGELDVWTYNAELLVLMENERTLDFHDSNVKNLYDKVRLQLRDNAKELGN
GCFEFYHKCDNECMESVRNGTYDYPKYSEEAILKREEISGVKLESIGTYQILSIYSTVAS
SLALAIIVAGLSLWMCSNGSLQCRICI

### EXAMPLE 24—ITERATIVELY DESIGNED H5NX ANTIGENS GENERATE BROAD IMMUNE RESPONSE IN MICE

### Background

Yearly outbreak of avian influenza (H5Nx) has a huge socio-economic impact worldwide. In addition, there is a constant threat of spill overs to naïve human population leading to a pandemic. Constant antigenic drift in the surface glycoprotein of influenza—hemagglutinin, and recombination with different neuraminidase subtypes add a complex dimension to design of universal H5 influenza vaccine antigens that can provide broad protection to H5Nx. Here we discuss our H5Nx antigen designs that has been iteratively optimised to increase the coverage of H5Nx.

### Methods

Available H5Nx sequences from NCBI virus database were downloaded, cleaned, and trimmed to generate a non-redundant dataset of H5 sequences. Phylogenetic relationship between these sequences were estimated and a phylogenetically optimised sequence was designed as our first vaccine candidate FLU_T2_HA_1 (referred to as DIOS-T2_HA_9 in FIG. 23). Immunogenicity of the vaccine design was confirmed in Balb/c mice. Mice sera were tested for neutralisation using pseudotype neutralisation assays against multiple H5 viruses. Based on these results, FLU_T2_HA_1 was further optimised to generate a panel of next tier vaccine designs FLU_T3_HA_1/2/3/4/5 (referred to as DIOS-T3_HA_1/2/3/4/5 in FIG. 23) using epitope optimisation to achieve broad neutralisation.

### Results

Our vaccine candidate FLU_T2_HA_1 generates better immune response in comparison to the wild-type control (A/whooper swan/Mongolia/244/2005) against different H5Nx, except a few H5Nx, where both our design and the WT do not show robust neutralisation. We further improved FLU_T2_HA_1 to broaden the immune response to H5Nx clades missed earlier. The improved designs (FLU_T3_HA_1/2/3/4/5) showed a better neutralisation profile against a panel of 9 antigenically different H5Nx (FIG. 23). American non-goose Guangdong (Am-nonGsGd) is a low pathogenic lineage of H5Nx viruses.

### Conclusion

A promising panel of platform independent vaccine candidates that can provide a broader protection against multiple antigenically different H5Nx. The vaccine candidate can be a useful tool in keeping in check the recurrent yearly avian influenza outbreak and preparedness of future spill over into human population.

### EXAMPLE 25—FLU_T2_HA_4

This example provides the amino acid and nucleic acid sequences of the influenza H1 region for an embodiment of the invention known as FLU_T2_HA_4.

FLU_T2_HA_4-amino acid sequence (SEQ ID NO: 68):
MKVKLLVLLCTFTATYADTICIGYHANNSTDTVDTVLEKNVTVTHSVNLLEDSHNGKLCLLKGIAPL
QLGNCSVAGWILGNPECELLISKESWSYIVETPNPENGTCYPGYFADYEELREQLSSVSSFERFEIF
PKESSWPNHTVTSGVSASCSHNGKSSFYRNLLWLTGKNGLYPNLSKSYANNKEKEVLVLWGVHHPPN
IGDQRALYHTENAYVSVVSSHYSRRFTPEIAKRPKVRDQEGRINYYWTLLEPGDTIIFEANGNLIAP
RYAFALSRGFGSGIINSNAPMDECDAKCQTPQGAINSSLPFQNVHPVTIGECPKYVRSAKLRMVTGL
RNIPSIQSRGLFGAIAGFIEGGWTGMVDGWYGYHHQNEQGSGYAADQKSTQNAINGITNKVNSVIEK
MNTQFTAVGKEFNKLERRMENLNKKVDDGFIDIWTYNAELLVLLENERTLDFHDSNVKNLYEKVKSQ
LKNNAKEIGNGCFEFYHKCNDECMESVKNGTYDYPKYSEESKLNREKIDGVKLESMGVYQILAIYST
VASSLVLLVSLGAISFWMCSNGSLQCRICI
FLU_T2_HA_4-nucleic acid sequence (SEQ ID NO: 69)
ATGAAGGTCAAACTGCTGGTGCTGCTGTGCACCTTCACCGCCACATACGCCGATACCATCTGTATCG
GCTACCACGCCAACAACAGCACCGACACCGTGGATACCGTGCTGGAAAAGAACGTGACCGTGACACA
CAGCGTGAACCTGCTGGAAGATAGCCACAACGGCAAGCTGTGCCTGCTGAAGGGAATTGCCCCTCTC
CAGCTGGGAAATTGCTCTGTGGCTGGCTGGATCCTGGGCAATCCTGAGTGCGAGCTGCTGATCTCCA
AAGAGAGCTGGTCCTACATCGTCGAGACACCCAATCCAGAGAACGGCACATGCTACCCCGGCTACTT
CGCCGACTATGAGGAACTGAGAGAGCAGCTGAGCAGCGTCAGCAGCTTCGAGAGATTCGAGATTTTC
CCCAAAGAGTCCAGCTGGCCCAACCACACAGTGACAAGCGGAGTGTCTGCCAGCTGTTCCCACAATG
GCAAGAGCAGCTTCTACAGAAACCTGCTGTGGCTGACCGGCAAGAACGGACTGTACCCCAACCTGAG
CAAGAGCTACGCTAACAACAAAGAGAAAGAGGTCCTGGTCCTCTGGGGCGTGCACCATCCTCCAAAT
ATCGGAGATCAGAGAGCCCTGTACCACACCGAGAATGCCTACGTGTCCGTGGTGTCCAGCCACTACA
GCAGAAGATTCACCCCTGAGATCGCCAAGCGGCCCAAAGTGCGAGATCAAGAGGGCAGAATCAACTA
CTACTGGACACTGCTGGAACCCGGCGACACCATCATCTTCGAGGCCAACGGAAACCTGATCGCCCCT
AGATACGCCTTCGCTCTGAGCAGAGGCTTTGGCAGCGGCATCATCAACAGCAACGCCCCTATGGATG
AGTGCGACGCCAAGTGTCAAACACCCCAGGGCGCTATCAACAGCTCCCTGCCTTTTCAGAACGTGCA
CCCTGTGACCATCGGCGAGTGTCCTAAATATGTGCGGAGCGCCAAGCTGAGAATGGTCACCGGCCTG
AGAAACATCCCCAGCATCCAGTCTAGAGGCCTGTTTGGCGCCATTGCCGGCTTTATCGAAGGCGGAT
GGACAGGCATGGTGGACGGATGGTACGGCTATCACCACCAGAATGAGCAAGGCAGCGGCTACGCCGC
CGATCAGAAATCTACCCAGAACGCCATCAACGGGATCACCAACAAAGTGAACAGCGTGATCGAGAAG
ATGAACACCCAGTTCACCGCCGTGGGCAAAGAGTTCAACAAGCTGGAAAGACGGATGGAAAACCTGA
ACAAGAAGGTGGACGACGGCTTCATCGACATCTGGACCTACAACGCTGAGCTGCTGGTCCTCCTGGA
AAACGAGAGAACCCTGGACTTCCACGACAGCAACGTGAAGAACCTGTACGAGAAAGTGAAGTCCCAG
CTGAAGAACAACGCCAAAGAGATCGGCAACGGCTGCTTCGAGTTCTACCACAAGTGCAACGACGAGT
GCATGGAAAGCGTGAAGAATGGCACCTACGACTACCCCAAGTACAGCGAGGAAAGCAAGCTGAACCG
CGAGAAGATCGACGGCGTGAAGCTGGAATCTATGGGCGTGTACCAGATCCTGGCCATCTACAGCACA
GTGGCTTCTAGCCTGGTGCTCCTGGTGTCTCTGGGAGCCATCAGCTTTTGGATGTGCAGCAATGGCA
GCCTCCAGTGCCGGATCTGCATC

### EXAMPLE 26—PEVAC-FLU_T2-HA_4

This example provides the nucleic acid sequence of pEVAC-FLU_T2-HA_4.

| pEVAC-FLU_T2_HA_4-nucleic acid sequence (SEQ ID NO: 70): | |
|---|---|
| LOCUS 17ADKK4C_I-3 pVRC8400EVAC_Ar 6083 bp DNA circular | |
| FEATURES | Location/Qualifiers |
| promoter | complement (5925 . . . 5953) |
| | /label = “AmpR_promoter” |
| promoter | 868 . . . 987 |
| | /label = “CMV2_promoter” |
| CDS | complement (4963 . . . 5778) |
| | /label = “Kana (R) ” |
| rep_origin | complement (3818 . . . 4437) |
| | /label = “pBR322_origin” |
| primer | complement (29 . . . 51) |
| | /label = “pGEX_3_primer” |
| primer | 855 . . . 875 |
| | /label = “CMV_fwd_primer” |
| primer | 899 . . . 918 |
| | /label = “pCEP_fwd_primer” |
| primer | 901 . . . 925 |
| | /label = “LNCX_primer” |
| polyA_site | 3071 . . . 3295 |
| | /label = “BGH\pA” |
| promoter | 394 . . . 904 |
| | /label = “CMV_Promoter” |
| CDS | 1343 . . . 3063 |
| | /label = “I-3”1/53 |

```
ORIGIN
      1 TCGCGCGTTT CGGTGATGAC GGTGAAAACC TCTGACACAT GCAGCTCCCG GAGACGGTCA
    61 CAGCTTGTCT GTAAGCGGAT GCCGGGAGCA GACAAGCCCG TCAGGGCGCG TCAGCGGGTG
  121 TTGGCGGGTG TCGGGGCTGG CTTAACTATG CGGCATCAGA GCAGATTGTA CTGAGAGTGC
  181 ACCATATGCG GTGTGAAATA CCGCACAGAT GCGTAAGGAG AAAATACCGC ATCAGATTGG
  241 CTATTGGCCA TTGCATACGT TGTATCCATA TCATAATATG TACATTTATA TTGGCTCATG
  301 TCCAACATTA CCGCCATGTT GACATTGATT ATTGACTAGT TATTAATAGT AATCAATTAC
  361 GGGGTCATTA GTTCATAGCC CATATATGGA GTTCCGCGTT ACATAACTTA CGGTAAATGG
  421 CCCGCCTGGC TGACCGCCCA ACGACCCCCG CCCATTGACG TCAATAATGA CGTATGTTCC
  481 CATAGTAACG CCAATAGGGA CTTTCCATTG ACGTCAATGG GTGGAGTATT TACGGTAAAC
  541 TGCCCACTTG GCAGTACATC AAGTGTATCA TATGCCAAGT ACGCCCCCTA TTGACGTCAA
  601 TGACGGTAAA TGGCCCGCCT GGCATTATGC CCAGTACATG ACCTTATGGG ACTTTCCTAC
  661 TTGGCAGTAC ATCTACGTAT TAGTCATCGC TATTACCATG GTGATGCGGT TTTGGCAGTA
  721 CATCAATGGG CGTGGATAGC GGTTTGACTC ACGGGGATTT CCAAGTCTCC ACCCCATTGA
  781 CGTCAATGGG AGTTTGTTTT GGCACCAAAA TCAACGGGAC TTTCCAAAAT GTCGTAACAA
  841 CTCCGCCCCA TTGACGCAAA TGGGCGGTAG GCGTGTACGG TGGGAGGTCT ATATAAGCAG
  901 AGCTCGTTTA GTGAACCGTC AGATCGCCTG GAGACGCCAT CCACGCTGTT TTGACCTCCA
  961 TAGAAGACAC CGGGACCGAT CCAGCCTCCA TCGGCTCGCA TCTCTCCTTC ACGCGCCCGC
1021 CGCCCTACCT GAGGCCGCCA TCCACGCCGG TTGAGTCGCG TTCTGCCGCC TCCCGCCTGT
1081 GGTGCCTCCT GAACTGCGTC CGCCGTCTAG GTAAGTTTAA AGCTCAGGTC GAGACCGGGC
1141 CTTTGTCCGG CGCTCCCTTG GAGCCTACCT AGACTCAGCC GGCTCTCCAC GCTTTGCCTG
1201 ACCCTGCTTG CTCAACTCTA GTTAACGGTG GAGGGCAGTG TAGTCTGAGC AGTACTCGTT
1261 GCTGCCGCGC GCGCCACCAG ACATAATAGC TGACAGACTA ACAGACTGTT CCTTTCCATG
1321 GGTCTTTTCT GCAGTCACCG TCGGTACCGC CACCATGAAG GTCAAACTGC TGGTGCTGCT
1381 GTGCACCTTC ACCGCCACAT ACGCCGATAC CATCTGTATC GGCTACCACG CCAACAACAG
1441 CACCGACACC GTGGATACCG TGCTGGAAAA GAACGTGACC GTGACACACA GCGTGAACCT
1501 GCTGGAAGAT AGCCACAACG GCAAGCTGTG CCTGCTGAAG GGAATTGCCC CTCTCCAGCT
1561 GGGAAATTGC TCTGTGGCTG GCTGGATCCT GGGCAATCCT GAGTGCGAGC TGCTGATCTC
1621 CAAAGAGAGC TGGTCCTACA TCGTCGAGAC ACCCAATCCA GAGAACGGCA CATGCTACCC
1681 CGGCTACTTC GCCGACTATG AGGAACTGAG AGAGCAGCTG AGCAGCGTCA GCAGCTTCGA
1741 GAGATTCGAG ATTTTCCCCA AAGAGTCCAG CTGGCCCAAC CACACAGTGA CAAGCGGAGT
1801 GTCTGCCAGC TGTTCCCACA ATGGCAAGAG CAGCTTCTAC AGAAACCTGC TGTGGCTGAC
1861 CGGCAAGAAC GGACTGTACC CCAACCTGAG CAAGAGCTAC GCTAACAACA AAGAGAAAGA
1921 GGTCCTGGTC CTCTGGGGCG TGCACCATCC TCCAAATATC GGAGATCAGA GAGCCCTGTA
1981 CCACACCGAG AATGCCTACG TGTCCGTGGT GTCCAGCCAC TACAGCAGAA GATTCACCCC
2041 TGAGATCGCC AAGCGGCCCA AAGTGCGAGA TCAAGAGGGC AGAATCAACT ACTACTGGAC
2101 ACTGCTGGAA CCCGGCGACA CCATCATCTT CGAGGCCAAC GGAAACCTGA TCGCCCCTAG
2161 ATACGCCTTC GCTCTGAGCA GAGGCTTTGG CAGCGGCATC ATCAACAGCA ACGCCCCTAT
2221 GGATGAGTGC GACGCCAAGT GTCAAACACC CCAGGGCGCT ATCAACAGCT CCCTGCCTTT
2281 TCAGAACGTG CACCCTGTGA CCATCGGCGA GTGTCCTAAA TATGTGCGGA GCGCCAAGCT
2341 GAGAATGGTC ACCGGCCTGA GAAACATCCC CAGCATCCAG TCTAGAGGCC TGTTTGGCGC
2401 CATTGCCGGC TTTATCGAAG GCGGATGGAC AGGCATGGTG GACGGATGGT ACGGCTATCA
2461 CCACCAGAAT GAGCAAGGCA GCGGCTACGC CGCCGATCAG AAATCTACCC AGAACGCCAT
2521 CAACGGGATC ACCAACAAAG TGAACAGCGT GATCGAGAAG ATGAACACCC AGTTCACCGC
2581 CGTGGGCAAA GAGTTCAACA AGCTGGAAAG ACGGATGGAA AACCTGAACA AGAAGGTGGA
2641 CGACGGCTTC ATCGACATCT GGACCTACAA CGCTGAGCTG CTGGTCCTCC TGGAAAACGA
2701 GAGAACCCTG GACTTCCACG ACAGCAACGT GAAGAACCTG TACGAGAAAG TGAAGTCCCA
2761 GCTGAAGAAC AACGCCAAAG AGATCGGCAA CGGCTGCTTC GAGTTCTACC ACAAGTGCAA
2821 CGACGAGTGC ATGGAAAGCG TGAAGAATGG CACCTACGAC TACCCCAAGT ACAGCGAGGA
2881 AAGCAAGCTG AACCGCGAGA AGATCGACGG CGTGAAGCTG GAATCTATGG GCGTGTACCA
2941 GATCCTGGCC ATCTACAGCA CAGTGGCTTC TAGCCTGGTG CTCCTGGTGT CTCTGGGAGC
3001 CATCAGCTTT TGGATGTGCA GCAATGGCAG CCTCCAGTGC CGGATCTGCA TCTGAGCGGC
3061 CGCAGATCTG CTGTGCCTTC TAGTTGCCAG CCATCTGTTG TTTGCCCCTC CCCCGTGCCT
3121 TCCTTGACCC TGGAAGGTGC CACTCCCACT GTCCTTTCCT AATAAAATGA GGAAATTGCA
3181 TCGCATTGTC TGAGTAGGTG TCATTCTATT CTGGGGGGTG GGGTGGGGCA GGACAGCAAG
3241 GGGGAGGATT GGGAAGACAA TAGCAGGCAT GCTGGGGATG CGGTGGGCTC TATGGCTACC
3301 CAGGTGCTGA AGAATTGACC CGGTTCCTCC TGGGCCAGAA AGAAGCAGGC ACATCCCCTT
3361 CTCTGTGACA CACCCTGTCC ACGCCCCTGG TTCTTAGTTC CAGCCCCACT CATAGGACAC
3421 TCATAGCTCA GGAGGGCTCC GCCTTCAATC CCACCCGCTA AAGTACTTGG AGCGGTCTCT
3481 CCCTCCCTCA TCAGCCCACC AAACCAAACC TAGCCTCCAA GAGTGGGAAG AAATTAAAGC
3541 AAGATAGGCT ATTAAGTGCA GAGGGAGAGA AAATGCCTCC AACATGTGAG GAAGTAATGA
3601 GAGAAATCAT AGAATTTTAA GGCCATGATT TAAGGCCATC ATGGCCTTAA TCTTCCGCTT
3661 CCTCGCTCAC TGACTCGCTG CGCTCGGTCG TTCGGCTGCG GCGAGCGGTA TCAGCTCACT
3721 CAAAGGCGGT AATACGGTTA TCCACAGAAT CAGGGGATAA CGCAGGAAAG AACATGTGAG
3781 CAAAAGGCCA GCAAAAGGCC AGGAACCGTA AAAAGGCCGC GTTGCTGGCG TTTTTCCATA
3841 GGCTCCGCCC CCCTGACGAG CATCACAAAA ATCGACGCTC AAGTCAGAGG TGGCGAAACC
3901 CGACAGGACT ATAAAGATAC CAGGCGTTTC CCCCTGGAAG CTCCCTCGTG CGCTCTCCTG
3961 TTCCGACCCT GCCGCTTACC GGATACCTGT CCGCCTTTCT CCCTTCGGGA AGCGTGGCGC
4021 TTTCTCATAG CTCACGCTGT AGGTATCTCA GTTCGGTGTA GGTCGTTCGC TCCAAGCTGG
4081 GCTGTGTGCA CGAACCCCCC GTTCAGCCCG ACCGCTGCGC CTTATCCGGT AACTATCGTC
4141 TTGAGTCCAA CCCGGTAAGA CACGACTTAT CGCCACTGGC AGCAGCCACT GGTAACAGGA
4201 TTAGCAGAGC GAGGTATGTA GGCGGTGCTA CAGAGTTCTT GAAGTGGTGG CCTAACTACG
4261 GCTACACTAG AAGAACAGTA TTTGGTATCT GCGCTCTGCT GAAGCCAGTT ACCTTCGGAA
4321 AAAGAGTTGG TAGCTCTTGA TCCGGCAAAC AAACCACCGC TGGTAGCGGT GGTTTTTTTG
4381 TTTGCAAGCA GCAGATTACG CGCAGAAAAA AAGGATCTCA AGAAGATCCT TTGATCTTTT
4441 CTACGGGGTC TGACGCTCAG TGGAACGAAA ACTCACGTTA AGGGATTTTG GTCATGAGAT
4501 TATCAAAAAG GATCTTCACC TAGATCCTTT TAAATTAAAA ATGAAGTTTT AAATCAATCT
4561 AAAGTATATA TGAGTAAACT TGGTCTGACA GTTACCAATG CTTAATCAGT GAGGCACCTA
4621 TCTCAGCGAT CTGTCTATTT CGTTCATCCA TAGTTGCCTG ACTCGGGGGG GGGGGGCGCT
4681 GAGGTCTGCC TCGTGAAGAA GGTGTTGCTG ACTCATACCA GGCCTGAATC GCCCCATCAT
4741 CCAGCCAGAA AGTGAGGGAG CCACGGTTGA TGAGAGCTTT GTTGTAGGTG GACCAGTTGG
4801 TGATTTTGAA CTTTTGCTTT GCCACGGAAC GGTCTGCGTT GTCGGGAAGA TGCGTGATCT
4861 GATCCTTCAA CTCAGCAAAA GTTCGATTTA TTCAACAAAG CCGCCGTCCC GTCAAGTCAG
4921 CGTAATGCTC TGCCAGTGTT ACAACCAATT AACCAATTCT GATTAGAAAA ACTCATCGAG
4981 CATCAAATGA AACTGCAATT TATTCATATC AGGATTATCA ATACCATATT TTTGAAAAAG
5041 CCGTTTCTGT AATGAAGGAG AAAACTCACC GAGGCAGTTC CATAGGATGG CAAGATCCTG
5101 GTATCGGTCT GCGATTCCGA CTCGTCCAAC ATCAATACAA CCTATTAATT TCCCCTCGTC
5161 AAAAATAAGG TTATCAAGTG AGAAATCACC ATGAGTGACG ACTGAATCCG GTGAGAATGG
5221 CAAAAGCTTA TGCATTTCTT TCCAGACTTG TTCAACAGGC CAGCCATTAC GCTCGTCATC
5281 AAAATCACTC GCATCAACCA AACCGTTATT CATTCGTGAT TGCGCCTGAG CGAGACGAAA
5341 TACGCGATCG CTGTTAAAAG GACAATTACA AACAGGAATC GAATGCAACC GGCGCAGGAA
5401 CACTGCCAGC GCATCAACAA TATTTTCACC TGAATCAGGA TATTCTTCTA ATACCTGGAA
5461 TGCTGTTTTC CCGGGGATCG CAGTGGTGAG TAACCATGCA TCATCAGGAG TACGGATAAA
5521 ATGCTTGATG GTCGGAAGAG GCATAAATTC CGTCAGCCAG TTTAGTCTGA CCATCTCATC
5581 TGTAACATCA TTGGCAACGC TACCTTTGCC ATGTTTCAGA AACAACTCTG GCGCATCGGG
5641 CTTCCCATAC AATCGATAGA TTGTCGCACC TGATTGCCCG ACATTATCGC GAGCCCATTT
5701 ATACCCATAT AAATCAGCAT CCATGTTGGA ATTTAATCGC GGCCTCGAGC AAGACGTTTC
5761 CCGTTGAATA TGGCTCATAA CACCCCTTGT ATTACTGTTT ATGTAAGCAG ACAGTTTTAT
5821 TGTTCATGAT GATATATTTT TATCTTGTGC AATGTAACAT CAGAGATTTT GAGACACAAC
5881 GTGGCTTTCC CCCCCCCCCC ATTATTGAAG CATTTATCAG GGTTATTGTC TCATGAGCGG
5941 ATACATATTT GAATGTATTT AGAAAAATAA ACAAATAGGG GTTCCGCGCA CATTTCCCCG
6001 AAAAGTGCCA CCTGACGTCT AAGAAACCAT TATTATCATG ACATTAACCT ATAAAAATAG
6061 GCGTATCACG AGGCCCTTTC GTC
//
```

### EXAMPLE 27—FLU_T4_HA_1

This example provides amino acid and nucleic acid sequences of the influenza haemagluttinin H5 head and stem regions for an embodiment of the invention known as FLU_T4_HA_1. In SEQ ID NO:71 below, the amino acid residues of the stem region are shown underlined. The amino acid residues of the head region are the remaining residues. Similarly, in SEQ ID NO:72 below, the nucleic acid residues of the stem region are shown underlined. The nucleic acid residues of the head region are the remaining residues.

FLU_T4_HA_1-HA0 amino acid sequence
(SEQ ID NO: 71)
MEKIVLLLAIVSLVKSDQICIGYHANNSTEQVDTIMEKNVTVTHAQDILEKTHNGKLCDLNGVKPLILKDCS
VAGWLLGNPMCDEFIRVPEWSYIVERANPANDLCYPGNLNDYEELKHLLSRINHFEKILIIPKSSWPNHETS
LGVSAACPYQGTPSFFRNVVWLIKKNDAYPTIKISYNNTNREDLLILWGIHHSNNAAEQTNLYKNPTTYISV
GTSTLNQRLVPKIATRSQVNGQRGRMDFFWTILKPNDAIHFESNGNFIAPEYAYKIVKKGDSTIMKSEVEYG
HCNTKCQTPIGAINSSMPFHNIHPLTIGECPKYVKSNKLVLATGLRNSPLREKRRRKKRGLFGAIAGFIEGG
WQGMVDGWYGYHHSNEQGSGYAADKESTQKAIDGVTNKVNSIIDKMNTQFEAVGREENNLERRIENLNKKME
DGFLDVWTYNAELLVLMENERTLDFHDSNVKNLYDKVRLQLRDNAKELGNGCFEFYHKCDNECMESVRNGTY
DYPQYSEEARLKREEISGVKLESIGTYQILSIYSTVASSLALAIMVAGLSLWMCSNGSLOCRICI
FLU_T4_HA_1-HA0 nucleic acid sequence
(SEQ ID NO: 72)
GTACCGCCACCATGGAAAAGATCGTGCTGCTGCTGGCCATCGTGTCCCTGGTCAAGAGCGACCAAATCTGCATC
GGCTACCACGCCAACAACAGCACCGAACAGGTGGACACCATTATGGAAAAGAACGTCACCGTGACACACGCCCA
GGACATCCTGGAAAAGACCCACAACGGCAAGCTGTGCGACCTGAACGGCGTGAAGCCTCTGATCCTGAAGGATT
GCTCTGTGGCCGGATGGCTGCTGGGCAATCCCATGTGCGACGAGTTCATCAGAGTGCCCGAGTGGTCCTACATC
GTGGAAAGAGCCAATCCTGCCAACGACCTGTGCTACCCCGGCAACCTGAACGACTACGAGGAACTGAAGCACCT
CCTGAGCCGGATCAACCACTTCGAGAAGATCCTGATCATCCCCAAGAGCAGCTGGCCCAACCACGAGACATCTC
TGGGAGTGTCTGCCGCATGTCCATACCAGGGCACCCCTAGCTTTTTCCGGAACGTCGTGTGGCTGATCAAGAAG
AACGACGCTTACCCCACCATCAAGATCAGCTACAACAACACCAACCGCGAGGACCTGCTGATCCTGTGGGGAAT
CCACCACAGCAACAATGCCGCCGAGCAGACCAACCTGTACAAGAACCCCACCACCTACATCAGCGTGGGCACCA
GCACACTGAACCAGAGACTGGTGCCTAAGATCGCCACACGGTCCCAAGTGAATGGCCAGAGGGGCAGAATGGAC
TTCTTCTGGACCATCCTGAAGCCTAACGACGCCATCCACTTTGAGAGCAACGGCAACTTTATCGCCCCTGAGTA
CGCCTACAAGATCGTGAAGAAGGGCGACAGCACCATCATGAAGTCCGAGGTGGAATACGGCCACTGCAACACCA
AGTGTCAGACCCCTATCGGCGCCATCAACTCCAGCATGCCCTTCCACAACATTCACCCTCTGACCATCGGCGAG
TGCCCCAAATACGTGAAGTCCAACAAGCTGGTGCTGGCTACCGGCCTGAGAAACAGCCCTCTGAGAGAGAAGCG
CAGACGGAAGAAGAGAGGCCTGTTTGGCGCCATTGCCGGCTTTATCGAAGGCGGCTGGCAAGGCATGGTGGACG
GATGGTACGGCTACCATCACAGCAACGAGCAAGGCTCTGGATACGCCGCCGACAAAGAGAGCACCCAGAAAGCC
ATTGACGGCGTGACCAACAAAGTGAACAGCATCATCGACAAGATGAACACCCAGTTCGAGGCCGTGGGCAGAGA
GTTCAACAACCTGGAACGGCGGATCGAGAATCTGAACAAGAAGATGGAGGACGGCTTCCTGGACGTGTGGACCT
ACAATGCCGAGCTGCTGGTCCTGATGGAAAACGAGAGAACCCTGGACTTCCACGACTCCAACGTGAAGAACCTG
TACGACAAAGTGCGGCTCCAGCTGCGGGACAACGCCAAAGAACTCGGCAACGGCTGCTTCGAGTTCTACCACAA
GTGCGACAACGAGTGCATGGAAAGCGTGCGGAACGGCACCTACGACTACCCTCAGTACAGCGAGGAAGCCCGGC
TGAAGAGAGAAGAGATCAGCGGAGTGAAGCTGGAATCCATCGGCACATACCAGATCCTGTCCATCTACAGCACC
GTGGCCTCTTCTCTGGCCCTGGCCATTATGGTGGCTGGCCTGTCTCTGTGGATGTGCAGCAATGGCAGCCTCCA
GTGCCGGATCTGCATCTGAGCGGCC
FLU_T4_HA_1-Head region amino acid sequence
(SEQ ID NO: 73)
THNGKLCDLNGVKPLILKDCSVAGWLLGNPMCDEFIRVPEWSYIVERANPANDLCYPGNLNDYEELKHLLSRIN
HFEKILIIPKSSWPNHETSLGVSAACPYQGTPSFFRNVVWLIKKNDAYPTIKISYNNTNREDLLILWGIHHSNN
AAEQTNLYKNPTTYISVGTSTLNQRLVPKIATRSQVNGQRGRMDFFWTILKPNDAIHFESNGNFIAPEYAYKIV
KKGDSTIMKSEVEYGHCNTKCQTPIGAINSSMPFHNIHPLTIGECP
FLU_T4_HA_1-Head region nucleic acid sequence
(SEQ ID NO: 74)
TCCTGGAAAAGACCCACAACGGCAAGCTGTGCGACCTGAACGGCGTGAAGCCTCTGATCCTGAAGGATTGCTCT
GTGGCCGGATGGCTGCTGGGCAATCCCATGTGCGACGAGTTCATCAGAGTGCCCGAGTGGTCCTACATCGTGGA
AAGAGCCAATCCTGCCAACGACCTGTGCTACCCCGGCAACCTGAACGACTACGAGGAACTGAAGCACCTCCTGA
GCCGGATCAACCACTTCGAGAAGATCCTGATCATCCCCAAGAGCAGCTGGCCCAACCACGAGACATCTCTGGGA
GTGTCTGCCGCATGTCCATACCAGGGCACCCCTAGCTTTTTCCGGAACGTCGTGTGGCTGATCAAGAAGAACGA
CGCTTACCCCACCATCAAGATCAGCTACAACAACACCAACCGCGAGGACCTGCTGATCCTGTGGGGAATCCACC
ACAGCAACAATGCCGCCGAGCAGACCAACCTGTACAAGAACCCCACCACCTACATCAGCGTGGGCACCAGCACA
CTGAACCAGAGACTGGTGCCTAAGATCGCCACACGGTCCCAAGTGAATGGCCAGAGGGGCAGAATGGACTTCTT
CTGGACCATCCTGAAGCCTAACGACGCCATCCACTTTGAGAGCAACGGCAACTTTATCGCCCCTGAGTACGCCT
ACAAGATCGTGAAGAAGGGCGACAGCACCATCATGAAGTCCGAGGTGGAATACGGCCACTGCAACACCAAGTGT
CAGACCCCTATCGGCGCCATCAACTCCAGCATGCCCTTCCACAACATTCACCCTCTGACCATCGGCGAGTGCCC
CAAATACGTG
FLU_T4_HA_1-First stem region amino acid sequence
(SEQ ID NO: 75)
MEKIVLLLAIVSLVKSDQICIGYHANNSTEQVDTIMEKNVTVTHAQDILEK
FLU_T4_HA_1-First stem region nucleic acid sequence
(SEQ ID NO: 76)
GTACCGCCACCATGGAAAAGATCGTGCTGCTGCTGGCCATCGTGTCCCTGGTCAAGAGCGACCAAATCTGCATC
GGCTACCACGCCAACAACAGCACCGAACAGGTGGACACCATTATGGAAAAGAACGTCACCGTGACACACGCCCA
GGACA
FLU_T4_HA_1-Second stem region amino acid sequence
(SEQ ID NO: 77)
KYVKSNKLVLATGLRNSPLREKRRRKKRGLFGAIAGFIEGGWQGMVDGWYGYHHSNEQGSGYAADKESTQKAID
GVTNKVNSIIDKMNTQFEAVGREENNLERRIENLNKKMEDGELDVWTYNAELLVLMENERTLDFHDSNVKNLYD
KVRLQLRDNAKELGNGCFEFYHKCDNECMESVRNGTYDYPQYSEEARLKREEISGVKLESIGTYQILSIYSTVA
SSLALAIMVAGLSLWMCSNGSLQCRICI
FLU_T4_HA_1-Second stem region nucleic acid sequence
(SEQ ID NO: 78)
AAGTCCAACAAGCTGGTGCTGGCTACCGGCCTGAGAAACAGCCCTCTGAGAGAGAAGCGCAGACGGAAGAAGAG
AGGCCTGTTTGGCGCCATTGCCGGCTTTATCGAAGGCGGCTGGCAAGGCATGGTGGACGGATGGTACGGCTACC
ATCACAGCAACGAGCAAGGCTCTGGATACGCCGCCGACAAAGAGAGCACCCAGAAAGCCATTGACGGCGTGACC
AACAAAGTGAACAGCATCATCGACAAGATGAACACCCAGTTCGAGGCCGTGGGCAGAGAGTTCAACAACCTGGA
ACGGCGGATCGAGAATCTGAACAAGAAGATGGAGGACGGCTTCCTGGACGTGTGGACCTACAATGCCGAGCTGC
TGGTCCTGATGGAAAACGAGAGAACCCTGGACTTCCACGACTCCAACGTGAAGAACCTGTACGACAAAGTGCGG
CTCCAGCTGCGGGACAACGCCAAAGAACTCGGCAACGGCTGCTTCGAGTTCTACCACAAGTGCGACAACGAGTG
CATGGAAAGCGTGCGGAACGGCACCTACGACTACCCTCAGTACAGCGAGGAAGCCCGGCTGAAGAGAGAAGAGA
TCAGCGGAGTGAAGCTGGAATCCATCGGCACATACCAGATCCTGTCCATCTACAGCACCGTGGCCTCTTCTCTG
GCCCTGGCCATTATGGTGGCTGGCCTGTCTCTGTGGATGTGCAGCAATGGCAGCCTCCAGTGCCGGATCTGCAT
CTGAGCGGCC

### EXAMPLE 28—PEVAC-FLU_T4_HA_1

This example provides the nucleic acid sequence of pEVAC-FLU_T4_HA_1.

| pEVAC-FLU_T4_HA_1-nucleic acid sequence (SEQ ID NO: 79): | | | | | | |
|---|---|---|---|---|---|---|
| LOCUS pVRC8400EVAC_Ar_pEVAC-Flu_T4_HA_1 6092 bp DNA | | | | | | |
| linear SYN 08-SEP-2022 | | | | | | |
| FEATURES Location/Qualifiers | | | | | | |
| CDS 1344 . . . 3070 | | | | | | |
| /label = “Flu_T4_HA_1” | | | | | | |
| /note = “CI:V1 = KpnI, V2 = NotI, I1 = KpnI, I2 = NotI” | | | | | | |
| ORIGIN | | | | | | |
| 1 | TCGCGCGTTT | CGGTGATGAC | GGTGAAAACC | TCTGACACAT | GCAGCTCCCG | GAGACGGTCA |
| 61 | CAGCTTGTCT | GTAAGCGGAT | GCCGGGAGCA | GACAAGCCCG | TCAGGGCGCG | TCAGCGGGTG |
| 121 | TTGGCGGGTG | TCGGGGCTGG | CTTAACTATG | CGGCATCAGA | GCAGATTGTA | CTGAGAGTGC |
| 181 | ACCATATGCG | GTGTGAAATA | CCGCACAGAT | GCGTAAGGAG | AAAATACCGC | ATCAGATTGG |
| 241 | CTATTGGCCA | TTGCATACGT | TGTATCCATA | TCATAATATG | TACATTTATA | TTGGCTCATG |
| 301 | TCCAACATTA | CCGCCATGTT | GACATTGATT | ATTGACTAGT | TATTAATAGT | AATCAATTAC |
| 361 | GGGGTCATTA | GTTCATAGCC | CATATATGGA | GTTCCGCGTT | ACATAACTTA | CGGTAAATGG |
| 421 | CCCGCCTGGC | TGACCGCCCA | ACGACCCCCG | CCCATTGACG | TCAATAATGA | CGTATGTTCC |
| 481 | CATAGTAACG | CCAATAGGGA | CTTTCCATTG | ACGTCAATGG | GTGGAGTATT | TACGGTAAAC |
| 541 | TGCCCACTTG | GCAGTACATC | AAGTGTATCA | TATGCCAAGT | ACGCCCCCTA | TTGACGTCAA |
| 601 | TGACGGTAAA | TGGCCCGCCT | GGCATTATGC | CCAGTACATG | ACCTTATGGG | ACTTTCCTAC |
| 661 | TTGGCAGTAC | ATCTACGTAT | TAGTCATCGC | TATTACCATG | GTGATGCGGT | TTTGGCAGTA |
| 721 | CATCAATGGG | CGTGGATAGC | GGTTTGACTC | ACGGGGATTT | CCAAGTCTCC | ACCCCATTGA |
| 781 | CGTCAATGGG | AGTTTGTTTT | GGCACCAAAA | TCAACGGGAC | TTTCCAAAAT | GTCGTAACAA |
| 841 | CTCCGCCCCA | TTGACGCAAA | TGGGCGGTAG | GCGTGTACGG | TGGGAGGTCT | ATATAAGCAG |
| 901 | AGCTCGTTTA | GTGAACCGTC | AGATCGCCTG | GAGACGCCAT | CCACGCTGTT | TTGACCTCCA |
| 961 | TAGAAGACAC | CGGGACCGAT | CCAGCCTCCA | TCGGCTCGCA | TCTCTCCTTC | ACGCGCCCGC |
| 1021 | CGCCCTACCT | GAGGCCGCCA | TCCACGCCGG | TTGAGTCGCG | TTCTGCCGCC | TCCCGCCTGT |
| 1081 | GGTGCCTCCT | GAACTGCGTC | CGCCGTCTAG | GTAAGTTTAA | AGCTCAGGTC | GAGACCGGGC |
| 1141 | CTTTGTCCGG | CGCTCCCTTG | GAGCCTACCT | AGACTCAGCC | GGCTCTCCAC | GCTTTGCCTG |
| 1201 | ACCCTGCTTG | CTCAACTCTA | GTTAACGGTG | GAGGGCAGTG | TAGTCTGAGC | AGTACTCGTT |
| 1261 | GCTGCCGCGC | GCGCCACCAG | ACATAATAGC | TGACAGACTA | ACAGACTGTT | CCTTTCCATG |
| 1321 | GGTCTTTTCT | GCAGTCACCG | TCGGTACCGC | CACCATGGAA | AAGATCGTGC | TGCTGCTGGC |
| 1381 | CATCGTGTCC | CTGGTCAAGA | GCGACCAAAT | CTGCATCGGC | TACCACGCCA | ACAACAGCAC |
| 1441 | CGAACAGGTG | GACACCATTA | TGGAAAAGAA | CGTCACCGTG | ACACACGCCC | AGGACATCCT |
| 1501 | GGAAAAGACC | CACAACGGCA | AGCTGTGCGA | CCTGAACGGC | GTGAAGCCTC | TGATCCTGAA |
| 1561 | GGATTGCTCT | GTGGCCGGAT | GGCTGCTGGG | CAATCCCATG | TGCGACGAGT | TCATCAGAGT |
| 1621 | GCCCGAGTGG | TCCTACATCG | TGGAAAGAGC | CAATCCTGCC | AACGACCTGT | GCTACCCCGG |
| 1681 | CAACCTGAAC | GACTACGAGG | AACTGAAGCA | CCTCCTGAGC | CGGATCAACC | ACTTCGAGAA |
| 1741 | GATCCTGATC | ATCCCCAAGA | GCAGCTGGCC | CAACCACGAG | ACATCTCTGG | GAGTGTCTGC |
| 1801 | CGCATGTCCA | TACCAGGGCA | CCCCTAGCTT | TTTCCGGAAC | GTCGTGTGGC | TGATCAAGAA |
| 1861 | GAACGACGCT | TACCCCACCA | TCAAGATCAG | CTACAACAAC | ACCAACCGCG | AGGACCTGCT |
| 1921 | GATCCTGTGG | GGAATCCACC | ACAGCAACAA | TGCCGCCGAG | CAGACCAACC | TGTACAAGAA |
| 1981 | CCCCACCACC | TACATCAGCG | TGGGCACCAG | CACACTGAAC | CAGAGACTGG | TGCCTAAGAT |
| 2041 | CGCCACACGG | TCCCAAGTGA | ATGGCCAGAG | GGGCAGAATG | GACTTCTTCT | GGACCATCCT |
| 2101 | GAAGCCTAAC | GACGCCATCC | ACTTTGAGAG | CAACGGCAAC | TTTATCGCCC | CTGAGTACGC |
| 2161 | CTACAAGATC | GTGAAGAAGG | GCGACAGCAC | CATCATGAAG | TCCGAGGTGG | AATACGGCCA |
| 2221 | CTGCAACACC | AAGTGTCAGA | CCCCTATCGG | CGCCATCAAC | TCCAGCATGC | CCTTCCACAA |
| 2281 | CATTCACCCT | CTGACCATCG | GCGAGTGCCC | CAAATACGTG | AAGTCCAACA | AGCTGGTGCT |
| 2341 | GGCTACCGGC | CTGAGAAACA | GCCCTCTGAG | AGAGAAGCGC | AGACGGAAGA | AGAGAGGCCT |
| 2401 | GTTTGGCGCC | ATTGCCGGCT | TTATCGAAGG | CGGCTGGCAA | GGCATGGTGG | ACGGATGGTA |
| 2461 | CGGCTACCAT | CACAGCAACG | AGCAAGGCTC | TGGATACGCC | GCCGACAAAG | AGAGCACCCA |
| 2521 | GAAAGCCATT | GACGGCGTGA | CCAACAAAGT | GAACAGCATC | ATCGACAAGA | TGAACACCCA |
| 2581 | GTTCGAGGCC | GTGGGCAGAG | AGTTCAACAA | CCTGGAACGG | CGGATCGAGA | ATCTGAACAA |
| 2641 | GAAGATGGAG | GACGGCTTCC | TGGACGTGTG | GACCTACAAT | GCCGAGCTGC | TGGTCCTGAT |
| 2701 | GGAAAACGAG | AGAACCCTGG | ACTTCCACGA | CTCCAACGTG | AAGAACCTGT | ACGACAAAGT |
| 2761 | GCGGCTCCAG | CTGCGGGACA | ACGCCAAAGA | ACTCGGCAAC | GGCTGCTTCG | AGTTCTACCA |
| 2821 | CAAGTGCGAC | AACGAGTGCA | TGGAAAGCGT | GCGGAACGGC | ACCTACGACT | ACCCTCAGTA |
| 2881 | CAGCGAGGAA | GCCCGGCTGA | AGAGAGAAGA | GATCAGCGGA | GTGAAGCTGG | AATCCATCGG |
| 2941 | CACATACCAG | ATCCTGTCCA | TCTACAGCAC | CGTGGCCTCT | TCTCTGGCCC | TGGCCATTAT |
| 3001 | GGTGGCTGGC | CTGTCTCTGT | GGATGTGCAG | CAATGGCAGC | CTCCAGTGCC | GGATCTGCAT |
| 3061 | CTGAGCGGCC | GCAGATCTGC | TGTGCCTTCT | AGTTGCCAGC | CATCTGTTGT | TTGCCCCTCC |
| 3121 | CCCGTGCCTT | CCTTGACCCT | GGAAGGTGCC | ACTCCCACTG | TCCTTTCCTA | ATAAAATGAG |
| 3181 | GAAATTGCAT | CGCATTGTCT | GAGTAGGTGT | CATTCTATTC | TGGGGGGTGG | GGTGGGGCAG |
| 3241 | GACAGCAAGG | GGGAGGATTG | GGAAGACAAT | AGCAGGCATG | CTGGGGATGC | GGTGGGCTCT |
| 3301 | ATGGCTACCC | AGGTGCTGAA | GAATTGACCC | GGTTCCTCCT | GGGCCAGAAA | GAAGCAGGCA |
| 3361 | CATCCCCTTC | TCTGTGACAC | ACCCTGTCCA | CGCCCCTGGT | TCTTAGTTCC | AGCCCCACTC |
| 3421 | ATAGGACACT | CATAGCTCAG | GAGGGCTCCG | CCTTCAATCC | CACCCGCTAA | AGTACTTGGA |
| 3481 | GCGGTCTCTC | CCTCCCTCAT | CAGCCCACCA | AACCAAACCT | AGCCTCCAAG | AGTGGGAAGA |
| 3541 | AATTAAAGCA | AGATAGGCTA | TTAAGTGCAG | AGGGAGAGAA | AATGCCTCCA | ACATGTGAGG |
| 3601 | AAGTAATGAG | AGAAATCATA | GAATTTTAAG | GCCATGATTT | AAGGCCATCA | TGGCCTTAAT |
| 3661 | CTTCCGCTTC | CTCGCTCACT | GACTCGCTGC | GCTCGGTCGT | TCGGCTGCGG | CGAGCGGTAT |
| 3721 | CAGCTCACTC | AAAGGCGGTA | ATACGGTTAT | CCACAGAATC | AGGGGATAAC | GCAGGAAAGA |
| 3781 | ACATGTGAGC | AAAAGGCCAG | CAAAAGGCCA | GGAACCGTAA | AAAGGCCGCG | TTGCTGGCGT |
| 3841 | TTTTCCATAG | GCTCCGCCCC | CCTGACGAGC | ATCACAAAAA | TCGACGCTCA | AGTCAGAGGT |
| 3901 | GGCGAAACCC | GACAGGACTA | TAAAGATACC | AGGCGTTTCC | CCCTGGAAGC | TCCCTCGTGC |
| 3961 | GCTCTCCTGT | TCCGACCCTG | CCGCTTACCG | GATACCTGTC | CGCCTTTCTC | CCTTCGGGAA |
| 4021 | GCGTGGCGCT | TTCTCATAGC | TCACGCTGTA | GGTATCTCAG | TTCGGTGTAG | GTCGTTCGCT |
| 4081 | CCAAGCTGGG | CTGTGTGCAC | GAACCCCCCG | TTCAGCCCGA | CCGCTGCGCC | TTATCCGGTA |
| 4141 | ACTATCGTCT | TGAGTCCAAC | CCGGTAAGAC | ACGACTTATC | GCCACTGGCA | GCAGCCACTG |
| 4201 | GTAACAGGAT | TAGCAGAGCG | AGGTATGTAG | GCGGTGCTAC | AGAGTTCTTG | AAGTGGTGGC |
| 4261 | CTAACTACGG | CTACACTAGA | AGAACAGTAT | TTGGTATCTG | CGCTCTGCTG | AAGCCAGTTA |
| 4321 | CCTTCGGAAA | AAGAGTTGGT | AGCTCTTGAT | CCGGCAAACA | AACCACCGCT | GGTAGCGGTG |
| 4381 | GTTTTTTTGT | TTGCAAGCAG | CAGATTACGC | GCAGAAAAAA | AGGATCTCAA | GAAGATCCTT |
| 4441 | TGATCTTTTC | TACGGGGTCT | GACGCTCAGT | GGAACGAAAA | CTCACGTTAA | GGGATTTTGG |
| 4501 | TCATGAGATT | ATCAAAAAGG | ATCTTCACCT | AGATCCTTTT | AAATTAAAAA | TGAAGTTTTA |
| 4561 | AATCAATCTA | AAGTATATAT | GAGTAAACTT | GGTCTGACAG | TTACCAATGC | TTAATCAGTG |
| 4621 | AGGCACCTAT | CTCAGCGATC | TGTCTATTTC | GTTCATCCAT | AGTTGCCTGA | CTCGGGGGGG |
| 4681 | GGGGGCGCTG | AGGTCTGCCT | CGTGAAGAAG | GTGTTGCTGA | CTCATACCAG | GCCTGAATCG |
| 4741 | CCCCATCATC | CAGCCAGAAA | GTGAGGGAGC | CACGGTTGAT | GAGAGCTTTG | TTGTAGGTGG |
| 4801 | ACCAGTTGGT | GATTTTGAAC | TTTTGCTTTG | CCACGGAACG | GTCTGCGTTG | TCGGGAAGAT |
| 4861 | GCGTGATCTG | ATCCTTCAAC | TCAGCAAAAG | TTCGATTTAT | TCAACAAAGC | CGCCGTCCCG |
| 4921 | TCAAGTCAGC | GTAATGCTCT | GCCAGTGTTA | CAACCAATTA | ACCAATTCTG | ATTAGAAAAA |
| 4981 | CTCATCGAGC | ATCAAATGAA | ACTGCAATTT | ATTCATATCA | GGATTATCAA | TACCATATTT |
| 5041 | TTGAAAAAGC | CGTTTCTGTA | ATGAAGGAGA | AAACTCACCG | AGGCAGTTCC | ATAGGATGGC |
| 5101 | AAGATCCTGG | TATCGGTCTG | CGATTCCGAC | TCGTCCAACA | TCAATACAAC | CTATTAATTT |
| 5161 | CCCCTCGTCA | AAAATAAGGT | TATCAAGTGA | GAAATCACCA | TGAGTGACGA | CTGAATCCGG |
| 5221 | TGAGAATGGC | AAAAGCTTAT | GCATTTCTTT | CCAGACTTGT | TCAACAGGCC | AGCCATTACG |
| 5281 | CTCGTCATCA | AAATCACTCG | CATCAACCAA | ACCGTTATTC | ATTCGTGATT | GCGCCTGAGC |
| 5341 | GAGACGAAAT | ACGCGATCGC | TGTTAAAAGG | ACAATTACAA | ACAGGAATCG | AATGCAACCG |
| 5401 | GCGCAGGAAC | ACTGCCAGCG | CATCAACAAT | ATTTTCACCT | GAATCAGGAT | ATTCTTCTAA |
| 5461 | TACCTGGAAT | GCTGTTTTCC | CGGGGATCGC | AGTGGTGAGT | AACCATGCAT | CATCAGGAGT |
| 5521 | ACGGATAAAA | TGCTTGATGG | TCGGAAGAGG | CATAAATTCC | GTCAGCCAGT | TTAGTCTGAC |
| 5581 | CATCTCATCT | GTAACATCAT | TGGCAACGCT | ACCTTTGCCA | TGTTTCAGAA | ACAACTCTGG |
| 5641 | CGCATCGGGC | TTCCCATACA | ATCGATAGAT | TGTCGCACCT | GATTGCCCGA | CATTATCGCG |
| 5701 | AGCCCATTTA | TACCCATATA | AATCAGCATC | CATGTTGGAA | TTTAATCGCG | GCCTCGAGCA |
| 5761 | AGACGTTTCC | CGTTGAATAT | GGCTCATAAC | ACCCCTTGTA | TTACTGTTTA | TGTAAGCAGA |
| 5821 | CAGTTTTATT | GTTCATGATG | ATATATTTTT | ATCTTGTGCA | ATGTAACATC | AGAGATTTTG |
| 5881 | AGACACAACG | TGGCTTTCCC | CCCCCCCCCA | TTATTGAAGC | ATTTATCAGG | GTTATTGTCT |
| 5941 | CATGAGCGGA | TACATATTTG | AATGTATTTA | GAAAAATAAA | CAAATAGGGG | TTCCGCGCAC |
| 6001 | ATTTCCCCGA | AAAGTGCCAC | CTGACGTCTA | AGAAACCATT | ATTATCATGA | CATTAACCTA |
| 6061 | TAAAAATAGG | CGTATCACGA | GGCCCTTTCG | TC | | |

### EXAMPLE 29—FLU_T4_HA_2

This example provides amino acid and nucleic acid sequences of the influenza haemagluttinin H5 head and stem regions for an embodiment of the invention known as FLU_T4_HA_2. In SEQ ID NO:80 below, the amino acid residues of the stem region are shown underlined. The amino acid residues of the head region are the remaining residues. Similarly, in SEQ ID NO:81 below, the nucleic acid residues of the stem region are shown underlined. The nucleic acid residues of the head region are the remaining residues.

FLU_T4_HA_2-HA0 amino acid sequence
(SEQ ID NO: 80)
MEKIVLLLAIVSLVKSDQICIGYHANNSTEQVDTIMEKNVTVTHAQDILEKTHNGKLCDLNGVKPLILKDCS
VAGWLLGNPMCDEFIRVPEWSYIVERANPANDLCFPGNLNDYEELKHLLSRINHFEKILIIPKSSWPNHETS
LGVSAACPYQGTPSFFRNVVWLIKKNDAYPTIKISYNNTNREDLLILWGIHHSNNAAEQTNLYKNPTTYISV
GTSTLNQRLVPKIATRSQVNGERGRMDFFWTILKPNDAIHFESNGNFIAPEYAYKIVKKGDSTIMKSEVEYG
HCNTKCQTPIGAINSSMPFHNIHPLTIGECPKYVKSNKLVLATGLRNSPLREKRRRKKRGLFGAIAGFIEGG
WQGMVDGWYGYHHSNEQGSGYAADKESTQKAIDGVINKVNSIIDKMNTQFEAVGREENNLERRIENLNKKME
DGFLDVWTYNAELLVLMENERTLDFHDSNVKNLYDKVRLOLRDNAKELGNGCFEFYHKCDNECMESVRNGTY
DYPQYSEEARLKREEISGVKLESIGTYQILSIYSTVASSLALAIMVAGLSLWMCSNGSLOCRICI
FLU_T4_HA_2-HA0 nucleic acid sequence
(SEQ ID NO: 81)
GTACCGCCACCATGGAAAAGATCGTGCTGCTGCTGGCCATCGTGTCCCTGGTCAAGAGCGACCAAATCTGCATC
GGCTACCACGCCAACAACAGCACCGAACAGGTGGACACCATTATGGAAAAGAACGTCACCGTGACACACGCCCA
GGACATCCTGGAAAAGACCCACAACGGCAAGCTGTGCGACCTGAACGGCGTGAAGCCTCTGATCCTGAAGGATT
GCTCTGTGGCCGGATGGCTGCTGGGCAATCCCATGTGCGACGAGTTCATCAGAGTGCCCGAGTGGTCCTACATC
GTGGAAAGAGCCAATCCTGCCAACGACCTGTGCTTCCCCGGCAACCTGAACGACTACGAGGAACTGAAGCACCT
CCTGAGCCGGATCAACCACTTCGAGAAGATCCTGATCATCCCCAAGAGCAGCTGGCCCAACCACGAGACATCTC
TGGGAGTGTCTGCCGCATGTCCATACCAGGGCACCCCTAGCTTTTTCCGGAACGTCGTGTGGCTGATCAAGAAG
AACGACGCTTACCCCACCATCAAGATCAGCTACAACAACACCAACCGCGAGGACCTGCTGATCCTGTGGGGAAT
CCACCACAGCAACAATGCCGCCGAGCAGACCAACCTGTACAAGAACCCCACCACCTACATCAGCGTGGGCACCA
GCACACTGAACCAGAGACTGGTGCCTAAGATCGCCACACGGTCCCAAGTGAATGGCGAGAGGGGCAGAATGGAC
TTCTTCTGGACCATCCTGAAGCCTAACGACGCCATCCACTTTGAGAGCAACGGCAACTTTATCGCCCCTGAGTA
CGCCTACAAGATCGTGAAGAAGGGCGACAGCACCATCATGAAGTCCGAGGTGGAATACGGCCACTGCAACACCA
AGTGTCAGACCCCTATCGGCGCCATCAACTCCAGCATGCCCTTCCACAACATTCACCCTCTGACCATCGGCGAG
TGCCCCAAATACGTGAAGTCCAACAAGCTGGTGCTGGCTACCGGCCTGAGAAACAGCCCTCTGAGAGAGAAGCG
CAGACGGAAGAAGAGAGGCCTGTTTGGCGCCATTGCCGGCTTTATCGAAGGCGGCTGGCAAGGCATGGTGGACG
GATGGTACGGCTACCATCACAGCAACGAGCAAGGCTCTGGATACGCCGCCGACAAAGAGAGCACCCAGAAAGCC
ATTGACGGCGTGACCAACAAAGTGAACAGCATCATCGACAAGATGAACACCCAGTTCGAGGCCGTGGGCAGAGA
GTTCAACAACCTGGAACGGCGGATCGAGAATCTGAACAAGAAGATGGAGGACGGCTTCCTGGACGTGTGGACCT
ACAATGCCGAGCTGCTGGTCCTGATGGAAAACGAGAGAACCCTGGACTTCCACGACTCCAACGTGAAGAACCTG
TACGACAAAGTGCGGCTCCAGCTGCGGGACAACGCCAAAGAACTCGGCAACGGCTGCTTCGAGTTCTACCACAA
GTGCGACAACGAGTGCATGGAAAGCGTGCGGAACGGCACCTACGACTACCCTCAGTACAGCGAGGAAGCCCGGC
TGAAGAGAGAAGAGATCAGCGGAGTGAAGCTGGAATCCATCGGCACATACCAGATCCTGTCCATCTACAGCACC
GTGGCCTCTTCTCTGGCCCTGGCCATTATGGTGGCTGGCCTGTCTCTGTGGATGTGCAGCAATGGCAGCCTCCA
GTGCCGGATCTGCATCTGAGCGGCC
FLU_T4_HA_2-Head region amino acid sequence
(SEQ ID NO: 82)
THNGKLCDLNGVKPLILKDCSVAGWLLGNPMCDEFIRVPEWSYIVERANPANDLCFPGNLNDYEELKHLLSRIN
HFEKILIIPKSSWPNHETSLGVSAACPYQGTPSFFRNVVWLIKKNDAYPTIKISYNNTNREDLLILWGIHHSNN
AAEQTNLYKNPTTYISVGTSTLNQRLVPKIATRSQVNGERGRMDFFWTILKPNDAIHFESNGNFIAPEYAYKIV
KKGDSTIMKSEVEYGHCNTKCQTPIGAINSSMPFHNIHPLTIGECP
FLU_T4_HA_2-Head region nucleic acid sequence
(SEQ ID NO: 83)
TCCTGGAAAAGACCCACAACGGCAAGCTGTGCGACCTGAACGGCGTGAAGCCTCTGATCCTGAAGGATTGCTCT
GTGGCCGGATGGCTGCTGGGCAATCCCATGTGCGACGAGTTCATCAGAGTGCCCGAGTGGTCCTACATCGTGGA
AAGAGCCAATCCTGCCAACGACCTGTGCTTCCCCGGCAACCTGAACGACTACGAGGAACTGAAGCACCTCCTGA
GCCGGATCAACCACTTCGAGAAGATCCTGATCATCCCCAAGAGCAGCTGGCCCAACCACGAGACATCTCTGGGA
GTGTCTGCCGCATGTCCATACCAGGGCACCCCTAGCTTTTTCCGGAACGTCGTGTGGCTGATCAAGAAGAACGA
CGCTTACCCCACCATCAAGATCAGCTACAACAACACCAACCGCGAGGACCTGCTGATCCTGTGGGGAATCCACC
ACAGCAACAATGCCGCCGAGCAGACCAACCTGTACAAGAACCCCACCACCTACATCAGCGTGGGCACCAGCACA
CTGAACCAGAGACTGGTGCCTAAGATCGCCACACGGTCCCAAGTGAATGGCGAGAGGGGCAGAATGGACTTCTT
CTGGACCATCCTGAAGCCTAACGACGCCATCCACTTTGAGAGCAACGGCAACTTTATCGCCCCTGAGTACGCCT
ACAAGATCGTGAAGAAGGGCGACAGCACCATCATGAAGTCCGAGGTGGAATACGGCCACTGCAACACCAAGTGT
CAGACCCCTATCGGCGCCATCAACTCCAGCATGCCCTTCCACAACATTCACCCTCTGACCATCGGCGAGTGCCC
CAAATACGTG
FLU_T4_HA_2-First stem region amino acid sequence
(SEQ ID NO: 84)
MEKIVLLLAIVSLVKSDQICIGYHANNSTEQVDTIMEKNVTVTHAQDILEK
FLU_T4_HA_2-First stem region nucleic acid sequence
(SEQ ID NO: 85)
GTACCGCCACCATGGAAAAGATCGTGCTGCTGCTGGCCATCGTGTCCCTGGTCAAGAGCGACCAAATCTGCATC
GGCTACCACGCCAACAACAGCACCGAACAGGTGGACACCATTATGGAAAAGAACGTCACCGTGACACACGCCCA
GGACA
FLU_T4_HA_2-Second stem region amino acid sequence
(SEQ ID NO: 86)
KYVKSNKLVLATGLRNSPLREKRRRKKRGLFGAIAGFIEGGWQGMVDGWYGYHHSNEQGSGYAADKESTQKAID
GVTNKVNSIIDKMNTQFEAVGREFNNLERRIENLNKKMEDGELDVWTYNAELLVLMENERTLDFHDSNVKNLYD
KVRLQLRDNAKELGNGCFEFYHKCDNECMESVRNGTYDYPQYSEEARLKREEISGVKLESIGTYQILSIYSTVA
SSLALAIMVAGLSLWMCSNGSLQCRICI
FLU_T4_HA_2-Second stem region nucleic acid sequence
(SEQ ID NO: 87)
AAGTCCAACAAGCTGGTGCTGGCTACCGGCCTGAGAAACAGCCCTCTGAGAGAGAAGCGCAGACGGAAGAAGAG
AGGCCTGTTTGGCGCCATTGCCGGCTTTATCGAAGGCGGCTGGCAAGGCATGGTGGACGGATGGTACGGCTACC
ATCACAGCAACGAGCAAGGCTCTGGATACGCCGCCGACAAAGAGAGCACCCAGAAAGCCATTGACGGCGTGACC
AACAAAGTGAACAGCATCATCGACAAGATGAACACCCAGTTCGAGGCCGTGGGCAGAGAGTTCAACAACCTGGA
ACGGCGGATCGAGAATCTGAACAAGAAGATGGAGGACGGCTTCCTGGACGTGTGGACCTACAATGCCGAGCTGC
TGGTCCTGATGGAAAACGAGAGAACCCTGGACTTCCACGACTCCAACGTGAAGAACCTGTACGACAAAGTGCGG
CTCCAGCTGCGGGACAACGCCAAAGAACTCGGCAACGGCTGCTTCGAGTTCTACCACAAGTGCGACAACGAGTG
CATGGAAAGCGTGCGGAACGGCACCTACGACTACCCTCAGTACAGCGAGGAAGCCCGGCTGAAGAGAGAAGAGA
TCAGCGGAGTGAAGCTGGAATCCATCGGCACATACCAGATCCTGTCCATCTACAGCACCGTGGCCTCTTCTCTG
GCCCTGGCCATTATGGTGGCTGGCCTGTCTCTGTGGATGTGCAGCAATGGCAGCCTCCAGTGCCGGATCTGCAT
CTGAGCGGCC

### EXAMPLE 30—PEVAC-FLU_T4_HA_2

This example provides the nucleic acid sequence of pEVAC-FLU_T4_HA_2.

```
pEVAC-FLU_T4_HA_2-nucleic acid sequence (SEQ ID NO: 88):
LOCUS pVRC8400EVAC_Ar_pEVAC-Flu_T4_HA_2 6092 bp DNA
linear SYN 08-SEP-2022
FEATURES Location/Qualifiers
          CDS 1344 . . . 3070
                                      /label = “Flu_T4_HA_2”
                                      /note = “CI: V1 = KpnI, V2-NotI, I1 = KpnI, I2 = NotI”
ORIGIN
      1 TCGCGCGTTT CGGTGATGAC GGTGAAAACC TCTGACACAT GCAGCTCCCG GAGACGGTCA
    61 CAGCTTGTCT GTAAGCGGAT GCCGGGAGCA GACAAGCCCG TCAGGGCGCG TCAGCGGGTG
  121 TTGGCGGGTG TCGGGGCTGG CTTAACTATG CGGCATCAGA GCAGATTGTA CTGAGAGTGC
  181 ACCATATGCG GTGTGAAATA CCGCACAGAT GCGTAAGGAG AAAATACCGC ATCAGATTGG
  241 CTATTGGCCA TTGCATACGT TGTATCCATA TCATAATATG TACATTTATA TTGGCTCATG
  301 TCCAACATTA CCGCCATGTT GACATTGATT ATTGACTAGT TATTAATAGT AATCAATTAC
  361 GGGGTCATTA GTTCATAGCC CATATATGGA GTTCCGCGTT ACATAACTTA CGGTAAATGG
  421 CCCGCCTGGC TGACCGCCCA ACGACCCCCG CCCATTGACG TCAATAATGA CGTATGTTCC
  481 CATAGTAACG CCAATAGGGA CTTTCCATTG ACGTCAATGG GTGGAGTATT TACGGTAAAC
  541 TGCCCACTTG GCAGTACATC AAGTGTATCA TATGCCAAGT ACGCCCCCTA TTGACGTCAA
  601 TGACGGTAAA TGGCCCGCCT GGCATTATGC CCAGTACATG ACCTTATGGG ACTTTCCTAC
  661 TTGGCAGTAC ATCTACGTAT TAGTCATCGC TATTACCATG GTGATGCGGT TTTGGCAGTA
  721 CATCAATGGG CGTGGATAGC GGTTTGACTC ACGGGGATTT CCAAGTCTCC ACCCCATTGA
  781 CGTCAATGGG AGTTTGTTTT GGCACCAAAA TCAACGGGAC TTTCCAAAAT GTCGTAACAA
  841 CTCCGCCCCA TTGACGCAAA TGGGCGGTAG GCGTGTACGG TGGGAGGTCT ATATAAGCAG
  901 AGCTCGTTTA GTGAACCGTC AGATCGCCTG GAGACGCCAT CCACGCTGTT TTGACCTCCA
  961 TAGAAGACAC CGGGACCGAT CCAGCCTCCA TCGGCTCGCA TCTCTCCTTC ACGCGCCCGC
1021 CGCCCTACCT GAGGCCGCCA TCCACGCCGG TTGAGTCGCG TTCTGCCGCC TCCCGCCTGT
1081 GGTGCCTCCT GAACTGCGTC CGCCGTCTAG GTAAGTTTAA AGCTCAGGTC GAGACCGGGC
1141 CTTTGTCCGG CGCTCCCTTG GAGCCTACCT AGACTCAGCC GGCTCTCCAC GCTTTGCCTG
1201 ACCCTGCTTG CTCAACTCTA GTTAACGGTG GAGGGCAGTG TAGTCTGAGC AGTACTCGTT
1261 GCTGCCGCGC GCGCCACCAG ACATAATAGC TGACAGACTA ACAGACTGTT CCTTTCCATG
1321 GGTCTTTTCT GCAGTCACCG TCGGTACCGC CACCATGGAA AAGATCGTGC TGCTGCTGGC
1381 CATCGTGTCC CTGGTCAAGA GCGACCAAAT CTGCATCGGC TACCACGCCA ACAACAGCAC
1441 CGAACAGGTG GACACCATTA TGGAAAAGAA CGTCACCGTG ACACACGCCC AGGACATCCT
1501 GGAAAAGACC CACAACGGCA AGCTGTGCGA CCTGAACGGC GTGAAGCCTC TGATCCTGAA
1561 GGATTGCTCT GTGGCCGGAT GGCTGCTGGG CAATCCCATG TGCGACGAGT TCATCAGAGT
1621 GCCCGAGTGG TCCTACATCG TGGAAAGAGC CAATCCTGCC AACGACCTGT GCTTCCCCGG
1681 CAACCTGAAC GACTACGAGG AACTGAAGCA CCTCCTGAGC CGGATCAACC ACTTCGAGAA
1741 GATCCTGATC ATCCCCAAGA GCAGCTGGCC CAACCACGAG ACATCTCTGG GAGTGTCTGC
1801 CGCATGTCCA TACCAGGGCA CCCCTAGCTT TTTCCGGAAC GTCGTGTGGC TGATCAAGAA
1861 GAACGACGCT TACCCCACCA TCAAGATCAG CTACAACAAC ACCAACCGCG AGGACCTGCT
1921 GATCCTGTGG GGAATCCACC ACAGCAACAA TGCCGCCGAG CAGACCAACC TGTACAAGAA
1981 CCCCACCACC TACATCAGCG TGGGCACCAG CACACTGAAC CAGAGACTGG TGCCTAAGAT
2041 CGCCACACGG TCCCAAGTGA ATGGCGAGAG GGGCAGAATG GACTTCTTCT GGACCATCCT
2101 GAAGCCTAAC GACGCCATCC ACTTTGAGAG CAACGGCAAC TTTATCGCCC CTGAGTACGC
2161 CTACAAGATC GTGAAGAAGG GCGACAGCAC CATCATGAAG TCCGAGGTGG AATACGGCCA
2221 CTGCAACACC AAGTGTCAGA CCCCTATCGG CGCCATCAAC TCCAGCATGC CCTTCCACAA
2281 CATTCACCCT CTGACCATCG GCGAGTGCCC CAAATACGTG AAGTCCAACA AGCTGGTGCT
2341 GGCTACCGGC CTGAGAAACA GCCCTCTGAG AGAGAAGCGC AGACGGAAGA AGAGAGGCCT
2401 GTTTGGCGCC ATTGCCGGCT TTATCGAAGG CGGCTGGCAA GGCATGGTGG ACGGATGGTA
2461 CGGCTACCAT CACAGCAACG AGCAAGGCTC TGGATACGCC GCCGACAAAG AGAGCACCCA
2521 GAAAGCCATT GACGGCGTGA CCAACAAAGT GAACAGCATC ATCGACAAGA TGAACACCCA
2581 GTTCGAGGCC GTGGGCAGAG AGTTCAACAA CCTGGAACGG CGGATCGAGA ATCTGAACAA
2641 GAAGATGGAG GACGGCTTCC TGGACGTGTG GACCTACAAT GCCGAGCTGC TGGTCCTGAT
2701 GGAAAACGAG AGAACCCTGG ACTTCCACGA CTCCAACGTG AAGAACCTGT ACGACAAAGT
2761 GCGGCTCCAG CTGCGGGACA ACGCCAAAGA ACTCGGCAAC GGCTGCTTCG AGTTCTACCA
2821 CAAGTGCGAC AACGAGTGCA TGGAAAGCGT GCGGAACGGC ACCTACGACT ACCCTCAGTA
2881 CAGCGAGGAA GCCCGGCTGA AGAGAGAAGA GATCAGCGGA GTGAAGCTGG AATCCATCGG
2941 CACATACCAG ATCCTGTCCA TCTACAGCAC CGTGGCCTCT TCTCTGGCCC TGGCCATTAT
3001 GGTGGCTGGC CTGTCTCTGT GGATGTGCAG CAATGGCAGC CTCCAGTGCC GGATCTGCAT
3061 CTGAGCGGCC GCAGATCTGC TGTGCCTTCT AGTTGCCAGC CATCTGTTGT TTGCCCCTCC
3121 CCCGTGCCTT CCTTGACCCT GGAAGGTGCC ACTCCCACTG TCCTTTCCTA ATAAAATGAG
3181 GAAATTGCAT CGCATTGTCT GAGTAGGTGT CATTCTATTC TGGGGGGTGG GGTGGGGCAG
3241 GACAGCAAGG GGGAGGATTG GGAAGACAAT AGCAGGCATG CTGGGGATGC GGTGGGCTCT
3301 ATGGCTACCC AGGTGCTGAA GAATTGACCC GGTTCCTCCT GGGCCAGAAA GAAGCAGGCA
3361 CATCCCCTTC TCTGTGACAC ACCCTGTCCA CGCCCCTGGT TCTTAGTTCC AGCCCCACTC
3421 ATAGGACACT CATAGCTCAG GAGGGCTCCG CCTTCAATCC CACCCGCTAA AGTACTTGGA
3481 GCGGTCTCTC CCTCCCTCAT CAGCCCACCA AACCAAACCT AGCCTCCAAG AGTGGGAAGA
3541 AATTAAAGCA AGATAGGCTA TTAAGTGCAG AGGGAGAGAA AATGCCTCCA ACATGTGAGG
3601 AAGTAATGAG AGAAATCATA GAATTTTAAG GCCATGATTT AAGGCCATCA TGGCCTTAAT
3661 CTTCCGCTTC CTCGCTCACT GACTCGCTGC GCTCGGTCGT TCGGCTGCGG CGAGCGGTAT
3721 CAGCTCACTC AAAGGCGGTA ATACGGTTAT CCACAGAATC AGGGGATAAC GCAGGAAAGA
3781 ACATGTGAGC AAAAGGCCAG CAAAAGGCCA GGAACCGTAA AAAGGCCGCG TTGCTGGCGT
3841 TTTTCCATAG GCTCCGCCCC CCTGACGAGC ATCACAAAAA TCGACGCTCA AGTCAGAGGT
3901 GGCGAAACCC GACAGGACTA TAAAGATACC AGGCGTTTCC CCCTGGAAGC TCCCTCGTGC
3961 GCTCTCCTGT TCCGACCCTG CCGCTTACCG GATACCTGTC CGCCTTTCTC CCTTCGGGAA
4021 GCGTGGCGCT TTCTCATAGC TCACGCTGTA GGTATCTCAG TTCGGTGTAG GTCGTTCGCT
4081 CCAAGCTGGG CTGTGTGCAC GAACCCCCCG TTCAGCCCGA CCGCTGCGCC TTATCCGGTA
4141 ACTATCGTCT TGAGTCCAAC CCGGTAAGAC ACGACTTATC GCCACTGGCA GCAGCCACTG
4201 GTAACAGGAT TAGCAGAGCG AGGTATGTAG GCGGTGCTAC AGAGTTCTTG AAGTGGTGGC
4261 CTAACTACGG CTACACTAGA AGAACAGTAT TTGGTATCTG CGCTCTGCTG AAGCCAGTTA
4321 CCTTCGGAAA AAGAGTTGGT AGCTCTTGAT CCGGCAAACA AACCACCGCT GGTAGCGGTG
4381 GTTTTTTTGT TTGCAAGCAG CAGATTACGC GCAGAAAAAA AGGATCTCAA GAAGATCCTT
4441 TGATCTTTTC TACGGGGTCT GACGCTCAGT GGAACGAAAA CTCACGTTAA GGGATTTTGG
4501 TCATGAGATT ATCAAAAAGG ATCTTCACCT AGATCCTTTT AAATTAAAAA TGAAGTTTTA
4561 AATCAATCTA AAGTATATAT GAGTAAACTT GGTCTGACAG TTACCAATGC TTAATCAGTG
4621 AGGCACCTAT CTCAGCGATC TGTCTATTTC GTTCATCCAT AGTTGCCTGA CTCGGGGGGG
4681 GGGGGCGCTG AGGTCTGCCT CGTGAAGAAG GTGTTGCTGA CTCATACCAG GCCTGAATCG
4741 CCCCATCATC CAGCCAGAAA GTGAGGGAGC CACGGTTGAT GAGAGCTTTG TTGTAGGTGG
4801 ACCAGTTGGT GATTTTGAAC TTTTGCTTTG CCACGGAACG GTCTGCGTTG TCGGGAAGAT
4861 GCGTGATCTG ATCCTTCAAC TCAGCAAAAG TTCGATTTAT TCAACAAAGC CGCCGTCCCG
4921 TCAAGTCAGC GTAATGCTCT GCCAGTGTTA CAACCAATTA ACCAATTCTG ATTAGAAAAA
4981 CTCATCGAGC ATCAAATGAA ACTGCAATTT ATTCATATCA GGATTATCAA TACCATATTT
5041 TTGAAAAAGC CGTTTCTGTA ATGAAGGAGA AAACTCACCG AGGCAGTTCC ATAGGATGGC
5101 AAGATCCTGG TATCGGTCTG CGATTCCGAC TCGTCCAACA TCAATACAAC CTATTAATTT
5161 CCCCTCGTCA AAAATAAGGT TATCAAGTGA GAAATCACCA TGAGTGACGA CTGAATCCGG
5221 TGAGAATGGC AAAAGCTTAT GCATTTCTTT CCAGACTTGT TCAACAGGCC AGCCATTACG
5281 CTCGTCATCA AAATCACTCG CATCAACCAA ACCGTTATTC ATTCGTGATT GCGCCTGAGC
5341 GAGACGAAAT ACGCGATCGC TGTTAAAAGG ACAATTACAA ACAGGAATCG AATGCAACCG
5401 GCGCAGGAAC ACTGCCAGCG CATCAACAAT ATTTTCACCT GAATCAGGAT ATTCTTCTAA
5461 TACCTGGAAT GCTGTTTTCC CGGGGATCGC AGTGGTGAGT AACCATGCAT CATCAGGAGT
5521 ACGGATAAAA TGCTTGATGG TCGGAAGAGG CATAAATTCC GTCAGCCAGT TTAGTCTGAC
5581 CATCTCATCT GTAACATCAT TGGCAACGCT ACCTTTGCCA TGTTTCAGAA ACAACTCTGG
5641 CGCATCGGGC TTCCCATACA ATCGATAGAT TGTCGCACCT GATTGCCCGA CATTATCGCG
5701 AGCCCATTTA TACCCATATA AATCAGCATC CATGTTGGAA TTTAATCGCG GCCTCGAGCA
5761 AGACGTTTCC CGTTGAATAT GGCTCATAAC ACCCCTTGTA TTACTGTTTA TGTAAGCAGA
5821 CAGTTTTATT GTTCATGATG ATATATTTTT ATCTTGTGCA ATGTAACATC AGAGATTTTG
5881 AGACACAACG TGGCTTTCCC CCCCCCCCCA TTATTGAAGC ATTTATCAGG GTTATTGTCT
5941 CATGAGCGGA TACATATTTG AATGTATTTA GAAAAATAAA CAAATAGGGG TTCCGCGCAC
6001 ATTTCCCCGA AAAGTGCCAC CTGACGTCTA AGAAACCATT ATTATCATGA CATTAACCTA
6061 TAAAAATAGG CGTATCACGA GGCCCTTTCG TC
```

### EXAMPLE 31—FLU_T4_HA_3

This example provides amino acid and nucleic acid sequences of the influenza haemagluttinin H5 head and stem regions for an embodiment of the invention known as FLU_T4_HA_3. In SEQ ID NO:89 below, the amino acid residues of the stem region are shown underlined. The amino acid residues of the head region are the remaining residues. Similarly, in SEQ ID NO:90 below, the nucleic acid residues of the stem region are shown underlined. The nucleic acid residues of the head region are the remaining residues.

FLU_T4_HA_3-HA0 amino acid sequence
(SEQ ID NO: 89)
MEKIVLLLAIVSLVKSDQICIGYHANNSTEQVDTIMEKNVTVTHAQDILEKTHNGKLCDLNGVKPLILKDCS
VAGWLLGNPMCDEFIRVPEWSYIVERANPANDLCFPGNLNDYEELKHLLSRINHFEKILIIPKSSWPNHNTS
LGVSAACPYQGTPSFFRNVVWLIKKNDTYPTIKISYNNTNREDLLILWGIHHSNNTAEQTNLYKNPTTYISV
GTSTLNQRLVPKIANRSQVNGQRGRMDFFWTILKPNDAIHFESNGNFIAPEYAYKIVKKGDSTIMKSEVEYG
HCNTKCQTPIGAINSSMPFHNIHPLTIGECPKYVKSNKLVLATGLRNSPLREKRRRKKRGLFGAIAGFIEGG
WQGMVDGWYGYHHSNEQGSGYAADKESTQKAIDGVTNKVNSIIDKMNTQFEAVGREFNNLERRIENLNKKME
DGFLDVWTYNAELLVLMENERTLDFHDSNVKNLYDKVRLQLRDNAKELGNGCFEFYHKCDNECMESVRNGTY
DYPQYSEEARLKREEISGVKLESIGTYQILSIYSTVASSLALAIMVAGLSLWMCSNGSLQCRICI
FLU_T4_HA_3-HA0 nucleic acid sequence
(SEQ ID NO: 90)
GTACCGCCACCATGGAAAAGATCGTGCTGCTGCTGGCCATCGTGTCCCTGGTCAAGAGCGACCAAAT
CTGCATCGGCTACCACGCCAACAACAGCACCGAACAGGTGGACACCATTATGGAAAAGAACGTCACC
GTGACACACGCCCAGGACATCCTGGAAAAGACCCACAACGGCAAGCTGTGCGACCTGAACGGCGTGA
AGCCTCTGATCCTGAAGGATTGCTCTGTGGCCGGATGGCTGCTGGGCAATCCCATGTGCGACGAGTT
CATCAGAGTGCCCGAGTGGTCCTACATCGTGGAAAGAGCCAATCCTGCCAACGACCTGTGCTTCCCC
GGCAACCTGAACGACTACGAGGAACTGAAGCACCTCCTGAGCCGGATCAACCACTTCGAGAAGATCC
TGATCATCCCCAAGAGCAGCTGGCCCAACCACAATACCAGCCTGGGAGTGTCTGCCGCATGTCCATA
TCAGGGCACCCCTAGCTTTTTCCGGAACGTCGTGTGGCTGATCAAGAAGAACGACACATACCCCACC
ATCAAGATCAGCTACAACAACACCAACCGCGAGGACCTGCTGATCCTGTGGGGAATCCACCACAGCA
ACAATACCGCCGAGCAGACCAACCTGTACAAGAACCCCACCACCTACATCAGCGTGGGCACCAGCAC
ACTGAACCAGAGACTGGTGCCTAAGATCGCCAACCGCAGCCAAGTGAATGGCCAGAGGGGCAGAATG
GACTTCTTCTGGACCATCCTGAAGCCTAACGACGCCATCCACTTTGAGAGCAACGGCAACTTTATCG
CCCCTGAGTACGCCTACAAGATCGTGAAGAAGGGCGACAGCACCATCATGAAGTCCGAGGTGGAATA
CGGCCACTGCAACACCAAGTGTCAGACCCCTATCGGCGCCATCAACTCCAGCATGCCCTTCCACAAC
ATTCACCCTCTGACCATCGGCGAGTGCCCCAAATACGTGAAGTCCAACAAGCTGGTGCTGGCTACCG
GCCTGAGAAACAGCCCTCTGAGAGAGAAGCGCAGACGGAAGAAGAGAGGCCTGTTTGGCGCCATTGC
CGGCTTTATCGAAGGCGGCTGGCAAGGCATGGTGGACGGATGGTACGGCTACCATCACAGCAACGAG
CAAGGCTCTGGCTACGCCGCCGACAAAGAGAGCACACAGAAAGCCATCGACGGCGTGACCAACAAAG
TGAACAGCATCATCGACAAGATGAACACCCAGTTCGAGGCCGTGGGCAGAGAGTTCAACAACCTGGA
ACGGCGGATCGAGAATCTGAACAAGAAGATGGAGGACGGCTTCCTGGACGTGTGGACCTACAATGCC
GAGCTGCTGGTCCTGATGGAAAACGAGAGAACCCTGGACTTCCACGACTCCAACGTGAAGAACCTGT
ACGACAAAGTGCGGCTCCAGCTGCGGGACAACGCCAAAGAACTCGGCAACGGCTGCTTCGAGTTCTA
CCACAAGTGCGACAACGAGTGCATGGAAAGCGTGCGGAACGGCACCTACGACTACCCTCAGTACAGC
GAGGAAGCCCGGCTGAAGAGAGAAGAGATCAGCGGAGTGAAGCTGGAATCCATCGGCACATACCAGA
TCCTGTCCATCTACAGCACCGTGGCCTCTTCTCTGGCCCTGGCCATTATGGTGGCTGGCCTGTCTCT
GTGGATGTGCAGCAATGGCAGCCTCCAGTGCCGGATCTGCATCTGAGCGGCC
FLU_T4_HA_3-Head region amino acid sequence
(SEQ ID NO: 91)
THNGKLCDLNGVKPLILKDCSVAGWLLGNPMCDEFIRVPEWSYIVERANPANDLCFPGNLNDYEELKHLLSRIN
HFEKILIIPKSSWPNHNTSLGVSAACPYQGTPSFFRNVVWLIKKNDTYPTIKISYNNTNREDLLILWGIHHSNN
TAEQTNLYKNPTTYISVGTSTLNQRLVPKIANRSQVNGQRGRMDFFWTILKPNDAIHFESNGNFIAPEYAYKIV
KKGDSTIMKSEVEYGHCNTKCQTPIGAINSSMPFHNIHPLTIGECP
FLU_T4_HA_3-Head region nucleic acid sequence
(SEQ ID NO: 92)
TCCTGGAAAAGACCCACAACGGCAAGCTGTGCGACCTGAACGGCGTGAAGCCTCTGATCCTGAAGGA
TTGCTCTGTGGCCGGATGGCTGCTGGGCAATCCCATGTGCGACGAGTTCATCAGAGTGCCCGAGTGG
TCCTACATCGTGGAAAGAGCCAATCCTGCCAACGACCTGTGCTTCCCCGGCAACCTGAACGACTACG
AGGAACTGAAGCACCTCCTGAGCCGGATCAACCACTTCGAGAAGATCCTGATCATCCCCAAGAGCAG
CTGGCCCAACCACAATACCAGCCTGGGAGTGTCTGCCGCATGTCCATATCAGGGCACCCCTAGCTTT
TTCCGGAACGTCGTGTGGCTGATCAAGAAGAACGACACATACCCCACCATCAAGATCAGCTACAACA
ACACCAACCGCGAGGACCTGCTGATCCTGTGGGGAATCCACCACAGCAACAATACCGCCGAGCAGAC
CAACCTGTACAAGAACCCCACCACCTACATCAGCGTGGGCACCAGCACACTGAACCAGAGACTGGTG
CCTAAGATCGCCAACCGCAGCCAAGTGAATGGCCAGAGGGGCAGAATGGACTTCTTCTGGACCATCC
TGAAGCCTAACGACGCCATCCACTTTGAGAGCAACGGCAACTTTATCGCCCCTGAGTACGCCTACAA
GATCGTGAAGAAGGGCGACAGCACCATCATGAAGTCCGAGGTGGAATACGGCCACTGCAACACCAAG
TGTCAGACCCCTATCGGCGCCATCAACTCCAGCATGCCCTTCCACAACATTCACCCTCTGACCATCG
GCGAGTGCCCCAAATACGTG
FLU_T4_HA_3-First stem region amino acid sequence
(SEQ ID NO: 93)
MEKIVLLLAIVSLVKSDQICIGYHANNSTEQVDTIMEKNVTVTHAQDILEK
FLU_T4_HA_3-First stem region nucleic acid sequence
(SEQ ID NO: 94)
GTACCGCCACCATGGAAAAGATCGTGCTGCTGCTGGCCATCGTGTCCCTGGTCAAGAGCGACCAAAT
CTGCATCGGCTACCACGCCAACAACAGCACCGAACAGGTGGACACCATTATGGAAAAGAACGTCACC
GTGACACACGCCCAGGACA
FLU_T4_HA_3-Second stem region amino acid sequence
(SEQ ID NO: 95)
KYVKSNKLVLATGLRNSPLREKRRRKKRGLFGAIAGFIEGGWQGMVDGWYGYHHSNEQGSGYAADKESTQKAID
GVTNKVNSIIDKMNTQFEAVGREFNNLERRIENLNKKMEDGELDVWTYNAELLVLMENERTLDFHDSNVKNLYD
KVRLQLRDNAKELGNGCFEFYHKCDNECMESVRNGTYDYPQYSEEARLKREEISGVKLESIGTYQILSIYSTVA
SSLALAIMVAGLSLWMCSNGSLQCRICI
FLU_T4_HA_3-Second stem region nucleic acid sequence
(SEQ ID NO: 96)
AAGTCCAACAAGCTGGTGCTGGCTACCGGCCTGAGAAACAGCCCTCTGAGAGAGAAGCGCAGACGGA
AGAAGAGAGGCCTGTTTGGCGCCATTGCCGGCTTTATCGAAGGCGGCTGGCAAGGCATGGTGGACGG
ATGGTACGGCTACCATCACAGCAACGAGCAAGGCTCTGGCTACGCCGCCGACAAAGAGAGCACACAG
AAAGCCATCGACGGCGTGACCAACAAAGTGAACAGCATCATCGACAAGATGAACACCCAGTTCGAGG
CCGTGGGCAGAGAGTTCAACAACCTGGAACGGCGGATCGAGAATCTGAACAAGAAGATGGAGGACGG
CTTCCTGGACGTGTGGACCTACAATGCCGAGCTGCTGGTCCTGATGGAAAACGAGAGAACCCTGGAC
TTCCACGACTCCAACGTGAAGAACCTGTACGACAAAGTGCGGCTCCAGCTGCGGGACAACGCCAAAG
AACTCGGCAACGGCTGCTTCGAGTTCTACCACAAGTGCGACAACGAGTGCATGGAAAGCGTGCGGAA
CGGCACCTACGACTACCCTCAGTACAGCGAGGAAGCCCGGCTGAAGAGAGAAGAGATCAGCGGAGTG
AAGCTGGAATCCATCGGCACATACCAGATCCTGTCCATCTACAGCACCGTGGCCTCTTCTCTGGCCC
TGGCCATTATGGTGGCTGGCCTGTCTCTGTGGATGTGCAGCAATGGCAGCCTCCAGTGCCGGATCTG
CATCTGAGCGGCC

### EXAMPLE 32—PEVAC-FLU_T4_HA_3

This example provides the nucleic acid sequence of pEVAC-FLU_T4_HA_3.

```
pEVAC-FLU_T4_HA 3-nucleic acid sequence (SEQ ID NO: 97):
LOCUS pVRC8400EVAC_Ar_pEVAC-Flu_T4_HA_3 6092 bp DNA
linear SYN 08-SEP-2022
FEATURES Location/Qualifiers
          CDS 1344 . . . 3070
                                      /label = “Flu_T4_HA_3”
                                      /note = “CI: V1 = KpnI, V2-NotI, I1 = KpnI, I2 = NotI”
ORIGIN
      1 TCGCGCGTTT CGGTGATGAC GGTGAAAACC TCTGACACAT GCAGCTCCCG GAGACGGTCA
    61 CAGCTTGTCT GTAAGCGGAT GCCGGGAGCA GACAAGCCCG TCAGGGCGCG TCAGCGGGTG
  121 TTGGCGGGTG TCGGGGCTGG CTTAACTATG CGGCATCAGA GCAGATTGTA CTGAGAGTGC
  181 ACCATATGCG GTGTGAAATA CCGCACAGAT GCGTAAGGAG AAAATACCGC ATCAGATTGG
  241 CTATTGGCCA TTGCATACGT TGTATCCATA TCATAATATG TACATTTATA TTGGCTCATG
  301 TCCAACATTA CCGCCATGTT GACATTGATT ATTGACTAGT TATTAATAGT AATCAATTAC
  361 GGGGTCATTA GTTCATAGCC CATATATGGA GTTCCGCGTT ACATAACTTA CGGTAAATGG
  421 CCCGCCTGGC TGACCGCCCA ACGACCCCCG CCCATTGACG TCAATAATGA CGTATGTTCC
  481 CATAGTAACG CCAATAGGGA CTTTCCATTG ACGTCAATGG GTGGAGTATT TACGGTAAAC
  541 TGCCCACTTG GCAGTACATC AAGTGTATCA TATGCCAAGT ACGCCCCCTA TTGACGTCAA
  601 TGACGGTAAA TGGCCCGCCT GGCATTATGC CCAGTACATG ACCTTATGGG ACTTTCCTAC
  661 TTGGCAGTAC ATCTACGTAT TAGTCATCGC TATTACCATG GTGATGCGGT TTTGGCAGTA
  721 CATCAATGGG CGTGGATAGC GGTTTGACTC ACGGGGATTT CCAAGTCTCC ACCCCATTGA
  781 CGTCAATGGG AGTTTGTTTT GGCACCAAAA TCAACGGGAC TTTCCAAAAT GTCGTAACAA
  841 CTCCGCCCCA TTGACGCAAA TGGGCGGTAG GCGTGTACGG TGGGAGGTCT ATATAAGCAG
  901 AGCTCGTTTA GTGAACCGTC AGATCGCCTG GAGACGCCAT CCACGCTGTT TTGACCTCCA
  961 TAGAAGACAC CGGGACCGAT CCAGCCTCCA TCGGCTCGCA TCTCTCCTTC ACGCGCCCGC
1021 CGCCCTACCT GAGGCCGCCA TCCACGCCGG TTGAGTCGCG TTCTGCCGCC TCCCGCCTGT
1081 GGTGCCTCCT GAACTGCGTC CGCCGTCTAG GTAAGTTTAA AGCTCAGGTC GAGACCGGGC
1141 CTTTGTCCGG CGCTCCCTTG GAGCCTACCT AGACTCAGCC GGCTCTCCAC GCTTTGCCTG
1201 ACCCTGCTTG CTCAACTCTA GTTAACGGTG GAGGGCAGTG TAGTCTGAGC AGTACTCGTT
1261 GCTGCCGCGC GCGCCACCAG ACATAATAGC TGACAGACTA ACAGACTGTT CCTTTCCATG
1321 GGTCTTTTCT GCAGTCACCG TCGGTACCGC CACCATGGAA AAGATCGTGC TGCTGCTGGC
1381 CATCGTGTCC CTGGTCAAGA GCGACCAAAT CTGCATCGGC TACCACGCCA ACAACAGCAC
1441 CGAACAGGTG GACACCATTA TGGAAAAGAA CGTCACCGTG ACACACGCCC AGGACATCCT
1501 GGAAAAGACC CACAACGGCA AGCTGTGCGA CCTGAACGGC GTGAAGCCTC TGATCCTGAA
1561 GGATTGCTCT GTGGCCGGAT GGCTGCTGGG CAATCCCATG TGCGACGAGT TCATCAGAGT
1621 GCCCGAGTGG TCCTACATCG TGGAAAGAGC CAATCCTGCC AACGACCTGT GCTTCCCCGG
1681 CAACCTGAAC GACTACGAGG AACTGAAGCA CCTCCTGAGC CGGATCAACC ACTTCGAGAA
1741 GATCCTGATC ATCCCCAAGA GCAGCTGGCC CAACCACAAT ACCAGCCTGG GAGTGTCTGC
1801 CGCATGTCCA TATCAGGGCA CCCCTAGCTT TTTCCGGAAC GTCGTGTGGC TGATCAAGAA
1861 GAACGACACA TACCCCACCA TCAAGATCAG CTACAACAAC ACCAACCGCG AGGACCTGCT
1921 GATCCTGTGG GGAATCCACC ACAGCAACAA TACCGCCGAG CAGACCAACC TGTACAAGAA
1981 CCCCACCACC TACATCAGCG TGGGCACCAG CACACTGAAC CAGAGACTGG TGCCTAAGAT
2041 CGCCAACCGC AGCCAAGTGA ATGGCCAGAG GGGCAGAATG GACTTCTTCT GGACCATCCT
2101 GAAGCCTAAC GACGCCATCC ACTTTGAGAG CAACGGCAAC TTTATCGCCC CTGAGTACGC
2161 CTACAAGATC GTGAAGAAGG GCGACAGCAC CATCATGAAG TCCGAGGTGG AATACGGCCA
2221 CTGCAACACC AAGTGTCAGA CCCCTATCGG CGCCATCAAC TCCAGCATGC CCTTCCACAA
2281 CATTCACCCT CTGACCATCG GCGAGTGCCC CAAATACGTG AAGTCCAACA AGCTGGTGCT
2341 GGCTACCGGC CTGAGAAACA GCCCTCTGAG AGAGAAGCGC AGACGGAAGA AGAGAGGCCT
2401 GTTTGGCGCC ATTGCCGGCT TTATCGAAGG CGGCTGGCAA GGCATGGTGG ACGGATGGTA
2461 CGGCTACCAT CACAGCAACG AGCAAGGCTC TGGCTACGCC GCCGACAAAG AGAGCACACA
2521 GAAAGCCATC GACGGCGTGA CCAACAAAGT GAACAGCATC ATCGACAAGA TGAACACCCA
2581 GTTCGAGGCC GTGGGCAGAG AGTTCAACAA CCTGGAACGG CGGATCGAGA ATCTGAACAA
2641 GAAGATGGAG GACGGCTTCC TGGACGTGTG GACCTACAAT GCCGAGCTGC TGGTCCTGAT
2701 GGAAAACGAG AGAACCCTGG ACTTCCACGA CTCCAACGTG AAGAACCTGT ACGACAAAGT
2761 GCGGCTCCAG CTGCGGGACA ACGCCAAAGA ACTCGGCAAC GGCTGCTTCG AGTTCTACCA
2821 CAAGTGCGAC AACGAGTGCA TGGAAAGCGT GCGGAACGGC ACCTACGACT ACCCTCAGTA
2881 CAGCGAGGAA GCCCGGCTGA AGAGAGAAGA GATCAGCGGA GTGAAGCTGG AATCCATCGG
2941 CACATACCAG ATCCTGTCCA TCTACAGCAC CGTGGCCTCT TCTCTGGCCC TGGCCATTAT
3001 GGTGGCTGGC CTGTCTCTGT GGATGTGCAG CAATGGCAGC CTCCAGTGCC GGATCTGCAT
3061 CTGAGCGGCC GCAGATCTGC TGTGCCTTCT AGTTGCCAGC CATCTGTTGT TTGCCCCTCC
3121 CCCGTGCCTT CCTTGACCCT GGAAGGTGCC ACTCCCACTG TCCTTTCCTA ATAAAATGAG
3181 GAAATTGCAT CGCATTGTCT GAGTAGGTGT CATTCTATTC TGGGGGGTGG GGTGGGGCAG
3241 GACAGCAAGG GGGAGGATTG GGAAGACAAT AGCAGGCATG CTGGGGATGC GGTGGGCTCT
3301 ATGGCTACCC AGGTGCTGAA GAATTGACCC GGTTCCTCCT GGGCCAGAAA GAAGCAGGCA
3361 CATCCCCTTC TCTGTGACAC ACCCTGTCCA CGCCCCTGGT TCTTAGTTCC AGCCCCACTC
3421 ATAGGACACT CATAGCTCAG GAGGGCTCCG CCTTCAATCC CACCCGCTAA AGTACTTGGA
3481 GCGGTCTCTC CCTCCCTCAT CAGCCCACCA AACCAAACCT AGCCTCCAAG AGTGGGAAGA
3541 AATTAAAGCA AGATAGGCTA TTAAGTGCAG AGGGAGAGAA AATGCCTCCA ACATGTGAGG
3601 AAGTAATGAG AGAAATCATA GAATTTTAAG GCCATGATTT AAGGCCATCA TGGCCTTAAT
3661 CTTCCGCTTC CTCGCTCACT GACTCGCTGC GCTCGGTCGT TCGGCTGCGG CGAGCGGTAT
3721 CAGCTCACTC AAAGGCGGTA ATACGGTTAT CCACAGAATC AGGGGATAAC GCAGGAAAGA
3781 ACATGTGAGC AAAAGGCCAG CAAAAGGCCA GGAACCGTAA AAAGGCCGCG TTGCTGGCGT
3841 TTTTCCATAG GCTCCGCCCC CCTGACGAGC ATCACAAAAA TCGACGCTCA AGTCAGAGGT
3901 GGCGAAACCC GACAGGACTA TAAAGATACC AGGCGTTTCC CCCTGGAAGC TCCCTCGTGC
3961 GCTCTCCTGT TCCGACCCTG CCGCTTACCG GATACCTGTC CGCCTTTCTC CCTTCGGGAA
4021 GCGTGGCGCT TTCTCATAGC TCACGCTGTA GGTATCTCAG TTCGGTGTAG GTCGTTCGCT
4081 CCAAGCTGGG CTGTGTGCAC GAACCCCCCG TTCAGCCCGA CCGCTGCGCC TTATCCGGTA
4141 ACTATCGTCT TGAGTCCAAC CCGGTAAGAC ACGACTTATC GCCACTGGCA GCAGCCACTG
4201 GTAACAGGAT TAGCAGAGCG AGGTATGTAG GCGGTGCTAC AGAGTTCTTG AAGTGGTGGC
4261 CTAACTACGG CTACACTAGA AGAACAGTAT TTGGTATCTG CGCTCTGCTG AAGCCAGTTA
4321 CCTTCGGAAA AAGAGTTGGT AGCTCTTGAT CCGGCAAACA AACCACCGCT GGTAGCGGTG
4381 GTTTTTTTGT TTGCAAGCAG CAGATTACGC GCAGAAAAAA AGGATCTCAA GAAGATCCTT
4441 TGATCTTTTC TACGGGGTCT GACGCTCAGT GGAACGAAAA CTCACGTTAA GGGATTTTGG
4501 TCATGAGATT ATCAAAAAGG ATCTTCACCT AGATCCTTTT AAATTAAAAA TGAAGTTTTA
4561 AATCAATCTA AAGTATATAT GAGTAAACTT GGTCTGACAG TTACCAATGC TTAATCAGTG
4621 AGGCACCTAT CTCAGCGATC TGTCTATTTC GTTCATCCAT AGTTGCCTGA CTCGGGGGGG
4681 GGGGGCGCTG AGGTCTGCCT CGTGAAGAAG GTGTTGCTGA CTCATACCAG GCCTGAATCG
4741 CCCCATCATC CAGCCAGAAA GTGAGGGAGC CACGGTTGAT GAGAGCTTTG TTGTAGGTGG
4801 ACCAGTTGGT GATTTTGAAC TTTTGCTTTG CCACGGAACG GTCTGCGTTG TCGGGAAGAT
4861 GCGTGATCTG ATCCTTCAAC TCAGCAAAAG TTCGATTTAT TCAACAAAGC CGCCGTCCCG
4921 TCAAGTCAGC GTAATGCTCT GCCAGTGTTA CAACCAATTA ACCAATTCTG ATTAGAAAAA
4981 CTCATCGAGC ATCAAATGAA ACTGCAATTT ATTCATATCA GGATTATCAA TACCATATTT
5041 TTGAAAAAGC CGTTTCTGTA ATGAAGGAGA AAACTCACCG AGGCAGTTCC ATAGGATGGC
5101 AAGATCCTGG TATCGGTCTG CGATTCCGAC TCGTCCAACA TCAATACAAC CTATTAATTT
5161 CCCCTCGTCA AAAATAAGGT TATCAAGTGA GAAATCACCA TGAGTGACGA CTGAATCCGG
5221 TGAGAATGGC AAAAGCTTAT GCATTTCTTT CCAGACTTGT TCAACAGGCC AGCCATTACG
5281 CTCGTCATCA AAATCACTCG CATCAACCAA ACCGTTATTC ATTCGTGATT GCGCCTGAGC
5341 GAGACGAAAT ACGCGATCGC TGTTAAAAGG ACAATTACAA ACAGGAATCG AATGCAACCG
5401 GCGCAGGAAC ACTGCCAGCG CATCAACAAT ATTTTCACCT GAATCAGGAT ATTCTTCTAA
5461 TACCTGGAAT GCTGTTTTCC CGGGGATCGC AGTGGTGAGT AACCATGCAT CATCAGGAGT
5521 ACGGATAAAA TGCTTGATGG TCGGAAGAGG CATAAATTCC GTCAGCCAGT TTAGTCTGAC
5581 CATCTCATCT GTAACATCAT TGGCAACGCT ACCTTTGCCA TGTTTCAGAA ACAACTCTGG
5641 CGCATCGGGC TTCCCATACA ATCGATAGAT TGTCGCACCT GATTGCCCGA CATTATCGCG
5701 AGCCCATTTA TACCCATATA AATCAGCATC CATGTTGGAA TTTAATCGCG GCCTCGAGCA
5761 AGACGTTTCC CGTTGAATAT GGCTCATAAC ACCCCTTGTA TTACTGTTTA TGTAAGCAGA
5821 CAGTTTTATT GTTCATGATG ATATATTTTT ATCTTGTGCA ATGTAACATC AGAGATTTTG
5881 AGACACAACG TGGCTTTCCC CCCCCCCCCA TTATTGAAGC ATTTATCAGG GTTATTGTCT
5941 CATGAGCGGA TACATATTTG AATGTATTTA GAAAAATAAA CAAATAGGGG TTCCGCGCAC
6001 ATTTCCCCGA AAAGTGCCAC CTGACGTCTA AGAAACCATT ATTATCATGA CATTAACCTA
6061 TAAAAATAGG CGTATCACGA GGCCCTTTCG TC
```

### EXAMPLE 33—RESIDUE DIFFERENCES IN AMINO ACID SEQUENCE OF INFLUENZA TIER 4 H5 VACCINE CANDIDATES FLU_T4_HA_1, FLU_T4_HA_2, AND FLU_T4_HA_3, COMPARED INFLUENZA TIER 2 AND TIER 3 H5 DESIGNS, AND WILD-TYPE H5 STRAINS

FIG. 25 summarises novel amino acid residue changes in influenza haemagluttinin H5 for embodiments of the invention relating to FLU_T4_HA_1, FLU_T4_HA_2, and FLU_T4_HA_3 designed sequences. These novel amino acid residue changes are shown in bold and underline for each of FLU_T4_HA_1, FLU_T4_HA_2, and FLU_T4_HA_3. In particular, amino acid residues at positions 107, 142, 200, and 231 of A/Sichuan/2014 H5 have been changed in some or all of the designed sequences according to the invention. These residue alterations are not present at the corresponding residue positions in the known wild type H5 sequences or previous H5 designed sequences shown in FIG. 25. Residue positions 107, 142, 200, and 231 are at epitope regions in the H5 head region, and the amino acid changes at these positions in the new T4 H5 designs alter the affinity of H5 towards binding antibodies.

FIG. 26 shows important amino acid residue positions of H5, in particular, residue positions 107, 142, 172, 200, 231, 238, 344, and 345, corresponding to amino acid residue positions of A/Sichuan/2014. Positions 142, 172, 200, and 231 of H5 are at epitope regions in the head region, and positions 344-345 are at an epitope region in the stem region. Position 107 is at a receptor binding site. Amino acid residue changes at these positions alter the affinity of H5 towards binding antibodies. Position 238 is at a receptor binding site in the head region. Mutation at this residue reduces the affinity of HA to its receptor (sialic acid) on the surface of target cells, thus increasing the bioavailability of HA for antigen presentation. The residues shown in bold and underline format are novel amino acid residues at the positions disclosed above, which are not present in the H5 wild type sequences shown or in previous designed H5 sequences.

FIG. 27 summarises amino acid residues of H5 FLU_T4_HA_1, FLU_T4_HA_2, and FLU_T4_HA_3, at important residue positions of H5. Positions A, B, and C of H5 are at epitope regions in the head region, and residue changes at these positions alter the affinity of H5 towards binding antibodies. Positions D and E are in the H5 stem region, and mutations at these positions alter the stability of the stem region both in the pre-fusion and post-fusion state. The amino acid residues at positions 148, 149, and 238 are at receptor binding sites. Amino acid changes at these residue positions alter the affinity of HA to its receptor (sialic acid) on the surface of target cells, thus increasing or decreasing the bioavailability of HA for antigen presentation.

FIG. 28 shows a multiple sequence alignment of H5 amino acid sequence for FLU_T4_HA_1, FLU_T4_HA_2, and FLU_T4_HA_3, known wild-type influenza H5 strains, and previously designed H5 sequences. The amino acid residue positions in the figure correspond to the amino acid residue positions of A/Sichuan/2014 (SEQ ID NO:100):

>EPI533583_A/Sichuan/26221/2014_H5N6
(SEQ ID NO: 100)
MEKIVLLLAIVSLVKGDQICIGYHANNSTEQVDTIMEKNVTVTHAQDILE
KTHNGKLCDLNGVKPLILKDCSVAGWLLGNPMCDEFIRVPEWSYIVERAN
PANDLCYPGNLNDYEELKHLLSRINHFEKILIIPKSSWTNHETSLGVSAA
CPYQGTPSFFRNVVWLIKKNDAYPTIKISYNNTNQEDLLILWGVHHSNNA
AEQTNLYKNPTTYISVGTSTLNQRLVPKIATRSQVNGQRGRMDFFWTILK
PNDAIHFESNGNFIAPEYAYKIVKKGDSTIMKSEMEYGHCNTKCQTPIGA
INSSMPFHNIHPLTIGECPKYVKSNKLVLATGLRNSPLREKRRKRGLFGA
IAGFIEGGWQGMVDGWYGYHHSNEQGSGYAADKESTQKAIDGVTNKVNSI
IDKMNTQFEAVGREFNNLERRIENLNKKMEDGFLDVWTYNAELLVLMENE
RTLDFHDSNVKNLYDKVRLQLRDNAKELGNGCFEFYHKCDNKCMESVRNG
TYDYPQYSEEARLKREEISGVKLESIGTYQILSIYSTVASSLALAIIVAG
LSLWMCSNGSLQCRICI

### EXAMPLE 34

### Designed Candidate H5 Vaccine Antigens Induce Broad Neutralising Responses Against Panel of Influenza H5 Clade 2.3.4.4.

Mice (n=6) immunised with our DIOS H5 DNA vaccines twice at 30 day interval:
H5_Anc_4 [T4_HA_1]
H5_Anc_4_mut1 [T4_HA_2]
H5_Anc_4_mut2 [T4_HA_3]

FIG. 29a is a summary of the neutralising activity of the candidate H5 vaccine antigens against a panel of clade 2.3.4.4. H5 viruses. The figure shows that the neutralising response elicited by immunisation by any one of the three designed sequences vs five clade 2.3.4.4 H5Nx strains, is comparable to the controls wherein the subjects were immunised with antigens from the same clade as the challenge strain (ns>0.05). These immune responses are broadly-neutralising and cover the 2.3.4.4 sub-clades. It is important to note that our designs generate good neutralising responses against one of the recent human H5 strains (A/Hangzhou/01/2021). The “ns” label denotes that the non-significant difference between our DIOS candidate and either the matched strain or the matched H5 clade is P<0.05 (Kruskal Wallis).

FIG. 29B shows neutralisation assay data for the vaccine designs vs controls. FIG. 29B shows that the DIOS candidates elicit equivalent responses to homologous strain viz. A/Sichuan/2014 but higher responses than the heterologous strain A/gyr/WSA and A/Anhui/2020 (left). The DIOS candidates also elicit equivalent responses to homologous strain viz. A/gyr/WSA but higher responses than the heterologous strain A/Anhui/2020 and A/Sichuan/2014 (right). Our best candidates from previous tier, FLU_T2_HA_9 (H5_ANC_1), and FLU_T3_HA_2 (H5_ANC_3), show poor response.

FIG. 29C shows that the DIOS candidates elicit equivalent responses to homologous strain viz. A/Anhui/2020 but higher responses than the heterologous strain A/gyr/WSA and A/Sichuan/2014 (left). The figure also shows that the DIOS candidates (H5_ANC_4 and H5_ANC_4_mut1) elicit better responses to clade 2.3.4.4b (A/mute swan/England/053054/2021) challenge in comparison to H5 controls viz. A/gyr/WSA, A/Anhui/2020 and A/Sichuan/2014, and the DIOS candidate H5_ANC_4_mut2 elicits a comparative neutralisation response to the challenge. Our best candidates from previous tier, FLU_T2_HA_9 (H5_ANC_1), and FLU_T3_HA_2 (H5 ANC_3), show poor response to clade 2.3.4.4b.

FIG. 29D is a repeat neutralisation assay of the assay performed in FIG. 29c right panel, and shows that the DIOS candidates (H5_ANC_4 and H5_ANC_4_mut1) again elicit better responses to clade 2.3.4.4b (A/mute swan/England/053054/2021) challenge in comparison to H5 controls viz. A/gyr/WSA, A/Anhui/2020 and A/Sichuan/2014, and the DIOS candidate H5_ANC_4_mut2 elicits a comparative neutralisation response to the challenge.

FIGS. 30A-I show individual neutralisation curves for mice immunised with a control PBS vaccine (A), DIOS candidate vaccine designs H5_ANC_4 (B), H5_ANC_4_mut1 (C), H5_ANC_4_mut2 (D), previous H5 vaccine designs H5_ANC1 (T2_HA_9)(E), H5_ANC_3 (T3_HA_2)(F), or homologous (H) or heterologous (G or 1) WT strains vs A/gyrfalcon/Washington/41088-6/2014) clade 2.3.4.4c. challenge strain.

FIGS. 31A-I similarly show individual neutralisation curves for mice immunised with either control PBS vaccine (A), new vaccine designs (B, C, D), previous DIOS vaccine candidates (E, F), or homologous (G) or heterologous WT strain (H and 1) vs A/Sichuan/26221/2014 clade 2.3.4.4a challenge strain.

FIGS. 32A-I similarly show individual neutralisation curves for mice immunised with either control PBS vaccine (A), new vaccine designs (B, C, D), previous DIOS vaccine candidates (E, F), or homologous (1) or heterologous strain (G, H) vs A/Anhui/2021-00011/2020 clade 2.3.4.4h challenge strain.

FIGS. 33A-I show individual neutralisation curves for mice immunised with a control PBS vaccine (A), DIOS candidate vaccine designs H5_ANC_4 (B), H5_ANC_4_mut1 (C), H5_ANC_4_mut2 (D), previous H5 vaccine designs H5_ANC_1 (T2_HA_9)(E), H5_ANC_3 (T3_HA_2)(F), or heterologous (G, H or 1) strains vs A/mute swan/England/053054/2021 clade 2.3.4.4b. challenge strain.

FIGS. 34A-I show individual neutralisation curves for mice immunised with a control PBS vaccine (A), DIOS candidate vaccine designs H5_ANC_4 (B), H5_ANC_4_mut1 (C), H5_ANC_4_mut2 (D), previous H5 vaccine designs H5_ANC_1 (T2_HA_9)(E), H5_ANC_3 (T3_HA_2)(F), or heterologous (G, H or 1) strains vs A/Hangzhou/01/2021 clade 2.3.4.4b. challenge strain.

### EXAMPLE 35—FLU_T3_NA_3

This example provides the amino acid and nucleic acid sequences of the influenza neuraminidase region for the embodiment of the invention known as FLU_T3_NA_3.

FLU_T3_NA_3 amino acid sequence
(SEQ ID NO: 98)
MNPNQKIITIGSICMVVGIISLILQIGNIISIWVSHSIQTGNONHPETCNQSIITYENNTWVNQTYV
NISNTNFVAEQDVTSVVLAGNSSLCPISGWAIYSKDNGIRIGSKGDVFVIREPFISCSHLECRTFFL
TQGALLNDKHSNGTVKDRSPYRTLMSCPVGEAPSPYNSRFESVAWSASACHDGMSWLTIGISGPDSG
AVAVLKYNGIITDTIKSWRNNILRTQESECACINGSCFTIMTDGPSDGQASYKIFKIEKGKVVKSVE
LNAPNYHYEECSCYPDAGKVMCVCRDNWHGSNRPWVSFDQNLEYQIGYICSGVFGDNPRPNDGTGSC
GPVSSNGANGVKGFSFRYGNGVWIGRTKSISSRKGFEMIWDPNGWTETDSSFSVKQDIVGINEWSGY
SGSFVQHPELTGLDCMRPCFWVELIRGRPEENTIWTSGSSISFCGVNSDTVGWSWPDGAELPFTIDK
FLU_T3_NA_3 nucleic acid sequence
(SEQ ID NO: 98)
ATGAACCCAAATCAGAAGATTATCACTATTGGTTCTATCTGTATGGTGGTAGGCATCATTTCACTTA
TCCTCCAGATTGGAAACATTATATCCATTTGGGTGTCACACAGTATTCAGACTGGGAACCAGAACCA
TCCTGAGACTTGTAATCAATCCATCATTACATACGAAAACAACACCTGGGTCAATCAGACCTATGTG
AACATAAGCAATACAAACTTTGTGGCCGAGCAGGACGTGACATCCGTGGTCCTTGCAGGAAACTCCA
GCCTGTGTCCCATTAGCGGTTGGGCAATTTACTCAAAGGATAACGGCATCAGGATTGGTTCCAAGGG
TGACGTGTTCGTAATCAGGGAGCCATTTATTTCCTGCTCACACCTCGAATGCAGAACCTTCTTCCTG
ACTCAGGGGGCACTCCTGAATGATAAGCATTCCAATGGAACAGTGAAAGACCGCTCCCCCTATAGGA
CATTGATGTCCTGTCCTGTTGGTGAGGCCCCATCTCCTTATAATAGTAGGTTTGAGAGTGTGGCCTG
GTCCGCAAGTGCTTGTCACGATGGGATGTCCTGGCTGACCATTGGTATTTCTGGTCCAGACTCTGGA
GCCGTGGCTGTTCTGAAATATAACGGAATAATCACTGACACAATCAAAAGTTGGCGAAATAATATCC
TGAGGACCCAGGAGAGCGAGTGTGCTTGCATAAATGGAAGTIGTTTCACTATTATGACCGATGGGCC
ATCCGATGGGCAGGCTTCATATAAAATCTTCAAAATCGAAAAGGGTAAGGTTGTGAAGTCCGTCGAA
CTGAATGCTCCTAATTACCATTACGAAGAATGCTCCTGCTACCCCGACGCTGGCAAAGTGATGTGCG
TATGTCGAGATAACTGGCACGGGAGTAATAGACCTTGGGTGTCCTTCGACCAAAACTTGGAATACCA
AATAGGCTACATTTGTTCAGGGGTGTTCGGCGACAATCCTCGGCCAAACGATGGGACAGGTTCCTGT
GGGCCAGTTTCTTCAAACGGAGCCAATGGGGTCAAAGGCTTCAGTTTCAGATACGGCAACGGGGTGT
GGATTGGCCGAACCAAGAGCATTTCCAGCCGAAAGGGATTTGAGATGATTTGGGACCCTAACGGGTG
GACCGAGACGGACAGTTCCTTTTCAGTGAAACAAGATATTGTGGGCATCAACGAATGGAGCGGATAT
AGCGGGTCCTTCGTGCAGCACCCAGAACTCACAGGACTGGATTGTATGCGGCCCTGTTTCTGGGTAG
AACTCATTAGAGGCAGACCCGAAGAGAACACAATCTGGACATCAGGCAGTTCCATTTCCTTCTGCGG
GGTGAATAGCGATACAGTGGGATGGTCTTGGCCTGATGGTGCCGAATTGCCATTCACAATAGATAAG

1. An isolated polypeptide comprising a haemagluttinin subtype 5 (H5) globular head domain, and optionally a haemagluttinin stem domain, wherein the polypeptide comprises an amino acid sequence in which an amino acid residue at a position corresponding to residue position 144 or 145 of a wild-type H5 globular head domain has been deleted.

2. An isolated polypeptide according to claim 1, wherein the polypeptide comprises an amino acid sequence in which an amino acid residue at a position corresponding to residue position 144 of the wild-type H5 globular head domain has been deleted.

3. An isolated polypeptide according to claim 1, wherein the polypeptide comprises an amino acid sequence in which an amino acid residue at a position corresponding to residue position 145 of the wild-type H5 globular head domain has been deleted.

4. An isolated polypeptide according to any of claims 1 to 3, wherein the polypeptide comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:3.

5. An isolated polypeptide according to any preceding claim, which retains at least some HA activity of a wild-type H5 globular head domain.

6. An isolated polypeptide according to any preceding claim, wherein the polypeptide comprises an amino acid sequence with the following amino acid residues at positions corresponding to residues 156, 157, 171, 172, and 205 of the wild-type H5 globular head domain:
156: R;
157: S;
171: N;
172: A; and
205: R

7. An isolated polypeptide according to any preceding claim, which comprises an amino acid sequence of SEQ ID NO:27 or 29, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:27 or 29.

8. An isolated polypeptide according to any preceding claim, which comprises an amino acid sequence of SEQ ID NO:29.

9. An isolated polypeptide according to any preceding claim, which comprises an amino acid sequence of SEQ ID NO:27.

10. An isolated polypeptide according to any preceding claim, wherein the polypeptide comprises a haemagluttinin stem domain, and wherein the polypeptide comprises an amino acid sequence with the following amino acid residues at positions corresponding to residue positions 416 and 434 of a wild-type H5 sequence:
416: F; and
434: F

11. An isolated polypeptide according to claim 10, wherein the polypeptide comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:27.

12. An isolated polypeptide comprising a haemagluttinin subtype 5 (H5) globular head domain, and optionally a haemagluttinin stem domain, wherein the polypeptide comprises an amino acid sequence with the following amino acid residues at positions corresponding to residue positions 148 and 149 of a wild-type H5 globular head domain:
148: V;
149: P.

13. An isolated polypeptide according to claim 12, wherein the polypeptide comprises an amino acid sequence with the following amino acid residue at a position corresponding to residue position 238 of a wild-type H5 globular head domain:
238: E.

14. An isolated polypeptide according to claim 12 or 13, wherein the polypeptide comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:3.

15. An isolated polypeptide according to any of claims 12 to 14, which retains at least some HA activity of a wild-type H5 globular head domain.

16. An isolated polypeptide according to any of claims 12 to 15, which has reduced affinity for its receptor compared with the wild-type H5 globular head domain.

17. An isolated polypeptide according to any of claims 12 to 16, wherein the polypeptide comprises an amino acid sequence with the following amino acid residues at positions corresponding to residues 156, 157, 171, 172, and 205 of the wild-type H5 globular head domain:
156: R;
157: S;
171: N;
172: A; and
205: R.

18. An isolated polypeptide according to any of claims 12 to 17, which comprises an amino acid sequence of SEQ ID NO:35 or 37, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:35 or 37.

19. An isolated polypeptide according to any of claims 12 to 18, which comprises an amino acid sequence of SEQ ID NO:37.

20. An isolated polypeptide according to any of claims 12 to 19, which comprises an amino acid sequence of SEQ ID NO:35.

21. An isolated polypeptide according to any of claims 12 to 20, wherein the polypeptide comprises a haemagluttinin stem domain, and wherein the polypeptide comprises an amino acid sequence with the following amino acid residues at positions corresponding to residue positions 416 and 434 of a wild-type H5 sequence:
416: F; and
434: F

22. An isolated polypeptide according to claim 21, wherein the polypeptide comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of SEQ ID NO:35.

23. An isolated polypeptide comprising a haemagluttinin subtype 5 (H5) globular head domain, and optionally a haemagluttinin stem domain, wherein the polypeptide comprises an amino acid sequence with the following amino acid residue at a position corresponding to residue position 238 of a wild-type H5 globular head domain:
238: E

24. An isolated polypeptide according to claim 23, wherein the polypeptide comprises an amino acid sequence with the following amino acid residues at positions corresponding to residue positions 148 and 149 of a wild-type H5 globular head domain:
148: S;
149: S.

25. An isolated polypeptide according to claim 23 or 24, wherein the polypeptide comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:3.

26. An isolated polypeptide according to any of claims 23 to 25, which retains at least some HA activity of a wild-type H5 globular head domain.

27. An isolated polypeptide according to any of claims 23 to 26, which has reduced affinity for its receptor compared with the wild-type H5 globular head domain.

28. An isolated polypeptide according to any of claims 23 to 27, wherein the polypeptide comprises an amino acid sequence with the following amino acid residues at positions corresponding to residues 156, 157, 171, 172, and 205 of the wild-type H5 globular head domain:
156: R;
157: S;
171: N;
172: A; and
205: R.

29. An isolated polypeptide according to any of claims 23 to 28, which comprises an amino acid sequence of SEQ ID NO:43 or 45, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:43 or 45.

30. An isolated polypeptide according to any of claims 23 to 29, which comprises an amino acid sequence of SEQ ID NO:45.

31. An isolated polypeptide according to any of claims 23 to 30, which comprises an amino acid sequence of SEQ ID NO:43.

32. An isolated polypeptide according to any preceding claim, wherein the polypeptide comprises an amino acid sequence with the following amino acid residues at positions corresponding to residue positions 279 and 298 of the wild-type H5 globular head domain:
279 A; and
298 M

33. An isolated polypeptide according to any preceding claim, wherein the polypeptide comprises a haemagluttinin stem domain, and wherein the polypeptide comprises an amino acid sequence with the following amino acid residues at positions corresponding to residue positions 416 and 434 of a wild-type H5 sequence:
416: F; and
434: F

34. An isolated polypeptide according to claim 33, wherein the polypeptide comprises an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the sequence of any of SEQ ID NO: 27, 35, or 43.

35. An isolated nucleic acid molecule which comprises a nucleotide sequence encoding a polypeptide according to any of claims 1 to 34, or the complement thereof.

36. An isolated nucleic acid molecule according to claim 35, which comprises a nucleotide sequence of SEQ ID NO:28, 30, 32, or 34, or which comprises nucleotide sequence of SEQ ID NOs:32 and 34, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO: 28, 30, 32, 34, or with SEQ ID NO:32 and 34, over its entire length, or the complement thereof.

37. An isolated nucleic acid molecule according to claim 35, which comprises a nucleotide sequence of SEQ ID NO:28, 30, 32, or 34, or which comprises nucleotide sequence of SEQ ID NOs:32 and 34, or the complement thereof.

38. An isolated nucleic acid molecule according to claim 35, which comprises a nucleotide sequence of SEQ ID NO:28, or the complement thereof.

39. An isolated nucleic acid molecule according to claim 35, which comprises a nucleotide sequence of SEQ ID NO:36, 38, 40, or 42, or which comprises nucleotide sequence of SEQ ID NOs:40 and 42, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO: 36, 38, 40, or 42, or with SEQ ID NO:40 and 42, over its entire length, or the complement thereof.

40. An isolated nucleic acid molecule according to claim 35, which comprises a nucleotide sequence of SEQ ID NO:36, 38, 40, or 42, or which comprises nucleotide sequence of SEQ ID NOs:40 and 42, or the complement thereof.

41. An isolated nucleic acid molecule according to claim 35, which comprises a nucleotide sequence of SEQ ID NO:36, or the complement thereof.

42. An isolated nucleic acid molecule according to claim 35, which comprises a nucleotide sequence of SEQ ID NO:44, 46, 48, or 50, or which comprises nucleotide sequence of SEQ ID NOs 48 and 50, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO: 44, 46, 48, or 50, or with SEQ ID NO: 48 and 50, over its entire length, or the complement thereof.

43. An isolated nucleic acid molecule according to claim 35, which comprises a nucleotide sequence of SEQ ID NO:44, 46, 48, or 50, or which comprises nucleotide sequence of SEQ ID NOs 48 and 50, or the complement thereof.

44. An isolated nucleic acid molecule according to claim 35, which comprises a nucleotide sequence of SEQ ID NO:44, or the complement thereof.

45. An isolated nucleic acid molecule which comprises a nucleotide sequence of SEQ ID NO:52, 54, 55, 56, or which comprises nucleotide sequence of SEQ ID NOs:52 and 54, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO: 52, 54, 55, 56, or with SEQ ID NO:52 and 54, over its entire length, or the complement thereof.

46. An isolated nucleic acid molecule which comprises a nucleotide sequence of SEQ ID NO:52, 54, 55, 56, or which comprises nucleotide sequence of SEQ ID NOs:52 and 54, or the complement thereof.

47. An isolated nucleic acid molecule which comprises a nucleotide sequence of SEQ ID NO:55, or the complement thereof.

48. An isolated nucleic acid molecule which comprises a nucleotide sequence of SEQ ID NO:58, 60, 61, 62, or which comprises nucleotide sequence of SEQ ID NOs:58 and 60, or a nucleotide sequence that is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identical with SEQ ID NO: 58, 60, 61, 62, or with SEQ ID NO:58 and 60, over its entire length, or the complement thereof.

49. An isolated nucleic acid molecule which comprises a nucleotide sequence of SEQ ID NO:58, 60, 61, 62, or which comprises nucleotide sequence of SEQ ID NOs:58 and 60, or the complement thereof.

50. An isolated nucleic acid molecule which comprises a nucleotide sequence of SEQ ID NO:61, or the complement thereof.

51. An isolated nucleic acid molecule according to any of claims 35 to 50, which comprises a messenger RNA (mRNA) molecule.

52. A vector comprising a nucleic acid molecule of any of claims 35 to 51.

53. A vector according to claim 52, comprising a nucleic acid molecule encoding a polypeptide of any of claims 1 to 34.

54. A vector according to claim 52 or 53, comprising a nucleic acid molecule encoding a polypeptide which comprises an amino acid sequence of SEQ ID NO:27 or 29.

55. A vector according to any of claims 52 to 54, comprising a nucleic acid molecule encoding a polypeptide which comprises an amino acid sequence of SEQ ID NO:35 or 37.

56. A vector according to any of claims 52 to 55, comprising a nucleic acid molecule encoding a polypeptide which comprises an amino acid sequence of SEQ ID NO:43 or 45.

57. A vector according to any of claims 52 to 56, comprising a nucleic acid molecule encoding a polypeptide which comprises an amino acid sequence of SEQ ID NO:7 or 8.

58. A vector according to any of claims 52 to 57, comprising a nucleic acid molecule encoding a polypeptide which comprises an amino acid sequence of SEQ ID NO:10 or 11.

59. A vector according to any of claims 52 to 57, which further comprises a promoter operably linked to the, or each nucleic acid molecule.

60. A vector according to claim 59, wherein the, or each promoter is for expression of a polypeptide encoded by the nucleic acid in mammalian cells.

61. A vector according to claim 59, wherein the, or each promoter is for expression of a polypeptide encoded by the nucleic acid in yeast or insect cells.

62. A vector according to any of claims 52 to 61, which is a vaccine vector.

63. A vector according to claim 62, which is a viral vaccine vector, a bacterial vaccine vector, an RNA vaccine vector, a messenger RNA (mRNA) vector, or a DNA vaccine vector.

64. An isolated cell comprising a vector of any of claims 52 to 63.

65. A fusion protein comprising a polypeptide according to any of claims 1 to 34.

66. A pharmaceutical composition comprising a polypeptide according to any of claims 1 to 34, and a pharmaceutically acceptable carrier, excipient, or diluent.

67. A pharmaceutical composition according to claim 66, comprising a polypeptide which comprises an amino acid sequence of SEQ ID NO:27 or 29.

68. A pharmaceutical composition according to claim 66, comprising a polypeptide which comprises an amino acid sequence of SEQ ID NO:27.

69. A pharmaceutical composition according to any of claims 66 to 68, comprising a polypeptide which comprises an amino acid sequence of SEQ ID NO:35 or 37.

70. A pharmaceutical composition according to any of claims 66 to 69, comprising a polypeptide which comprises an amino acid sequence of SEQ ID NO:35.

71. A pharmaceutical composition according to any of claims 66 to 70, comprising a polypeptide which comprises an amino acid sequence of SEQ ID NO:43 or 45.

72. A pharmaceutical composition according to any of claims 66 to 71, comprising a polypeptide which comprises an amino acid sequence of SEQ ID NO:43.

73. A pharmaceutical composition comprising a nucleic acid according to any of claims 35 to 51, and a pharmaceutically acceptable carrier, excipient, or diluent.

74. A pharmaceutical composition according to claim 73, comprising a nucleic acid molecule encoding a polypeptide of any of claims 1 to 34.

75. A pharmaceutical composition according to claim 73 or 74, comprising a nucleic acid molecule encoding a polypeptide which comprises an amino acid sequence of SEQ ID NO: 27 or 29.

76. A pharmaceutical composition according to claim 75, which comprises a nucleic acid molecule comprising a nucleotide sequence of SEQ ID NO:28 or 30.

77. A pharmaceutical composition according to any of claims 73 to 76, comprising a nucleic acid molecule encoding a polypeptide which comprises an amino acid sequence of SEQ ID NO: 27.

78. A pharmaceutical composition according to claim 77, which comprises a nucleic acid molecule comprising a nucleotide sequence of SEQ ID NO:28.

79. A pharmaceutical composition according to any of claims 73 to 78, comprising a nucleic acid molecule encoding a polypeptide which comprises an amino acid sequence of SEQ ID NO: 35 or 37.

80. A pharmaceutical composition according to any of claims 73 to 79, comprising a nucleic acid molecule comprising a nucleotide sequence of SEQ ID NO: 36 or 38.

81. A pharmaceutical composition according to any of claims 73 to 80, comprising a nucleic acid molecule encoding a polypeptide which comprises an amino acid sequence of SEQ ID NO: 35.

82. A pharmaceutical composition according to any of claims 73 to 81, comprising a nucleic acid molecule comprising a nucleotide sequence of SEQ ID NO: 36.

83. A pharmaceutical composition according to any of claims 73 to 82, comprising a nucleic acid molecule encoding a polypeptide which comprises an amino acid sequence of SEQ ID NO: 43 or 45.

84. A pharmaceutical composition according to any of claims 73 to 83, comprising a nucleic acid molecule comprising a nucleotide sequence of SEQ ID NO: 44 or 46.

85. A pharmaceutical composition according to any of claims 73 to 84, comprising a nucleic acid molecule encoding a polypeptide which comprises an amino acid sequence of SEQ ID NO: 43.

86. A pharmaceutical composition according to any of claims 73 to 85, comprising a nucleic acid molecule comprising a nucleotide sequence of SEQ ID NO: 44.

87. A pharmaceutical composition comprising a vector according to any of claims 52 to 63, and a pharmaceutically acceptable carrier, excipient, or diluent.

88. A pharmaceutical composition according to any of claims 73 to 86, wherein the nucleic acid comprises a messenger RNA (mRNA) molecule.

89. A pharmaceutical composition according to claim 87 or 88, wherein the vector is a messenger (mRNA) vector.

90. A pharmaceutical composition according to any of claims 66 to 89, which further comprises an adjuvant for enhancing an immune response in a subject to the polypeptide, or to a polypeptide encoded by the nucleic acid, of the composition.

91. A method of inducing an immune response to an influenza virus in a subject, which comprises administering to the subject an effective amount of a polypeptide according to any of claims 1 to 34, a nucleic acid according to any of claims 35 to 51, a vector according to any of claims 52 to 63, or a pharmaceutical composition according to any of claims 66 to 90.

92. A method of immunising a subject against an influenza virus, which comprises administering to the subject an effective amount of a polypeptide according to any of claims 1 to 34, a nucleic acid according to any of claims 35 to 51, a vector according to any of claims 52 to 63, or a pharmaceutical composition according to any of claims 66 to 90.

93. A polypeptide according to any of claims 1 to 34, a nucleic acid according to any of claims 35 to 51, a vector according to any of claims 52 to 63, or a pharmaceutical composition according to any of claims 66 to 90, for use as a medicament.

94. A polypeptide according to any of claims 1 to 34, a nucleic acid according to any of claims 35 to 51, a vector according to any of claims 52 to 63, or a pharmaceutical composition according to any of claims 66 to 90, for use in the prevention, treatment, or amelioration of an influenza viral infection.

95. Use of a polypeptide according to any of claims 1 to 34, a nucleic acid according to any of claims 35 to 51, a vector according to any of claims 52 to 63, or a pharmaceutical composition according to any of claims 66 to 90, in the manufacture of a medicament for the prevention, treatment, or amelioration of an influenza viral infection.

96. An isolated polypeptide which comprises an amino acid sequence of SEQ ID NO:63, or an amino acid sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% amino acid identity along its entire length with the amino acid sequence of SEQ ID NO:63.

97. An isolated polypeptide which comprises an amino acid sequence of SEQ ID NO:63.

98. An isolated nucleic acid molecule, which comprises a nucleotide sequence encoding a polypeptide according to claim 96 or 97, or the complement thereof.

99. An isolated nucleic acid molecule according to claim 98, which comprises a nucleotide sequence of SEQ ID NO:25, or a nucleotide sequence which has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity along its entire length with the nucleotide sequence of SEQ ID NO:25, or the complement thereof.

100. An isolated nucleic acid molecule which comprises a nucleotide sequence of SEQ ID NO:25, or a nucleotide sequence which has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity along its entire length with the nucleotide sequence of SEQ ID NO:25, or the complement thereof.

101. An isolated nucleic acid molecule which comprises a nucleotide sequence of SEQ ID NO:25, or the complement thereof.

102. An isolated nucleic acid molecule according to any of claims 98 to 101, which comprises a DNA molecule.

103. An isolated nucleic acid molecule according to any of claims 98 to 101, which comprises a messenger RNA (mRNA) molecule.

104. A vector comprising a nucleic acid molecule according to any of claims 98 to 103.

105. A vector according to claim 104, which comprises a nucleotide sequence of SEQ ID NO:26.

106. A vector according to claim 104 or 105, comprising a nucleic acid molecule encoding a polypeptide which comprises an amino acid sequence of SEQ ID NO:63.

107. A vector according to any of claims 104 to 106, which further comprises a promoter operably linked to the nucleic acid molecule.

108. A vector according to claim 107, wherein the promoter is for expression of a polypeptide encoded by the nucleic acid in mammalian cells.

109. A vector according to claim 108, wherein the promoter is for expression of a polypeptide encoded by the nucleic acid in yeast or insect cells.

110. A vector according to any of claims 104 to 109, which is a vaccine vector.

111. A vector according to claim 110, which is a viral vaccine vector, a bacterial vaccine vector, an RNA vaccine vector, a messenger RNA (mRNA) vector, or a DNA vaccine vector.

112. An isolated cell comprising a vector of any of claims 104 to 111.

113. A fusion protein comprising a polypeptide according to claim 96 or 97.

114. A pharmaceutical composition which comprises an isolated polypeptide according to claim 96 or 97, and a pharmaceutically acceptable carrier, excipient, or diluent.

115. A pharmaceutical composition which comprises an isolated nucleic acid molecule according to any of claims 98 to 103, and a pharmaceutically acceptable carrier, excipient, or diluent.

116. A pharmaceutical composition according to claim 115, comprising a nucleic acid molecule encoding a polypeptide comprising an amino acid sequence of SEQ ID NO:63, or the complement thereof.

117. A pharmaceutical composition comprising a vector according to any of claims 104 to 111, and a pharmaceutically acceptable carrier, excipient, or diluent.

118. A pharmaceutical composition according to claim 117, comprising a vector comprising a nucleotide sequence of SEQ ID NO:26.

119. A pharmaceutical composition according to claim 115 or 116, wherein the nucleic acid comprises a messenger RNA (mRNA) molecule.

120. A pharmaceutical composition according to claim 117 or 118, wherein the vector is a messenger (mRNA) vector.

121. A pharmaceutical composition according to any of claims 114 to 120, which further comprises an adjuvant for enhancing an immune response in a subject to the polypeptide, or to a polypeptide encoded by the nucleic acid, of the composition.

122. A method of inducing an immune response to an influenza virus in a subject, which comprises administering to the subject an effective amount of a polypeptide according to claim 96 or 97, a nucleic acid molecule according to any of claims 98 to 103, a vector according to any of claims 104 to 111, or a pharmaceutical composition according to any of claims 114 to 121.

123. A method of immunising a subject against an influenza virus, which comprises administering to the subject an effective amount of a polypeptide according to claim 96 or 97, a nucleic acid molecule according to any of claims 98 to 103, a vector according to any of claims 104 to 111, or a pharmaceutical composition according to any of claims 114 to 121.

124. A polypeptide according to claim 96 or 97, a nucleic acid molecule according to any of claims 98 to 103, a vector according to any of claims 104 to 111, or a pharmaceutical composition according to any of claims 114 to 121, for use as a medicament.

125. A polypeptide according to claim 96 or 97, a nucleic acid molecule according to any of claims 98 to 103, a vector according to any of claims 104 to 111, or a pharmaceutical composition according to any of claims 114 to 121, for use in the prevention, treatment, or amelioration of an influenza viral infection.

126. Use of a polypeptide according to claim 96 or 97, a nucleic acid molecule according to any of claims 98 to 103, a vector according to any of claims 104 to 111, or a pharmaceutical composition according to any of claims 114 to 121, in the manufacture of a medicament for the prevention, treatment, or amelioration of an influenza viral infection.


SEQUENCE LISTING (100 sequences)

SEQ ID NO: 1 - protein, 568 aa, synthetic construct
  REGION 1..568: note = Influenza H5 designed sequence
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGRSSFFRNVVWLIKKNNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 2 - DNA, 1704 bp, synthetic construct
  misc_feature 1..1704: note = Influenza H5 designed sequence
  source 1..1704: mol_type = other DNA
atggaaaagattgtgctgctgctggccatcgtgtccctggtcaagagcgatcaaatctgcatcggctaccacgccaacaacagcaccgaacaggtggacaccattatggaaaagaacgtgaccgtgacacacgcccaggacatcctggaaaagacccacaacggcaagctgtgcgacctggatggcgtgaagcctctgatcctgagagattgctctgtggccggctggctgctgggcaatcctatgtgcgacgagttcatcaacgtgcccgagtggtcctatatcgtggaaaaggccaatcctgccaacgacctgtgctaccccggcaacttcaacgactacgaggaactgaaacatctgctgagccggatcaaccacttcgagaagatccagatcatccccaagtcctcttggagcgatcacgaggcctctagcggagtgtctagcgcctgtccttaccaaggcagaagcagcttcttccggaacgtcgtgtggctgatcaagaagaacaacgcttaccccaccatcaagcggagctacaacaacaccaatcaagaggacctgctggtgctgtggggcatccaccatcctaatgatgccgccgagcagacccggctgtaccagaatcctacaacctacatcagcgtgggcaccagcacactgaaccagagactggtgcctaagatcgccaccagatccaaagtgaacggccagagcggccggatggaattcttctggaccatcctgaagcctaacgacgccatcaacttcgagagcaacggcaactttatcgcccctgagtacgcctacaagatcgtgaagaagggcgacagcgccatcatgaagtccgagctggaatacggcaactgcaacaccaagtgtcagacccctatgggcgccatcaatagcagcatgcccttccacaacattcaccctctgaccatcggcgagtgccccaaatacgtgaagtccaacagactggtcctggccaccggcctgagaaattctccacagagagagcggcgcagaaagaagagaggcctgtttggagccattgccggctttatcgaaggcggctggcaaggcatggttgacggatggtacggctatcaccacagcaatgagcaaggctctggctacgccgccgacaaagagagcacacagaaagccatcgacggcgtgaccaacaaagtgaatagcatcatcgacaagatgaacacccagttcgaggccgtgggcagagagttcaacaacctggaaagacggatcgagaacctgaacaagaagatggaggacggcttcctggacgtgtggacctataatgccgagctgctggtcctgatggaaaacgagagaaccctggacttccacgacagcaacgtgaagaacctgtacgacaaagtgcggctccagctgcgggacaatgccaaagaactcggcaacggctgcttcgagttctaccacaagtgcgacaacgagtgcatggaaagcgtgcggaacggcacctacgactaccctcagtactctgaggaagcccggctgaagagagaagagatcagcggagtgaagctggaatccatcggcacataccagatcctgagcatctacagcaccgtggcctcttctctggccctggctattatggtggctggcctgagcctgtggatgtgctctaatggcagcctccagtgccggatctgcatc

SEQ ID NO: 3 - protein, 268 aa, synthetic construct
  REGION 1..268: note = Influenza H5 Head region designed sequence
THNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGRSSFFRNVVWLIKKNNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECP

SEQ ID NO: 4 - DNA, 804 bp, synthetic construct
  misc_feature 1..804: note = Influenza H5 Head region designed sequence
  source 1..804: mol_type = other DNA
acccacaacggcaagctgtgcgacctggatggcgtgaagcctctgatcctgagagattgctctgtggccggctggctgctgggcaatcctatgtgcgacgagttcatcaacgtgcccgagtggtcctatatcgtggaaaaggccaatcctgccaacgacctgtgctaccccggcaacttcaacgactacgaggaactgaaacatctgctgagccggatcaaccacttcgagaagatccagatcatccccaagtcctcttggagcgatcacgaggcctctagcggagtgtctagcgcctgtccttaccaaggcagaagcagcttcttccggaacgtcgtgtggctgatcaagaagaacaacgcttaccccaccatcaagcggagctacaacaacaccaatcaagaggacctgctggtgctgtggggcatccaccatcctaatgatgccgccgagcagacccggctgtaccagaatcctacaacctacatcagcgtgggcaccagcacactgaaccagagactggtgcctaagatcgccaccagatccaaagtgaacggccagagcggccggatggaattcttctggaccatcctgaagcctaacgacgccatcaacttcgagagcaacggcaactttatcgcccctgagtacgcctacaagatcgtgaagaagggcgacagcgccatcatgaagtccgagctggaatacggcaactgcaacaccaagtgtcagacccctatgggcgccatcaatagcagcatgcccttccacaacattcaccctctgaccatcggcgagtgcccc

SEQ ID NO: 5 - protein, 300 aa, synthetic construct
  REGION 1..300: note = Influenza H5 Stem region designed sequence
MEKIVLLLAIVSLVKSDQICIGYHANNSTEQVDTIMEKNVTVTHAQDILEKKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 6 - DNA, 900 bp, synthetic construct
  misc_feature 1..900: note = Influenza H5 stem region designed sequence
  source 1..900: mol_type = other DNA
atggaaaagattgtgctgctgctggccatcgtgtccctggtcaagagcgatcaaatctgcatcggctaccacgccaacaacagcaccgaacaggtggacaccattatggaaaagaacgtgaccgtgacacacgcccaggacatcctggaaaagaaatacgtgaagtccaacagactggtcctggccaccggcctgagaaattctccacagagagagcggcgcagaaagaagagaggcctgtttggagccattgccggctttatcgaaggcggctggcaaggcatggttgacggatggtacggctatcaccacagcaatgagcaaggctctggctacgccgccgacaaagagagcacacagaaagccatcgacggcgtgaccaacaaagtgaatagcatcatcgacaagatgaacacccagttcgaggccgtgggcagagagttcaacaacctggaaagacggatcgagaacctgaacaagaagatggaggacggcttcctggacgtgtggacctataatgccgagctgctggtcctgatggaaaacgagagaaccctggacttccacgacagcaacgtgaagaacctgtacgacaaagtgcggctccagctgcgggacaatgccaaagaactcggcaacggctgcttcgagttctaccacaagtgcgacaacgagtgcatggaaagcgtgcggaacggcacctacgactaccctcagtactctgaggaagcccggctgaagagagaagagatcagcggagtgaagctggaatccatcggcacataccagatcctgagcatctacagcaccgtggcctcttctctggccctggctattatggtggctggcctgagcctgtggatgtgctctaatggcagcctccagtgccggatctgcatc

SEQ ID NO: 7 - protein, 568 aa, synthetic construct
  REGION 1..568: note = Influenza H5 designed sequence
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGRPSFFRNVVWLIKKNDTYPTIKRSYNNTNQEDLLVLWGIHHPNDAAEQTKLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 8 - protein, 268 aa, synthetic construct
  REGION 1..268: note = Influenza H5 head region designed sequence
THNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGRPSFFRNVVWLIKKNDTYPTIKRSYNNTNQEDLLVLWGIHHPNDAAEQTKLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECP

SEQ ID NO: 9 - protein, 300 aa, synthetic construct; identical to SEQ ID NO: 5
  REGION 1..300: note = Influenza H5 stem region designed sequence

SEQ ID NO: 10 - protein, 568 aa, synthetic construct
  REGION 1..568: note = Influenza H5 designed sequence
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGRPSFFRNVVWLIKKNNTYPTIKRSYNNTNQEDLLVLWGIHHPNDAAEQTKLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 11 - protein, 268 aa, synthetic construct
  REGION 1..268: note = Influenza H5 head region designed sequence
THNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGRPSFFRNVVWLIKKNNTYPTIKRSYNNTNQEDLLVLWGIHHPNDAAEQTKLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECP

SEQ ID NO: 12 - protein, 300 aa, synthetic construct; identical to SEQ ID NO: 5
  REGION 1..300: note = Influenza H5 stem region designed sequence

SEQ ID NO: 13 - protein, 50 aa, synthetic construct
  REGION 1..50: note = Influenza H5 head domain fragment designed sequence
  SITE 2: note = MISC_FEATURE - Residue 2 is P or S
  SITE 16: note = MISC_FEATURE - Residue 16 is D or N
  SITE 17: note = MISC_FEATURE - Residue 17 is T or A
  SITE 50: note = MISC_FEATURE - Residue 50 is K or R
RXSFFRNVVWLIKKNXXYPTIKRSYNNTNQEDLLVLWGIHHPNDAAEQTX

SEQ ID NO: 14 - protein, 97 aa, synthetic construct
  REGION 1..97: note = Influenza M2 designed sequence
MSLLTEVETPTRNGWECRCSDSSDPLVIAASIIGILHLILWILDRLFFKCIYRRLKYGLKRGPSTEGVPESMREEYRQKQQSAVDVDDGHFVNIELE

SEQ ID NO: 15 - DNA, 291 bp, synthetic construct
  misc_feature 1..291: note = Influenza M2 designed sequence
  source 1..291: mol_type = other DNA
atgtctctgctgaccgaggtggaaacccctaccagaaatggctgggagtgcagatgcagcgacagcagcgatcctctggttatcgccgccagcatcatcggcatcctgcacctgatcctgtggatcctggaccggctgttcttcaagtgcatctaccggcggctgaagtacggcctgaagagaggcccttctacagagggcgtgcccgagagcatgcgggaagagtacagacagaaacagcagagcgccgtggacgtggacgatggccacttcgtgaacatcgagctggaa

SEQ ID NO: 16 - protein, 469 aa, synthetic construct
  REGION 1..469: note = Influenza NA designed sequence
MNPNQKIITIGSICMVVGIISLILQIGNIISIWVSHSIQTGNQNQPETCNQSIITYENNTWVNQTYVNISNTNFVAEQAVASVALAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACINGSCFTIMTDGPSNGQASYKIFKIEKGKVVKSVELNAPNYHYEECSCYPDAGEVMCVCRDNWHGSNRPWVSFNQNLEYQIGYICSGVFGDNPRPNDGTGSCGPVSSNGAYGVKGFSFKYGKGVWIGRTKSTSSRSGFEMIWDPNGWTETDSSFSVKQDIVAITDWSGYSGSFVQHPELTGLDCMRPCFWVELIRGRPKENTIWTSGSSISFCGVNSDTVGWSWPDGAELPFTIDK

SEQ ID NO: 17 - DNA, 1407 bp, synthetic construct
  misc_feature 1..1407: note = Influenza NA designed sequence
  source 1..1407: mol_type = other DNA
atgaatccaaatcagaaaataataaccattgggtcaatctgtatggtagttggaataatcagcctaatattacaaattgggaacataatctcaatatgggttagccattcaattcagactggaaatcaaaaccaacctgaaacatgcaaccaaagcatcattacttatgaaaacaacacttgggtgaatcaaacatatgttaacatcagcaataccaattttgttgctgaacaggctgtagcttcagtggcattagcgggcaattcctctctctgccccattagtgggtgggctatatacagcaaggacaatggcataaggattggttccaagggagatgtatttgtcataagagagccattcatttcatgctcccacttggaatgcaggaccttttttctgactcaaggagccttgttgaatgacaaacattccaatggaaccgttaaagacagaagcccctacagaaccttaatgagctgtcctgttggtgaggctccctctccatacaattcaaggtttgagtcggttgcttggtcagcaagtgcttgccatgatggcattagctggttgacaattggaatttccgggccagacaatggggcagtggctgtattgaaatacaatggcataataacagacactatcaaaagttggagaaacaacatattgaggacacaagagtctgaatgtgcctgcataaatggttcttgctttactataatgaccgatggaccaagtaatgggcaggcctcatacaagattttcaagatagagaaggggaaggtagtcaaatcagtcgagttgaatgcccctaattaccactacgaggaatgttcctgttatcctgatgctggcgaagtaatgtgtgtgtgcagggataattggcatggttcgaatcgaccatgggtgtctttcaatcaaaatctggagtatcaaataggatacatatgcagtggggttttcggagacaatccacgccccaatgatggaacaggcagctgtggtccagtgtcttctaatggagcatatggagtaaagggattttcatttaagtacggcaagggtgtttggatagggagaactaagagcactagttccaggagtggatttgagatgatttgggatcccaatggatggacagagacagatagtagtttctcagtgaagcaagatattgtagcaataactgattggtcaggatatagcgggagttttgtccaacatccagaattaacagggctggactgcatgaggccttgcttctgggttgaactaatcagaggacggcctaaggagaacacaatctggactagtgggagcagcatttccttctgtggtgtaaatagcgacactgtgggttggtcttggccagacggtgctgagttgccattcaccattgacaag

SEQ ID NO: 18 - protein, 469 aa, synthetic construct
  REGION 1..469: note = Influenza NA designed sequence
MNPNQKIITIGSICMVVGIISLILQIGNIISIWVSHSIQTGNQNHPETCNQSIITYENNTWVNQTYVNISNTNVVAGQDATSVILAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGMGWLTIGISGPDNGAVAVLKYNGIITDTIKSWRNNILRTQESECACVNGSCFTIMTDGPSNGQASYKIFKIEKGKVIKSIELNAPNYHYEECSCYPDTGKVMCVCRDNWHGSNRPWVSFDQNLDYQIGYICSGVFGDNPRPNDGTGSCGPVSSNGANGVKGFSFRYGNGVWIGRTKSTSSRSGFEMIWDPNGWTETDSSFSVKQDIVAITDWSGYSGSFVQHPELTGLDCMRPCFWVELIRGQPKENTIWTSGSSISFCGVNSDTVGWSWPDGAELPFTIDK

SEQ ID NO: 19 - DNA, 1407 bp, synthetic construct
  misc_feature 1..1407: note = Influenza NA designed sequence
  source 1..1407: mol_type = other DNA
atgaatccaaatcaaaaaataataaccattgggtcaatctgtatggtagttggaataattagcctaatattgcaaatagggaatataatctcaatatgggttagccattcaattcaaactggaaatcaaaaccatcctgaaacatgcaaccaaagcatcattacctatgaaaataacacctgggtgaatcaaacatatgttaacattagcaatactaacgttgttgctggacaggatgcaacttcagtgatattagccggcaattcctctctttgccccatcagtgggtgggctatatacagcaaagacaatggcataagaattggttccaaaggagacgtttttgtcataagagagccatttatttcatgctctcacttggaatgcaggaccttttttctgactcaaggcgccttgctgaatgacaagcattcaaatgggaccgtcaaggacagaagcccctatagaaccttaatgagctgccctgttggtgaagctccgtctccgtacaattcaaggttcgaatcggttgcttggtcagcaagtgcatgccatgatggcatgggctggctaacaatcggaatttccggtccagataatggagcagtggctgtattaaaatacaatggtataataacagacaccatcaaaagttggaggaacaacatattgagaacgcaagagtctgaatgtgcctgtgtaaatggttcatgttttactataatgaccgatggcccaagtaatgggcaggcctcgtacaaaattttcaagatagagaaggggaaggttattaaatcaattgagttgaatgcacctaattaccactacgaggaatgttcctgttaccctgatacaggtaaagtgatgtgtgtgtgcagagacaattggcatggttcgaatcgaccatgggtgtctttcgatcaaaatctggattatcaaataggatacatctgcagtggggttttcggtgacaatccgcgtcccaatgatggaacaggcagctgtggtccagtgtcttctaatggagcaaatggagtaaagggattttcatttaggtatggtaatggtgtttggataggaagaactaaaagtaccagttccagaagcgggtttgagatgatttgggatcctaatggatggacagagactgatagtagtttctctgtgaaacaagatattgtagcaataactgattggtcagggtacagcgggagtttcgttcaacatcctgagctaacagggctggactgcatgaggccttgcttctgggttgaattaatcaggggacaacctaaagagaacacaatctggactagtgggagcagcatttccttttgtggcgtaaatagtgatactgtaggttggtcttggccagacggtgctgagttgccattcaccattgacaag

SEQ ID NO: 20 - DNA, 91 bp, synthetic construct
  misc_feature 1..91: note = pEVAC Multiple Cloning Site sequence
  source 1..91: mol_type = other DNA
acagactgttcctttccatgggtcttttctgcagtcaccgtcggtaccgtcgacacgtgtgatcatctagaggatccgcggccgcagatct

SEQ ID NO: 21 - DNA, 4405 bp, synthetic construct
  misc_feature 1..4405: note = pEVAC sequence
  source 1..4405: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtaccgtcgacacgtgtgatcatctagaggatccgcggccgcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 22 - protein, 566 aa, synthetic construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFINYEELREQLSSVSSFERFEIFPKTSSWPNHDSNKGVTAACPHAGAKSFYKNLIWLVKKGNSYPKLSKSYINDKGKEVLVLWGIHHPSTTADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICI

SEQ ID NO: 23 - DNA, 1698 bp, synthetic construct
  source 1..1698: mol_type = other DNA
atgaaggctattctggtggtgctgctgtacaccttcgccaccgccaatgccgatacactgtgtattggctaccacgccaacaacagcaccgacaccgtggataccgtgctggaaaagaacgtgaccgtgacacacagcgtgaacctgctggaagataagcacaacggcaagctgtgcaagctgagaggcgttgcacctctgcacctgggcaagtgtaatatcgccggctggatcctgggcaaccctgagtgtgaaagcctgagcacagccagcagctggtcctacatcgtggaaaccagcagcagcgacaacggcacatgctaccccggcgacttcatcaactacgaggaactgagagagcagctgagcagcgtcagcagcttcgagagattcgagattttccccaagacctccagctggcccaaccacgattctaacaagggcgtgacagccgcctgtcctcatgccggcgctaagagcttctacaagaacctgatctggctggtcaagaagggcaacagctaccccaagctgagcaagagctacatcaacgacaagggcaaagaggtgctggtcctctggggcatccaccatccttctacaacagccgaccagcagagcctgtaccagaatgccgatgcctacgtgttcgtgggcaccagcagatacagcaagaagttcaagcccgagatcgccatcagacccaaagtgcgggatcaagagggcagaatgaactactactggaccctggtggaacccggcgacaagatcacatttgaggccacaggcaacctggtggtccctagatacgccttcgccatggaaagaaatgccggcagcggcatcatcatcagcgacacacctgtgcacgactgcaacaccacctgtcagacacctgagggcgccatcaataccagcctgcctttccagaacattcaccccatcaccatcggcaagtgccccaaatacgtgaagtccacaaagctgagactggccaccggcctgagaaatgtgcctagcatccagagcagaggcctgtttggagccattgccggctttatcgaaggcggctggacaggcatggttgacggatggtacggctaccaccatcagaatgagcaaggcagcggatacgccgccgatctgaagtctacacagaacgccatcgataagatcaccaacaaagtgaacagcgtgatcgagaagatgaacacccagttcaccgccgtgggaaaagagttcaaccacctggaaaagcgcatcgagaacctgaacaagaaggtggacgacggcttcctggacatctggacctataatgccgagctgctcgtgctgctcgagaacgagagaaccctggactaccacgacagcaacgtgaagaacctgtacgagaaagtgcggaaccagctgaagaacaacgccaaagagatcggcaacggctgcttcgagttctaccacaagtgcgacaatacctgcatggaaagcgtgaagaatggcacctacgactaccctaagtacagcgaggaagccaagctgaaccgcgagaagattgacggcgtgaagctggaaagcacccggatctatcagatcctggccatctacagcacagtggcctctagcctggtgctggtggtgtctctgggagccatcagcttttggatgtgcagcaatggcagcctccagtgccggatctgcatc

SEQ ID NO: 24 - DNA, 6083 bp, synthetic construct
  source 1..6083: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtaccgccaccatgaaggctattctggtggtgctgctgtacaccttcgccaccgccaatgccgatacactgtgtattggctaccacgccaacaacagcaccgacaccgtggataccgtgctggaaaagaacgtgaccgtgacacacagcgtgaacctgctggaagataagcacaacggcaagctgtgcaagctgagaggcgttgcacctctgcacctgggcaagtgtaatatcgccggctggatcctgggcaaccctgagtgtgaaagcctgagcacagccagcagctggtcctacatcgtggaaaccagcagcagcgacaacggcacatgctaccccggcgacttcatcaactacgaggaactgagagagcagctgagcagcgtcagcagcttcgagagattcgagattttccccaagacctccagctggcccaaccacgattctaacaagggcgtgacagccgcctgtcctcatgccggcgctaagagcttctacaagaacctgatctggctggtcaagaagggcaacagctaccccaagctgagcaagagctacatcaacgacaagggcaaagaggtgctggtcctctggggcatccaccatccttctacaacagccgaccagcagagcctgtaccagaatgccgatgcctacgtgttcgtgggcaccagcagatacagcaagaagttcaagcccgagatcgccatcagacccaaagtgcgggatcaagagggcagaatgaactactactggaccctggtggaacccggcgacaagatcacatttgaggccacaggcaacctggtggtccctagatacgccttcgccatggaaagaaatgccggcagcggcatcatcatcagcgacacacctgtgcacgactgcaacaccacctgtcagacacctgagggcgccatcaataccagcctgcctttccagaacattcaccccatcaccatcggcaagtgccccaaatacgtgaagtccacaaagctgagactggccaccggcctgagaaatgtgcctagcatccagagcagaggcctgtttggagccattgccggctttatcgaaggcggctggacaggcatggttgacggatggtacggctaccaccatcagaatgagcaaggcagcggatacgccgccgatctgaagtctacacagaacgccatcgataagatcaccaacaaagtgaacagcgtgatcgagaagatgaacacccagttcaccgccgtgggaaaagagttcaaccacctggaaaagcgcatcgagaacctgaacaagaaggtggacgacggcttcctggacatctggacctataatgccgagctgctcgtgctgctcgagaacgagagaaccctggactaccacgacagcaacgtgaagaacctgtacgagaaagtgcggaaccagctgaagaacaacgccaaagagatcggcaacggctgcttcgagttctaccacaagtgcgacaatacctgcatggaaagcgtgaagaatggcacctacgactaccctaagtacagcgaggaagccaagctgaaccgcgagaagattgacggcgtgaagctggaaagcacccggatctatcagatcctggccatctacagcacagtggcctctagcctggtgctggtggtgtctctgggagccatcagcttttggatgtgcagcaatggcagcctccagtgccggatctgcatctgagcggccgcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 25 - DNA, 3528 bp, synthetic construct
  source 1..3528: mol_type = other DNA
atgaaggctattctggtggtgctgctgtacaccttcgccaccgccaatgccgatacactgtgtattggctaccacgccaacaacagcaccgacaccgtggataccgtgctggaaaagaacgtgaccgtgacacacagcgtgaacctgctggaagataagcacaacggcaagctgtgcaagctgagaggcgttgcacctctgcacctgggcaagtgtaatatcgccggctggatcctgggcaaccctgagtgtgaaagcctgagcacagccagcagctggtcctacatcgtggaaaccagcagcagcgacaacggcacatgctaccccggcgacttcatcaactacgaggaactgagagagcagctgagcagcgtcagcagcttcgagagattcgagattttccccaagacctccagctggcccaaccacgattctaacaagggcgtgacagccgcctgtcctcatgccggcgctaagagcttctacaagaacctgatctggctggtcaagaagggcaacagctaccccaagctgagcaagagctacatcaacgacaagggcaaagaggtgctggtcctctggggcatccaccatccttctacaacagccgaccagcagagcctgtaccagaatgccgatgcctacgtgttcgtgggcaccagcagatacagcaagaagttcaagcccgagatcgccatcagacccaaagtgcgggatcaagagggcagaatgaactactactggaccctggtggaacccggcgacaagatcacatttgaggccacaggcaacctggtggtccctagatacgccttcgccatggaaagaaatgccggcagcggcatcatcatcagcgacacacctgtgcacgactgcaacaccacctgtcagacacctgagggcgccatcaataccagcctgcctttccagaacattcaccccatcaccatcggcaagtgccccaaatacgtgaagtccacaaagctgagactggccaccggcctgagaaatgtgcctagcatccagagcagaggcctgtttggagccattgccggctttatcgaaggcggctggacaggcatggttgacggatggtacggctaccaccatcagaatgagcaaggcagcggatacgccgccgatctgaagtctacacagaacgccatcgataagatcaccaacaaagtgaacagcgtgatcgagaagatgaacacccagttcaccgccgtgggaaaagagttcaaccacctggaaaagcgcatcgagaacctgaacaagaaggtggacgacggcttcctggacatctggacctataatgccgagctgctcgtgctgctcgagaacgagagaaccctggactaccacgacagcaacgtgaagaacctgtacgagaaagtgcggaaccagctgaagaacaacgccaaagagatcggcaacggctgcttcgagttctaccacaagtgcgacaatacctgcatggaaagcgtgaagaatggcacctacgactaccctaagtacagcgaggaagccaagctgaaccgcgagaagattgacggcgtgaagctggaaagcacccggatctatcagatcctggccatctacagcacagtggcctctagcctggtgctggtggtgtctctgggagccatcagcttttggatgtgcagcaatggcagcctccagtgccggatctgcatcggaagcggagaaggcagaggcagcctgctgacatgcggagatgtggaagagaatcccggacctatgaatcccaaccagaagatcatcaccatcggcagcatctgcatggtcgtgggcatcatcagcctgatcctccagatcggcaacatcatctccatctgggtgtcccacagcatccagaccggcaatcagaaccagcctgagacatgcaaccagtccatcatcacctacgagaacaacacctgggtcaaccagacctacgtgaacatcagcaacaccaacttcgtggccgaacaggccgtggcttctgttgccctggccggaaatagctctctgtgccctattagcggctgggccatctacagcaaggacaacggcatccggatcggctctaagggcgacgtgttcgtgatcagagagcccttcatcagctgctcccacctggaatgccggacattctttctgacccaaggcgccctgctgaacgacaagcacagcaatggcaccgtgaaggacagaagcccctacagaaccctgatgagctgccctgtgggagaagccccatctccttacaacagcagattcgagtccgtggcttggagcgcctctgcctgtcacgatggaatcagctggctgacaatcggcatcagcggccctgataatggcgctgtggccgtgctgaagtacaacggaatcatcaccgacaccatcaagagctggcggaacaacatcctgcggacccaagagtccgagtgcgcctgtatcaatggcagctgcttcaccatcatgacagacggccctagcaatggccaggccagctacaagattttcaagatcgagaagggcaaagtggtcaagagcgtggaactgaacgcccctaactaccactacgaggaatgcagctgctaccccgatgccggcgaagtgatgtgcgtgtgcagagacaattggcacggcagcaacagaccttgggtgtccttcaaccagaacctggaatatcagatcggctatatctgctccggcgtgttcggcgacaaccccagacctaatgatggcacaggcagctgtggccccgtgtcatctaatggcgcctatggcgtgaagggcttcagctttaagtacggcaaaggcgtgtggatcggccggaccaagagcacctctagcagatccggcttcgagatgatctgggaccccaacggctggaccgagacagatagcagcttcagcgtgaagcaggacatcgtggccatcaccgattggagcggctacagcggaagcttcgtgcagcaccctgaactgacaggcctggactgcatgaggccctgcttttgggtcgagctgatccggggcagacccaaagagaacaccatctggacaagcggcagcagcatcagcttttgcggcgtgaacagcgataccgtcggctggtcttggcctgatggtgccgagctgcctttcaccatcgacaaaggatccggcgccaccaactttagtctgctgaaacaggccggcgacgtcgaagagaacccaggtcctatgtctctgctgaccgaggtggaaacccctaccagaaatggctgggagtgcagatgcagcgacagcagcgatcctctggttatcgccgccagcatcatcggcatcctgcacctgatcctgtggatcctggaccggctgttcttcaagtgcatctaccggcggctgaagtacggcctgaagagaggcccttctacagagggcgtgcccgagagcatgcgggaagagtacagacagaaacagcagagcgccgtggacgtggacgatggccacttcgtgaacatcgagctggaatga

SEQ ID NO: 26 - DNA, 7910 bp, synthetic construct
  source 1..7910: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtaccgccaccatgaaggctattctggtggtgctgctgtacaccttcgccaccgccaatgccgatacactgtgtattggctaccacgccaacaacagcaccgacaccgtggataccgtgctggaaaagaacgtgaccgtgacacacagcgtgaacctgctggaagataagcacaacggcaagctgtgcaagctgagaggcgttgcacctctgcacctgggcaagtgtaatatcgccggctggatcctgggcaaccctgagtgtgaaagcctgagcacagccagcagctggtcctacatcgtggaaaccagcagcagcgacaacggcacatgctaccccggcgacttcatcaactacgaggaactgagagagcagctgagcagcgtcagcagcttcgagagattcgagattttccccaagacctccagctggcccaaccacgattctaacaagggcgtgacagccgcctgtcctcatgccggcgctaagagcttctacaagaacctgatctggctggtcaagaagggcaacagctaccccaagctgagcaagagctacatcaacgacaagggcaaagaggtgctggtcctctggggcatccaccatccttctacaacagccgaccagcagagcctgtaccagaatgccgatgcctacgtgttcgtgggcaccagcagatacagcaagaagttcaagcccgagatcgccatcagacccaaagtgcgggatcaagagggcagaatgaactactactggaccctggtggaacccggcgacaagatcacatttgaggccacaggcaacctggtggtccctagatacgccttcgccatggaaagaaatgccggcagcggcatcatcatcagcgacacacctgtgcacgactgcaacaccacctgtcagacacctgagggcgccatcaataccagcctgcctttccagaacattcaccccatcaccatcggcaagtgccccaaatacgtgaagtccacaaagctgagactggccaccggcctgagaaatgtgcctagcatccagagcagaggcctgtttggagccattgccggctttatcgaaggcggctggacaggcatggttgacggatggtacggctaccaccatcagaatgagcaaggcagcggatacgccgccgatctgaagtctacacagaacgccatcgataagatcaccaacaaagtgaacagcgtgatcgagaagatgaacacccagttcaccgccgtgggaaaagagttcaaccacctggaaaagcgcatcgagaacctgaacaagaaggtggacgacggcttcctggacatctggacctataatgccgagctgctcgtgctgctcgagaacgagagaaccctggactaccacgacagcaacgtgaagaacctgtacgagaaagtgcggaaccagctgaagaacaacgccaaagagatcggcaacggctgcttcgagttctaccacaagtgcgacaatacctgcatggaaagcgtgaagaatggcacctacgactaccctaagtacagcgaggaagccaagctgaaccgcgagaagattgacggcgtgaagctggaaagcacccggatctatcagatcctggccatctacagcacagtggcctctagcctggtgctggtggtgtctctgggagccatcagcttttggatgtgcagcaatggcagcctccagtgccggatctgcatcggaagcggagaaggcagaggcagcctgctgacatgcggagatgtggaagagaatcccggacctatgaatcccaaccagaagatcatcaccatcggcagcatctgcatggtcgtgggcatcatcagcctgatcctccagatcggcaacatcatctccatctgggtgtcccacagcatccagaccggcaatcagaaccagcctgagacatgcaaccagtccatcatcacctacgagaacaacacctgggtcaaccagacctacgtgaacatcagcaacaccaacttcgtggccgaacaggccgtggcttctgttgccctggccggaaatagctctctgtgccctattagcggctgggccatctacagcaaggacaacggcatccggatcggctctaagggcgacgtgttcgtgatcagagagcccttcatcagctgctcccacctggaatgccggacattctttctgacccaaggcgccctgctgaacgacaagcacagcaatggcaccgtgaaggacagaagcccctacagaaccctgatgagctgccctgtgggagaagccccatctccttacaacagcagattcgagtccgtggcttggagcgcctctgcctgtcacgatggaatcagctggctgacaatcggcatcagcggccctgataatggcgctgtggccgtgctgaagtacaacggaatcatcaccgacaccatcaagagctggcggaacaacatcctgcggacccaagagtccgagtgcgcctgtatcaatggcagctgcttcaccatcatgacagacggccctagcaatggccaggccagctacaagattttcaagatcgagaagggcaaagtggtcaagagcgtggaactgaacgcccctaactaccactacgaggaatgcagctgctaccccgatgccggcgaagtgatgtgcgtgtgcagagacaattggcacggcagcaacagaccttgggtgtccttcaaccagaacctggaatatcagatcggctatatctgctccggcgtgttcggcgacaaccccagacctaatgatggcacaggcagctgtggccccgtgtcatctaatggcgcctatggcgtgaagggcttcagctttaagtacggcaaaggcgtgtggatcggccggaccaagagcacctctagcagatccggcttcgagatgatctgggaccccaacggctggaccgagacagatagcagcttcagcgtgaagcaggacatcgtggccatcaccgattggagcggctacagcggaagcttcgtgcagcaccctgaactgacaggcctggactgcatgaggccctgcttttgggtcgagctgatccggggcagacccaaagagaacaccatctggacaagcggcagcagcatcagcttttgcggcgtgaacagcgataccgtcggctggtcttggcctgatggtgccgagctgcctttcaccatcgacaaaggatccggcgccaccaactttagtctgctgaaacaggccggcgacgtcgaagagaacccaggtcctatgtctctgctgaccgaggtggaaacccctaccagaaatggctgggagtgcagatgcagcgacagcagcgatcctctggttatcgccgccagcatcatcggcatcctgcacctgatcctgtggatcctggaccggctgttcttcaagtgcatctaccggcggctgaagtacggcctgaagagaggcccttctacagagggcgtgcccgagagcatgcgggaagagtacagacagaaacagcagagcgccgtggacgtggacgatggccacttcgtgaacatcgagctggaatgagcggccgcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 27 - protein, 567 aa, synthetic construct
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASGVSSACPYQGRSSFFRNVVWLIKKNNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 28 - DNA, 1704 bp, synthetic construct
  source 1..1704: mol_type = other DNA
atggaaaagattgtgctgctgctggccatcgtgtccctggtcaagagcgatcaaatctgcatcggctaccacgccaacaacagcaccgaacaggtggacaccattatggaaaagaacgtgaccgtgacacacgcccaggacatcctggaaaagacccacaacggcaagctgtgcgacctggatggcgtgaagcctctgatcctgagagattgctctgtggccggctggctgctgggcaatcctatgtgcgacgagttcatcaacgtgcccgagtggtcctatatcgtggaaaaggccaatcctgccaacgacctgtgctaccccggcaacttcaacgactacgaggaactgaaacatctgctgagccggatcaaccacttcgagaagatccagatcatccccaagtcctcttggagcgatcacgaggcctctggagtgtctagcgcctgtccttaccaaggcagaagcagcttcttccggaacgtcgtgtggctgatcaagaagaacaacgcttaccccaccatcaagcggagctacaacaacaccaatcaagaggacctgctggtgctgtggggcatccaccatcctaatgatgccgccgagcagacccggctgtaccagaatcctacaacctacatcagcgtgggcaccagcacactgaaccagagactggtgcctaagatcgccaccagatccaaagtgaacggccagagcggccggatggaattcttctggaccatcctgaagcctaacgacgccatcaacttcgagagcaacggcaactttatcgcccctgagtacgcctacaagatcgtgaagaagggcgacagcgccatcatgaagtccgagctggaatacggcaactgcaacaccaagtgtcagacccctatgggcgccatcaatagcagcatgcccttccacaacattcaccctctgaccatcggcgagtgccccaaatacgtgaagtccaacagactggtcctggccaccggcctgagaaattctccacagagagagcggcgcagaaagaagagaggcctgtttggagccattgccggctttatcgaaggcggctggcaaggcatggttgacggatggtacggctatcaccacagcaatgagcaaggctctggctacgccgccgacaaagagagcacacagaaagccatcgacggcgtgaccaacaaagtgaatagcatcatcgacaagatgaacacccagttcgaggccgtgggcagagagttcaacaacctggaaagacggatcgagaacctgaacaagaagatggaggacggcttcctggacgtgtggacctataatgccgagctgctggtcctgatggaaaacgagagaaccctggacttccacgacagcaacgtgaagaacctgtacgacaaagtgcggctccagctgcgggacaatgccaaagaactcggcaacggctgcttcgagttctaccacaagtgcgacaacgagtgcatggaaagcgtgcggaacggcacctacgactaccctcagtactctgaggaagcccggctgaagagagaagagatcagcggagtgaagctggaatccatcggcacataccagatcctgagcatctacagcaccgtggcctcttctctggccctggctattatggtggctggcctgagcctgtggatgtgctctaatggcagcctccagtgccggatctgcatctga

SEQ ID NO: 29 - protein, 267 aa, synthetic construct
THNGKLCDLDGVKPLILRDCSVAGWLLGNPMCDEFINVPEWSYIVEKANPANDLCYPGNFNDYEELKHLLSRINHFEKIQIIPKSSWSDHEASGVSSACPYQGRSSFFRNVVWLIKKNNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPEYAYKIVKKGDSAIMKSELEYGNCNTKCQTPMGAINSSMPFHNIHPLTIGECP

SEQ ID NO: 30 - DNA, 801 bp, synthetic construct
  source 1..801: mol_type = other DNA
acccacaacggcaagctgtgcgacctggatggcgtgaagcctctgatcctgagagattgctctgtggccggctggctgctgggcaatcctatgtgcgacgagttcatcaacgtgcccgagtggtcctatatcgtggaaaaggccaatcctgccaacgacctgtgctaccccggcaacttcaacgactacgaggaactgaaacatctgctgagccggatcaaccacttcgagaagatccagatcatccccaagtcctcttggagcgatcacgaggcctctggagtgtctagcgcctgtccttaccaaggcagaagcagcttcttccggaacgtcgtgtggctgatcaagaagaacaacgcttaccccaccatcaagcggagctacaacaacaccaatcaagaggacctgctggtgctgtggggcatccaccatcctaatgatgccgccgagcagacccggctgtaccagaatcctacaacctacatcagcgtgggcaccagcacactgaaccagagactggtgcctaagatcgccaccagatccaaagtgaacggccagagcggccggatggaattcttctggaccatcctgaagcctaacgacgccatcaacttcgagagcaacggcaactttatcgcccctgagtacgcctacaagatcgtgaagaagggcgacagcgccatcatgaagtccgagctggaatacggcaactgcaacaccaagtgtcagacccctatgggcgccatcaatagcagcatgcccttccacaacattcaccctctgaccatcggcgagtgcccc

SEQ ID NO: 31 - protein, 51 aa, synthetic construct
MEKIVLLLAIVSLVKSDQICIGYHANNSTEQVDTIMEKNVTVTHAQDILEK

SEQ ID NO: 32 - DNA, 153 bp, synthetic construct
  source 1..153: mol_type = other DNA
atggaaaagattgtgctgctgctggccatcgtgtccctggtcaagagcgatcaaatctgcatcggctaccacgccaacaacagcaccgaacaggtggacaccattatggaaaagaacgtgaccgtgacacacgcccaggacatcctggaaaag

SEQ ID NO: 33 - protein, 249 aa, synthetic construct
KYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 34 - DNA, 749 bp, synthetic construct
  source 1..749: mol_type = other DNA
aaatacgtgaagtccaacagactggtcctggccaccggcctgagaaattctccacagagagagcggcgcagaaagaagagaggcctgtttggagccattgccggctttatcgaaggcggctggcaaggcatggttgacggatggtacggctatcaccacagcaatgagcaaggctctggctacgccgccgacaaagagagcacacagaaagccatcgacggcgtgaccaacaaagtgaatagcatcatcgacaagatgaacacccagttcgaggccgtgggcagagagttcaacaacctggaaagacggatcgagaacctgaacaagaagatggaggacggcttcctggacgtgtggacctataatgccgagctgctggtcctgatggaaaacgagagaaccctggacttccacgacagcaacgtgaagaacctgtacgacaaagtgcggctccagctgcgggacaatgccaaagaactcggcaacggctgcttcgagttctaccacaagtgcgacaacgagtgcatggaaagcgtgcggaacggcacctacgactaccctcagtactctgaggaagcccggctgaagagagaagagatcagcggagtgaagctggaatccatcggcacataccagatcctgagcatctacagcaccgtggcctctctctggccctggctattatggtggctggcctgagcctgtggatgtgctctaatggcagcctccagtgccggatctgcatctga

SEQ ID NO: 35 - protein, 568 aa, synthetic construct
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVVPACPYQGRSSFFRNVVWLIKKNNAYPTIKRSYNNTNQEDLLVLWGIHHPNDAAEQTRLYQNPTTYISVGTSTLNQRLVPKIATRSKVNGESGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 36 - DNA, 1707 bp, synthetic construct
  source 1..1707: mol_type = other DNA
atggaaaagattgtgctgctgctggccatcgtgtccctggtcaagagcgatcaaatctgcatcggctaccacgccaacaacagcaccgaacaggtggacaccattatggaaaagaacgtgaccgtgacacacgcccaggacatcctggaaaagacccacaacggcaagctgtgcgacctggatggcgtgaagcctctgatcctgagagattgctctgtggccggctggctgctgggcaatcctatgtgcgacgagttcatcaacgtgcccgagtggtcctatatcgtggaaaaggccaatcctgccaacgacctgtgctaccccggcaacttcaacgactacgaggaactgaaacatctgctgagccggatcaaccacttcgagaagatccagatcatccccaagtcctcttggagcgatcacgaggcctctagcggagtggtgccggcctgtccttaccaaggcagaagcagcttcttccggaacgtcgtgtggctgatcaagaagaacaacgcttaccccaccatcaagcggagctacaacaacaccaatcaagaggacctgctggtgctgtggggcatccaccatcctaatgatgccgccgagcagacccggctgtaccagaatcctacaacctacatcagcgtgggcaccagcacactgaaccagagactggtgcctaagatcgccaccagatccaaagtgaacggcgaaagcggccggatggaattcttctggaccatcctgaagcctaacgacgccatcaacttcgagagcaacggcaactttatcgcccctgagtacgcctacaagatcgtgaagaagggcgacagcgccatcatgaagtccgagctggaatacggcaactgcaacaccaagtgtcagacccctatgggcgccatcaatagcagcatgcccttccacaacattcaccctctgaccatcggcgagtgccccaaatacgtgaagtccaacagactggtcctggccaccggcctgagaaattctccacagagagagcggcgcagaaagaagagaggcctgtttggagccattgccggctttatcgaaggcggctggcaaggcatggttgacggatggtacggctatcaccacagcaatgagcaaggctctggctacgccgccgacaaagagagcacacagaaagccatcgacggcgtgaccaacaaagtgaatagcatcatcgacaagatgaacacccagttcgaggccgtgggcagagagttcaacaacctggaaagacggatcgagaacctgaacaagaagatggaggacggcttcctggacgtgtggacctataatgccgagctgctggtcctgatggaaaacgagagaaccctggacttccacgacagcaacgtgaagaacctgtacgacaaagtgcggctccagctgcgggacaatgccaaagaactcggcaacggctgcttcgagttctaccacaagtgcgacaacgagtgcatggaaagcgtgcggaacggcacctacgactaccctcagtactctgaggaagcccggctgaagagagaagagatcagcggagtgaagctggaatccatcggcacataccagatcctgagcatctacagcaccgtggcctcttctctggccctggctattatggtggctggcctgagcctgtggatgtgctctaatggcagcctccagtgccggatctgcatctga

SEQ ID NO: 37 - protein, 268 aa, synthetic construct
THNGKLCDLDGVKPLILRDCSVAGWLLGNPMCDEFINVPEWSYIVEKANPANDLCYPGNFNDYEELKHLLSRINHFEKIQIIPKSSWSDHEASSGVVPACPYQGRSSFFRNVVWLIKKNNAYPTIKRSYNNTNQEDLLVLWGIHHPNDAAEQTRLYQNPTTYISVGTSTLNQRLVPKIATRSKVNGESGRMEFFWTILKPNDAINFESNGNFIAPEYAYKIVKKGDSAIMKSELEYGNCNTKCQTPMGAINSSMPFHNIHPLTIGECP

SEQ ID NO: 38 - DNA, 804 bp, synthetic construct
  source 1..804: mol_type = other DNA
acccacaacggcaagctgtgcgacctggatggcgtgaagcctctgatcctgagagattgctctgtggccggctggctgctgggcaatcctatgtgcgacgagttcatcaacgtgcccgagtggtcctatatcgtggaaaaggccaatcctgccaacgacctgtgctaccccggcaacttcaacgactacgaggaactgaaacatctgctgagccggatcaaccacttcgagaagatccagatcatccccaagtcctcttggagcgatcacgaggcctctagcggagtggtgccggcctgtccttaccaaggcagaagcagcttcttccggaacgtcgtgtggctgatcaagaagaacaacgcttaccccaccatcaagcggagctacaacaacaccaatcaagaggacctgctggtgctgtggggcatccaccatcctaatgatgccgccgagcagacccggctgtaccagaatcctacaacctacatcagcgtgggcaccagcacactgaaccagagactggtgcctaagatcgccaccagatccaaagtgaacggcgaaagcggccggatggaattcttctggaccatcctgaagcctaacgacgccatcaacttcgagagcaacggcaactttatcgcccctgagtacgcctacaagatcgtgaagaagggcgacagcgccatcatgaagtccgagctggaatacggcaactgcaacaccaagtgtcagacccctatgggcgccatcaatagcagcatgcccttccacaacattcaccctctgaccatcggcgagtgcccc

SEQ ID NO: 39 - protein, 51 aa, synthetic construct; identical to SEQ ID NO: 31

SEQ ID NO: 40 - DNA, 153 bp, synthetic construct; identical to SEQ ID NO: 32
  source 1..153: mol_type = other DNA

SEQ ID NO: 41 - protein, 249 aa, synthetic construct; identical to SEQ ID NO: 33

SEQ ID NO: 42 - DNA, 750 bp, synthetic construct
  source 1..750: mol_type = other DNA
aaatacgtgaagtccaacagactggtcctggccaccggcctgagaaattctccacagagagagcggcgcagaaagaagagaggcctgtttggagccattgccggctttatcgaaggcggctggcaaggcatggttgacggatggtacggctatcaccacagcaatgagcaaggctctggctacgccgccgacaaagagagcacacagaaagccatcgacggcgtgaccaacaaagtgaatagcatcatcgacaagatgaacacccagttcgaggccgtgggcagagagttcaacaacctggaaagacggatcgagaacctgaacaagaagatggaggacggcttcctggacgtgtggacctataatgccgagctgctggtcctgatggaaaacgagagaaccctggacttccacgacagcaacgtgaagaacctgtacgacaaagtgcggctccagctgcgggacaatgccaaagaactcggcaacggctgcttcgagttctaccacaagtgcgacaacgagtgcatggaaagcgtgcggaacggcacctacgactaccctcagtactctgaggaagcccggctgaagagagaagagatcagcggagtgaagctggaatccatcggcacataccagatcctgagcatctacagcaccgtggcctcttctctggccctggctattatggtggctggcctgagcctgtggatgtgctctaatggcagcctccagtgccggatctgcatctga

SEQ ID NO: 43 - protein, 568 aa, synthetic construct
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGRSSFFRNVVWLIKKNNAYPTIKRSYNNTNQEDLLVLWGIHHPNDAAEQTRLYQNPTTYISVGTSTLNQRLVPKIATRSKVNGESGRMEFFWTILKPNDAINFESNGNFIAPEYAYKIVKKGDSAIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 44 - DNA, 1707 bp, synthetic construct
  source 1..1707: mol_type = other DNA
atggaaaagattgtgctgctgctggccatcgtgtccctggtcaagagcgatcaaatctgcatcggctaccacgccaacaacagcaccgaacaggtggacaccattatggaaaagaacgtgaccgtgacacacgcccaggacatcctggaaaagacccacaacggcaagctgtgcgacctggatggcgtgaagcctctgatcctgagagattgctctgtggccggctggctgctgggcaatcctatgtgcgacgagttcatcaacgtgcccgagtggtcctatatcgtggaaaaggccaatcctgccaacgacctgtgctaccccggcaacttcaacgactacgaggaactgaaacatctgctgagccggatcaaccacttcgagaagatccagatcatccccaagtcctcttggagcgatcacgaggcctctagcggagtgtctagcgcctgtccttaccaaggcagaagcagcttcttccggaacgtcgtgtggctgatcaagaagaacaacgcttaccccaccatcaagcggagctacaacaacaccaatcaagaggacctgctggtgctgtggggcatccaccatcctaatgatgccgccgagcagacccggctgtaccagaatcctacaacctacatcagcgtgggcaccagcacactgaaccagagactggtgcctaagatcgccaccagatccaaagtgaacggcgaaagcggccggatggaattcttctggaccatcctgaagcctaacgacgccatcaacttcgagagcaacggcaactttatcgcccctgagtacgcctacaagatcgtgaagaagggcgacagcgccatcatgaagtccgagctggaatacggcaactgcaacaccaagtgtcagacccctatgggcgccatcaatagcagcatgcccttccacaacattcaccctctgaccatcggcgagtgccccaaatacgtgaagtccaacagactggtcctggccaccggcctgagaaattctccacagagagagcggcgcagaaagaagagaggcctgtttggagccattgccggctttatcgaaggcggctggcaaggcatggttgacggatggtacggctatcaccacagcaatgagcaaggctctggctacgccgccgacaaagagagcacacagaaagccatcgacggcgtgaccaacaaagtgaatagcatcatcgacaagatgaacacccagttcgaggccgtgggcagagagttcaacaacctggaaagacggatcgagaacctgaacaagaagatggaggacggcttcctggacgtgtggacctataatgccgagctgctggtcctgatggaaaacgagagaaccctggacttccacgacagcaacgtgaagaacctgtacgacaaagtgcggctccagctgcgggacaatgccaaagaactcggcaacggctgcttcgagttctaccacaagtgcgacaacgagtgcatggaaagcgtgcggaacggcacctacgactaccctcagtactctgaggaagcccggctgaagagagaagagatcagcggagtgaagctggaatccatcggcacataccagatcctgagcatctacagcaccgtggcctcttctctggccctggctattatggtggctggcctgagcctgtggatgtgctctaatggcagcctccagtgccggatctgcatctga

SEQ ID NO: 45 - protein, 268 aa, synthetic construct
THNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGRSSFFRNVVWLIKKNNAYPTIKRSYNNTNQEDLLVLWGIHHPNDAAEQTRLYQNPTTYISVGTSTLNQRLVPKIATRSKVNGESGRMEFFWTILKPNDAINFESNGNFIAPEYAYKIVKKGDSAIMKSELEYGNCNTKCQTPMGAINSSMPFHNIHPLTIGECP

SEQ ID NO: 46 - DNA, 804 bp, synthetic construct
  source 1..804: mol_type = other DNA
acccacaacggcaagctgtgcgacctggatggcgtgaagcctctgatcctgagagattgctctgtggccggctggctgctgggcaatcctatgtgcgacgagttcatcaacgtgcccgagtggtcctatatcgtggaaaaggccaatcctgccaacgacctgtgctaccccggcaacttcaacgactacgaggaactgaaacatctgctgagccggatcaaccacttcgagaagatccagatcatccccaagtcctcttggagcgatcacgaggcctctagcggagtgtctagcgcctgtccttaccaaggcagaagcagcttcttccggaacgtcgtgtggctgatcaagaagaacaacgcttaccccaccatcaagcggagctacaacaacaccaatcaagaggacctgctggtgctgtggggcatccaccatcctaatgatgccgccgagcagacccggctgtaccagaatcctacaacctacatcagcgtgggcaccagcacactgaaccagagactggtgcctaagatcgccaccagatccaaagtgaacggcgaaagcggccggatggaattcttctggaccatcctgaagcctaacgacgccatcaacttcgagagcaacggcaactttatcgcccctgagtacgcctacaagatcgtgaagaagggcgacagcgccatcatgaagtccgagctggaatacggcaactgcaacaccaagtgtcagacccctatgggcgccatcaatagcagcatgcccttccacaacattcaccctctgaccatcggcgagtgcccc

SEQ ID NO: 47 - protein, 51 aa, synthetic construct; identical to SEQ ID NO: 31

SEQ ID NO: 48 - DNA, 153 bp, synthetic construct; identical to SEQ ID NO: 32
  source 1..153: mol_type = other DNA

SEQ ID NO: 49 - protein, 249 aa, synthetic construct; identical to SEQ ID NO: 33

SEQ ID NO: 50 - DNA, 750 bp, synthetic construct; identical to SEQ ID NO: 42
  source 1..750: mol_type = other DNA

SEQ ID NO: 51 - protein, 51 aa, synthetic construct; identical to SEQ ID NO: 31

SEQ ID NO: 52 - DNA, 153 bp, synthetic construct
  source 1..153: mol_type = other DNA
atggaaaagatcgtgctgctgctggccatcgtgtccctggtcaagagcgaccaaatctgcatcggctaccacgccaacaacagcaccgaacaggtggacaccattatggaaaagaacgtcaccgtgacacacgcccaggacatcctggaaaag

SEQ ID NO: 53 - protein, 249 aa, synthetic construct; identical to SEQ ID NO: 33

SEQ ID NO: 54 - DNA, 750 bp, synthetic construct
  source 1..750: mol_type = other DNA
aaatacgtgaagtccaacagactggtcctggccaccggcctgagaaactctccccagcgcgagcggagaagaaagaagagaggcctgtttggcgccattgccggctttatcgaaggcggctggcaaggcatggtggacggatggtacggctatcaccacagcaacgagcaaggctctggatacgccgccgacaaagagagcacccagaaagccattgacggcgtgaccaacaaagtcaacagcatcatcgacaagatgaacacccagttcgaggccgtgggcagagagttcaacaacctggaacggcggatcgagaacctgaacaagaagatggaggacggcttcctggacgtgtggacctacaatgccgagctgctggtcctgatggaaaacgagagaaccctggacttccacgacagcaacgtgaagaacctgtacgacaaagtgcggctccagctgcgggacaacgccaaagaactcggcaacggctgcttcgagttctaccacaagtgcgacaacgagtgcatggaaagcgtgcggaacggcacctacgactaccctcagtacagcgaggaagcccggctgaagagggaagagatcagcggagtgaagctggaatccatcggcacataccagatcctgagcatctacagcaccgtggcctcttctctggccctggccattatggtggctggcctgtctctgtggatgtgcagcaatggcagcctccagtgccggatctgcatctga

SEQ ID NO: 55 - DNA, 1707 bp, synthetic construct
  source 1..1707: mol_type = other DNA
atggaaaagatcgtgctgctgctggccatcgtgtccctggtcaagagcgaccaaatctgcatcggctaccacgccaacaacagcaccgaacaggtggacaccattatggaaaagaacgtcaccgtgacacacgcccaggacatcctggaaaagacccacaacggcaagctgtgcgacctggatggcgtgaagcctctgatcctgagagattgctctgtggccggatggctgctgggcaatcccatgtgcgacgagttcatcaacgtgcccgagtggtcctatatcgtggaaaaggccaatcctgccaacgacctgtgctaccccggcaacttcaacgactacgaggaactgaagcacctcctgagccggatcaaccacttcgagaagatccagatcatccccaagtcctcttggagcgaccacgaagcctctagcggagtgtctagcgcctgtccttaccaaggcagacccagcttcttccggaacgtcgtgtggctgatcaagaagaacgacacataccccaccatcaagcggagctacaacaacaccaatcaagaggacctgctggtgctgtggggcatccaccatcctaatgatgccgccgagcagaccaagctgtatcagaaccccaccacctacatcagcgtgggcaccagcacactgaaccagagactggtgcctaagatcgccaccagatccaaagtgaacggccagagcggcagaatggaattcttctggaccatcctgaagcctaacgacgccatcaacttcgagagcaacggcaactttatcgcccctgagtacgcctacaagatcgtgaagaagggcgacagcgccatcatgaagtccgagctggaatacggcaactgcaacaccaagtgtcagacccctatgggcgccatcaatagcagcatgcccttccacaacattcaccctctgaccatcggcgagtgccccaaatacgtgaagtccaacagactggtcctggccaccggcctgagaaactctccccagcgcgagcggagaagaaagaagagaggcctgtttggcgccattgccggctttatcgaaggcggctggcaaggcatggtggacggatggtacggctatcaccacagcaacgagcaaggctctggatacgccgccgacaaagagagcacccagaaagccattgacggcgtgaccaacaaagtcaacagcatcatcgacaagatgaacacccagttcgaggccgtgggcagagagttcaacaacctggaacggcggatcgagaacctgaacaagaagatggaggacggcttcctggacgtgtggacctacaatgccgagctgctggtcctgatggaaaacgagagaaccctggacttccacgacagcaacgtgaagaacctgtacgacaaagtgcggctccagctgcgggacaacgccaaagaactcggcaacggctgcttcgagttctaccacaagtgcgacaacgagtgcatggaaagcgtgcggaacggcacctacgactaccctcagtacagcgaggaagcccggctgaagagggaagagatcagcggagtgaagctggaatccatcggcacataccagatcctgagcatctacagcaccgtggcctcttctctggccctggccattatggtggctggcctgtctctgtggatgtgcagcaatggcagcctccagtgccggatctgcatctga

SEQ ID NO: 56 - DNA, 804 bp, synthetic construct
  source 1..804: mol_type = other DNA
acccacaacggcaagctgtgcgacctggatggcgtgaagcctctgatcctgagagattgctctgtggccggatggctgctgggcaatcccatgtgcgacgagttcatcaacgtgcccgagtggtcctatatcgtggaaaaggccaatcctgccaacgacctgtgctaccccggcaacttcaacgactacgaggaactgaagcacctcctgagccggatcaaccacttcgagaagatccagatcatccccaagtcctcttggagcgaccacgaagcctctagcggagtgtctagcgcctgtccttaccaaggcagacccagcttcttccggaacgtcgtgtggctgatcaagaagaacgacacataccccaccatcaagcggagctacaacaacaccaatcaagaggacctgctggtgctgtggggcatccaccatcctaatgatgccgccgagcagaccaagctgtatcagaaccccaccacctacatcagcgtgggcaccagcacactgaaccagagactggtgcctaagatcgccaccagatccaaagtgaacggccagagcggcagaatggaattcttctggaccatcctgaagcctaacgacgccatcaacttcgagagcaacggcaactttatcgcccctgagtacgcctacaagatcgtgaagaagggcgacagcgccatcatgaagtccgagctggaatacggcaactgcaacaccaagtgtcagacccctatgggcgccatcaatagcagcatgcccttccacaacattcaccctctgaccatcggcgagtgcccc

SEQ ID NO: 57 - protein, 51 aa, synthetic construct; identical to SEQ ID NO: 31

SEQ ID NO: 58 - DNA, 153 bp, synthetic construct; identical to SEQ ID NO: 52
  source 1..153: mol_type = other DNA

SEQ ID NO: 59 - protein, 249 aa, synthetic construct; identical to SEQ ID NO: 33

SEQ ID NO: 60 - DNA, 750 bp, synthetic construct; identical to SEQ ID NO: 54
  source 1..750: mol_type = other DNA

SEQ ID NO: 61 - DNA, 1707 bp, synthetic construct
  source 1..1707: mol_type = other DNA
atggaaaagatcgtgctgctgctggccatcgtgtccctggtcaagagcgaccaaatctgcatcggctaccacgccaacaacagcaccgaacaggtggacaccattatggaaaagaacgtcaccgtgacacacgcccaggacatcctggaaaagacccacaacggcaagctgtgcgacctggatggcgtgaagcctctgatcctgagagattgctctgtggccggatggctgctgggcaatcccatgtgcgacgagttcatcaacgtgcccgagtggtcctatatcgtggaaaaggccaatcctgccaacgacctgtgctaccccggcaacttcaacgactacgaggaactgaagcacctcctgagccggatcaaccacttcgagaagatccagatcatccccaagtcctcttggagcgaccacgaagcctctagcggagtgtctagcgcctgtccttaccaaggcagacccagcttcttccggaacgtcgtgtggctgatcaagaagaacaacacataccccaccatcaagcggagctacaacaacaccaatcaagaggacctgctggtgctgtggggcatccaccatcctaatgatgccgccgagcagaccaagctgtatcagaaccccaccacctacatcagcgtgggcaccagcacactgaaccagagactggtgcctaagatcgccaccagatccaaagtgaacggccagagcggcagaatggaattcttctggaccatcctgaagcctaacgacgccatcaacttcgagagcaacggcaactttatcgcccctgagtacgcctacaagatcgtgaagaagggcgacagcgccatcatgaagtccgagctggaatacggcaactgcaacaccaagtgtcagacccctatgggcgccatcaatagcagcatgcccttccacaacattcaccctctgaccatcggcgagtgccccaaatacgtgaagtccaacagactggtcctggccaccggcctgagaaactctccccagcgcgagcggagaagaaagaagagaggcctgtttggcgccattgccggctttatcgaaggcggctggcaaggcatggtggacggatggtacggctatcaccacagcaacgagcaaggctctggatacgccgccgacaaagagagcacccagaaagccattgacggcgtgaccaacaaagtcaacagcatcatcgacaagatgaacacccagttcgaggccgtgggcagagagttcaacaacctggaacggcggatcgagaacctgaacaagaagatggaggacggcttcctggacgtgtggacctacaatgccgagctgctggtcctgatggaaaacgagagaaccctggacttccacgacagcaacgtgaagaacctgtacgacaaagtgcggctccagctgcgggacaacgccaaagaactcggcaacggctgcttcgagttctaccacaagtgcgacaacgagtgcatggaaagcgtgcggaacggcacctacgactaccctcagtacagcgaggaagcccggctgaagagggaagagatcagcggagtgaagctggaatccatcggcacataccagatcctgagcatctacagcaccgtggcctcttctctggccctggccattatggtggctggcctgtctctgtggatgtgcagcaatggcagcctccagtgccggatctgcatctga

SEQ ID NO: 62 - DNA, 804 bp, synthetic construct
  source 1..804: mol_type = other DNA
acccacaacggcaagctgtgcgacctggatggcgtgaagcctctgatcctgagagattgctctgtggccggatggctgctgggcaatcccatgtgcgacgagttcatcaacgtgcccgagtggtcctatatcgtggaaaaggccaatcctgccaacgacctgtgctaccccggcaacttcaacgactacgaggaactgaagcacctcctgagccggatcaaccacttcgagaagatccagatcatccccaagtcctcttggagcgaccacgaagcctctagcggagtgtctagcgcctgtccttaccaaggcagacccagcttcttccggaacgtcgtgtggctgatcaagaagaacaacacataccccaccatcaagcggagctacaacaacaccaatcaagaggacctgctggtgctgtggggcatccaccatcctaatgatgccgccgagcagaccaagctgtatcagaaccccaccacctacatcagcgtgggcaccagcacactgaaccagagactggtgcctaagatcgccaccagatccaaagtgaacggccagagcggcagaatggaattcttctggaccatcctgaagcctaacgacgccatcaacttcgagagcaacggcaactttatcgcccctgagtacgcctacaagatcgtgaagaagggcgacagcgccatcatgaagtccgagctggaatacggcaactgcaacaccaagtgtcagacccctatgggcgccatcaatagcagcatgcccttccacaacattcaccctctgaccatcggcgagtgcccc

SEQ ID NO: 63 - protein, 1175 aa, synthetic construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFINYEELREQLSSVSSFERFEIFPKTSSWPNHDSNKGVTAACPHAGAKSFYKNLIWLVKKGNSYPKLSKSYINDKGKEVLVLWGIHHPSTTADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLESTRIYQILAIYSTVASSLVLVVSLGAISFWMCSNGSLQCRICIGSGEGRGSLLTCGDVEENPGPMNPNQKIITIGSICMVVGIISLILQIGNIISIWVSHSIQTGNQNQPETCNQSIITYENNTWVNQTYVNISNTNFVAEQAVASVALAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGISWLTIGISGPDNGAVAVLKYNGIITDTIKSWRNNILRTQESECACINGSCFTIMTDGPSNGQASYKIFKIEKGKVVKSVELNAPNYHYEECSCYPDAGEVMCVCRDNWHGSNRPWVSFNQNLEYQIGYICSGVFGDNPRPNDGTGSCGPVSSNGAYGVKGFSFKYGKGVWIGRTKSTSSRSGFEMIWDPNGWTETDSSFSVKQDIVAITDWSGYSGSFVQHPELTGLDCMRPCFWVELIRGRPKENTIWTSGSSISFCGVNSDTVGWSWPDGAELPFTIDKGSGATNFSLLKQAGDVEENPGPMSLLTEVETPTRNGWECRCSDSSDPLVIAASIIGILHLILWILDRLFFKCIYRRLKYGLKRGPSTEGVPESMREEYRQKQQSAVDVDDGHFVNIELE

SEQ ID NO: 64 - protein, 568 aa, Influenza A
  source 1..568: note = strain: A/whooper swan/Mongolia/244/2005
MEKIVLLLAIVSLVKSDQICIGYHANNSTEQVDTIMEKNVTVTHAQDILEKTHNGKLCDLDGVKPLILRDCSVAGWLLGNPMCDEFLNVPEWSYIVEKINPANDLCYPGNFNDYEELKHLLSRINHFEKIQIIPKSSWSDHEASSGVSSACPYQGRSSFFRNVVWLIKKDNAYPTIKRSYNNTNQEDLLVLWGIHHPNDAAEQTRLYQNPTTYISVGTSTLNQRLVPKIATRSKVNGQSGRMEFFWTILKPNDAINFESNGNFIAPENAYKIVKKGDSTIMKSELEYGNCNTKCQTPIGAINSSMPFHNIHPLTIGECPKYVKSNRLVLATGLRNSPQGERRRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHRCDNECMESVRNGTYDYPQYSEEARLKREEISGVKLESIGTYQILSIYSTVASSLALAIMVAGLSLWMCSNGSLQCRICI

SEQ ID NO: 65 - protein, 567 aa, Influenza A
  source 1..567: note = strain: A/gyrfalcon/Washington/41088-6/2014
MEKIVLLLAVISLVKSDQICIGYHANNSTKQVDTIMEKNVTVTHAQDILEKTHNGKLCDLNGVKPLILKDCSVAGWLLGNPMCDEFIRVPEWSYIVERANPANDLCYPGTLNDYEELKHLLSRINHFEKTLIIPRSSWPNHETSLGVSAACPYQGASSFFRNVVWLIKKNDAYPTIKISYNNTNREDLLILWGIHHSNNAAEQTNLYKNPDTYVSVGTSTLNQRLVPKIATRSQVNGQSGRMDFFWTILKPNDAIHFESNGNFIAPEYAYKIVKKGDSTIMKSEMEYGHCNTKCQTPIGAINSSMPFHNIHPLTIGECPKYVKSNKLVLATGLRNSPLRERRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPKYSEEAILKREEISGVKLESIGTYQILSIYSTVASSLALAIIVAGLSLWMCSNGSLQCRICI

SEQ ID NO: 66 - protein, 21 aa, synthetic construct
GSGEGRGSLLTCGDVEENPGP

SEQ ID NO: 67 - protein, 22 aa, synthetic construct
GSGATNFSLLKQAGDVEENPGP

SEQ ID NO: 68 - protein, 566 aa, synthetic construct
MKVKLLVLLCTFTATYADTICIGYHANNSTDTVDTVLEKNVTVTHSVNLLEDSHNGKLCLLKGIAPLQLGNCSVAGWILGNPECELLISKESWSYIVETPNPENGTCYPGYFADYEELREQLSSVSSFERFEIFPKESSWPNHTVTSGVSASCSHNGKSSFYRNLLWLTGKNGLYPNLSKSYANNKEKEVLVLWGVHHPPNIGDQRALYHTENAYVSVVSSHYSRRFTPEIAKRPKVRDQEGRINYYWTLLEPGDTIIFEANGNLIAPRYAFALSRGFGSGIINSNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFIDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 69 - DNA, 1698 bp, synthetic construct
  source 1..1698: mol_type = other DNA
atgaaggtcaaactgctggtgctgctgtgcaccttcaccgccacatacgccgataccatctgtatcggctaccacgccaacaacagcaccgacaccgtggataccgtgctggaaaagaacgtgaccgtgacacacagcgtgaacctgctggaagatagccacaacggcaagctgtgcctgctgaagggaattgcccctctccagctgggaaattgctctgtggctggctggatcctgggcaatcctgagtgcgagctgctgatctccaaagagagctggtcctacatcgtcgagacacccaatccagagaacggcacatgctaccccggctacttcgccgactatgaggaactgagagagcagctgagcagcgtcagcagcttcgagagattcgagattttccccaaagagtccagctggcccaaccacacagtgacaagcggagtgtctgccagctgttcccacaatggcaagagcagcttctacagaaacctgctgtggctgaccggcaagaacggactgtaccccaacctgagcaagagctacgctaacaacaaagagaaagaggtcctggtcctctggggcgtgcaccatcctccaaatatcggagatcagagagccctgtaccacaccgagaatgcctacgtgtccgtggtgtccagccactacagcagaagattcacccctgagatcgccaagcggcccaaagtgcgagatcaagagggcagaatcaactactactggacactgctggaacccggcgacaccatcatcttcgaggccaacggaaacctgatcgcccctagatacgccttcgctctgagcagaggctttggcagcggcatcatcaacagcaacgcccctatggatgagtgcgacgccaagtgtcaaacaccccagggcgctatcaacagctccctgccttttcagaacgtgcaccctgtgaccatcggcgagtgtcctaaatatgtgcggagcgccaagctgagaatggtcaccggcctgagaaacatccccagcatccagtctagaggcctgtttggcgccattgccggctttatcgaaggcggatggacaggcatggtggacggatggtacggctatcaccaccagaatgagcaaggcagcggctacgccgccgatcagaaatctacccagaacgccatcaacgggatcaccaacaaagtgaacagcgtgatcgagaagatgaacacccagttcaccgccgtgggcaaagagttcaacaagctggaaagacggatggaaaacctgaacaagaaggtggacgacggcttcatcgacatctggacctacaacgctgagctgctggtcctcctggaaaacgagagaaccctggacttccacgacagcaacgtgaagaacctgtacgagaaagtgaagtcccagctgaagaacaacgccaaagagatcggcaacggctgcttcgagttctaccacaagtgcaacgacgagtgcatggaaagcgtgaagaatggcacctacgactaccccaagtacagcgaggaaagcaagctgaaccgcgagaagatcgacggcgtgaagctggaatctatgggcgtgtaccagatcctggccatctacagcacagtggcttctagcctggtgctcctggtgtctctgggagccatcagcttttggatgtgcagcaatggcagcctccagtgccggatctgcatc

SEQ ID NO: 70 - DNA, 6083 bp, synthetic construct
  source 1..6083: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtaccgccaccatgaaggtcaaactgctggtgctgctgtgcaccttcaccgccacatacgccgataccatctgtatcggctaccacgccaacaacagcaccgacaccgtggataccgtgctggaaaagaacgtgaccgtgacacacagcgtgaacctgctggaagatagccacaacggcaagctgtgcctgctgaagggaattgcccctctccagctgggaaattgctctgtggctggctggatcctgggcaatcctgagtgcgagctgctgatctccaaagagagctggtcctacatcgtcgagacacccaatccagagaacggcacatgctaccccggctacttcgccgactatgaggaactgagagagcagctgagcagcgtcagcagcttcgagagattcgagattttccccaaagagtccagctggcccaaccacacagtgacaagcggagtgtctgccagctgttcccacaatggcaagagcagcttctacagaaacctgctgtggctgaccggcaagaacggactgtaccccaacctgagcaagagctacgctaacaacaaagagaaagaggtcctggtcctctggggcgtgcaccatcctccaaatatcggagatcagagagccctgtaccacaccgagaatgcctacgtgtccgtggtgtccagccactacagcagaagattcacccctgagatcgccaagcggcccaaagtgcgagatcaagagggcagaatcaactactactggacactgctggaacccggcgacaccatcatcttcgaggccaacggaaacctgatcgcccctagatacgccttcgctctgagcagaggctttggcagcggcatcatcaacagcaacgcccctatggatgagtgcgacgccaagtgtcaaacaccccagggcgctatcaacagctccctgccttttcagaacgtgcaccctgtgaccatcggcgagtgtcctaaatatgtgcggagcgccaagctgagaatggtcaccggcctgagaaacatccccagcatccagtctagaggcctgtttggcgccattgccggctttatcgaaggcggatggacaggcatggtggacggatggtacggctatcaccaccagaatgagcaaggcagcggctacgccgccgatcagaaatctacccagaacgccatcaacgggatcaccaacaaagtgaacagcgtgatcgagaagatgaacacccagttcaccgccgtgggcaaagagttcaacaagctggaaagacggatggaaaacctgaacaagaaggtggacgacggcttcatcgacatctggacctacaacgctgagctgctggtcctcctggaaaacgagagaaccctggacttccacgacagcaacgtgaagaacctgtacgagaaagtgaagtcccagctgaagaacaacgccaaagagatcggcaacggctgcttcgagttctaccacaagtgcaacgacgagtgcatggaaagcgtgaagaatggcacctacgactaccccaagtacagcgaggaaagcaagctgaaccgcgagaagatcgacggcgtgaagctggaatctatgggcgtgtaccagatcctggccatctacagcacagtggcttctagcctggtgctcctggtgtctctgggagccatcagcttttggatgtgcagcaatggcagcctccagtgccggatctgcatctgagcggccgcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 71 - protein, 569 aa, synthetic construct
MEKIVLLLAIVSLVKSDQICIGYHANNSTEQVDTIMEKNVTVTHAQDILEKTHNGKLCDLNGVKPLILKDCSVAGWLLGNPMCDEFIRVPEWSYIVERANPANDLCYPGNLNDYEELKHLLSRINHFEKILIIPKSSWPNHETSLGVSAACPYQGTPSFFRNVVWLIKKNDAYPTIKISYNNTNREDLLILWGIHHSNNAAEQTNLYKNPTTYISVGTSTLNQRLVPKIATRSQVNGQRGRMDFFWTILKPNDAIHFESNGNFIAPEYAYKIVKKGDSTIMKSEVEYGHCNTKCQTPIGAINSSMPFHNIHPLTIGECPKYVKSNKLVLATGLRNSPLREKRRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 72 - DNA, 1727 bp, synthetic construct
  source 1..1727: mol_type = other DNA
gtaccgccaccatggaaaagatcgtgctgctgctggccatcgtgtccctggtcaagagcgaccaaatctgcatcggctaccacgccaacaacagcaccgaacaggtggacaccattatggaaaagaacgtcaccgtgacacacgcccaggacatcctggaaaagacccacaacggcaagctgtgcgacctgaacggcgtgaagcctctgatcctgaaggattgctctgtggccggatggctgctgggcaatcccatgtgcgacgagttcatcagagtgcccgagtggtcctacatcgtggaaagagccaatcctgccaacgacctgtgctaccccggcaacctgaacgactacgaggaactgaagcacctcctgagccggatcaaccacttcgagaagatcctgatcatccccaagagcagctggcccaaccacgagacatctctgggagtgtctgccgcatgtccataccagggcacccctagctttttccggaacgtcgtgtggctgatcaagaagaacgacgcttaccccaccatcaagatcagctacaacaacaccaaccgcgaggacctgctgatcctgtggggaatccaccacagcaacaatgccgccgagcagaccaacctgtacaagaaccccaccacctacatcagcgtgggcaccagcacactgaaccagagactggtgcctaagatcgccacacggtcccaagtgaatggccagaggggcagaatggacttcttctggaccatcctgaagcctaacgacgccatccactttgagagcaacggcaactttatcgcccctgagtacgcctacaagatcgtgaagaagggcgacagcaccatcatgaagtccgaggtggaatacggccactgcaacaccaagtgtcagacccctatcggcgccatcaactccagcatgcccttccacaacattcaccctctgaccatcggcgagtgccccaaatacgtgaagtccaacaagctggtgctggctaccggcctgagaaacagccctctgagagagaagcgcagacggaagaagagaggcctgtttggcgccattgccggctttatcgaaggcggctggcaaggcatggtggacggatggtacggctaccatcacagcaacgagcaaggctctggatacgccgccgacaaagagagcacccagaaagccattgacggcgtgaccaacaaagtgaacagcatcatcgacaagatgaacacccagttcgaggccgtgggcagagagttcaacaacctggaacggcggatcgagaatctgaacaagaagatggaggacggcttcctggacgtgtggacctacaatgccgagctgctggtcctgatggaaaacgagagaaccctggacttccacgactccaacgtgaagaacctgtacgacaaagtgcggctccagctgcgggacaacgccaaagaactcggcaacggctgcttcgagttctaccacaagtgcgacaacgagtgcatggaaagcgtgcggaacggcacctacgactaccctcagtacagcgaggaagcccggctgaagagagaagagatcagcggagtgaagctggaatccatcggcacataccagatcctgtccatctacagcaccgtggcctcttctctggccctggccattatggtggctggcctgtctctgtggatgtgcagcaatggcagcctccagtgccggatctgcatctgagcggcc

SEQ ID NO: 73 - protein, 268 aa, synthetic construct
THNGKLCDLNGVKPLILKDCSVAGWLLGNPMCDEFIRVPEWSYIVERANPANDLCYPGNLNDYEELKHLLSRINHFEKILIIPKSSWPNHETSLGVSAACPYQGTPSFFRNVVWLIKKNDAYPTIKISYNNTNREDLLILWGIHHSNNAAEQTNLYKNPTTYISVGTSTLNQRLVPKIATRSQVNGQRGRMDFFWTILKPNDAIHFESNGNFIAPEYAYKIVKKGDSTIMKSEVEYGHCNTKCQTPIGAINSSMPFHNIHPLTIGECP

SEQ ID NO: 74 - DNA, 824 bp, synthetic construct
  source 1..824: mol_type = other DNA
tcctggaaaagacccacaacggcaagctgtgcgacctgaacggcgtgaagcctctgatcctgaaggattgctctgtggccggatggctgctgggcaatcccatgtgcgacgagttcatcagagtgcccgagtggtcctacatcgtggaaagagccaatcctgccaacgacctgtgctaccccggcaacctgaacgactacgaggaactgaagcacctcctgagccggatcaaccacttcgagaagatcctgatcatccccaagagcagctggcccaaccacgagacatctctgggagtgtctgccgcatgtccataccagggcacccctagctttttccggaacgtcgtgtggctgatcaagaagaacgacgcttaccccaccatcaagatcagctacaacaacaccaaccgcgaggacctgctgatcctgtggggaatccaccacagcaacaatgccgccgagcagaccaacctgtacaagaaccccaccacctacatcagcgtgggcaccagcacactgaaccagagactggtgcctaagatcgccacacggtcccaagtgaatggccagaggggcagaatggacttcttctggaccatcctgaagcctaacgacgccatccactttgagagcaacggcaactttatcgcccctgagtacgcctacaagatcgtgaagaagggcgacagcaccatcatgaagtccgaggtggaatacggccactgcaacaccaagtgtcagacccctatcggcgccatcaactccagcatgcccttccacaacattcaccctctgaccatcggcgagtgccccaaatacgtg

SEQ ID NO: 75 - protein, 51 aa, synthetic construct; identical to SEQ ID NO: 31

SEQ ID NO: 76 - DNA, 153 bp, synthetic construct
  source 1..153: mol_type = other DNA
gtaccgccaccatggaaaagatcgtgctgctgctggccatcgtgtccctggtcaagagcgaccaaatctgcatcggctaccacgccaacaacagcaccgaacaggtggacaccattatggaaaagaacgtcaccgtgacacacgcccaggaca

SEQ ID NO: 77 - protein, 250 aa, synthetic construct
KYVKSNKLVLATGLRNSPLREKRRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 78 - DNA, 750 bp, synthetic construct
  source 1..750: mol_type = other DNA
aagtccaacaagctggtgctggctaccggcctgagaaacagccctctgagagagaagcgcagacggaagaagagaggcctgtttggcgccattgccggctttatcgaaggcggctggcaaggcatggtggacggatggtacggctaccatcacagcaacgagcaaggctctggatacgccgccgacaaagagagcacccagaaagccattgacggcgtgaccaacaaagtgaacagcatcatcgacaagatgaacacccagttcgaggccgtgggcagagagttcaacaacctggaacggcggatcgagaatctgaacaagaagatggaggacggcttcctggacgtgtggacctacaatgccgagctgctggtcctgatggaaaacgagagaaccctggacttccacgactccaacgtgaagaacctgtacgacaaagtgcggctccagctgcgggacaacgccaaagaactcggcaacggctgcttcgagttctaccacaagtgcgacaacgagtgcatggaaagcgtgcggaacggcacctacgactaccctcagtacagcgaggaagcccggctgaagagagaagagatcagcggagtgaagctggaatccatcggcacataccagatcctgtccatctacagcaccgtggcctcttctctggccctggccattatggtggctggcctgtctctgtggatgtgcagcaatggcagcctccagtgccggatctgcatctgagcggcc

SEQ ID NO: 79 - DNA, 6092 bp, synthetic construct
  source 1..6092: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtaccgccaccatggaaaagatcgtgctgctgctggccatcgtgtccctggtcaagagcgaccaaatctgcatcggctaccacgccaacaacagcaccgaacaggtggacaccattatggaaaagaacgtcaccgtgacacacgcccaggacatcctggaaaagacccacaacggcaagctgtgcgacctgaacggcgtgaagcctctgatcctgaaggattgctctgtggccggatggctgctgggcaatcccatgtgcgacgagttcatcagagtgcccgagtggtcctacatcgtggaaagagccaatcctgccaacgacctgtgctaccccggcaacctgaacgactacgaggaactgaagcacctcctgagccggatcaaccacttcgagaagatcctgatcatccccaagagcagctggcccaaccacgagacatctctgggagtgtctgccgcatgtccataccagggcacccctagctttttccggaacgtcgtgtggctgatcaagaagaacgacgcttaccccaccatcaagatcagctacaacaacaccaaccgcgaggacctgctgatcctgtggggaatccaccacagcaacaatgccgccgagcagaccaacctgtacaagaaccccaccacctacatcagcgtgggcaccagcacactgaaccagagactggtgcctaagatcgccacacggtcccaagtgaatggccagaggggcagaatggacttcttctggaccatcctgaagcctaacgacgccatccactttgagagcaacggcaactttatcgcccctgagtacgcctacaagatcgtgaagaagggcgacagcaccatcatgaagtccgaggtggaatacggccactgcaacaccaagtgtcagacccctatcggcgccatcaactccagcatgcccttccacaacattcaccctctgaccatcggcgagtgccccaaatacgtgaagtccaacaagctggtgctggctaccggcctgagaaacagccctctgagagagaagcgcagacggaagaagagaggcctgtttggcgccattgccggctttatcgaaggcggctggcaaggcatggtggacggatggtacggctaccatcacagcaacgagcaaggctctggatacgccgccgacaaagagagcacccagaaagccattgacggcgtgaccaacaaagtgaacagcatcatcgacaagatgaacacccagttcgaggccgtgggcagagagttcaacaacctggaacggcggatcgagaatctgaacaagaagatggaggacggcttcctggacgtgtggacctacaatgccgagctgctggtcctgatggaaaacgagagaaccctggacttccacgactccaacgtgaagaacctgtacgacaaagtgcggctccagctgcgggacaacgccaaagaactcggcaacggctgcttcgagttctaccacaagtgcgacaacgagtgcatggaaagcgtgcggaacggcacctacgactaccctcagtacagcgaggaagcccggctgaagagagaagagatcagcggagtgaagctggaatccatcggcacataccagatcctgtccatctacagcaccgtggcctcttctctggccctggccattatggtggctggcctgtctctgtggatgtgcagcaatggcagcctccagtgccggatctgcatctgagcggccgcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 80 - protein, 569 aa, synthetic construct
MEKIVLLLAIVSLVKSDQICIGYHANNSTEQVDTIMEKNVTVTHAQDILEKTHNGKLCDLNGVKPLILKDCSVAGWLLGNPMCDEFIRVPEWSYIVERANPANDLCFPGNLNDYEELKHLLSRINHFEKILIIPKSSWPNHETSLGVSAACPYQGTPSFFRNVVWLIKKNDAYPTIKISYNNTNREDLLILWGIHHSNNAAEQTNLYKNPTTYISVGTSTLNQRLVPKIATRSQVNGERGRMDFFWTILKPNDAIHFESNGNFIAPEYAYKIVKKGDSTIMKSEVEYGHCNTKCQTPIGAINSSMPFHNIHPLTIGECPKYVKSNKLVLATGLRNSPLREKRRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 81 - DNA, 1727 bp, synthetic construct
  source 1..1727: mol_type = other DNA
gtaccgccaccatggaaaagatcgtgctgctgctggccatcgtgtccctggtcaagagcgaccaaatctgcatcggctaccacgccaacaacagcaccgaacaggtggacaccattatggaaaagaacgtcaccgtgacacacgcccaggacatcctggaaaagacccacaacggcaagctgtgcgacctgaacggcgtgaagcctctgatcctgaaggattgctctgtggccggatggctgctgggcaatcccatgtgcgacgagttcatcagagtgcccgagtggtcctacatcgtggaaagagccaatcctgccaacgacctgtgcttccccggcaacctgaacgactacgaggaactgaagcacctcctgagccggatcaaccacttcgagaagatcctgatcatccccaagagcagctggcccaaccacgagacatctctgggagtgtctgccgcatgtccataccagggcacccctagctttttccggaacgtcgtgtggctgatcaagaagaacgacgcttaccccaccatcaagatcagctacaacaacaccaaccgcgaggacctgctgatcctgtggggaatccaccacagcaacaatgccgccgagcagaccaacctgtacaagaaccccaccacctacatcagcgtgggcaccagcacactgaaccagagactggtgcctaagatcgccacacggtcccaagtgaatggcgagaggggcagaatggacttcttctggaccatcctgaagcctaacgacgccatccactttgagagcaacggcaactttatcgcccctgagtacgcctacaagatcgtgaagaagggcgacagcaccatcatgaagtccgaggtggaatacggccactgcaacaccaagtgtcagacccctatcggcgccatcaactccagcatgcccttccacaacattcaccctctgaccatcggcgagtgccccaaatacgtgaagtccaacaagctggtgctggctaccggcctgagaaacagccctctgagagagaagcgcagacggaagaagagaggcctgtttggcgccattgccggctttatcgaaggcggctggcaaggcatggtggacggatggtacggctaccatcacagcaacgagcaaggctctggatacgccgccgacaaagagagcacccagaaagccattgacggcgtgaccaacaaagtgaacagcatcatcgacaagatgaacacccagttcgaggccgtgggcagagagttcaacaacctggaacggcggatcgagaatctgaacaagaagatggaggacggcttcctggacgtgtggacctacaatgccgagctgctggtcctgatggaaaacgagagaaccctggacttccacgactccaacgtgaagaacctgtacgacaaagtgcggctccagctgcgggacaacgccaaagaactcggcaacggctgcttcgagttctaccacaagtgcgacaacgagtgcatggaaagcgtgcggaacggcacctacgactaccctcagtacagcgaggaagcccggctgaagagagaagagatcagcggagtgaagctggaatccatcggcacataccagatcctgtccatctacagcaccgtggcctcttctctggccctggccattatggtggctggcctgtctctgtggatgtgcagcaatggcagcctccagtgccggatctgcatctgagcggcc

SEQ ID NO: 82 - protein, 268 aa, synthetic construct
THNGKLCDLNGVKPLILKDCSVAGWLLGNPMCDEFIRVPEWSYIVERANPANDLCFPGNLNDYEELKHLLSRINHFEKILIIPKSSWPNHETSLGVSAACPYQGTPSFFRNVVWLIKKNDAYPTIKISYNNTNREDLLILWGIHHSNNAAEQTNLYKNPTTYISVGTSTLNQRLVPKIATRSQVNGERGRMDFFWTILKPNDAIHFESNGNFIAPEYAYKIVKKGDSTIMKSEVEYGHCNTKCQTPIGAINSSMPFHNIHPLTIGECP

SEQ ID NO: 83 - DNA, 824 bp, synthetic construct
  source 1..824: mol_type = other DNA
tcctggaaaagacccacaacggcaagctgtgcgacctgaacggcgtgaagcctctgatcctgaaggattgctctgtggccggatggctgctgggcaatcccatgtgcgacgagttcatcagagtgcccgagtggtcctacatcgtggaaagagccaatcctgccaacgacctgtgcttccccggcaacctgaacgactacgaggaactgaagcacctcctgagccggatcaaccacttcgagaagatcctgatcatccccaagagcagctggcccaaccacgagacatctctgggagtgtctgccgcatgtccataccagggcacccctagctttttccggaacgtcgtgtggctgatcaagaagaacgacgcttaccccaccatcaagatcagctacaacaacaccaaccgcgaggacctgctgatcctgtggggaatccaccacagcaacaatgccgccgagcagaccaacctgtacaagaaccccaccacctacatcagcgtgggcaccagcacactgaaccagagactggtgcctaagatcgccacacggtcccaagtgaatggcgagaggggcagaatggacttcttctggaccatcctgaagcctaacgacgccatccactttgagagcaacggcaactttatcgcccctgagtacgcctacaagatcgtgaagaagggcgacagcaccatcatgaagtccgaggtggaatacggccactgcaacaccaagtgtcagacccctatcggcgccatcaactccagcatgcccttccacaacattcaccctctgaccatcggcgagtgccccaaatacgtg

SEQ ID NO: 84 - protein, 51 aa, synthetic construct; identical to SEQ ID NO: 31

SEQ ID NO: 85 - DNA, 153 bp, synthetic construct; identical to SEQ ID NO: 76
  source 1..153: mol_type = other DNA

SEQ ID NO: 86 - protein, 250 aa, synthetic construct; identical to SEQ ID NO: 77

SEQ ID NO: 87 - DNA, 750 bp, synthetic construct; identical to SEQ ID NO: 78
  source 1..750: mol_type = other DNA

SEQ ID NO: 88 - DNA, 6092 bp, synthetic construct
  source 1..6092: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtaccgccaccatggaaaagatcgtgctgctgctggccatcgtgtccctggtcaagagcgaccaaatctgcatcggctaccacgccaacaacagcaccgaacaggtggacaccattatggaaaagaacgtcaccgtgacacacgcccaggacatcctggaaaagacccacaacggcaagctgtgcgacctgaacggcgtgaagcctctgatcctgaaggattgctctgtggccggatggctgctgggcaatcccatgtgcgacgagttcatcagagtgcccgagtggtcctacatcgtggaaagagccaatcctgccaacgacctgtgcttccccggcaacctgaacgactacgaggaactgaagcacctcctgagccggatcaaccacttcgagaagatcctgatcatccccaagagcagctggcccaaccacgagacatctctgggagtgtctgccgcatgtccataccagggcacccctagctttttccggaacgtcgtgtggctgatcaagaagaacgacgcttaccccaccatcaagatcagctacaacaacaccaaccgcgaggacctgctgatcctgtggggaatccaccacagcaacaatgccgccgagcagaccaacctgtacaagaaccccaccacctacatcagcgtgggcaccagcacactgaaccagagactggtgcctaagatcgccacacggtcccaagtgaatggcgagaggggcagaatggacttcttctggaccatcctgaagcctaacgacgccatccactttgagagcaacggcaactttatcgcccctgagtacgcctacaagatcgtgaagaagggcgacagcaccatcatgaagtccgaggtggaatacggccactgcaacaccaagtgtcagacccctatcggcgccatcaactccagcatgcccttccacaacattcaccctctgaccatcggcgagtgccccaaatacgtgaagtccaacaagctggtgctggctaccggcctgagaaacagccctctgagagagaagcgcagacggaagaagagaggcctgtttggcgccattgccggctttatcgaaggcggctggcaaggcatggtggacggatggtacggctaccatcacagcaacgagcaaggctctggatacgccgccgacaaagagagcacccagaaagccattgacggcgtgaccaacaaagtgaacagcatcatcgacaagatgaacacccagttcgaggccgtgggcagagagttcaacaacctggaacggcggatcgagaatctgaacaagaagatggaggacggcttcctggacgtgtggacctacaatgccgagctgctggtcctgatggaaaacgagagaaccctggacttccacgactccaacgtgaagaacctgtacgacaaagtgcggctccagctgcgggacaacgccaaagaactcggcaacggctgcttcgagttctaccacaagtgcgacaacgagtgcatggaaagcgtgcggaacggcacctacgactaccctcagtacagcgaggaagcccggctgaagagagaagagatcagcggagtgaagctggaatccatcggcacataccagatcctgtccatctacagcaccgtggcctcttctctggccctggccattatggtggctggcctgtctctgtggatgtgcagcaatggcagcctccagtgccggatctgcatctgagcggccgcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 89 - protein, 569 aa, synthetic construct
MEKIVLLLAIVSLVKSDQICIGYHANNSTEQVDTIMEKNVTVTHAQDILEKTHNGKLCDLNGVKPLILKDCSVAGWLLGNPMCDEFIRVPEWSYIVERANPANDLCFPGNLNDYEELKHLLSRINHFEKILIIPKSSWPNHNTSLGVSAACPYQGTPSFFRNVVWLIKKNDTYPTIKISYNNTNREDLLILWGIHHSNNTAEQTNLYKNPTTYISVGTSTLNQRLVPKIANRSQVNGQRGRMDFFWTILKPNDAIHFESNGNFIAPEYAYKIVKKGDSTIMKSEVEYGHCNTKCQTPIGAINSSMPFHNIHPLTIGECPKYVKSNKLVLATGLRNSPLREKRRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICI

SEQ ID NO: 90 - DNA, 1727 bp, synthetic construct
  source 1..1727: mol_type = other DNA
gtaccgccaccatggaaaagatcgtgctgctgctggccatcgtgtccctggtcaagagcgaccaaatctgcatcggctaccacgccaacaacagcaccgaacaggtggacaccattatggaaaagaacgtcaccgtgacacacgcccaggacatcctggaaaagacccacaacggcaagctgtgcgacctgaacggcgtgaagcctctgatcctgaaggattgctctgtggccggatggctgctgggcaatcccatgtgcgacgagttcatcagagtgcccgagtggtcctacatcgtggaaagagccaatcctgccaacgacctgtgcttccccggcaacctgaacgactacgaggaactgaagcacctcctgagccggatcaaccacttcgagaagatcctgatcatccccaagagcagctggcccaaccacaataccagcctgggagtgtctgccgcatgtccatatcagggcacccctagctttttccggaacgtcgtgtggctgatcaagaagaacgacacataccccaccatcaagatcagctacaacaacaccaaccgcgaggacctgctgatcctgtggggaatccaccacagcaacaataccgccgagcagaccaacctgtacaagaaccccaccacctacatcagcgtgggcaccagcacactgaaccagagactggtgcctaagatcgccaaccgcagccaagtgaatggccagaggggcagaatggacttcttctggaccatcctgaagcctaacgacgccatccactttgagagcaacggcaactttatcgcccctgagtacgcctacaagatcgtgaagaagggcgacagcaccatcatgaagtccgaggtggaatacggccactgcaacaccaagtgtcagacccctatcggcgccatcaactccagcatgcccttccacaacattcaccctctgaccatcggcgagtgccccaaatacgtgaagtccaacaagctggtgctggctaccggcctgagaaacagccctctgagagagaagcgcagacggaagaagagaggcctgtttggcgccattgccggctttatcgaaggcggctggcaaggcatggtggacggatggtacggctaccatcacagcaacgagcaaggctctggctacgccgccgacaaagagagcacacagaaagccatcgacggcgtgaccaacaaagtgaacagcatcatcgacaagatgaacacccagttcgaggccgtgggcagagagttcaacaacctggaacggcggatcgagaatctgaacaagaagatggaggacggcttcctggacgtgtggacctacaatgccgagctgctggtcctgatggaaaacgagagaaccctggacttccacgactccaacgtgaagaacctgtacgacaaagtgcggctccagctgcgggacaacgccaaagaactcggcaacggctgcttcgagttctaccacaagtgcgacaacgagtgcatggaaagcgtgcggaacggcacctacgactaccctcagtacagcgaggaagcccggctgaagagagaagagatcagcggagtgaagctggaatccatcggcacataccagatcctgtccatctacagcaccgtggcctcttctctggccctggccattatggtggctggcctgtctctgtggatgtgcagcaatggcagcctccagtgccggatctgcatctgagcggcc

SEQ ID NO: 91 - protein, 268 aa, synthetic construct
THNGKLCDLNGVKPLILKDCSVAGWLLGNPMCDEFIRVPEWSYIVERANPANDLCFPGNLNDYEELKHLLSRINHFEKILIIPKSSWPNHNTSLGVSAACPYQGTPSFFRNVVWLIKKNDTYPTIKISYNNTNREDLLILWGIHHSNNTAEQTNLYKNPTTYISVGTSTLNQRLVPKIANRSQVNGQRGRMDFFWTILKPNDAIHFESNGNFIAPEYAYKIVKKGDSTIMKSEVEYGHCNTKCQTPIGAINSSMPFHNIHPLTIGECP

SEQ ID NO: 92 - DNA, 824 bp, synthetic construct
  source 1..824: mol_type = other DNA
tcctggaaaagacccacaacggcaagctgtgcgacctgaacggcgtgaagcctctgatcctgaaggattgctctgtggccggatggctgctgggcaatcccatgtgcgacgagttcatcagagtgcccgagtggtcctacatcgtggaaagagccaatcctgccaacgacctgtgcttccccggcaacctgaacgactacgaggaactgaagcacctcctgagccggatcaaccacttcgagaagatcctgatcatccccaagagcagctggcccaaccacaataccagcctgggagtgtctgccgcatgtccatatcagggcacccctagctttttccggaacgtcgtgtggctgatcaagaagaacgacacataccccaccatcaagatcagctacaacaacaccaaccgcgaggacctgctgatcctgtggggaatccaccacagcaacaataccgccgagcagaccaacctgtacaagaaccccaccacctacatcagcgtgggcaccagcacactgaaccagagactggtgcctaagatcgccaaccgcagccaagtgaatggccagaggggcagaatggacttcttctggaccatcctgaagcctaacgacgccatccactttgagagcaacggcaactttatcgcccctgagtacgcctacaagatcgtgaagaagggcgacagcaccatcatgaagtccgaggtggaatacggccactgcaacaccaagtgtcagacccctatcggcgccatcaactccagcatgcccttccacaacattcaccctctgaccatcggcgagtgccccaaatacgtg

SEQ ID NO: 93 - protein, 51 aa, synthetic construct; identical to SEQ ID NO: 31

SEQ ID NO: 94 - DNA, 153 bp, synthetic construct; identical to SEQ ID NO: 76
  source 1..153: mol_type = other DNA

SEQ ID NO: 95 - protein, 250 aa, synthetic construct; identical to SEQ ID NO: 77

SEQ ID NO: 96 - DNA, 750 bp, synthetic construct
  source 1..750: mol_type = other DNA
aagtccaacaagctggtgctggctaccggcctgagaaacagccctctgagagagaagcgcagacggaagaagagaggcctgtttggcgccattgccggctttatcgaaggcggctggcaaggcatggtggacggatggtacggctaccatcacagcaacgagcaaggctctggctacgccgccgacaaagagagcacacagaaagccatcgacggcgtgaccaacaaagtgaacagcatcatcgacaagatgaacacccagttcgaggccgtgggcagagagttcaacaacctggaacggcggatcgagaatctgaacaagaagatggaggacggcttcctggacgtgtggacctacaatgccgagctgctggtcctgatggaaaacgagagaaccctggacttccacgactccaacgtgaagaacctgtacgacaaagtgcggctccagctgcgggacaacgccaaagaactcggcaacggctgcttcgagttctaccacaagtgcgacaacgagtgcatggaaagcgtgcggaacggcacctacgactaccctcagtacagcgaggaagcccggctgaagagagaagagatcagcggagtgaagctggaatccatcggcacataccagatcctgtccatctacagcaccgtggcctcttctctggccctggccattatggtggctggcctgtctctgtggatgtgcagcaatggcagcctccagtgccggatctgcatctgagcggcc

SEQ ID NO: 97 - DNA, 6092 bp, synthetic construct
  source 1..6092: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtaccgccaccatggaaaagatcgtgctgctgctggccatcgtgtccctggtcaagagcgaccaaatctgcatcggctaccacgccaacaacagcaccgaacaggtggacaccattatggaaaagaacgtcaccgtgacacacgcccaggacatcctggaaaagacccacaacggcaagctgtgcgacctgaacggcgtgaagcctctgatcctgaaggattgctctgtggccggatggctgctgggcaatcccatgtgcgacgagttcatcagagtgcccgagtggtcctacatcgtggaaagagccaatcctgccaacgacctgtgcttccccggcaacctgaacgactacgaggaactgaagcacctcctgagccggatcaaccacttcgagaagatcctgatcatccccaagagcagctggcccaaccacaataccagcctgggagtgtctgccgcatgtccatatcagggcacccctagctttttccggaacgtcgtgtggctgatcaagaagaacgacacataccccaccatcaagatcagctacaacaacaccaaccgcgaggacctgctgatcctgtggggaatccaccacagcaacaataccgccgagcagaccaacctgtacaagaaccccaccacctacatcagcgtgggcaccagcacactgaaccagagactggtgcctaagatcgccaaccgcagccaagtgaatggccagaggggcagaatggacttcttctggaccatcctgaagcctaacgacgccatccactttgagagcaacggcaactttatcgcccctgagtacgcctacaagatcgtgaagaagggcgacagcaccatcatgaagtccgaggtggaatacggccactgcaacaccaagtgtcagacccctatcggcgccatcaactccagcatgcccttccacaacattcaccctctgaccatcggcgagtgccccaaatacgtgaagtccaacaagctggtgctggctaccggcctgagaaacagccctctgagagagaagcgcagacggaagaagagaggcctgtttggcgccattgccggctttatcgaaggcggctggcaaggcatggtggacggatggtacggctaccatcacagcaacgagcaaggctctggctacgccgccgacaaagagagcacacagaaagccatcgacggcgtgaccaacaaagtgaacagcatcatcgacaagatgaacacccagttcgaggccgtgggcagagagttcaacaacctggaacggcggatcgagaatctgaacaagaagatggaggacggcttcctggacgtgtggacctacaatgccgagctgctggtcctgatggaaaacgagagaaccctggacttccacgactccaacgtgaagaacctgtacgacaaagtgcggctccagctgcgggacaacgccaaagaactcggcaacggctgcttcgagttctaccacaagtgcgacaacgagtgcatggaaagcgtgcggaacggcacctacgactaccctcagtacagcgaggaagcccggctgaagagagaagagatcagcggagtgaagctggaatccatcggcacataccagatcctgtccatctacagcaccgtggcctcttctctggccctggccattatggtggctggcctgtctctgtggatgtgcagcaatggcagcctccagtgccggatctgcatctgagcggccgcagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggctacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatgatttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 98 - protein, 469 aa, synthetic construct
MNPNQKIITIGSICMVVGIISLILQIGNIISIWVSHSIQTGNQNHPETCNQSIITYENNTWVNQTYVNISNTNFVAEQDVTSVVLAGNSSLCPISGWAIYSKDNGIRIGSKGDVFVIREPFISCSHLECRTFFLTQGALLNDKHSNGTVKDRSPYRTLMSCPVGEAPSPYNSRFESVAWSASACHDGMSWLTIGISGPDSGAVAVLKYNGIITDTIKSWRNNILRTQESECACINGSCFTIMTDGPSDGQASYKIFKIEKGKVVKSVELNAPNYHYEECSCYPDAGKVMCVCRDNWHGSNRPWVSFDQNLEYQIGYICSGVFGDNPRPNDGTGSCGPVSSNGANGVKGFSFRYGNGVWIGRTKSISSRKGFEMIWDPNGWTETDSSFSVKQDIVGINEWSGYSGSFVQHPELTGLDCMRPCFWVELIRGRPEENTIWTSGSSISFCGVNSDTVGWSWPDGAELPFTIDK

SEQ ID NO: 99 - DNA, 1407 bp, synthetic construct
  source 1..1407: mol_type = other DNA
atgaacccaaatcagaagattatcactattggttctatctgtatggtggtaggcatcatttcacttatcctccagattggaaacattatatccatttgggtgtcacacagtattcagactgggaaccagaaccatcctgagacttgtaatcaatccatcattacatacgaaaacaacacctgggtcaatcagacctatgtgaacataagcaatacaaactttgtggccgagcaggacgtgacatccgtggtccttgcaggaaactccagcctgtgtcccattagcggttgggcaatttactcaaaggataacggcatcaggattggttccaagggtgacgtgttcgtaatcagggagccatttatttcctgctcacacctcgaatgcagaaccttcttcctgactcagggggcactcctgaatgataagcattccaatggaacagtgaaagaccgctccccctataggacattgatgtcctgtcctgttggtgaggccccatctccttataatagtaggtttgagagtgtggcctggtccgcaagtgcttgtcacgatgggatgtcctggctgaccattggtatttctggtccagactctggagccgtggctgttctgaaatataacggaataatcactgacacaatcaaaagttggcgaaataatatcctgaggacccaggagagcgagtgtgcttgcataaatggaagttgtttcactattatgaccgatgggccatccgatgggcaggcttcatataaaatcttcaaaatcgaaaagggtaaggttgtgaagtccgtcgaactgaatgctcctaattaccattacgaagaatgctcctgctaccccgacgctggcaaagtgatgtgcgtatgtcgagataactggcacgggagtaatagaccttgggtgtccttcgaccaaaacttggaataccaaataggctacatttgttcaggggtgttcggcgacaatcctcggccaaacgatgggacaggttcctgtgggccagtttcttcaaacggagccaatggggtcaaaggcttcagtttcagatacggcaacggggtgtggattggccgaaccaagagcatttccagccgaaagggatttgagatgatttgggaccctaacgggtggaccgagacggacagttccttttcagtgaaacaagatattgtgggcatcaacgaatggagcggatatagcgggtccttcgtgcagcacccagaactcacaggactggattgtatgcggccctgtttctgggtagaactcattagaggcagacccgaagagaacacaatctggacatcaggcagttccatttccttctgcggggtgaatagcgatacagtgggatggtcttggcctgatggtgccgaattgccattcacaatagataag

SEQ ID NO: 100 - protein, 567 aa, Influenza A
  source 1..567: note = strain: A/Sichuan/26221/2014
MEKIVLLLAIVSLVKGDQICIGYHANNSTEQVDTIMEKNVTVTHAQDILEKTHNGKLCDLNGVKPLILKDCSVAGWLLGNPMCDEFIRVPEWSYIVERANPANDLCYPGNLNDYEELKHLLSRINHFEKILIIPKSSWTNHETSLGVSAACPYQGTPSFFRNVVWLIKKNDAYPTIKISYNNTNQEDLLILWGVHHSNNAAEQTNLYKNPTTYISVGTSTLNQRLVPKIATRSQVNGQRGRMDFFWTILKPNDAIHFESNGNFIAPEYAYKIVKKGDSTIMKSEMEYGHCNTKCQTPIGAINSSMPFHNIHPLTIGECPKYVKSNKLVLATGLRNSPLREKRRKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNKCMESVRNGTYDYPQYSEEARLKREEISGVKLESIGTYQILSIYSTVASSLALAIIVAGLSLWMCSNGSLQCRICI